(19) United States
(12) Patent Application Publication
(10) Pub. No.: US 2024/0391912 A1
(43) Pub. Date: Nov. 28, 2024

(54) MORPHIC FORMS OF CFT7455 AND METHODS OF MANUFACTURE THEREOF

(71) Applicant: C4 THERAPEUTICS, INC., Watertown, MA (US)

(72) Inventors: James A. Henderson, Weston, MA (US); Matthew J. Schnaderbeck, Methuen, MA (US); Minsheng He, Andover, MA (US); Danmei Dai, Shanghai (CN); He Li, Shanghai (CN); Bing Hu, Shanghai (CN); Yu Zhang, Huai'an (CN); Man Ding, Shanghai (CN); Siyi Jiang, Shanghai (CN); Meiqi Li, Shanghai (CN); Juanjuan Shi, Qianshan (CN)

(73) Assignee: C4 THERAPEUTICS, INC., Watertown, MA (US)

(21) Appl. No.: 18/797,261

(22) Filed: Aug. 7, 2024

Related U.S. Application Data
(63) Continuation of application No. PCT/US2023/012734, filed on Feb. 9, 2023.
(60) Provisional application No. 63/308,883, filed on Feb. 10, 2022.
(60) Provisional application No. 63/328,659, filed on Apr. 7, 2022.
(60) Provisional application No. 63/437,718, filed on Jan. 8, 2023.

(30) Foreign Application Priority Data
Feb. 9, 2022 (CN) 202210119643.2

(51) Int. Cl. C07D 413/14; A61K 31/5377; A61P 35/00

(52) U.S. Cl. CPC C07D 413/14; A61K 31/5377; A61P 35/00

(57) ABSTRACT

An advantageous morphic form of Compound 1 which is also known as CFT7455 and is (S)-3-(6-(4-(morpholinomethyl)benzyl)-2-oxobenzo[cd]indol-1(2H)-yl)piperidine-2,6-dione and methods to prepare Compound 1 for therapeutic applications are provided. This invention also provides new dosage regimens for administering Compound 1.

### CROSS-REFERENCE TO RELATED APPLICATIONS

This application is a continuation of U.S. International Patent Application No. PCT/US2023/012734, filed in the U.S. Receiving Office on Feb. 9, 2023, which claims the benefit of Chinese Patent Application No. 202210119643.2 filed Feb. 9, 2022, U.S. Provisional Application No. 63/308,883 filed Feb. 10, 2022, U.S. Provisional Application No. 63/328,659 filed Apr. 7, 2022, and U.S. Provisional Application No. 63/437,718 filed Jan. 8, 2023. The entirety of each of these applications is hereby incorporated by reference herein for all purposes.

### FIELD OF THE INVENTION

This invention provides an advantageous isolated morphic form of Compound 1 which is also known as CFT7455 or (S)-3-(6-(4-(morpholinomethyl)benzyl)-2-oxobenzo[cd]indol-1(2H)-yl)piperidine-2,6-dione and methods to prepare Compound 1 for therapeutic applications as described further herein. This invention also provides new dosage regimens for administering Compound 1.

### BACKGROUND

The Ikaros family is a series of zinc-finger protein transcription factors that are important for certain physiological processes, particularly hematopoietic cells, and lymphocyte development (see Fan, Y. and Lu, D. “The Ikaros family of zinc-finger proteins” Acta Pharmaceutica Sinica B, 2016, 6:513-521). Ikaros (IKZF1) was first discovered in 1992 (see Georgopoulos, K. et al. “Ikaros, an early lymphoid-specific transcription factor and a putative mediator for T cell commitment” Science, 1992, 258:802-812), and over the subsequent two decades four additional homologs have been identified: Helios (IKZF2), Aiolos (IKZF3), Eos (IKZF4), and Pegasus (IKZF5) (see John, L. B., and Ward, A. C. “The Ikaros gene family: transcriptional regulators of hematopoiesis and immunity” Mol Immunol, 2011, 48:1272-1278). The distribution of various members of the Ikaros protein family within the body varies significantly.

Ikaros, Helios, and Aiolos are primarily found in lymphoid cells and their corresponding progenitors, with Ikaros additionally detected in the brain, and Ikaros and Helios detected in erythroid cells. Eos and Pegasus are more wide spread, and found in skeletal muscle, the liver, the brain, and the heart (see Perdomo, J. et al. “Eos and Pegasus, two members of the Ikaros family of proteins with distinct DNA binding activities: J Biol Chem, 2000, 275:38347-38354; Schmitt, C. et al. “Aiolos and Ikaros: regulators of lymphocyte development, homeostasis and lymphoproliferation” Apoptosis, 2002, 7:277-284; Yoshida, T. and Georgopoulos, K. “Ikaros fingers on lymphocyte differentiation” Int J Hematol, 2014, 100:220-229).

Ikaros is important for proper lymphocyte development. Deletion of the exons encoding the first three N-terminal zinc fingers leads to mice lacking T-cells, B-cells, natural killer (NK) cells, and their progenitors. Genetic alterations in Ikaros are correlated with a poor outcome in the treatment of acute lymphoblastic leukemia (ALL). Ikaros and Aiolos are involved in the proliferation of multiple myeloma cells and lymphoma cells.

Multiple myeloma (MM) is a plasma cell malignancy typically characterized by the abnormal production of monoclonal immunoglobulin, bone marrow involvement, renal dysfunction, immune dysfunction, and skeletal damage. In the United States, MM represents nearly 1.8% of all new cancers. Although outcomes for subjects with MM have improved substantially over the past several decades, the disease remains incurable with a predicted five-year relative survival rate currently of 53.9%. As available therapies are not curative, almost all patients ultimately progress.

Management of MM historically included chemotherapy, including alkylating agents together with corticosteroids. Treatment was further advanced in the 1980's with autologous stem cell transplants. In the 1990's the discovery of thalidomide (first-in-class immunomodulatory imide drug, (“IID”) also referred to as IMiD® or IMiDs®) efficacy in myeloma led to large shifts in treatment regimens and improvements in patient outcomes. The follow-on approved IIDs, lenalidomide and pomalidomide, are now widely used to treat MM, as is the first-in-class agent thalidomide. This class of agents binds the E3 ligase substrate-recognition adapter protein cereblon (CRBN) and promote the degradation of Ikaros (IKZF1) and Aiolos (IKZF3), resulting in antitumor effects, effects on the tumor microenvironment and immune modulation resulting in T-cell priming and antitumor activity. Many patients with MM are treated with multiple regimens containing one of these IIDs. These drugs are now considered standard of care for the treatment of MM in numerous lines of therapy, in combination with agents including dexamethasone, anti-cluster of differentiation 38 (CD38) antibodies, and proteasome inhibitors, such as bortezomib. Although these agents have been successful in prolonging the progression free survival of patients with MM, patients generally relapse with shorter periods of progression free survival after each relapse. Recent studies with novel agents, belantamab and selinexor displayed improved outcomes in multiclass refractory myeloma resulting in recent FDA accelerated approvals; however, response rates were low (26-31%) and progression free survival intervals short (3.7-4.9 months), thus underscoring the continued unmet medical need among these patients.

Non-Hodgkin's Lymphoma (NHL) is a heterogenous group of lymphoid malignancies originating from T, B or NK cells. It includes diffuse large B-cell lymphoma, anaplastic large-cell lymphoma, Burkitt lymphoma, lymphoblastic lymphoma, mantle cell lymphoma, peripheral T-cell lymphoma, follicular lymphoma, cutaneous T-cell lymphoma, lymphoplasmacytic lymphoma, marginal zone B-cell lymphoma, MALT lymphoma, and small-cell lymphocytic lymphoma.

B-cell NHL predominates, while T-cell lymphomas are less common. Among new diagnosed patients with aggressive NHL, chemotherapy-based regimens including cyclophosphamide, vincristine, prednisone and daunorubicin (referred to as CHOP) remain the mainstay of therapy. In B-cell aggressive lymphomas, rituximab in combination with CHOP is the main therapy administered to newly diagnosed patients. In some patients, particularly those with T-cell NHL, initial chemotherapy is followed by autologous stem cell rescue. In relapsed refractory populations, various targeted agents have been developed, thus improving treatment options in multiple subtypes of NHL, however these treatment options tend not to be curative. Additionally, the NHL subtypes are biologically heterogeneous limiting the development of therapeutic agents broadly across indications.

In newly diagnosed patients with aggressive lymphoma, initial treatment is frequently intense and administered with curative intent. Other than the addition of rituximab to CHOP in B-cell NHL and brentuximab to CHOP in anaplastic large cell lymphoma (ALCL), no other novel targeted agent has demonstrated survival improvements and therefore no others have been approved in treatment of naïve patients. Recent therapeutic advances in relapsed NHL include Bruton Tyrosine Kinase (BTK) inhibitors, particularly for mantle cell lymphoma (MCL) and more indolent forms of NHL, chimeric antigen receptors T-cell (CAR-T) therapies which have been approved for diffuse large B-cell lymphoma (DLBCL) and MCL, and novel antibody drug conjugates like polatuzumab, belantamab, or tafasitamab which have been approved for DLBCL. Drugs recently approved for T-cell NHL include romidepsin, belinostat, and brentuximab. Relevant to the NHL population being studied in this protocol, lenalidomide has demonstrated clinical activity in both B-cell and T-cell NHL, including MCL, DLBCL and peripheral T-cell lymphoma (PTCL). Lenalidomide was studied in the relapsed/refractory (r/r) MCL population and was approved by the Food and Drug Administration (FDA) in June 2013 following results from the Phase II EMERGE study which examined the efficacy and safety of lenalidomide in r/r subjects with MCL following bortezomib (overall response rate [ORR] 28%; median duration of response [DOR] 16.6 months). Lenalidomide is also active in DLBCL and PTCL. While lenalidomide and other novel targeted therapies have a modest to good response rate, durability of responses tends to be short across most NHL subtypes. Once patients relapse following 1-2 treatment regimens, the median duration of response tends to be low, and is dependent on patients having sufficient performance status and organ function to tolerate these therapies. Therefore, there remains an unmet medical need among patients with r/r NHL.

Protein degradation is a highly regulated and essential process that maintains cellular homeostasis. The selective identification and removal of damaged, misfolded, or excess proteins is achieved via the ubiquitin-proteasome pathway (UPP). The UPP is central to the regulation of almost all cellular processes, including antigen processing, apoptosis, biogenesis of organelles, cell cycling, DNA transcription and repair, differentiation and development, immune response and inflammation, neural and muscular degeneration, morphogenesis of neural networks, modulation of cell surface receptors, ion channels and the secretory pathway, the response to stress and extracellular modulators, ribosome biogenesis and viral infection. Covalent attachment of multiple ubiquitin molecules by an E3 ubiquitin ligase to a terminal lysine residue marks the protein for proteasome degradation, where the protein is digested into small peptides and eventually into its constituent amino acids that serve as building blocks for new proteins. Defective proteasomal degradation has been linked to a variety of clinical disorders including Alzheimer's disease, Parkinson's disease, Huntington's disease, muscular dystrophies, cardiovascular disease, and cancer among others.

Patent applications that describe certain protein degraders include WO/2022/081928, WO/2022/081928, WO/2022/081927, WO2022/081925, WO/2020/210630, WO/2020/006262, WO/2020/010227, and WO/2020/010177.

Patent applications filed by C4 Therapeutics, Inc., that describe compounds capable of binding to an E3 ubiquitin ligase and targeting proteins for degradation include. WO/2022/032026 titled “Compounds For The Targeted Degradation Of RET”; WO/2022/235945 titled “Compounds For Targeting Degradation Of Bruton's Tyrosine Kinase”; WO/2022/251539 titled “EGFR Degraders To Treat Cancer Metastasis To The Brain Or CNS”; WO/2022/261250 titled “Therapeutics For The Degradation Of Mutant BRAF”; WO/2021/127561 titled “Isoindolinone And Indazole Compounds For The Degradation Of EGFR”; WO/2021/086785 titled “Bifunctional Compounds”; WO/2021/083949 titled “Bifunctional Compounds for the Treatment of Cancer”; WO/2020/210630 titled “Tricyclic Degraders of Ikaros and Aiolos”; WO/2020/181232 titled “Heterocyclic Compounds for Medical Treatment”; WO/2020/132561 titled “Targeted Protein Degradation”; WO/2019/236483 titled “Spirocyclic Compounds”; WO2020/051235 titled “Compounds for the degradation of BRD9 or MTH1”; WO/2019/191112 titled “Cereblon binders for the Degradation of Ikaros”; WO/2019/204354 titled “Spirocyclic Compounds”; WO/2019/099868 titled “Degraders and Degrons for Targeted Protein Degradation”; WO/2018/237026 titled “N/O-Linked Degrons and Degronimers for Protein Degradation”; WO 2017/197051 titled “Amine-Linked C3-Glutarimide Degronimers for Target Protein Degradation”; WO 2017/197055 titled “Heterocyclic Degronimers for Target Protein Degradation”; WO 2017/197036 titled “Spirocyclic Degronimers for Target Protein Degradation”; WO 2017/197046 titled “C3-Carbon Linked Glutarimide Degronimers for Target Protein Degradation”; and WO 2017/197056 titled “Bromodomain Targeting Degronimers for Target Protein Degradation.”

Despite these efforts there remains a need for new therapeutic approaches that include compounds that bind cereblon to degrade Ikaros (IKZF1) or Aiolos (IKZF3) for use in the treatment of abnormal cellular proliferation.

### SUMMARY OF THE INVENTION

A highly stable and crystalline morphic form of Compound 1 has been discovered. This morphic form (Form I) is superior to the other morphic forms of Compound 1 because of its high stability, scalability, and reproducibility. For example, when Compound 1 Form I is tested in solvents with a high-water content including for example acetone/water, DMSO/water, or pure water there are no significant changes to the morphic form (see Example 9). The stability of Compound 1 Form I in high-water content systems is a useful property that increases Compound 1 Form I's shelf life and allows for increased purity and reproducibility in manufacturing scale preparations. This property can also be used to modulate the delivery rate of Compound 1 for improved therapeutic effect. A method of producing Compound 1 Form I on manufacturing scale has also been discovered (see Example 1)

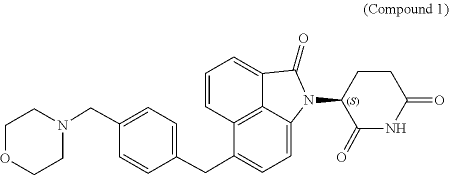

Compound 1 (also known as CFT7455) is a small molecule anti-cancer agent that binds with high affinity to the cereblon E3 ligase thereby creating a new surface on cereblon that interacts with IKZF1 and IKZF3 (see WO 2020/210630). As a result, IKZF1 and IKZF3 are efficiently ubiquitinated by the cereblon E3 ligase and degraded by the proteasome. The high cereblon binding affinity of Compound 1 enables rapid, deep, and durable degradation of IKZF1/3 resulting in potent activity in cancer cells, for example including but not limited to, hematopoietic cancers such as multiple myeloma and multiple types of Non-Hodgkin's Lymphoma.

Compound 1 is in clinical trials for the oral low dose treatment of multiple myeloma and non-Hodgkin's lymphoma. Compound 1 can be administered at microgram doses to treat cancer, and or particular hematological cancers that are IKZF 1/3 sensitive for example at a dose of not more than about 100, 95, 90, 85, 80, 75, 70, 62.5, 65, 60, 55, 50, 45, 40, 37.5, 35, 30, 25, 20, 15, 12.5, 10, 5, or 1 micrograms (μg). The present invention provides an advantageous stable, crystalline, morphic form of Compound 1 for use in therapeutic applications, for example low dose treatments of cancer, and also provides additional morphic forms for a desired use.

In one aspect Compound 1 Form I is administered to a patient in need thereof to treat a Ikaros and/or Aiolos-mediated disorder, for example as part of a pharmaceutical composition. In other aspects Compound 1 Form I is used in the production of a pharmaceutical composition for administration to a patient in need thereof to treat a Ikaros and/or Aiolos-mediated disorder. For example, Compound 1 Form I can be dissolved in an appropriate excipient and then loaded into a gel capsule. Non-limiting examples of excipients that can be used in a gel capsule include polyethylene glycols, surfactants such as polysorbates, alcohols such as ethanol, and glycerin. In certain embodiments Compound 1 Form I is dissolved in PEG-400 and loaded in a gel capsule.

Other morphic forms of Compound 1 are also described herein, including methanol solvate Form II, and hydrates Form III and IV. In certain embodiments Compound 1 Form I can be selectively prepared by the antisolvent addition of ethanol or 2-propanol to DMSO.

Morphic forms of racemic Compound 5 have also been discovered. Compound 5 can be separated by chiral chromatography to prepare Compound 1. The present invention provides a morphic form of Compound 5 with high stability and purity for the preparation of Compound 1. Four crystalline forms of Compound 5, named Form V, Form VI, Form VII, and Form VIII, were identified as polymorphs or pseudo-polymorphs. Among these four crystalline forms, Form VI is a potential hydrate with advantageous solubility, chemical stability, and physical stability. Form V is also highly stable and can be obtained from all tested solvent systems by equilibration (see Example 14), and from benzyl alcohol and THF by fast evaporation (see Example 15).

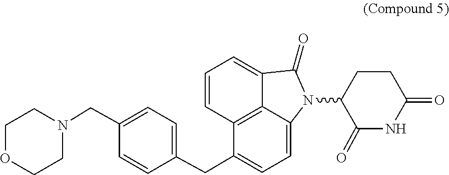 [SMILES: O=C1CCC(N2C(=O)c3cccc4c(Cc5ccc(CN6CCOCC6)cc5)ccc2c34)C(=O)N1]

In another aspect a method for the manufacturing scale production of Compound 1 and Compound 5 is provided. This method includes a reactor scale cross coupling reaction (Step 1), nucleophilic substitution (Step 2), preparative chiral high-performance liquid chromatography (HPLC) separation (Step 3), and selective crystallization (Step 4).

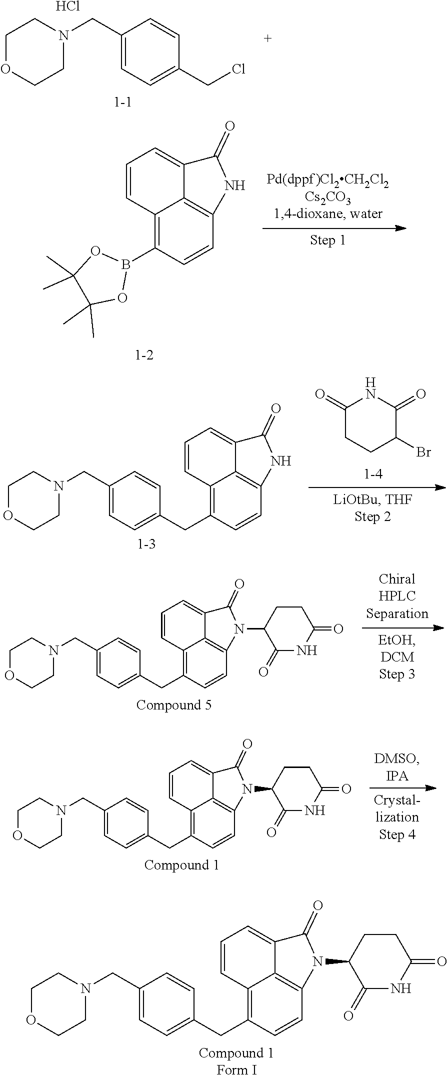

Compound 1 Form I or a pharmaceutical composition prepared from Compound 1 Form I may be used for oral or parenteral delivery. In certain embodiments Compound 1 Form I or a pharmaceutical composition prepared from Compound 1 Form I is used for oral delivery. In certain embodiments, a pharmaceutical composition prepared from or comprising Compound 1 Form I is provided in a solid, gel, or liquid dosage form for oral delivery. In certain embodiments, the solid dosage form is a tablet. In certain embodiments, the dose strengths of the pharmaceutical composition is at least about 1 μg, 5 μg, 10 μg, 12.5 μg, 15 μg, 20 μg, 25 μg, 30 μg, 35 μg, 37.5 μg, 40 μg, 45 μg, 50 μg, 55 μg, 60 μg, 62.5 μg, 65 μg, 70 μg, 75 μg, 80 μg, 85 μg, 87.5 μg, 90 μg, 95 μg, 100 μg, 125 μg, 150 μg, 175 μg, 200 μg, 225 μg, 250 μg, 275 μg, 300 μg, 325 μg, 350 μg, 375 μg, 400 μg, 425 μg, 450 μg, 475 μg, or 500 μg, which may in one nonlimiting aspect be given once weekly, twice weekly, three times weekly, four times weekly, five times weekly, six times weekly, or once daily (QD) with treatment occurring on days 1-14 or 1-21 of a 28-day treatment cycle. In certain embodiments, the dose strengths of the pharmaceutical composition prepared form or comprising Compound 1 or Compound 5 is at least about 1 μg, 5 μg, 10 μg, 12.5 μg, 15 μg, 20 μg, 25 μg, 30 μg, 35 μg, 37.5 μg, 40 μg, 45 μg, 50 μg, 55 μg, 60 μg, 65 μg, 62.5 μg, 70 μg, 75 μg, 80 μg, 85 μg, 87.5 μg, 90 μg, 95 μg, 100 μg, 125 μg, 150 μg, 175 μg, 200 μg, 225 μg, 250 μg, 275 μg, 300 μg, 325 μg, 350 μg, 375 μg, 400 μg, 425 μg, 450 μg, 475 μg, or 500 μg, which may in one nonlimiting aspect be given once weekly, twice weekly, three times weekly, four times weekly, five times weekly, six times weekly, or once daily (QD) with treatment occurring on days 1-14 or 1-21 of a 28-day treatment cycle.

The compound may for example be provided for oral or parenteral delivery. In certain embodiments, the compound is provided in a solid, gel, or liquid dosage form for oral delivery, or may be provided intravenously or otherwise systemically. In some embodiments, Compound 1 Form I is provided as a softshell capsule or tablet for oral administration. In certain embodiments, the dose strengths of the solid or gel dosage form is about 1 μg, 5 μg, 10 μg, 12.5 μg, 15 μg, 20 μg, 25 μg, 37.5 μg, 50 μg, 62.5 μg, 75 μg, 87.5 μg, or 100 μg, which may in one nonlimiting aspect be given once daily (QD) on days 1-14 of a 28-day cycle, QD on Monday, Wednesday, and Friday during the first two weeks of a 28-day cycle, QD on days 1-7 of a 21-day cycle, or QD on days 1-21 of a 28-day cycle.

In certain aspects the selected dosage form for Compound 1, Compound 1 Form I, or a pharmaceutical composition comprising Compound 1 is determined by monitoring blood draws to confirm neutrophil status and then the dose can be modulated as necessary to avoid unacceptable neutropenia or other blood cell level depression. The resulting dosage may be administered less than 21 days in the cycle, for example, several days on followed by several days off for the full 28 days or 21 days, for example 1, 2, 3, 4, 5, or 6 days on per week repeated for 1, 2, 3, or 4 weeks. In other embodiments Compound 1 is administered several days on and several days off for 1, 2, or 3 weeks in the treatment cycle followed by a drug holiday of 1, 2, or 3 weeks. In certain embodiments Compound 1 is administered every other day. In certain embodiments Compound 1 is administered every day of the treatment cycle at a very low dose for example a dose of less than about 87.5 μg, 75 μg, 62.5 μg, 50 μg, 37.5 μg, 25 μg, 20 μg, 15 μg, 12.5 μg, 10 μg, 5 μg, and 1 μg.

In certain aspects a new dosage regimen for the treatment of cancer is provided comprising administering Compound 1 or a pharmaceutically acceptable salt thereof, Compound 1 Form I, or a pharmaceutical composition comprising Compound 1 to a patient in need thereof (a) once a day for 14 consecutive days followed by a 14 day dose holiday in a 28 day treatment cycle; (b) once a day for three selected days per week for example on Monday, Wednesday, and Friday for fourteen days followed by a 14 day dose holiday in a 28 day treatment cycle; or (c) once a day for 7 consecutive days followed by a 14 day dose holiday in a 21 day treatment cycle. In certain embodiments the treatment cycle is repeated 2, 3, 4, 5, 6, 7, 8, 9, 10, 11, 12, or more times. In certain embodiments Compound 1 is provided in a dose of about 1 μg, 5 μg, 10 μg, 12.5 μg, 15 μg, 20 μg, 25 μg, 30 μg, 35 μg, 37.5 μg, 40 μg, 45 μg, 50 μg, 62.5 μg, 75 μg, 87.5 μg, or 100 μg. In certain embodiments Compound 1 is provided in a dose of at least about 1 μg, 5 μg, 10 μg, 15 μg, 20 μg, 25 μg, 30 μg, 35 μg, 40 μg, 45 μg, 50 μg, 75 μg, 87.5 μg, or 100 μg. In certain embodiments Compound 1 is provided in a dose of less than about 1 μg, 5 μg, 10 μg, 15 μg, 20 μg, 25 μg, 30 μg, 35 μg, 40 μg, 45 μg, 50 μg, 75 μg, 87.5 μg, or 100 μg. In certain embodiments, Compound 1 is provided in a dose of about 50 μg, 62.5 μg, or 75 μg.

In certain embodiments Compound 1 is used to modulate immune system activity, for example activating IFN-alpha, IFN-beta, or IFN-gamma. By activating the immune system, the compound can more effectively treat cancer. This immunomodulatory activity may increase the efficacy of Compound 1 in combination with another anti-cancer agent such as daratumumab.

In certain embodiments Compound 1 is used to cause the degradation of IKZF1 and IKZF3 from malignant B or T cells resulting in tumor cell death. The resulting depletion of IKZF1 and IKZF3 from the tumor microenvironment may result in T-cell activation.

The dosage regimens of Compound 1 described herein can be used to treat cancer. Non-limiting examples of cancer include multiple myeloma, Non-Hodgkin's lymphoma, peripheral T-cell lymphoma, mantel cell lymphoma, and diffuse large B cell lymphoma. In certain embodiments Compound 1 is administered as a monotherapy. In other embodiments Compound 1 is administered as part of a two or three agent combination.

Other features and advantages of the present invention will be apparent from the following detailed description and claims.

Thus, the present invention includes at least the following features:
(a) a morphic form of Compound 1 or Compound 5 as described herein;
(b) Compound 1 Form I;
(c) Compound 5 Form V;
(d) Compound 5 Form VI;
(e) a pharmaceutical composition comprising a compound of any one of embodiments (a)-(d);
(f) a pharmaceutical composition prepared from a compound of any one of embodiments (a)-(d), for example a composition with a liquid excipient wherein Compound 1 Form I is dissolved in the excipient;
(g) a method for the treatment of a disorder that is mediated by Ikaros or Aiolos comprising administering an effective amount of a compound or pharmaceutical composition of any one of embodiments (a)-(f);
(h) the method of (g) wherein the compound or pharmaceutical composition of any one of embodiments (a)-(f) is administered (a) once a day for 21 days followed by a 7 day dose holiday in a 28 day treatment cycle;
(i) a method for the treatment of a disorder that is mediated by Ikaros or Aiolos comprising administering an effective amount of Compound 1 or a compound or pharmaceutical composition of any one of embodiments (a)-(f), wherein the compound or pharmaceutical composition is administered (a) once a day at a dose between about 12.5 and about 100 μg, for example about 12.5, about 25, about 37.5, about 50, about 62.5, or about 75 μg for 14 consecutive days followed by a 14 day dose holiday in a 28 day treatment cycle; (b) once a day for three selected days per week for example on Monday, Wednesday, and Friday for 14 days followed by a 14 day dose holiday in a 28 day treatment cycle; (c) once a day for 7 consecutive days followed by a 14 day dose holiday in a 21 day treatment cycle; or (d) once a day on Monday, Wednesday, and Friday followed by a 14 day dose holiday in a 28 day treatment cycle;
(j) the method of (i) wherein Compound 1 is administered at a dose between about 10 and about 75 μg.
(k) the method of (i) wherein Compound 1 is administered at a dose between about 25 and about 75 μg.
(l) the method of (i) wherein Compound 1 is administered at a dose between about 50 and about 75 μg.
(m) the method of (i) wherein Compound 1 is administered at a dose between about 50 and about 100 μg.
(n) a compound or pharmaceutical composition of any one of embodiments (a)-(f) for use in the treatment of a disorder that is mediated by Ikaros or Aiolos;
(o) Compound 1 or a compound or pharmaceutical composition of any one of embodiments (a)-(f) for use in the treatment of a disorder that is mediated by Ikaros or Aiolos, wherein the compound or pharmaceutical composition is administered (a) once a day at a dose between about 12.5 and about 100 μg, for example about 12.5, about 25, about 37.5, about 50, about 62.5, or about 75 μg for 14 consecutive days followed by a 14 day dose holiday in a 28 day treatment cycle; (b) once a day for three selected days per week for example on Monday, Wednesday, and Friday for fourteen days followed by a 14 day dose holiday in a 28 day treatment cycle; (c) once a day for 7 consecutive days followed by a 14 day dose holiday in a 21 day treatment cycle; or (d) once a day on Monday, Wednesday, and Friday followed by a 14 day dose holiday in a 28 day treatment cycle;
(p) use of a compound or pharmaceutical composition of any one of embodiments (a)-(f) for the treatment of a disorder that is mediated by Ikaros or Aiolos;
(q) use of a compound or pharmaceutical composition of any one of embodiments (a)-(f) in the manufacture of a medicament for the treatment of a disorder that is mediated by Ikaros or Aiolos;
(r) use of Compound 1 or a compound or pharmaceutical composition of any one of embodiments (a)-(f) for the treatment of a disorder that is mediated by Ikaros or Aiolos, wherein the compound or pharmaceutical composition is administered (a) once a day at a dose between about 12.5 and about 100 μg, for example about 12.5, about 25, about 37.5, about 50, about 62.5, or about 75 μg for 14 consecutive days followed by a 14 day dose holiday in a 28 day treatment cycle; (b) once a day for three selected days per week for example on Monday, Wednesday, and Friday for fourteen days followed by a 14 day dose holiday in a 28 day treatment cycle; (c) once a day for 7 consecutive days followed by a 14 day dose holiday in a 21 day treatment cycle; or (d) once a day on Monday, Wednesday, and Friday followed by a 14 day dose holiday in a 28 day treatment cycle;
(s) use of Compound 1 or a compound or pharmaceutical composition of any one of embodiments (a)-(f) in the manufacture of a medicament for the treatment of a disorder that is mediated by Ikaros or Aiolos, wherein the compound or pharmaceutical composition is administered (a) once a day at a dose between about 12.5 and about 100 μg, for example about 12.5, about 25, about 37.5, about 50, about 62.5, or about 75 μg for 14 consecutive days followed by a 14 day dose holiday in a 28 day treatment cycle; (b) once a day for three selected days per week for example on Monday, Wednesday, and Friday for fourteen days followed by a 14 day dose holiday in a 28 day treatment cycle; (c) once a day for 7 consecutive days followed by a 14 day dose holiday in a 21 day treatment cycle; or (d) once a day on Monday, Wednesday, and Friday followed by a 14 day dose holiday in a 28 day treatment cycle;
(t) a method for the treatment of a cancer comprising administering an effective amount of a compound or pharmaceutical composition of any one of embodiments (a)-(f); (u) the method of (t) wherein the compound or pharmaceutical composition of any one of embodiments (a)-(f) is administered (a) once a day for 14 consecutive days followed by a 14 day dose holiday in a 28 day treatment cycle or (b) once a day for 21 days followed by a 7 day dose holiday in a 28 day treatment cycle;
(v) a compound or pharmaceutical composition of any one of embodiments (a)-(f) for use in the treatment of a cancer;
(w) use of a compound or pharmaceutical composition of any one of embodiments (a)-(f) for the treatment of a cancer;
(x) use of a compound or pharmaceutical composition of any one of embodiments (a)-(f) in the manufacture of a medicament for the treatment of a cancer;
(y) a method for the treatment of a cancer comprising administering an effective amount of Compound 1 or a compound or pharmaceutical composition of any one of embodiments (a)-(f), wherein the compound or pharmaceutical composition is administered (a) once a day at a dose between about 12.5 and about 100 μg, for example about 12.5, about 25, about 37.5, about 50, about 62.5, or about 75 μg for 14 consecutive days followed by a 14 day dose holiday in a 28 day treatment cycle; (b) once a day for three selected days per week for example on Monday, Wednesday, and Friday for fourteen days followed by a 14 day dose holiday in a 28 day treatment cycle; (c) once a day for 7 consecutive days followed by a 14 day dose holiday in a 21 day treatment cycle; or (d) once a day on Monday, Wednesday, and Friday followed by a 14 day dose holiday in a 28 day treatment cycle;
(z) Compound 1 or a compound or pharmaceutical composition of any one of embodiments (a)-(f) for use in the treatment of a cancer, wherein the compound or pharmaceutical composition is administered (a) once a day at a dose between about 12.5 and about 100 μg, for example about 12.5, about 25, about 37.5, about 50, about 62.5, or about 75 μg for 14 consecutive days followed by a 14 day dose holiday in a 28 day treatment cycle; (b) once a day for three selected days per week for example on Monday, Wednesday, and Friday for fourteen days followed by a 14 day dose holiday in a 28 day treatment cycle; (c) once a day for 7 consecutive days followed by a 14 day dose holiday in a 21 day treatment cycle; or (d) once a day on Monday, Wednesday, and Friday followed by a 14 day dose holiday in a 28 day treatment cycle;
(aa) use of Compound 1 or a compound or pharmaceutical composition of any one of embodiments (a)-(f) for the treatment of a cancer, wherein the compound or pharmaceutical composition is administered (a) once a day at a dose between about 12.5 and about 100 μg, for example about 12.5, about 25, about 37.5, about 50, about 62.5, or about 75 μg for 14 consecutive days followed by a 14 day dose holiday in a 28 day treatment cycle; (b) once a day for three selected days per week for example on Monday, Wednesday, and Friday for fourteen days followed by a 14 day dose holiday in a 28 day treatment cycle; (c) once a day for 7 consecutive days followed by a 14 day dose holiday in a 21 day treatment cycle; or (d) once a day on Monday, Wednesday, and Friday followed by a 14 day dose holiday in a 28 day treatment cycle;
(bb) use of Compound 1 or a compound or pharmaceutical composition of any one of embodiments (a)-(f) in the manufacture of a medicament for the treatment of a cancer, wherein the compound or pharmaceutical composition is administered (a) once a day at a dose between about 12.5 and about 100 μg, for example about 12.5, about 25, about 37.5, about 50, about 62.5, or about 75 μg for 14 consecutive days followed by a 14 day dose holiday in a 28 day treatment cycle; (b) once a day for three selected days per week for example on Monday, Wednesday, and Friday for fourteen days followed by a 14 day dose holiday in a 28 day treatment cycle; (c) once a day for 7 consecutive days followed by a 14 day dose holiday in a 21 day treatment cycle; or (d) once a day on Monday, Wednesday, and Friday followed by a 14 day dose holiday in a 28 day treatment cycle;
(cc) a method for the treatment of a hematological malignancy such as multiple myeloma, leukemia, lymphoblastic leukemia, chronic lymphocytic leukemia, Hodgkin's lymphoma, or non-Hodgkin's lymphoma comprising administering an effective amount of a compound or pharmaceutical composition of any one of embodiments (a)-(f);
(dd) a compound or pharmaceutical composition of any one of embodiments (a)-(f) for use in the treatment of a hematological malignancy such as multiple myeloma, leukemia, lymphoblastic leukemia, chronic lymphocytic leukemia, Hodgkin's lymphoma, or non-Hodgkin's lymphoma;
(ee) use of a compound or pharmaceutical composition of any one of embodiments (a)-(f) for the treatment of a hematological malignancy such as multiple myeloma, leukemia, lymphoblastic leukemia, chronic lymphocytic leukemia, Hodgkin's lymphoma, or non-Hodgkin's lymphoma;
(ff) use of a compound or pharmaceutical composition of any one of embodiments (a)-(f) in the manufacture of a medicament for the treatment of a hematological malignancy such as multiple myeloma, leukemia, lymphoblastic leukemia, chronic lymphocytic leukemia, Hodgkin's lymphoma, or non-Hodgkin's lymphoma; and
(gg) a method to prepared Compound 1 Form I as described herein.

### BRIEF DESCRIPTION OF THE FIGURES

FIG. 1 depicts the XRPD pattern of Compound 1 Form I. The Form I XRPD pattern exhibits sharp peaks, indicating the sample was composed of crystalline material.

FIG. 2 depicts the XRPD pattern of Compound 1 Form II.

FIG. 3 depicts the XRPD pattern of Compound 1 Form III.

FIG. 4 depicts the XRPD pattern of Compound 1 Form IV.

FIG. 5 depicts the XRPD pattern of Compound 5 Form V.

FIG. 6 depicts the XRPD pattern of Compound 5 Form VI.

FIG. 7 depicts the XRPD pattern of Compound 5 Form VII.

FIG. 8 depicts the XRPD pattern of Compound 5 Form VIII.

FIG. 9 depicts the XRPD overlay pattern of Compound 5 Form V, Form VI, Form VII and Form VIII.

FIG. 10 depicts the treatment regimens used for oral dosing of Compound 1 to five patients and the responses observed. The patients were treated as described in Example 24. The responses (SD=Stable Disease and PD=Progressive Disease) were determined using the international myeloma working group (IMWG) Uniform Response Criteria. The x-axis is time in weeks.

FIG. 11 depicts the neutrophil concentration of five patients treated with Compound 1. The patients were treated as described in Example 24. The x-axis is time in days and the y-axis is concentration in 10⁹/L.

FIG. 12 depicts the concentration of Compound 1 for five patients treated with Compound 1. The patients were treated as described in Example 24. The x-axis is time in hours and the y-axis is concentration in ng/mL.

FIG. 13, FIG. 14, and FIG. 15 depict the concentration of Ikaros and Aiolos in Patients 2, 4, and 5 respectively. The patients were treated as described in Example 24. The y-axis is degradation as compared to baseline in % and the x-axis is time measured in days.

FIG. 16 depicts the percent change of dFLC as defined in Example 25 for patients treated with Compound 1 (see Example 24). The y-axis is percent change dFLC and the x-axis is the concentration of Compound 1 measured by Area Under the Curve (AUC) on day 15 measured in hour*ng/mL.

FIG. 17 depicts the concentration of Compound 1 and CC-92480 in the tumor or plasma when dosed as described in Example 26 in a xenograft study. The y-axis is concentration in ng/mL (plasma) and ng/g (tumor) the x-axis is time measured in hours.

FIG. 18 depicts the concentration of IKZF3 remaining after administering Compound 1, CC-92480, or pomalidomide in the xenograft study described in Example 26. The y-axis is concentration of IKZF3 remaining measured in percent and the x-axis is time measured in hours.

FIG. 19 depicts tumor volume in mice after administering Compound 1, CC-92480, or pomalidomide in the xenograft study described in Example 26. The y-axis is tumor volume measured in mm³ and the x-axis is time measured in days.

FIG. 20 depicts body weight changes after administering of the testing articles to female CB17 SCID mice bearing OCI-LY10 intracranial xenografts. Data points represent group mean body weight. Error bars represent standard error of the mean (SEM). The experimental procedure is described in Example 27.

FIG. 21 depicts percent body weight (BW) change in efficacy study. BW change was calculated based on animal weight on the first day of grouping. Data points represent percent group mean change in BW. Error bars represent standard error of the mean (SEM). The experimental procedure is described in Example 27.

FIG. 22 depicts the Kaplan-Meier survival curves of mice treated with vehicle, Compound 1 at a dose of 10 mg/kg QD, Ibrutinib at a dose of 25 mg/kg QD, or the combination of 10 mg/kg QD Compound 1, and 25 mg/kg QD ibrutinib. The experimental procedure is described in Example 27.

### DETAILED DESCRIPTION OF THE INVENTION

Compound 1 (also known as CFT7455) is a small molecule anti-cancer agent that binds with high affinity to the cereblon E3 ligase thereby creating a new surface on cereblon that interacts with IKZF1 and IKZF3 (see WO 2020/210630). As a result, IKZF1 and IKZF3 are efficiently ubiquitinated by the cereblon E3 ligase and degraded by the proteasome. The high cereblon binding affinity of Compound 1 enables rapid, deep, and durable degradation of IKZF1/3 resulting in potent activity in cancer cells, for example including but not limited to, hematopoietic cancers such as multiple myeloma and multiple types of Non-Hodgkin's Lymphoma.

Compound 1 is in clinical trials for the oral low dose treatment of multiple myeloma and non-Hodgkin's lymphoma. Compound 1 can be administered at microgram doses to treat cancer, for example at a dose of not more than about 100, 95, 90, 85, 80, 75, 70, 67.5, 65, 60, 55, 50, 45, 40, 37.5, 35, 30, 25, 20, 15, 10, 12.5, 5, or 1 micrograms (μg). The present invention provides stable, crystalline, morphic forms of Compound 1 for use in therapeutic applications, for example low dose treatments of cancer.

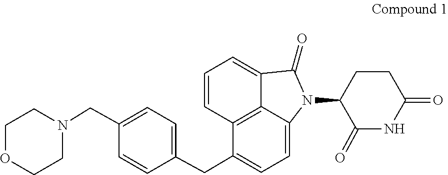 [SMILES: O=C1CC[C@H](N2C(=O)c3cccc4c(Cc5ccc(CN6CCOCC6)cc5)ccc2c34)C(=O)N1]

Compound 5 is the racemic mixture which is separated to prepare Compound 1. The present invention provides a morphic form of Compound 5 with high stability and purity for the preparation of Compound 1.

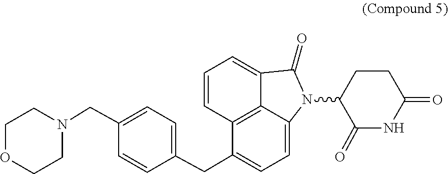 [SMILES: O=C1CCC(N2C(=O)c3cccc4c(Cc5ccc(CN6CCOCC6)cc5)ccc2c34)C(=O)N1]

### Compound 1 Morphic Forms

Compound 1 as a free base was investigated in a variety of crystallization conditions. Unless specified otherwise the starting material in each experiment is Form I. Conditions tested included equilibration at 25° C. and 50° C., precipitation by addition of anti-solvent, slow evaporation, and heating and cooling crystallization.

Four crystalline morphic forms were identified, including 1 anhydrate, named Form I, 2 hydrates, named Form III and Form IV, and 1 methanol solvate, named Form II.

### Compound 1 Form I

Form I is a highly crystalline anhydrate. In certain embodiments Form I is characterized by an XRPD pattern with one or more peaks within +/−0.4° 2theta of the peaks listed in Peak List

Peak List #1

| Angle | d Value | Net Intensity | Gross Intensity | Rel. Intensity |
|---|---|---|---|---|
| 4.839° | 18.24596 Å | 5935.35 | 6007.09 | 100.0% |
| 9.493° | 9.30929 Å | 350.578 | 447.906 | 5.9% |
| 11.443° | 7.72664 Å | 121.206 | 239.469 | 2.0% |
| 12.589° | 7.02582 Å | 435.170 | 588.553 | 7.3% |
| 13.183° | 6.71066 Å | 586.459 | 767.979 | 9.9% |
| 13.508° | 6.55002 Å | 2034.22 | 2228.22 | 34.3% |
| 13.995° | 6.32280 Å | 72.3640 | 281.253 | 1.2% |
| 14.360° | 6.16294 Å | 292.209 | 509.197 | 4.9% |
| 15.411° | 5.74487 Å | 47.0664 | 289.945 | 0.8% |
| 15.974° | 5.54375 Å | 820.430 | 1095.66 | 13.8% |
| 16.367° | 5.41157 Å | 2616.51 | 2914.58 | 44.1% |
| 17.061° | 5.19281 Å | 289.538 | 620.641 | 4.9% |
| 17.472° | 5.07168 Å | 481.451 | 830.872 | 8.1% |
| 17.847° | 4.96592 Å | 1288.94 | 1653.67 | 21.7% |
| 17.934° | 4.94204 Å | 795.040 | 1162.94 | 13.4% |
| 18.295° | 4.84529 Å | 366.045 | 745.487 | 6.2% |
| 18.795° | 4.71765 Å | 68.6179 | 465.816 | 1.2% |
| 19.172° | 4.62575 Å | 2344.08 | 2754.74 | 39.5% |
| 19.626° | 4.51972 Å | 1574.26 | 1997.46 | 26.5% |
| 20.243° | 4.38319 Å | 42.5060 | 476.297 | 0.7% |
| 20.466° | 4.33600 Å | 180.328 | 616.113 | 3.0% |
| 20.856° | 4.25576 Å | 753.886 | 1190.84 | 12.7% |
| 21.540° | 4.12220 Å | 1038.35 | 1470.18 | 17.5% |
| 21.819° | 4.07014 Å | 673.549 | 1100.67 | 11.3% |
| 22.260° | 3.99048 Å | 301.141 | 717.726 | 5.1% |
| 22.539° | 3.94170 Å | 86.8583 | 494.814 | 1.5% |
| 22.920° | 3.87702 Å | 2341.17 | 2734.90 | 39.4% |
| 23.179° | 3.83433 Å | 1376.31 | 1758.76 | 23.2% |
| 24.022° | 3.70164 Å | 391.151 | 758.489 | 6.6% |
| 24.168° | 3.67950 Å | 262.061 | 632.012 | 4.4% |
| 24.527° | 3.62652 Å | 291.797 | 666.373 | 4.9% |
| 24.825° | 3.58372 Å | 664.895 | 1041.39 | 11.2% |
| 25.092° | 3.54607 Å | 149.165 | 525.928 | 2.5% |
| 25.405° | 3.50314 Å | 81.6576 | 456.952 | 1.4% |
| 25.543° | 3.48454 Å | 215.536 | 589.577 | 3.6% |
| 25.925° | 3.43407 Å | 373.243 | 741.882 | 6.3% |
| 26.463° | 3.36546 Å | 1276.56 | 1632.76 | 21.5% |
| 26.747° | 3.33038 Å | 422.316 | 769.677 | 7.1% |
| 27.122° | 3.28514 Å | 582.011 | 915.276 | 9.8% |
| 27.842° | 3.20181 Å | 518.598 | 838.739 | 8.7% |
| 28.031° | 3.18067 Å | 518.976 | 838.316 | 8.7% |
| 28.521° | 3.12710 Å | 229.905 | 546.391 | 3.9% |
| 28.669° | 3.11124 Å | 406.442 | 721.857 | 6.8% |
| 29.103° | 3.06586 Å | 309.609 | 619.429 | 5.2% |
| 29.316° | 3.04407 Å | 475.347 | 781.072 | 8.0% |
| 29.642° | 3.01132 Å | 147.085 | 444.833 | 2.5% |
| 30.113° | 2.96532 Å | 215.049 | 497.624 | 3.6% |
| 30.596° | 2.91958 Å | 172.008 | 434.520 | 2.9% |
| 31.092° | 2.87413 Å | 62.2168 | 312.450 | 1.0% |
| 31.343° | 2.85170 Å | 741.565 | 994.781 | 12.5% |
| 31.627° | 2.82675 Å | 67.1404 | 322.249 | 1.1% |
| 31.930° | 2.80061 Å | 151.384 | 407.716 | 2.6% |
| 32.301° | 2.76922 Å | 259.290 | 516.325 | 4.4% |
| 32.721° | 2.73466 Å | 148.263 | 402.859 | 2.5% |
| 33.058° | 2.70758 Å | 203.310 | 456.500 | 3.4% |
| 33.430° | 2.67830 Å | 103.314 | 356.590 | 1.7% |
| 33.682° | 2.65883 Å | 305.143 | 556.944 | 5.1% |
| 33.799° | 2.64987 Å | 462.505 | 713.202 | 7.8% |
| 34.466° | 2.60013 Å | 83.6017 | 327.866 | 1.4% |
| 34.851° | 2.57227 Å | 164.004 | 409.101 | 2.8% |
| 35.402° | 2.53347 Å | 111.720 | 354.819 | 1.9% |
| 36.038° | 2.49021 Å | 47.5257 | 290.533 | 0.8% |
| 36.124° | 2.48448 Å | 88.7372 | 332.242 | 1.5% |
| 36.750° | 2.44355 Å | 177.393 | 423.471 | 3.0% |
| 37.036° | 2.42536 Å | 238.750 | 484.651 | 4.0% |
| 37.741° | 2.38164 Å | 60.7597 | 311.076 | 1.0% |
| 38.420° | 2.34114 Å | 108.661 | 359.989 | 1.8% |
| 38.542° | 2.33398 Å | 99.2534 | 349.815 | 1.7% |
| 38.807° | 2.31864 Å | 422.346 | 670.224 | 7.1% |
| 39.251° | 2.29344 Å | 48.1775 | 291.056 | 0.8% |
| 39.727° | 2.26703 Å | 171.521 | 407.808 | 2.9% |

1. In certain embodiments Compound 1 Form I is characterized by an XRPD pattern which has at least three peaks selected from 4.8, 13.5, 16.0, 16.4, 17.8, 17.9, 19.2, 19.6, 20.9, 21.5, 21.8, 22.9, 23.2, 24.8, 26.5, and 31.3+/−0.4° 2theta.
2. The morphic form of embodiment 1, wherein Compound 1 Form I is characterized by an XRPD pattern which has at least three peaks selected from 4.8, 13.5, 16.0, 16.4, 17.8, 17.9, 19.2, 19.6, 20.9, 21.5, 21.8, 22.9, 23.2, 24.8, 26.5, and 31.3+/−0.3° 2theta.
3. The morphic form of embodiment 1, wherein Compound 1 Form I is characterized by an XRPD pattern which has at least three peaks selected from 4.8, 13.5, 16.0, 16.4, 17.8, 17.9, 19.2, 19.6, 20.9, 21.5, 21.8, 22.9, 23.2, 24.8, 26.5, and 31.3+/−0.2° 2theta.
4. The morphic form of any one of embodiments 1-3, wherein there are at least four peaks selected from the listed peaks.
5. The morphic form of any one of embodiments 1-3, wherein there are at least five peaks selected from the listed peaks.
6. The morphic form of any one of embodiments 1-3, wherein there are at least six peaks selected from the listed peaks.
7. The morphic form of any one of embodiments 1-3, wherein there are at least seven peaks selected from the listed peaks.
8. The morphic form of any one of embodiments 1-3, wherein there are at least eight peaks selected from the listed peaks.
9. The morphic form of any one of embodiments 1-3, wherein there are at least nine peaks selected from the listed peaks.
10. The morphic form of any one of embodiments 1-3, wherein there are at least ten peaks selected from the listed peaks.
11. The morphic form of any one of embodiments 1-10, wherein the XRPD includes a peak at 4.8+/−0.2° 2theta.
12. The morphic form of any one of embodiments 1-11, wherein the XRPD includes a peak at 16.4+/−0.2° 2theta.
13. The morphic form of any one of embodiments 1-12, wherein the XRPD includes a peak at 19.2+/−0.2° 2theta.
14. The morphic form of any one of embodiments 1-13, wherein the XRPD includes a peak at 22.9+/−0.2° 2theta.
15. The morphic form of any one of embodiments 1-14, wherein the XRPD includes a peak at 13.5+/−0.2° 2theta.
16. The morphic form of any one of embodiments 1-15, wherein the XRPD includes a peak at 19.6+/−0.2° 2theta.
17. The morphic form of any one of embodiments 1-16, wherein the XRPD includes a peak at 23.2+/−0.2° 2theta.
18. The morphic form of any one of embodiments 1-17, wherein the XRPD includes a peak at 17.8+/−0.2° 2theta.
19. The morphic form of any one of embodiments 1-18, wherein the XRPD includes a peak at 26.5+/−0.2° 2theta.
20. The morphic form of any one of embodiments 1-18, wherein the XRPD includes a peak at 21.5+/−0.2° 2theta.

In certain embodiments Form I is characterized by a melting onset of about 235° C. and/or a melting enthalpy of about 115 J/g on DSC thermogram. In certain embodiments Form I is characterized by a weight loss of about 2% at about 225° C. as measured by TGA.

In certain embodiments Form I is characterized by a solubility of at least about 50 mg/mL in DMSO when solubilized at about 25° C. as described in Example 2.

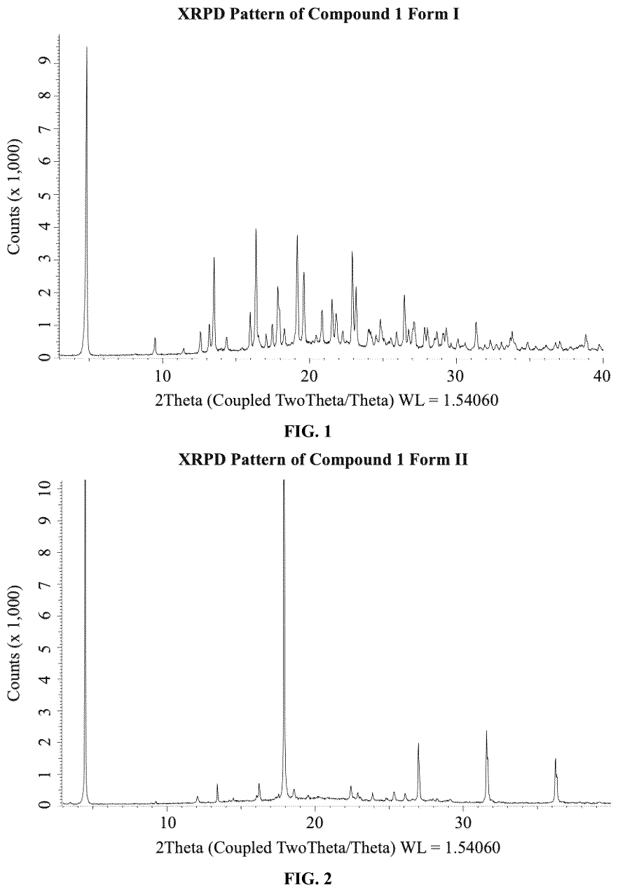

When tested by DSC (see Example 8) Form I had a melting onset of 233.9° C. and a melting enthalpy of 114 J/g. When tested by TGA (see Example 8) Form I had a 2.0% weight loss at 225° C. The XRPD of Form I is provided in FIG. 1.

### Precipitation of Compound 1 Form I by Addition of Anti-Solvent

Compound 1 Form I was prepared by anti-solvent crystallization (see Example 5 and Example 12) of Compound 1 in DMSO, when methanol, water, ethanol, or 2-propanol (IPA) was used as anti-solvent. In certain embodiments, Compound 1 Form I is prepared from the antisolvent addition of ethanol or 2-propanol to DMSO.

### Water Activity Study at 25° C.

A water activity study described in Example 9 was conducted to evaluate the stability of Form I when water was present. In this study, only Form I was obtained in acetone/water, DMSO/water system, and pure water, which indicated that Form I is stable under high water activity condition.

### Compound 1 Form II

Form II is a crystalline methanol solvate. It was obtained after slow evaporation in MeOH/DCM (v:v=1:1) system. About 1.0 equiv. methanol was detected by ¹H-NMR. When tested by DSC (see Example 8), Form II had a desolvation onset at 103.1° C. When tested by TGA (see Example 8), Form II showed about 7.1% weight loss at 125° C. After desolvation, Form I was obtained. The XRPD of Form II is provided in FIG. 2. Form II is characterized by an XRPD pattern (XRPD method 1 in Example 23) with peaks with +/−0.4° 2theta of the peaks listed in Peak List #2.

Peak List #2

| Angle | d Value | Net Intensity | Gross Intensity | Rel. Intensity |
|---|---|---|---|---|
| 3.480° | 25.37173 Å | 22.6684 | 80.4211 | 0.2% |
| 4.492° | 19.65521 Å | 14772.4 | 14835.3 | 100.0% |
| 8.959° | 9.86242 Å | 19.1863 | 85.4840 | 0.1% |
| 9.265° | 9.53782 Å | 54.1173 | 120.728 | 0.4% |
| 9.823° | 8.99669 Å | 14.7399 | 80.3800 | 0.1% |
| 12.072° | 7.32551 Å | 152.520 | 248.988 | 1.0% |
| 13.422° | 6.59163 Å | 446.966 | 563.439 | 3.0% |
| 14.220° | 6.22329 Å | 25.1592 | 160.772 | 0.2% |
| 14.495° | 6.10599 Å | 77.6058 | 216.542 | 0.5% |
| 16.075° | 5.50923 Å | 89.0934 | 254.798 | 0.6% |
| 16.227° | 5.45777 Å | 424.789 | 593.873 | 2.9% |
| 17.401° | 5.09212 Å | 33.7314 | 229.976 | 0.2% |
| 17.543° | 5.05136 Å | 131.050 | 333.299 | 0.9% |
| 17.912° | 4.94807 Å | 10417.7 | 10632.8 | 70.5% |
| 18.594° | 4.76805 Å | 203.333 | 431.209 | 1.4% |
| 19.558° | 4.53524 Å | 65.2252 | 297.047 | 0.4% |
| 20.852° | 4.25659 Å | 33.2161 | 251.584 | 0.2% |
| 22.429° | 3.96078 Å | 334.666 | 537.248 | 2.3% |
| 22.897° | 3.88088 Å | 197.027 | 386.070 | 1.3% |
| 23.063° | 3.85323 Å | 71.4862 | 254.097 | 0.5% |
| 23.887° | 3.72222 Å | 203.254 | 362.612 | 1.4% |
| 25.337° | 3.51245 Å | 241.141 | 406.173 | 1.6% |
| 26.087° | 3.41309 Å | 198.374 | 364.828 | 1.3% |
| 26.987° | 3.30126 Å | 1569.22 | 1736.61 | 10.6% |
| 27.968° | 3.18766 Å | 68.1211 | 222.227 | 0.5% |
| 29.146° | 3.06140 Å | 43.0084 | 168.224 | 0.3% |
| 31.584° | 2.83049 Å | 2051.86 | 2160.31 | 13.9% |
| 31.918° | 2.80164 Å | 60.8328 | 168.478 | 0.4% |
| 32.420° | 2.75936 Å | 43.6097 | 145.320 | 0.3% |
| 33.980° | 2.63613 Å | 21.4164 | 103.735 | 0.1% |
| 36.235° | 2.47711 Å | 1301.34 | 1401.79 | 8.8% |
| 36.735° | 2.44455 Å | 25.8030 | 123.197 | 0.2% |
| 39.212° | 2.29563 Å | 34.8849 | 114.986 | 0.2% |

1. In certain embodiments Compound 1 Form II is characterized by an XRPD pattern which has at least three peaks selected from 4.5, 17.9, 27.0, and 31.6+/−0.4° 2theta.
2. The morphic form of embodiment 1, wherein Compound 1 Form II is characterized by an XRPD pattern which has at least three peaks selected from 4.5, 17.9, 27.0, and 31.6+/−0.3° 2theta.
3. The morphic form of embodiment 1, wherein Compound 1 Form II is characterized by an XRPD pattern which has at least three peaks selected from 4.5, 17.9, 27.0, and 31.6+/−0.2° 2theta.
4. The morphic form of any one of embodiments 1-3, wherein the XRPD includes a peak at 4.5+/−0.2° 2theta.
5. The morphic form of any one of embodiments 1-4, wherein the XRPD includes a peak at 17.9+/−0.2° 2theta.
6. The morphic form of any one of embodiments 1-5, wherein the XRPD includes a peak at 31.6+/−0.2° 2theta.
7. The morphic form of any one of embodiments 1-6, wherein the XRPD includes a peak at 27.0+/−0.2° 2theta.

In certain embodiments Form II is characterized by methanol desolvation at about 105° C. on DSC thermogram. In certain embodiments Form II is characterized by a weight loss of about 7% at about 125° C. as measured by TGA.

### Compound 1 Form III

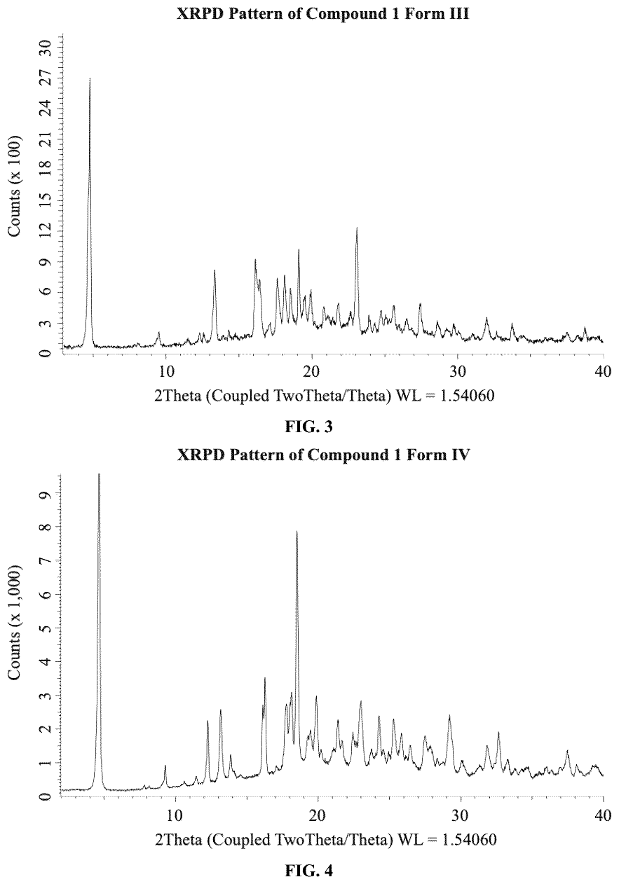

Form III is a crystalline hydrate. It was obtained after slow evaporation of THF/water (v:v=95:5). No residual solvent was detected by ¹H-NMR. When tested by DSC (see Example 8), Form III showed a dehydration peak before melting. When tested by TGA (see Example 8), Form III had a weight loss of about 5.8% (0.4 equiv. water) at 150° C. After water evaporation by heating to 150° C., no form change was observed. Sometimes repeated conditions result in Form IV instead of Form III. For example, Form IV was obtained even though Form III seeds were added during a evaporation repeat experiment. This suggests that Form III could be the metastable form compared with Form IV. The XRPD of Form III is provided in FIG. 3. Form III is characterized by an XRPD pattern (XRPD method 1 in Example 23) with peaks with +/−0.4° 2theta of the peaks listed in Peak List #3.

Peak List #3

| Angle | d Value | Net Intensity | Gross Intensity | Rel. Intensity |
|---|---|---|---|---|
| 4.777° | 18.48364 Å | 1624.05 | 1684.75 | 100.0% |
| 9.509° | 9.29389 Å | 93.9899 | 167.435 | 5.8% |
| 10.900° | 8.11004 Å | 16.6179 | 95.2996 | 1.0% |
| 11.504° | 7.68614 Å | 40.9321 | 129.630 | 2.5% |
| 12.313° | 7.18292 Å | 70.4312 | 174.925 | 4.3% |
| 13.345° | 6.62918 Å | 475.837 | 601.599 | 29.3% |
| 14.315° | 6.18235 Å | 61.7909 | 199.331 | 3.8% |
| 14.762° | 5.99617 Å | 38.6435 | 179.940 | 2.4% |
| 16.138° | 5.48785 Å | 543.138 | 710.024 | 33.4% |
| 16.427° | 5.39202 Å | 377.330 | 548.352 | 23.2% |
| 17.147° | 5.16723 Å | 77.4393 | 265.299 | 4.8% |
| 17.637° | 5.02458 Å | 357.085 | 560.823 | 22.0% |
| 18.142° | 4.88592 Å | 377.834 | 595.465 | 23.3% |
| 18.543° | 4.78117 Å | 286.359 | 513.138 | 17.6% |
| 19.111° | 4.64027 Å | 554.677 | 791.559 | 34.2% |
| 19.507° | 4.54706 Å | 204.432 | 446.371 | 12.6% |
| 19.930° | 4.45131 Å | 240.192 | 485.737 | 14.8% |
| 20.194° | 4.39376 Å | 49.5975 | 296.442 | 3.1% |
| 20.827° | 4.26168 Å | 154.506 | 401.529 | 9.5% |
| 21.096° | 4.20790 Å | 91.0338 | 336.870 | 5.6% |
| 21.431° | 4.14299 Å | 80.3921 | 323.702 | 5.0% |
| 21.839° | 4.06641 Å | 169.164 | 407.813 | 10.4% |
| 22.635° | 3.92522 Å | 148.130 | 372.718 | 9.1% |
| 23.088° | 3.84921 Å | 670.310 | 883.950 | 41.3% |
| 23.918° | 3.71748 Å | 150.285 | 360.000 | 9.3% |
| 24.325° | 3.65621 Å | 54.3698 | 266.954 | 3.3% |
| 24.755° | 3.59365 Å | 156.214 | 369.961 | 9.6% |
| 25.067° | 3.54954 Å | 114.778 | 328.162 | 7.1% |
| 25.607° | 3.47596 Å | 181.856 | 392.227 | 11.2% |
| 25.978° | 3.42717 Å | 50.1903 | 256.731 | 3.1% |
| 26.533° | 3.35670 Å | 96.3725 | 294.511 | 5.9% |
| 27.430° | 3.24894 Å | 177.920 | 362.697 | 11.0% |
| 28.248° | 3.15673 Å | 35.6091 | 214.736 | 2.2% |
| 28.611° | 3.11749 Å | 81.9900 | 256.374 | 5.0% |
| 29.252° | 3.05059 Å | 72.1454 | 234.811 | 4.4% |
| 29.740° | 3.00161 Å | 59.9468 | 210.823 | 3.7% |
| 30.069° | 2.96949 Å | 68.6231 | 210.153 | 4.2% |
| 31.005° | 2.88196 Å | 49.7720 | 182.648 | 3.1% |
| 32.003° | 2.79436 Å | 158.582 | 295.912 | 9.8% |
| 32.681° | 2.73790 Å | 83.4681 | 218.001 | 5.1% |
| 33.729° | 2.65521 Å | 148.230 | 277.296 | 9.1% |
| 36.022° | 2.49130 Å | 32.6366 | 146.633 | 2.0% |
| 37.500° | 2.39640 Å | 70.7190 | 191.163 | 4.4% |
| 38.225° | 2.35259 Å | 38.5246 | 159.506 | 2.4% |
| 38.728° | 2.32322 Å | 125.994 | 248.294 | 7.8% |
| 39.642° | 2.27170 Å | 24.4198 | 142.373 | 1.5% |

1. In certain embodiments Compound 1 Form III is characterized by an XRPD pattern which has at least three peaks selected from 4.8, 13.3, 16.1, 16.4, 17.6, 18.1, 18.5, 19.1, 19.5, 19.9, 21.8, 23.1, 25.6, and 27.4+/−0.4° 2theta.
2. The morphic form of embodiment 1, wherein Compound 1 Form III is characterized by an XRPD pattern which has at least three peaks selected from 4.8, 13.3, 16.1, 16.4, 17.6, 18.1, 18.5, 19.1, 19.5, 19.9, 21.8, 23.1, 25.6, and 27.4+/−0.3° 2theta.
3. The morphic form of embodiment 1, wherein Compound 1 Form III is characterized by an XRPD pattern which has at least three peaks selected from 4.8, 13.3, 16.1, 16.4, 17.6, 18.1, 18.5, 19.1, 19.5, 19.9, 21.8, 23.1, 25.6, and 27.4+/−0.2° 2theta.
4. The morphic form of any one of embodiments 1-3, wherein there are at least four peaks selected from the listed peaks.
5. The morphic form of any one of embodiments 1-3, wherein there are at least five peaks selected from the listed peaks.
6. The morphic form of any one of embodiments 1-3, wherein there are at least six peaks selected from the listed peaks.
7. The morphic form of any one of embodiments 1-3, wherein there are at least seven peaks selected from the listed peaks.
8. The morphic form of any one of embodiments 1-3, wherein there are at least eight peaks selected from the listed peaks.
9. The morphic form of any one of embodiments 1-3, wherein there are at least nine peaks selected from the listed peaks.
10. The morphic form of any one of embodiments 1-3, wherein there are at least ten peaks selected from the listed peaks.
11. The morphic form of any one of embodiments 1-10, wherein the XRPD includes a peak at 4.8+/−0.2° 2theta.
12. The morphic form of any one of embodiments 1-11, wherein the XRPD includes a peak at 23.1+/−0.2° 2theta.
13. The morphic form of any one of embodiments 1-12, wherein the XRPD includes a peak at 19.1+/−0.2° 2theta.
14. The morphic form of any one of embodiments 1-13, wherein the XRPD includes a peak at 16.1+/−0.2° 2theta.
15. The morphic form of any one of embodiments 1-14, wherein the XRPD includes a peak at 13.3+/−0.2° 2theta.
16. The morphic form of any one of embodiments 1-15, wherein the XRPD includes a peak at 18.1+/−0.2° 2theta.
17. The morphic form of any one of embodiments 1-16, wherein the XRPD includes a peak at 16.4+/−0.2° 2theta.
18. The morphic form of any one of embodiments 1-17, wherein the XRPD includes a peak at 17.6+/−0.2° 2theta.
19. The morphic form of any one of embodiments 1-18, wherein the XRPD includes a peak at 18.5+/−0.2° 2theta.

In certain embodiments Form III is characterized by dehydration before melting on DSC thermogram. In certain embodiments Form III is characterized by a weight loss of about 6% at about 120° C. as measured by TGA.

### Compound 1 Form IV

Form IV is a crystalline hydrate. No residual solvent was detected by ¹H-NMR. Form IV showed a dehydration peak before melting by DSC (see Example 8) and about 1.3% weight loss (0.4 equiv. water) at 105° C. by TGA (see Example 8). After dehydration (heating to 150° C.), no form change was observed. The XRPD of Form IV is provided in FIG. 4. Form IV is characterized by an XRPD pattern (XRPD method 1 in Example 23) with peaks with +/−0.4° 2theta of the peaks listed in Peak List #4.

Peak List #4

| Angle | d Value | Net Intensity | Gross Intensity | Rel. Intensity |
|---|---|---|---|---|
| 4.652° | 18.98059 Å | 6802.05 | 6995.65 | 100.0% |
| 7.834° | 11.27653 Å | 77.3633 | 290.014 | 1.1% |
| 8.154° | 10.83508 Å | 61.4829 | 283.819 | 0.9% |
| 9.293° | 9.50895 Å | 463.732 | 720.597 | 6.8% |
| 10.618° | 8.32494 Å | 80.6318 | 387.519 | 1.2% |
| 11.463° | 7.71342 Å | 178.829 | 533.266 | 2.6% |
| 12.261° | 7.21273 Å | 1264.31 | 1678.59 | 18.6% |
| 13.174° | 6.71523 Å | 1419.97 | 1894.86 | 20.9% |
| 13.869° | 6.38029 Å | 496.485 | 1005.64 | 7.3% |
| 14.565° | 6.07696 Å | 86.0924 | 619.235 | 1.3% |
| 16.274° | 5.44231 Å | 2053.58 | 2702.59 | 30.2% |
| 17.089° | 5.18454 Å | 110.400 | 823.529 | 1.6% |
| 17.779° | 4.98476 Å | 1342.61 | 2119.60 | 19.7% |
| 18.100° | 4.89707 Å | 1509.93 | 2313.14 | 22.2% |
| 18.514° | 4.78859 Å | 4927.03 | 5760.80 | 72.4% |
| 19.285° | 4.59871 Å | 603.201 | 1484.24 | 8.9% |
| 19.469° | 4.55583 Å | 663.198 | 1553.59 | 9.7% |
| 19.875° | 4.46351 Å | 1479.53 | 2388.13 | 21.8% |
| 20.207° | 4.39099 Å | 341.367 | 1262.22 | 5.0% |
| 21.098° | 4.20747 Å | 310.647 | 1252.78 | 4.6% |
| 21.401° | 4.14867 Å | 837.757 | 1783.25 | 12.3% |
| 21.676° | 4.09670 Å | 485.342 | 1432.21 | 7.1% |
| 22.442° | 3.95848 Å | 660.142 | 1603.17 | 9.7% |
| 22.993° | 3.86486 Å | 1279.83 | 2213.34 | 18.8% |
| 23.741° | 3.74469 Å | 327.576 | 1253.21 | 4.8% |
| 24.271° | 3.66421 Å | 1015.72 | 1942.29 | 14.9% |
| 24.565° | 3.62101 Å | 321.639 | 1246.15 | 4.7% |
| 25.020° | 3.55619 Å | 214.392 | 1132.08 | 3.2% |
| 25.293° | 3.51838 Å | 972.569 | 1884.04 | 14.3% |
| 25.837° | 3.44551 Å | 658.355 | 1552.70 | 9.7% |
| 26.169° | 3.40253 Å | 138.475 | 1019.27 | 2.0% |
| 26.453° | 3.36669 Å | 453.822 | 1321.18 | 6.7% |
| 27.508° | 3.23991 Å | 660.609 | 1476.62 | 9.7% |
| 27.888° | 3.19662 Å | 492.653 | 1293.11 | 7.2% |
| 28.356° | 3.14495 Å | 240.166 | 1017.26 | 3.5% |
| 29.212° | 3.05466 Å | 1200.06 | 1922.26 | 17.6% |
| 30.090° | 2.96748 Å | 274.871 | 924.554 | 4.0% |
| 31.327° | 2.85314 Å | 189.241 | 810.815 | 2.8% |
| 31.839° | 2.80836 Å | 621.459 | 1251.49 | 9.1% |
| 32.649° | 2.74051 Å | 956.084 | 1588.06 | 14.1% |
| 33.271° | 2.69072 Å | 332.822 | 956.807 | 4.9% |
| 33.809° | 2.64911 Å | 90.1097 | 700.515 | 1.3% |
| 34.289° | 2.61311 Å | 142.732 | 735.815 | 2.1% |
| 34.661° | 2.58590 Å | 152.661 | 728.930 | 2.2% |
| 35.492° | 2.52726 Å | 133.616 | 707.438 | 2.0% |
| 35.987° | 2.49365 Å | 168.776 | 758.720 | 2.5% |
| 36.382° | 2.46745 Å | 151.511 | 750.598 | 2.2% |
| 36.994° | 2.42800 Å | 160.302 | 766.952 | 2.4% |
| 37.469° | 2.39834 Å | 537.283 | 1144.30 | 7.9% |
| 38.120° | 2.35885 Å | 256.488 | 864.751 | 3.8% |
| 39.339° | 2.28851 Å | 198.728 | 823.916 | 2.9% |
| 39.473° | 2.28106 Å | 242.198 | 867.751 | 3.6% |

1. In certain embodiments Compound 1 Form IV is characterized by an XRPD pattern which has at least three peaks selected from 4.7, 12.3, 13.2, 16.3, 17.8, 18.1, 18.5, 19.9, 21.4, 23.0, 24.3, 25.3, 29.2, and 32.6+/−0.4° 2theta.
2. The morphic form of embodiment 1, wherein Compound 1 Form IV is characterized by an XRPD pattern which has at least three peaks selected from 4.7, 12.3, 13.2, 16.3, 17.8, 18.1, 18.5, 19.9, 21.4, 23.0, 24.3, 25.3, 29.2, and 32.6+/−0.3° 2theta.
3. The morphic form of embodiment 1, wherein Compound 1 Form IV is characterized by an XRPD pattern which has at least three peaks selected from 4.7, 12.3, 13.2, 16.3, 17.8, 18.1, 18.5, 19.9, 21.4, 23.0, 24.3, 25.3, 29.2, and 32.6+/−0.2° 2theta.
4. The morphic form of any one of embodiments 1-3, wherein there are at least four peaks selected from the listed peaks.
5. The morphic form of any one of embodiments 1-3, wherein there are at least five peaks selected from the listed peaks.
6. The morphic form of any one of embodiments 1-3, wherein there are at least six peaks selected from the listed peaks.
7. The morphic form of any one of embodiments 1-3, wherein there are at least seven peaks selected from the listed peaks.
8. The morphic form of any one of embodiments 1-3, wherein there are at least eight peaks selected from the listed peaks.
9. The morphic form of any one of embodiments 1-3, wherein there are at least nine peaks selected from the listed peaks.
10. The morphic form of any one of embodiments 1-3, wherein there are at least ten peaks selected from the listed peaks.
11. The morphic form of any one of embodiments 1-10, wherein the XRPD includes a peak at 4.7+/−0.2° 2theta.
12. The morphic form of any one of embodiments 1-11, wherein the XRPD includes a peak at 18.5+/−0.2° 2theta.
13. The morphic form of any one of embodiments 1-12, wherein the XRPD includes a peak at 16.3+/−0.2° 2theta.
14. The morphic form of any one of embodiments 1-13, wherein the XRPD includes a peak at 18.1+/−0.2° 2theta.
15. The morphic form of any one of embodiments 1-14, wherein the XRPD includes a peak at 19.9+/−0.2° 2theta.
16. The morphic form of any one of embodiments 1-15, wherein the XRPD includes a peak at 13.2+/−0.2° 2theta.
17. The morphic form of any one of embodiments 1-16, wherein the XRPD includes a peak at 17.8+/−0.2° 2theta.
18. The morphic form of any one of embodiments 1-17, wherein the XRPD includes a peak at 23.0+/−0.2° 2theta.
19. The morphic form of any one of embodiments 1-18, wherein the XRPD includes a peak at 12.3+/−0.2° 2theta.
20. The morphic form of any one of embodiments 1-19, wherein the XRPD includes a peak at 29.2+/−0.2° 2theta.

In certain embodiments Form IV is characterized by dehydration before melting on DSC thermogram. In certain embodiments Form IV is characterized by a weight loss of about 1% at about 105° C. as measured by TGA.

### Competitive Equilibration Experiments

Competitive equilibration experiments of Form I and Form IV were conducted in THF/water and DMSO/water system with different water activity to study the interconversion relationship between hydrate and anhydrate (see Example 10). In THF/water system, Form IV (hydrate) was obtained when water activity (a.w.)≥0.6 while Form I (anhydrate) was obtained when a.w.≤0.3. In DMSO/water system, physical mixtures of Form I and Form IV were obtained from all selected water activity mixtures, and Form I was dominant in solid mixtures. It is worth noting that both hydrate forms were only obtained in THF/water system through recrystallization and anhydrate Form I was very stable alone in water. Therefore, Form IV formation is solvent and water activity dependent. Form IV is stable in THF/water systems with high water activity.

Based on these results above, THF plays an important role in the formation of Form IV and the transformation between Form I and Form IV. The dehydration temperature of Form IV was below 30° C., which is relatively low. The crystallinity of Form IV decreased after dehydration even though its hydration-dehydration process is reversible. Form I was stable in equilibration experiments even in pure water.

### Compound 5 Morphic Forms

Compound 5 was investigated in a variety of crystallization conditions to identify potential polymorphs and pseudo-polymorphs and select a metastable polymorph in terms of solubility and stability for chiral separation. Unless specified otherwise, the starting material in each experiment is Form V. Conditions tested included equilibration at 25° C., fast cooling, fast evaporation, and anti-solvent addition.

Four crystalline forms of Compound 5 were identified as polymorphs or pseudo-polymorphs. They are named Form V, Form VI, Form VII, and Form VIII. An amorphous form was also obtained from chloroform by fast evaporation.

### Compound 5 Form V

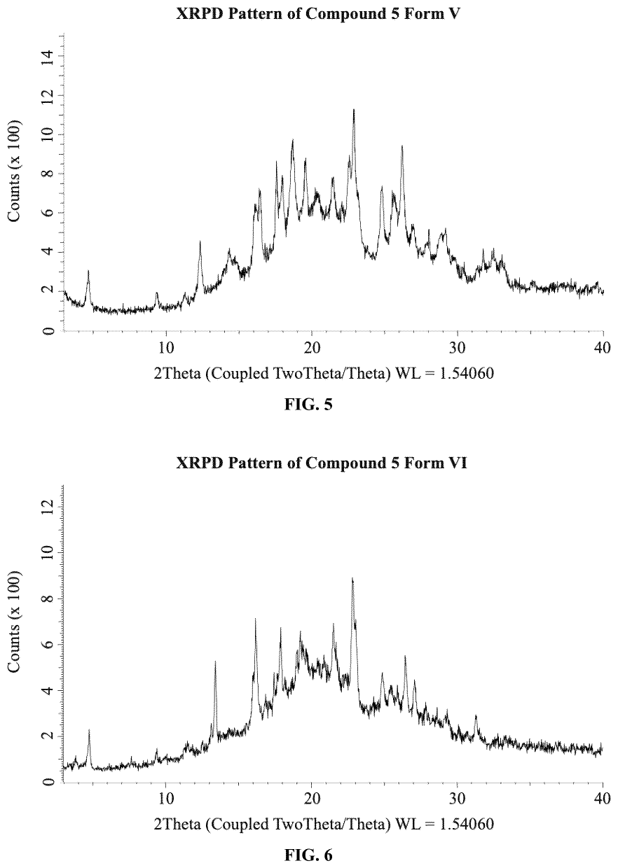

Form V is a potential hydrate. It was obtained from all tested solvent systems by equilibration (see Example 14), and from benzyl alcohol and THF by fast evaporation (see Example 15). Form V is crystalline. DSC (see Example 21) showed a dehydration peak at 38.5° C. and an endothermic peak at 220.0° C. TGA (see Example 21) showed about 5.1% weight loss at about 140° C. The XRPD of Form V is provided in FIG. 5. In certain embodiments Form V is characterized by an XRPD pattern (XRPD method 2 in Example 23) with peaks with +/−0.4

Peak List #5

| | | Net | Gross | Rel. |
|---|---|---|---|---|
| Angle | d Value | Intensity | Intensity | Intensity |
| 4.674° | 18.88945 Å | 112.589 | 227.805 | 21.7% |
| 9.381° | 9.42014 Å | 55.2203 | 166.999 | 10.7% |
| 11.303° | 7.82226 Å | 45.8820 | 180.260 | 8.9% |
| 12.345° | 7.16392 Å | 199.286 | 372.972 | 38.5% |
| 14.024° | 6.30993 Å | 37.5819 | 282.903 | 7.3% |
| 14.348° | 6.16822 Å | 119.256 | 377.197 | 23.0% |
| 14.718° | 6.01385 Å | 36.4057 | 307.912 | 7.0% |
| 16.153° | 5.48289 Å | 235.093 | 556.951 | 45.4% |
| 16.432° | 5.39040 Å | 226.778 | 557.832 | 43.8% |
| 17.584° | 5.03959 Å | 384.345 | 747.833 | 74.2% |
| 17.985° | 4.92811 Å | 259.139 | 631.817 | 50.0% |
| 18.681° | 4.74616 Å | 367.635 | 753.692 | 71.0% |
| 19.574° | 4.53154 Å | 306.382 | 704.846 | 59.2% |
| 20.329° | 4.36498 Å | 206.515 | 611.281 | 39.9% |
| 21.482° | 4.13323 Å | 269.924 | 676.924 | 52.1% |
| 22.598° | 3.93156 Å | 313.221 | 713.872 | 60.5% |
| 22.866° | 3.88604 Å | 517.829 | 915.706 | 100.0% |
| 24.823° | 3.58399 Å | 241.688 | 618.459 | 46.7% |
| 25.614° | 3.47497 Å | 221.863 | 596.634 | 42.8% |
| 26.199° | 3.39871 Å | 408.437 | 779.025 | 78.9% |
| 26.928° | 3.30837 Å | 98.8027 | 460.960 | 19.1% |
| 27.973° | 3.18710 Å | 63.2320 | 407.068 | 12.2% |
| 28.897° | 3.08729 Å | 117.034 | 438.561 | 22.6% |
| 29.172° | 3.05881 Å | 146.105 | 459.886 | 28.2% |
| 29.789° | 2.99679 Å | 59.4093 | 353.937 | 11.5% |
| 31.759° | 2.81528 Å | 135.096 | 388.178 | 26.1% |
| 32.472° | 2.75506 Å | 140.475 | 385.889 | 27.1% |
| 33.039° | 2.70907 Å | 92.8257 | 329.706 | 17.9% |
| 33.226° | 2.69421 Å | 91.4822 | 325.066 | 17.7% |
| 38.838° | 2.31684 Å | 33.0606 | 229.882 | 6.4% |

1. In certain embodiments Compound 1 Form V is characterized by an XRPD pattern which has at least three peaks selected from 4.7, 9.4, 12.3, 14.3, 16.2, 16.4, 17.6, 18.0, 18.7, 19.6, 20.3, 21.5, 22.6, 22.9, 24.8, 25.6, 26.2, 26.9, 28.0, 28.9, 29.2, 29.8, 31.8, 32.5, 33.0, and 33.2+/−0.4° 2theta.
2. The morphic form of embodiment 1, wherein Compound 1 Form V is characterized by an XRPD pattern which has at least three peaks selected from 4.7, 9.4, 12.3, 14.3, 16.2, 16.4, 17.6, 18.0, 18.7, 19.6, 20.3, 21.5, 22.6, 22.9, 24.8, 25.6, 26.2, 26.9, 28.0, 28.9, 29.2, 29.8, 31.8, 32.5, 33.0, and 33.2+/−0.3° 2theta.
3. The morphic form of embodiment 1, wherein Compound 1 Form V is characterized by an XRPD pattern which has at least three peaks selected from 4.7, 9.4, 12.3, 14.3, 16.2, 16.4, 17.6, 18.0, 18.7, 19.6, 20.3, 21.5, 22.6, 22.9, 24.8, 25.6, 26.2, 26.9, 28.0, 28.9, 29.2, 29.8, 31.8, 32.5, 33.0, and 33.2+/−0.2° 2theta.
4. The morphic form of any one of embodiments 1-3, wherein there are at least four peaks selected from the listed peaks.
5. The morphic form of any one of embodiments 1-3, wherein there are at least five peaks selected from the listed peaks.
6. The morphic form of any one of embodiments 1-3, wherein there are at least six peaks selected from the listed peaks.
7. The morphic form of any one of embodiments 1-3, wherein there are at least seven peaks selected from the listed peaks.
8. The morphic form of any one of embodiments 1-3, wherein there are at least eight peaks selected from the listed peaks.
9. The morphic form of any one of embodiments 1-3, wherein there are at least nine peaks selected from the listed peaks.
10. The morphic form of any one of embodiments 1-3, wherein there are at least ten peaks selected from the listed peaks.
11. The morphic form of any one of embodiments 1-10, wherein the XRPD includes a peak at 22.9+/−0.2° 2theta.
12. The morphic form of any one of embodiments 1-11, wherein the XRPD includes a peak at 26.2+/−0.2° 2theta.
13. The morphic form of any one of embodiments 1-12, wherein the XRPD includes a peak at 17.6+/−0.2° 2theta.
14. The morphic form of any one of embodiments 1-13, wherein the XRPD includes a peak at 18.7+/−0.2° 2theta.
15. The morphic form of any one of embodiments 1-14, wherein the XRPD includes a peak at 22.6+/−0.2° 2theta.
16. The morphic form of any one of embodiments 1-15, wherein the XRPD includes a peak at 19.6+/−0.2° 2theta.
17. The morphic form of any one of embodiments 1-16, wherein the XRPD includes a peak at 21.5+/−0.2° 2theta.
18. The morphic form of any one of embodiments 1-17, wherein the XRPD includes a peak at 18.0+/−0.2° 2theta.
19. The morphic form of any one of embodiments 1-18, wherein the XRPD includes a peak at 24.8+/−0.2° 2theta.
20. The morphic form of any one of embodiments 1-19, wherein the XRPD includes a peak at 16.2+/−0.2° 2theta.
21. The morphic form of any one of embodiments 1-20, wherein the XRPD includes a peak at 16.4+/−0.2° 2theta.
22. The morphic form of any one of embodiments 1-21, wherein the XRPD includes a peak at 25.6+/−0.2° 2theta.
23. The morphic form of any one of embodiments 1-22, wherein the XRPD includes a peak at 20.3+/−0.2° 2theta.
24. The morphic form of any one of embodiments 1-23, wherein the XRPD includes a peak at 12.3+/−0.2° 2theta.
25. The morphic form of any one of embodiments 1-24, wherein the XRPD includes a peak at 29.2+/−0.2° 2theta.
26. The morphic form of any one of embodiments 1-25, wherein the XRPD includes a peak at 32.5+/−0.2° 2theta.
27. The morphic form of any one of embodiments 1-26, wherein the XRPD includes a peak at 31.8+/−0.2° 2theta.
28. The morphic form of any one of embodiments 1-27, wherein the XRPD includes a peak at 14.3+/−0.2° 2theta.
29. The morphic form of any one of embodiments 1-28, wherein the XRPD includes a peak at 28.9+/−0.2° 2theta.
30. The morphic form of any one of embodiments 1-29, wherein the XRPD includes a peak at 4.7+/−0.2° 2theta.

In certain embodiments Form V is characterized by a dehydration onset at about 40° C. and/or endothermic onset at about 220° C. on DSC thermogram. In certain embodiments Form V is characterized by a weight loss of about 5% at about 140° C. as measured by TGA.

In certain embodiments Form V is characterized by a solubility of at least about 50 mg/mL in DMSO and/or 1-methyl-2-pyrrolidinone when solubilized at about 25° C. as described in Example 13.

### Compound 5 Form VI

Form VI is a potential hydrate. It was obtained from THF/IPA, THF/heptane mixture by equilibration and from DMSO by fast evaporation. Form VI is crystalline. When tested by DSC (see Example 21), Form VI showed a dehydration peak at 5.9° C. and an evaporation peak starting at about 154° C. Form VI also showed a melting onset of 226.4° C. and a melting enthalpy of 35 J/g. When tested by TGA (see Example 21), Form VI showed about 1.4% weight loss at about 85° C., about 8.3% weight loss from about 85° C. to 215° C. and about 1.4% weight loss from about 215° C. to 250° C. The XRPD of Form VI is provided in FIG. 6. Form VI is characterized by an XRPD pattern (XRPD method 2 in Example 23) with peaks with +/−0.4° 2theta of the peaks listed in Peak List #6.

Peak List #6

| | | Net | Gross | Rel. |
|---|---|---|---|---|
| Angle | d Value | Intensity | Intensity | Intensity |
| 4.759° | 18.55271 Å | 105.621 | 166.769 | 30.1% |
| 9.383° | 9.41766 Å | 29.3555 | 112.369 | 8.4% |
| 12.532° | 7.05746 Å | 25.8282 | 163.018 | 7.4% |
| 13.142° | 6.73141 Å | 69.0888 | 223.347 | 19.7% |
| 13.415° | 6.59511 Å | 251.212 | 414.499 | 71.6% |
| 16.182° | 5.47315 Å | 328.415 | 568.998 | 93.6% |
| 16.861° | 5.25402 Å | 73.2947 | 333.753 | 20.9% |
| 17.442° | 5.08049 Å | 155.910 | 431.153 | 44.4% |
| 17.894° | 4.95311 Å | 271.924 | 557.290 | 77.5% |
| 19.019° | 4.66257 Å | 169.637 | 474.889 | 48.3% |
| 19.262° | 4.60427 Å | 188.109 | 496.664 | 53.6% |
| 19.642° | 4.51594 Å | 173.956 | 486.973 | 49.6% |
| 20.866° | 4.25388 Å | 169.205 | 490.702 | 48.2% |
| 21.520° | 4.12600 Å | 243.093 | 565.454 | 69.3% |
| 21.709° | 4.09056 Å | 171.012 | 493.148 | 48.7% |
| 22.854° | 3.88805 Å | 351.014 | 667.211 | 100.0% |
| 23.036° | 3.85777 Å | 261.825 | 576.358 | 74.6% |
| 24.870° | 3.57722 Å | 121.960 | 423.441 | 34.7% |
| 25.448° | 3.49733 Å | 65.0797 | 362.421 | 18.5% |
| 25.901° | 3.43722 Å | 115.136 | 407.843 | 32.8% |
| 26.441° | 3.36814 Å | 212.451 | 498.012 | 60.5% |
| 27.089° | 3.28912 Å | 118.976 | 393.690 | 33.9% |
| 29.248° | 3.05104 Å | 37.2294 | 257.652 | 10.6% |
| 30.652° | 2.91436 Å | 26.2019 | 210.446 | 7.5% |
| 31.297° | 2.85579 Å | 69.6182 | 242.930 | 19.8% |
| 33.346° | 2.68483 Å | 38.3667 | 190.809 | 10.9% |

1. In certain embodiments Compound 1 Form VI is characterized by an XRPD pattern which has at least three peaks selected from 4.8, 13.1, 13.4, 16.2, 16.9, 17.4, 17.9, 19.0, 19.3, 19.6, 20.9, 21.5, 21.7, 22.9, 23.0, 24.9, 25.4, 25.9, 26.4, 27.1, 29.2, 31.3, and 33.3+/−0.4° 2theta.
2. The morphic form of embodiment 1, wherein Compound 1 Form VI is characterized by an XRPD pattern which has at least three peaks selected from 4.8, 13.1, 13.4, 16.2, 16.9, 17.4, 17.9, 19.0, 19.3, 19.6, 20.9, 21.5, 21.7, 22.9, 23.0, 24.9, 25.4, 25.9, 26.4, 27.1, 29.2, 31.3, and 33.3+/−0.3° 2theta.
3. The morphic form of embodiment 1, wherein Compound 1 Form VI is characterized by an XRPD pattern which has at least three peaks selected from 4.8, 13.1, 13.4, 16.2, 16.9, 17.4, 17.9, 19.0, 19.3, 19.6, 20.9, 21.5, 21.7, 22.9, 23.0, 24.9, 25.4, 25.9, 26.4, 27.1, 29.2, 31.3, and 33.3+/−0.2° 2theta.
4. The morphic form of any one of embodiments 1-3, wherein there are at least four peaks selected from the listed peaks.
5. The morphic form of any one of embodiments 1-3, wherein there are at least five peaks selected from the listed peaks.
6. The morphic form of any one of embodiments 1-3, wherein there are at least six peaks selected from the listed peaks.
7. The morphic form of any one of embodiments 1-3, wherein there are at least seven peaks selected from the listed peaks.
8. The morphic form of any one of embodiments 1-3, wherein there are at least eight peaks selected from the listed peaks.
9. The morphic form of any one of embodiments 1-3, wherein there are at least nine peaks selected from the listed peaks.
10. The morphic form of any one of embodiments 1-3, wherein there are at least ten peaks selected from the listed peaks.
11. The morphic form of any one of embodiments 1-10, wherein the XRPD includes a peak at 22.9+/−0.2° 2theta.
12. The morphic form of any one of embodiments 1-11, wherein the XRPD includes a peak at 16.2+/−0.2° 2theta.
13. The morphic form of any one of embodiments 1-12, wherein the XRPD includes a peak at 17.9+/−0.2° 2theta.
14. The morphic form of any one of embodiments 1-13, wherein the XRPD includes a peak at 23.0+/−0.2° 2theta.
15. The morphic form of any one of embodiments 1-14, wherein the XRPD includes a peak at 13.4+/−0.2° 2theta.
16. The morphic form of any one of embodiments 1-15, wherein the XRPD includes a peak at 21.5+/−0.2° 2theta.
17. The morphic form of any one of embodiments 1-16, wherein the XRPD includes a peak at 26.4+/−0.2° 2theta.
18. The morphic form of any one of embodiments 1-17, wherein the XRPD includes a peak at 19.3+/−0.2° 2theta.
19. The morphic form of any one of embodiments 1-18, wherein the XRPD includes a peak at 19.6+/−0.2° 2theta.
20. The morphic form of any one of embodiments 1-19, wherein the XRPD includes a peak at 21.7+/−0.2° 2theta.
21. The morphic form of any one of embodiments 1-20, wherein the XRPD includes a peak at 19.0+/−0.2° 2theta.
22. The morphic form of any one of embodiments 1-21, wherein the XRPD includes a peak at 20.9+/−0.2° 2theta.
23. The morphic form of any one of embodiments 1-22, wherein the XRPD includes a peak at 17.4+/−0.2° 2theta.
24. The morphic form of any one of embodiments 1-23, wherein the XRPD includes a peak at 24.9+/−0.2° 2theta.
25. The morphic form of any one of embodiments 1-24, wherein the XRPD includes a peak at 27.1+/−0.2° 2theta.
26. The morphic form of any one of embodiments 1-25, wherein the XRPD includes a peak at 25.9+/−0.2° 2theta.
27. The morphic form of any one of embodiments 1-26, wherein the XRPD includes a peak at 4.8+/−0.2° 2theta.
28. The morphic form of any one of embodiments 1-27, wherein the XRPD includes a peak at 16.9+/−0.2° 2theta.

In certain embodiments Form VI is characterized by a dehydration onset at about 6° C. and/or an evaporation onset at about 155° C. and/or a melting onset of about 225° C. and/or a melting enthalpy of about 35 J/g on DSC thermogram. In certain embodiments Form VI is characterized by a weight loss of about 1.5% at about 85° C., and about 8% weight loss from about 85° C. to 215° C. and about 1.5% weight loss from about 215° C. to 250° C. as measured by TGA.

In certain embodiments Form VI is characterized by a solubility of about 30 mg/mL in MeOH/DCM (v:v=1:2) and/or 27 mg/mL in EtOH/DCM (v:v=1:4) when solubilized at about 25° C. as described in Example 20.

### Compound 5 Form VII

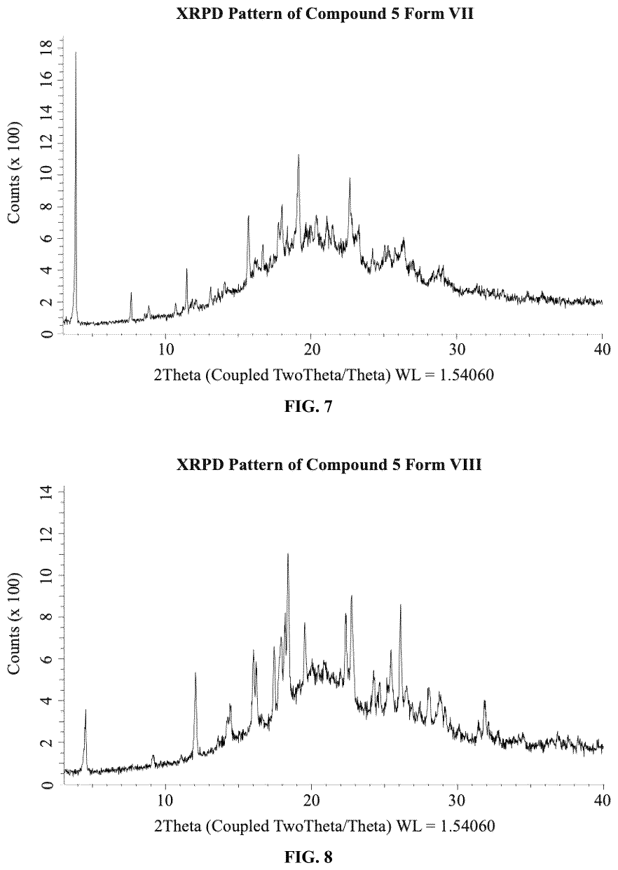

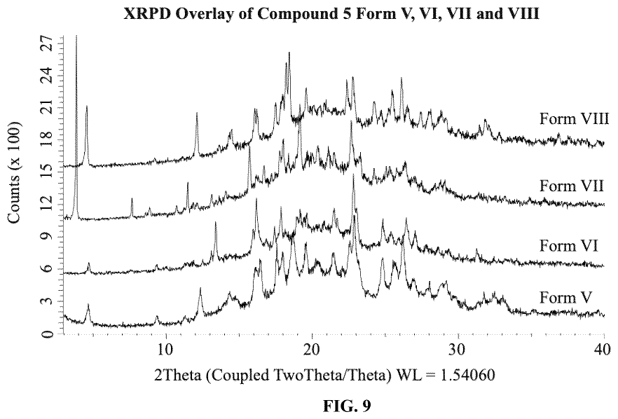

Form VII is a potential NMP solvate. It was obtained from NMP by fast evaporation. Form VII is crystalline. ¹H-NMR shows about 25.2% (1.6 equiv.) of NMP residue by weight. The XRPD of Form VII is provided in FIG. 7. Form VII is characterized by an XRPD pattern (XRPD method 2 in Example 23) with peaks with +/−0.4° 2theta of the peaks listed in Peak List #7.

Peak List #7

| | | Net | Gross | Rel. |
|---|---|---|---|---|
| Angle | d Value | Intensity | Intensity | Intensity |
| 3.854° | 22.90698 Å | 1013.05 | 1076.00 | 100.0% |
| 7.653° | 11.54211 Å | 128.022 | 198.783 | 12.6% |
| 8.589° | 10.28626 Å | 30.7290 | 110.786 | 3.0% |
| 8.875° | 9.95605 Å | 53.7373 | 137.240 | 5.3% |
| 10.715° | 8.25013 Å | 52.0005 | 161.327 | 5.1% |
| 11.207° | 7.88912 Å | 35.4492 | 160.116 | 3.5% |
| 11.478° | 7.70317 Å | 198.533 | 332.457 | 19.6% |
| 11.853° | 7.46049 Å | 47.6258 | 193.307 | 4.7% |
| 12.105° | 7.30574 Å | 37.9182 | 190.842 | 3.7% |
| 13.118° | 6.74389 Å | 78.0952 | 262.170 | 7.7% |
| 13.628° | 6.49243 Å | 60.1303 | 268.267 | 5.9% |
| 14.092° | 6.27957 Å | 65.1634 | 295.246 | 6.4% |
| 15.709° | 5.63661 Å | 300.166 | 601.622 | 29.6% |
| 16.174° | 5.47553 Å | 113.365 | 435.780 | 11.2% |
| 16.704° | 5.30324 Å | 154.337 | 498.368 | 15.2% |
| 17.208° | 5.14886 Å | 84.6822 | 447.129 | 8.4% |
| 17.776° | 4.98576 Å | 219.306 | 599.898 | 21.6% |
| 18.001° | 4.92383 Å | 284.772 | 671.820 | 28.1% |
| 18.378° | 4.82363 Å | 214.333 | 611.226 | 21.2% |
| 18.888° | 4.69466 Å | 164.251 | 572.534 | 16.2% |
| 19.153° | 4.63030 Å | 478.756 | 892.097 | 47.3% |
| 19.675° | 4.50842 Å | 165.893 | 587.482 | 16.4% |
| 20.016° | 4.43256 Å | 183.658 | 609.374 | 18.1% |
| 20.373° | 4.35557 Å | 212.606 | 641.610 | 21.0% |
| 21.102° | 4.20681 Å | 223.795 | 656.162 | 22.1% |
| 21.490° | 4.13157 Å | 154.704 | 587.035 | 15.3% |
| 22.202° | 4.00083 Å | 133.098 | 562.065 | 13.1% |
| 22.676° | 3.91822 Å | 405.761 | 830.115 | 40.1% |
| 23.300° | 3.81458 Å | 186.119 | 601.505 | 18.4% |
| 24.232° | 3.67005 Å | 112.304 | 511.253 | 11.1% |
| 25.072° | 3.54886 Å | 121.531 | 511.717 | 12.0% |
| 25.298° | 3.51774 Å | 131.002 | 517.824 | 12.9% |
| 25.777° | 3.45346 Å | 132.513 | 510.771 | 13.1% |
| 26.325° | 3.38281 Å | 140.958 | 507.046 | 13.9% |
| 27.041° | 3.29484 Å | 71.8471 | 418.210 | 7.1% |
| 27.494° | 3.24154 Å | 74.0419 | 405.690 | 7.3% |
| 28.419° | 3.13807 Å | 65.5421 | 371.348 | 6.5% |
| 28.765° | 3.10112 Å | 74.7617 | 373.893 | 7.4% |
| 29.055° | 3.07085 Å | 81.3561 | 374.118 | 8.0% |
| 31.898° | 2.80330 Å | 38.5669 | 277.519 | 3.8% |
| 32.102° | 2.78601 Å | 53.2113 | 289.284 | 5.3% |
| 32.571° | 2.74690 Å | 29.7687 | 260.055 | 2.9% |
| 32.825° | 2.72624 Å | 50.7136 | 277.761 | 5.0% |
| 33.189° | 2.69717 Å | 53.2870 | 275.500 | 5.3% |
| 34.856° | 2.57192 Å | 54.0743 | 255.176 | 5.3% |
| 38.836° | 2.31699 Å | 41.9273 | 231.735 | 4.1% |

1. In certain embodiments Compound 1 Form VII is characterized by an XRPD pattern which has at least three peaks selected from 3.9, 7.7, 11.5, 15.7, 16.2, 16.7, 17.8, 18.0, 18.4, 18.9, 19.2, 19.7, 20.0, 20.4, 21.1, 21.5, 22.2, 22.7, 23.3, 24.2, 25.1, 25.3, 25.8, and 26.3+/−0.4° 2theta.
2. The morphic form of embodiment 1, wherein Compound 1 Form VII is characterized by an XRPD pattern which has at least three peaks selected from 3.9, 7.7, 11.5, 15.7, 16.2, 16.7, 17.8, 18.0, 18.4, 18.9, 19.2, 19.7, 20.0, 20.4, 21.1, 21.5, 22.2, 22.7, 23.3, 24.2, 25.1, 25.3, 25.8, and 26.3+/−0.3° 2theta.
3. The morphic form of embodiment 1, wherein Compound 1 Form VII is characterized by an XRPD pattern which has at least three peaks selected from 3.9, 7.7, 11.5, 15.7, 16.2, 16.7, 17.8, 18.0, 18.4, 18.9, 19.2, 19.7, 20.0, 20.4, 21.1, 21.5, 22.2, 22.7, 23.3, 24.2, 25.1, 25.3, 25.8, and 26.3+/−0.2° 2theta.
4. The morphic form of any one of embodiments 1-3, wherein there are at least four peaks selected from the listed peaks.
5. The morphic form of any one of embodiments 1-3, wherein there are at least five peaks selected from the listed peaks.
6. The morphic form of any one of embodiments 1-3, wherein there are at least six peaks selected from the listed peaks.
7. The morphic form of any one of embodiments 1-3, wherein there are at least seven peaks selected from the listed peaks.
8. The morphic form of any one of embodiments 1-3, wherein there are at least eight peaks selected from the listed peaks.
9. The morphic form of any one of embodiments 1-3, wherein there are at least nine peaks selected from the listed peaks.
10. The morphic form of any one of embodiments 1-3, wherein there are at least ten peaks selected from the listed peaks.
11. The morphic form of any one of embodiments 1-10, wherein the XRPD includes a peak at 3.9+/−0.2° 2theta.
12. The morphic form of any one of embodiments 1-11, wherein the XRPD includes a peak at 19.2+/−0.2° 2theta.
13. The morphic form of any one of embodiments 1-12, wherein the XRPD includes a peak at 22.7+/−0.2° 2theta.
14. The morphic form of any one of embodiments 1-13, wherein the XRPD includes a peak at 15.7+/−0.2° 2theta.
15. The morphic form of any one of embodiments 1-14, wherein the XRPD includes a peak at 18.0+/−0.2° 2theta.
16. The morphic form of any one of embodiments 1-15, wherein the XRPD includes a peak at 21.1+/−0.2° 2theta.
17. The morphic form of any one of embodiments 1-16, wherein the XRPD includes a peak at 17.8+/−0.2° 2theta.
18. The morphic form of any one of embodiments 1-17, wherein the XRPD includes a peak at 18.4+/−0.2° 2theta.
19. The morphic form of any one of embodiments 1-18, wherein the XRPD includes a peak at 20.4+/−0.2° 2theta.

### Compound 5 Form VIII

Form VIII is a potential hydrate. It was obtained from DMF/H₂O solvent mixture by antisolvent addition. Form VIII is crystalline. When tested by DSC (see Example 21), Form VIII showed a dehydration and evaporation peak from about 8° C., a melting onset of 231.4° C. and an enthalpy of about 105 J/g. When tested by TGA (see Example 21), Form VIII had a weight loss of about 4.7% at about 200° C. ¹H-NMR shows about 1.6% (0.1 equiv.) of DMF residue by weight. The XRPD of Form VIII is provided in FIG. 8. Form VIII is characterized by an XRPD pattern (XRPD method 2 in Example 23) with peaks with +/−0.4° 2theta of the peaks listed in Peak List

Peak List #8

| | | Net | Gross | Rel. |
|---|---|---|---|---|
| Angle | d Value | Intensity | Intensity | Intensity |
| 4.548° | 19.41368 Å | 181.558 | 234.995 | 33.7% |
| 9.172° | 9.63452 Å | 33.0569 | 118.199 | 6.1% |
| 11.093° | 7.96984 Å | 25.3710 | 131.118 | 4.7% |
| 12.074° | 7.32429 Å | 277.225 | 405.957 | 51.5% |
| 14.448° | 6.12554 Å | 116.085 | 317.291 | 21.5% |
| 16.101° | 5.50021 Å | 98.0776 | 358.650 | 18.2% |
| 16.198° | 5.46746 Å | 203.709 | 467.893 | 37.8% |
| 16.606° | 5.33412 Å | 50.3135 | 328.978 | 9.3% |
| 17.452° | 5.07737 Å | 237.048 | 542.234 | 44.0% |
| 17.937° | 4.94122 Å | 262.231 | 580.468 | 48.7% |
| 18.397° | 4.81874 Å | 538.761 | 867.931 | 100.0% |
| 19.547° | 4.53782 Å | 279.978 | 630.332 | 52.0% |
| 20.032° | 4.42906 Å | 162.985 | 519.639 | 30.3% |
| 20.916° | 4.24381 Å | 133.338 | 497.451 | 24.7% |
| 22.000° | 4.03708 Å | 114.695 | 480.855 | 21.3% |
| 22.368° | 3.97141 Å | 269.566 | 634.643 | 50.0% |
| 22.751° | 3.90537 Å | 385.494 | 748.489 | 71.6% |
| 24.265° | 3.66504 Å | 127.359 | 480.673 | 23.6% |
| 24.668° | 3.60613 Å | 118.815 | 469.681 | 22.1% |
| 25.447° | 3.49741 Å | 210.935 | 553.999 | 39.2% |
| 26.102° | 3.41114 Å | 377.164 | 710.552 | 70.0% |
| 26.521° | 3.35820 Å | 99.7499 | 425.452 | 18.5% |
| 26.910° | 3.31051 Å | 57.7672 | 375.282 | 10.7% |
| 27.437° | 3.24819 Å | 69.4528 | 375.896 | 12.9% |
| 28.042° | 3.17943 Å | 112.127 | 404.479 | 20.8% |
| 28.800° | 3.09740 Å | 85.6968 | 356.955 | 15.9% |
| 29.160° | 3.05999 Å | 63.8392 | 323.750 | 11.8% |
| 29.514° | 3.02410 Å | 55.0689 | 302.984 | 10.2% |
| 30.107° | 2.96585 Å | 45.3171 | 274.151 | 8.4% |
| 31.840° | 2.80826 Å | 163.323 | 364.805 | 30.3% |
| 32.793° | 2.72885 Å | 34.1858 | 222.495 | 6.3% |
| 34.526° | 2.59572 Å | 34.1789 | 210.946 | 6.3% |
| 35.579° | 2.52128 Å | 30.5723 | 204.689 | 5.7% |

1. In certain embodiments Compound 1 Form VIII is characterized by an XRPD pattern which has at least three peaks selected from 4.5, 12.1, 14.4, 16.1, 16.2, 17.5, 17.9, 18.4, 19.5, 20.0, 20.9, 22.0, 22.4, 22.8, 24.3, 24.7, 25.4, 26.1, 26.5, 26.9, 27.4, 28.0, 28.8, 29.2, 29.5, and 31.8+/−0.4° 2theta.
2. The morphic form of embodiment 1, wherein Compound 1 Form VIII is characterized by an XRPD pattern which has at least three peaks selected from 4.5, 12.1, 14.4, 16.1, 16.2, 17.5, 17.9, 18.4, 19.5, 20.0, 20.9, 22.0, 22.4, 22.8, 24.3, 24.7, 25.4, 26.1, 26.5, 26.9, 27.4, 28.0, 28.8, 29.2, 29.5, and 31.8+/−0.3° 2theta.
3. The morphic form of embodiment 1, wherein Compound 1 Form VIII is characterized by an XRPD pattern which has at least three peaks selected from 4.5, 12.1, 14.4, 16.1, 16.2, 17.5, 17.9, 18.4, 19.5, 20.0, 20.9, 22.0, 22.4, 22.8, 24.3, 24.7, 25.4, 26.1, 26.5, 26.9, 27.4, 28.0, 28.8, 29.2, 29.5, and 31.8+/−0.2° 2theta.
4. The morphic form of any one of embodiments 1-3, wherein there are at least four peaks selected from the listed peaks.
5. The morphic form of any one of embodiments 1-3, wherein there are at least five peaks selected from the listed peaks.
6. The morphic form of any one of embodiments 1-3, wherein there are at least six peaks selected from the listed peaks.
7. The morphic form of any one of embodiments 1-3, wherein there are at least seven peaks selected from the listed peaks.
8. The morphic form of any one of embodiments 1-3, wherein there are at least eight peaks selected from the listed peaks.
9. The morphic form of any one of embodiments 1-3, wherein there are at least nine peaks selected from the listed peaks.
10. The morphic form of any one of embodiments 1-3, wherein there are at least ten peaks selected from the listed peaks.
11. The morphic form of any one of embodiments 1-10, wherein the XRPD includes a peak at 18.4+/−0.2° 2theta.
12. The morphic form of any one of embodiments 1-11, wherein the XRPD includes a peak at 22.8+/−0.2° 2theta.
13. The morphic form of any one of embodiments 1-12, wherein the XRPD includes a peak at 26.1+/−0.2° 2theta.
14. The morphic form of any one of embodiments 1-13, wherein the XRPD includes a peak at 19.5+/−0.2° 2theta.
15. The morphic form of any one of embodiments 1-14, wherein the XRPD includes a peak at 12.1+/−0.2° 2theta.
16. The morphic form of any one of embodiments 1-15, wherein the XRPD includes a peak at 22.4+/−0.2° 2theta.
17. The morphic form of any one of embodiments 1-16, wherein the XRPD includes a peak at 17.9+/−0.2° 2theta.
18. The morphic form of any one of embodiments 1-17, wherein the XRPD includes a peak at 17.5+/−0.2° 2theta.
19. The morphic form of any one of embodiments 1-18, wherein the XRPD includes a peak at 25.4+/−0.2° 2theta.
20. The morphic form of any one of embodiments 1-19, wherein the XRPD includes a peak at 16.2+/−0.2° 2theta.
21. The morphic form of any one of embodiments 1-20, wherein the XRPD includes a peak at 4.5+/−0.2° 2theta.
22. The morphic form of any one of embodiments 1-21, wherein the XRPD includes a peak at 20.0+/−0.2° 2theta.
23. The morphic form of any one of embodiments 1-22, wherein the XRPD includes a peak at 31.8+/−0.2° 2theta.
24. The morphic form of any one of embodiments 1-23, wherein the XRPD includes a peak at 20.9+/−0.2° 2theta.
25. The morphic form of any one of embodiments 1-24, wherein the XRPD includes a peak at 24.3+/−0.2° 2theta.
26. The morphic form of any one of embodiments 1-25, wherein the XRPD includes a peak at 24.7+/−0.2° 2theta.
27. The morphic form of any one of embodiments 1-26, wherein the XRPD includes a peak at 14.4+/−0.2° 2theta.
28. The morphic form of any one of embodiments 1-27, wherein the XRPD includes a peak at 22.0+/−0.2° 2theta.
29. The morphic form of any one of embodiments 1-28, wherein the XRPD includes a peak at 28.0+/−0.2° 2theta.

In certain embodiments Form VIII is characterized by a dehydration and evaporation onset at about 8° C. and/or a melting onset at about 230° C. and/or a melting enthalpy of about 105 J/g on DSC thermogram. In certain embodiments Form VIII is characterized by a weight loss of about 5% at about 200° C. as measured by TGA.

In certain embodiments Form VIII is characterized by a solubility of about 30 mg/mL in MeOH/DCM (v:v=1:2) and/or 39 mg/mL in EtOH/DCM (v:v=1:4) when solubilized at about 25° C. as described in Example 20.

### Approximate Solubility

The four Compound 5 polymorphs, Form V, Form VI, Form VII, and Form VIII were tested in EtOH:DCM (v:v=1:4) for approximate solubility in Example 18. Form VI and Form VIII showed higher solubility in EtOH:DCM (v:v=1:4) than Form V and Form VII by visual observation.

### Solubility

Solubility of Compound 5 Form VI and Form VIII was investigated in MeOH:DCM (v:v=1:2), EtOH:DCM (v:v=1:4), THF:2-propanol (v:v=1:2), THF:Heptane (v:v=1:2) and THF:water (v:v=1:2.5) at 25° C. for 24 h in Example 20. Both Form VI (˜30 mg/mL) and Form VIII (˜30-40 mg/mL) showed high solubility in MeOH:DCM (v:v=1:2) and EtOH:DCM (v:v=1:4) but low solubility (˜1 mg/mL) in THF:IPA (v:v=1:2), THF:Heptane (v:v=1:2) and THF:Heptane (v:v=1:2.5).

After the solubility test, Form VI converted to almost the amorphous form in MeOH:DCM (v:v=1:2) and EtOH:DCM (v:v=1:4) but there was no form change in THF:2-propanol (v:v=1:2), THF:Heptane (v:v=1:2) and THF:water (v:v=1:2.5).

After solubility test, Form VIII converted to Form V in MeOH:DCM (v:v=1:2) and THF:water (v:v=1:2.5) and it converted to Form VI in THF:2-propanol (v:v=1:2) and THF:Heptane (v:v=1:2). Physical mixture of Form V and Form VI were obtained in EtOH:DCM (v:v=1:4).

### Bulk Stability

Bulk stability of Compound 5 Form VI and Form VIII was evaluated at 25° C./60% RH in an open container over 1 week as described in Example 19. Compound 5 Form VI and Form VIII are chemically stable under this condition. Form VI also showed good physical stability. Form VIII converted to Form V after exposure to 25° C./60% RH over 1 week.

In certain embodiments Compound 5 Form VI is used in the preparation of Compound 1, for example Compound 1 Form I.

### New Dosage Regimes for Compound 1

In another aspect of the present invention a new dosage regimen for the treatment of cancer is provided comprising administering Compound 1 or a pharmaceutically acceptable salt thereof, Compound 1 Form II, or a pharmaceutical composition comprising Compound 1 to a patient in need thereof (a) once a day for three selected days per week for fourteen days followed by a 14 day dose holiday in a 28 day treatment cycle; or (b) once a day for 7 consecutive days followed by a 14 day dose holiday in a 21 day treatment cycle. In certain embodiments the treatment cycle is repeated 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, or more times. In certain embodiments Compound 1 is provided in a dose of about 1 μg, 5 μg, 10 μg, 15 μg, 20 μg, 25 μg, 37.5 μg, 50 μg, 62.5 μg, 75 μg, 87.5 μg, 100 μg, 125 μg, 150 μg, 175 μg, 200 μg, 225 μg, 250 μg, 275 μg, 300 μg, 325 μg, 350 μg, 375 μg, or 400 μg. In certain embodiments Compound 1 is provided in a dose of at least about 1 μg, 5 μg, 10 μg, 15 μg, 20 μg, 25 μg, 37.5 μg, 50 μg, 75 μg, 87.5 μg, 100 μg, 125 μg, 150 μg, 175 μg, 200 μg, 225 μg, 250 μg, 275 μg, 300 μg, 325 μg, 350 μg, 375 μg, or 400 μg. In certain embodiments Compound 1 is provided in a dose of less than about 1 μg, 5 μg, 10 μg, 15 μg, 20 μg, 25 μg, 37.5 μg, 50 μg, 75 μg, 87.5 μg, 100 μg, 125 μg, 150 μg, 175 μg, 200 μg, 225 μg, 250 μg, 275 μg, 300 μg, 325 μg, 350 μg, 375 μg, or 400 μg.

In certain embodiments, Compound 1 is administered at a dose of less than about 50 μg. In certain embodiments, the therapy is administered at a dose of less than about 25 μg.

In certain embodiments, the therapy is administered at a dose of about 50 μg.

In certain embodiments, the therapy is administered at a dose of about 45 μg.

In certain embodiments, the therapy is administered at a dose of about 40 μg.

In certain embodiments, the therapy is administered at a dose of about 35 μg.

In certain embodiments, the therapy is administered at a dose of about 30 μg.

In certain embodiments, the therapy is administered at a dose of about 25 μg.

In certain embodiments, the therapy is administered at a dose of about 20 μg.

In certain embodiments, the therapy is administered at a dose of about 15 μg.

In certain embodiments, the therapy is administered at a dose of about 10 μg.

In certain embodiments, the therapy is administered at a dose of about 5 μg.

In certain embodiments, the therapy is administered at a dose of about 1 μg.

In certain embodiments, the therapy is given at a dose of less than about 800 μg. In certain embodiments, the therapy is given at a dose of less than about 600 μg. In certain embodiments, the therapy is given at a dose of less than about 400 μg. In certain embodiments, the therapy is given at a dose of less than about 300 μg. In certain embodiments, the therapy is given at a dose of less than about 200 μg. In certain embodiments, the therapy is given at a dose of less than about 100 μg.

In certain embodiments, Compound 1 is administered at a dose of not more than about 25, 50, 75, 100, 125, 150, 175, 200, 225, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, or 1,000 μg.

Treatment of Disorders Mediated by Ikaros and/or Aiolos

Advantageous treatments of disorders mediated by Ikaros and/or Aiolos are provided comprising administering Compound 1 Form I, a pharmaceutical composition comprising Compound 1 Form I, a pharmaceutical composition prepared from Compound 1 Form I (for example a liquid dosage form or gel capsule that comprises Compound 1 that was Form I before being dissolved), or Compound 1 as part of a new dosage regimen described herein. In certain embodiments the method of treatment uses a low effective dose of Compound 1. For example, a dose of Compound 1 of not more than about 500, 450, 400, 350, 325, 300, 275, 250, 225, 200, 175, 150, 125 100, 75, 62.5, 50, 37.5, 25, 20, 15, 10, 5, or 1 micrograms (μg) once weekly, twice weekly, three times weekly, four times weekly, five times weekly, six times weekly, or once daily (QD) optionally with a treatment holiday. For example, a regimen with treatment occurring on days 1-14 or 1-21 of a 28-day treatment cycle. In some embodiments the patient is an adult (typically a human of at least 100 pounds or more, and typically 18 years old or more). In an alternative embodiment, the patient is pediatric (and may be less than 100 pounds and typically less than 18 years old).

Compound 1 may be administered as monotherapy or may be combined with a standard of care therapy for the target tumor or cancer, including but not limited to any of those described in the Background of the Invention, or such as a proteasome inhibitor and/or an anti-CD38 monoclonal antibody (mAbs). In MCL, Compound 1 can be used, for example, in combination with a Bruton tyrosine kinase (BTK) inhibitor, or an anti-CD20 monoclonal antibody. In PTCL, in particular ALCL, Compound 1 may be administered, for example, in combination with anti-CD30 or anti-CD38 monoclonal antibody.

In certain embodiments, Compound 1 is used to treat a disorder mediated by Ikaros or Aiolos according to a treatment regimen described herein.

In certain embodiments Compound 1, is used to treat a cancer that has metastasized. In certain embodiments Compound 1, is used to treat a cancer that has metastasized to the brain. In certain embodiments Compound 1, is used to treat multiple myeloma that has metastasized to the brain. In certain embodiments Compound 1, is used to treat non-Hodgkin's lymphoma that has metastasized to the brain. In certain embodiments Compound 1, is used to treat diffuse large B-cell lymphoma that has metastasized to the brain.

### Treatment Cycles

In certain embodiments, the treatment includes a drug holiday. A drug holiday is a time period during which the patient is not administered the active compound. For example, the patient may be administered the pharmaceutical composition for 14 or 21 continuous days and not administered the chemotherapeutic for 14 or 7 days during a 28-day cycle, and then optionally the regimen is repeated once, several or more times.

In certain embodiments, the treatment cycles are provided to allow for recovery of blood cell levels and in a non-limiting embodiments the depression of neutrophil count. The health care provider can monitor blood cell levels during therapy, and place the patient on a drug holiday for a certain period during the 28 day treatment cycle. Alternatively, the health care provider can reduce the administered dose until the depressed blood cell count suitably recovers. A combination of these two techniques can be used if desired.

In certain embodiments, Compound 1 is administered once a day for 14 continuous days of a 28-day treatment cycle. In certain embodiments, Compound 1 is administered three times a week, for example on Monday, Wednesday, and Friday and then Compound 1 is not administered for 7 or 14 consecutive days of a 28-day treatment cycle.

In certain embodiments, Compound 1 is administered once a day for 1 day in a seven-day treatment cycle.

In certain embodiments, Compound 1 is administered once a day for 2 days in a seven-day treatment cycle.

In certain embodiments, Compound 1 is administered once a day for 3 days in a seven-day treatment cycle.

In certain embodiments, Compound 1 is administered once a day for 4 days in a seven-day treatment cycle.

In certain embodiments, Compound 1 is administered once a day for 5 days in a seven-day treatment cycle.

In certain embodiments, Compound 1 is administered once a day for 6 days in a seven-day treatment cycle.

In certain embodiments, Compound 1 is administered once a day for 7 days in a seven-day treatment cycle.

In certain embodiments, Compound 1 is administered once a day for 1 day a week in a fourteen-day treatment cycle.

In certain embodiments, Compound 1 is administered once a day for 2 days a week in a fourteen-day treatment cycle.

In certain embodiments, Compound 1 is administered once a day for 3 days a week in a fourteen-day treatment cycle.

In certain embodiments, Compound 1 is administered once a day for 4 days a week in a fourteen-day treatment cycle.

In certain embodiments, Compound 1 is administered once a day for 5 days a week in a fourteen-day treatment cycle.

In certain embodiments, Compound 1 is administered once a day for 6 days a week in a fourteen-day treatment cycle.

In certain embodiments, Compound 1 is administered once a day for 1 day a week in a 21-day treatment cycle.

In certain embodiments, Compound 1 is administered once a day for 2 days a week in a 21-day treatment cycle.

In certain embodiments, Compound 1 is administered once a day for 3 days a week in a 21-day treatment cycle.

In certain embodiments, Compound 1 is administered once a day for 4 days a week in a 21-day treatment cycle.

In certain embodiments, Compound 1 is administered once a day for 5 days a week in a 21-day treatment cycle.

In certain embodiments, Compound 1 is administered once a day for 6 days a week in a 21-day treatment cycle.

In certain embodiments, Compound 1 is administered once a day for 1 day a week in a 28-day treatment cycle.

In certain embodiments, Compound 1 is administered once a day for 2 days a week in a 28-day treatment cycle.

In certain embodiments, Compound 1 is administered once a day for 3 days a week in a 28-day treatment cycle.

In certain embodiments, Compound 1 is administered once a day for 4 days a week in a 28-day treatment cycle.

In certain embodiments, Compound 1 is administered once a day for 5 days a week in a 28-day treatment cycle.

In certain embodiments, Compound 1 is administered once a day for 6 days a week in a 28-day treatment cycle.

In certain embodiments, Compound 1 is administered once a day for 7 days a week for one week in a 28-day treatment cycle.

In certain embodiments, Compound 1 is administered once a day for 7 days a week for two weeks in a 28-day treatment cycle.

In certain embodiments the treatment schedule above is modified to include a one week holiday for example Compound 1 is administered once a day for 5 days a week in a 28-day treatment cycle wherein Compound 1 is only administered in the first three weeks of the 28-day treatment cycle.

In certain embodiments the treatment schedule above is modified to include a two week holiday for example Compound 1 is administered once a day for 5 days a week in a 28-day treatment cycle wherein Compound 1 is only administered in the first two weeks of the 28-day treatment cycle.

In certain embodiments the three selected days for treatment are Monday, Wednesday, and Friday. In certain embodiments the three selected days for treatment are Tuesday, Thursday, and Saturday. In certain embodiments the three selected days for treatment are Wednesday, Friday, and Sunday.

In certain embodiments Compound 1 is administered orally. In certain embodiments Compound 1 is administered as a gel capsule or a tablet. In certain embodiments Compound 1 is administered as a tablet. For example, in certain embodiments Compound 1 is administered in a tablet made in a spray dried dispersion.

### Dosing

In certain embodiments, Compound 1 is administered at a dose of about 75 μg or less. In certain embodiments, the therapy (i.e., Compound 1 Form I or a pharmaceutical composition comprising or prepared from Compound 1 Form I, or Compound 1 as part of a new dosing regimen described herein) is administered at a dose of 75 μg or less. In certain embodiments, the therapy (i.e., Compound 1 Form I or a pharmaceutical composition comprising or prepared from Compound 1 Form I, or Compound 1 as part of a new dosing regimen described herein) is administered at a dose of about 50 μg or less. In certain embodiments, the therapy (i.e., Compound 1 Form I or a pharmaceutical composition comprising or prepared from Compound 1 Form I, or Compound 1 as part of a new dosing regimen described herein) is administered at a dose of less than about 25 μg.

In certain embodiments, the therapy is administered at a dose of about 75 μg.

In certain embodiments, the therapy is administered at a dose of about 62.5 μg.

In certain embodiments, the therapy is administered at a dose of about 50 μg.

In certain embodiments, the therapy is administered at a dose of about 45 μg.

In certain embodiments, the therapy is administered at a dose of about 40 μg.

In certain embodiments, the therapy is administered at a dose of about 37.5 g.

In certain embodiments, the therapy is administered at a dose of about 35 μg.

In certain embodiments, the therapy is administered at a dose of about 30 μg.

In certain embodiments, the therapy is administered at a dose of about 25 μg.

In certain embodiments, the therapy is administered at a dose of about 20 μg.

In certain embodiments, the therapy is administered at a dose of about 15 μg.

In certain embodiments, the therapy is administered at a dose of about 12.5 g.

In certain embodiments, the therapy is administered at a dose of about 10 μg.

In certain embodiments, the therapy is administered at a dose of about 5 μg.

In certain embodiments, the therapy is administered at a dose of about 1 μg.

In certain embodiments, the therapy is given at a dose of less than about 800 μg. In certain embodiments, the therapy is given at a dose of less than about 600 μg. In certain embodiments, the therapy is given at a dose of less than about 400 μg. In certain embodiments, the therapy is given at a dose of less than about 300 μg. In certain embodiments, the therapy is given at a dose of less than about 200 μg. In certain embodiments, the therapy is given at a dose of less than about 100 μg, 75 μg, 50 μg, or 25 μg.

In certain embodiments, Compound 1 is administered at a dose of at least about or between 10, 12.5, 25, 37.5, 50, 62.5, 75, 100, 125, 150, 175, 200, 225, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, or 1,000 μg.

In certain aspects Compound 1 is administered at a dose of 50 μg once a day for fourteen days followed by a fourteen-day treatment holiday in a twenty eight day treatment cycle. In certain embodiments Compound 1 is administered at a dose of 62.5 μg once a day for fourteen days followed by a fourteen-day treatment holiday in a twenty eight day treatment cycle. In certain embodiments Compound 1 is administered at a dose of 75 μg once a day for fourteen days followed by a fourteen-day treatment holiday in a twenty eight day treatment cycle.

In certain aspects Compound 1 is administered at a dose of 50 μg once a day three days a week for the first two weeks of a fourteen-day treatment holiday in a twenty eight day treatment cycle. In certain embodiments Compound 1 is administered at a dose of 62.5 μg once a day three days a week for the first two weeks of a fourteen-day treatment holiday in a twenty eight day treatment cycle. In certain embodiments Compound 1 is administered at a dose of 75 μg once a day three days a week for the first two weeks of a fourteen-day treatment holiday in a twenty eight day treatment cycle.

In certain aspects Compound 1 is administered at a dose of 50 μg once a day on Monday, Wednesday, and Friday for the first two weeks of a fourteen-day treatment holiday in a twenty eight day treatment cycle. In certain embodiments Compound 1 is administered at a dose of 62.5 μg once a day on Monday, Wednesday, and Friday for the first two weeks of a fourteen-day treatment holiday in a twenty eight day treatment cycle. In certain embodiments Compound 1 is administered at a dose of 75 μg on Monday, Wednesday, and Friday for the first two weeks of a fourteen-day treatment holiday in a twenty eight day treatment cycle.

In certain aspects Compound 1 is administered at a dose of 50 μg once a day for seven days followed by a fourteen-day treatment holiday in a twenty one day treatment cycle. In certain embodiments Compound 1 is administered at a dose of 62.5 μg once a day for seven days followed by a fourteen-day treatment holiday in a twenty one day treatment cycle. In certain embodiments Compound 1 is administered at a dose of 75 μg once a day for seven days followed by a fourteen-day treatment holiday in a twenty one day treatment cycle.

Disorders Mediated by Ikaros and/or Aiolos

In one aspect of the present invention a disorder mediated by Ikaros or Aiolos is treated by Compound 1. In certain embodiments, the cancer is a hematopoietic cancer. In some embodiments, the cancer is a lymphoma, leukemia or myeloma. In certain aspects, the cancer is multiple myeloma. In certain aspects, the cancer is a Non-Hodgkin's Lymphoma or a Hodgkin's lymphoma.

In certain embodiments, the cancer is diffuse large B-cell lymphoma. In certain embodiments, the diffuse large B-cell lymphoma is an activated B-cell lymphoma or a germinal center B-cell lymphoma. In certain embodiments, the diffuse large B-cell lymphoma is a BCL2/6 translocation bearing cancer. In certain embodiments, the diffuse large B-cell lymphoma is a double hit bearing cancer. In certain embodiments, the diffuse large B-cell lymphoma is a BCL2/6 MYC wild type cancer.

In certain embodiments Compound 1 increases the concentration of Caspase-3 and/or Caspase-7. In certain embodiments this increased concentration of Caspase-3 and/or caspase-7 drives cancer cell death, for example mediating the treatment of diffuse large B-cell lymphoma.

In certain embodiments, the cancer is anaplastic large cell lymphoma.

In certain embodiments, the cancer is cutaneous T-cell lymphoma.

In certain embodiments, the cancer is mantle cell lymphoma.

In certain embodiments, the cancer is multiple myeloma.

In certain embodiments, the patient has an extramedullary disease.

In certain embodiments the patient has multiple myeloma and an extramedullary disease.

Extramedullary diseases can occur when the cancer cells form tumors outside of the bone marrow, for example in the soft tissues or organs of the body. In certain embodiments the extramedullary disease occurs in the CNS, skull, orbit, vertebrae, breast, thyroid, soft tissue of the neck, lungs, spleen, heart, liver, pancreas, gastrointestinal tract, omentum, testis, skin, subcutaneous tissue, lymph note, muscle, female reproductive system, or adrenal glands. In certain embodiments the extramedullary disease is selected from multiple plasmacytomas, lytic bone lesions, or the combination of plasmacytomas and bone lesions.

In certain embodiments the patient has multiple myeloma and multiple plasmacytomas.

In certain embodiments the patient has multiple myeloma and lytic bone lesions.

In certain embodiments the patient has multiple myeloma, plasmacytomas, and bone lesions.

In certain embodiments the patient is administered surgical therapy either before, during treatment, or after treatment with Compound 1.

In certain embodiments, Compound 1 is administered to a patient in need thereof in an effective amount to treat a disorder that is resistant to treatment with other cereblon ligands. In certain embodiments, Compound 1 is used to treat an immunomodulatory imide drug- or IID-refractory disorder. In certain embodiments, Compound 1 is used to treat relapsed or refractory multiple myeloma. In certain embodiments, Compound 1 is used to treat relapsed or refractory non-Hodgkins's lymphoma.

In one aspect the patient has a CNS involved cancer, for example a lymphoma in the CNS. In certain embodiments, the patient with a CNS involved cancer, for example a lymphoma in the CNS, is also administered one or more additional therapeutic agents, for example ibrutinib or rituximab. In certain embodiments, a compound described herein is used in the treatment of a peripheral central nervous system lymphoma.

In certain embodiments, the cancer is PTCL-NOS (i.e. PTCL that is not otherwise specified). In other embodiments the cancer is PTCL with a specified subtype for example anaplastic large cell lymphoma (ALCL), angioimmunoblastic T-Cell lymphoma (AITL), enteropathy-type T-cell lymphoma, extranodal natural killer (NK) cell lymphoma, or extranodal T-cell lymphoma.

In certain embodiments, the disorder is an immunomodulatory disorder. In certain embodiments, the disorder treated as described herein is mediated by angiogenesis. In certain embodiments, the disorder is related to the lymphatic system.

In certain embodiments, the disorder is selected from benign growth, neoplasm, tumor, cancer, abnormal cellular proliferation, immune disorder, inflammatory disorder, graft-versus-host rejection, viral infection, bacterial infection, an amyloid-based proteinopathy, a proteinopathy, or a fibrotic disorder.

In certain embodiments the term “disease state” or “condition” when used herein is meant to refer to any disease state or condition that is mediated by Ikaros or Aiolos, such as cellular proliferation, or by proteins that are downstream of Ikaros or Aiolos, and where degradation of such protein in a patient may provide beneficial therapy or relief of symptoms to a patient in need thereof. In certain instances, the disease state or condition may be cured.

For example, Compound 1 can be administered to a host suffering from a Hodgkin Lymphoma or a Non-Hodgkin Lymphoma. For example, the host can be suffering from a Non-Hodgkin Lymphoma such as, but not limited to: an AIDS-Related Lymphoma; Anaplastic Large-Cell Lymphoma; Angioimmunoblastic Lymphoma; Blastic NK-Cell Lymphoma; Burkitt's Lymphoma; Burkitt-like Lymphoma (Small Non-Cleaved Cell Lymphoma); diffuse small-cleaved cell lymphoma (DSCCL); Chronic Lymphocytic Leukemia, Small Lymphocytic Lymphoma; Non-Hodgkin lymphoma NOS, Cutaneous T-Cell Lymphoma; Diffuse Large B-Cell Lymphoma; Enteropathy-Type T-Cell Lymphoma; Follicular Lymphoma; Hepatosplenic Gamma-Delta T-Cell Lymphoma; Lymphoblastic Lymphoma; Mantle Cell Lymphoma; Marginal Zone Lymphoma; Nasal T-Cell Lymphoma; Pediatric Lymphoma; Peripheral Lymphoma, Peripheral T-Cell Lymphomas; Primary Central Nervous System Lymphoma; T-Cell Leukemias; Transformed Lymphomas; Treatment-Related T-Cell Lymphomas; Langerhans cell histiocytosis; or Waldenstrom's Macroglobulinemia.

In certain embodiments the disorder is a Hodgkin lymphoma, such as, but not limited to: Nodular Sclerosis Classical Hodgkin's Lymphoma (CHL); Mixed Cellularity CHL; Lymphocyte-depletion CHL; Lymphocyte-rich CHL; Lymphocyte Predominant Hodgkin Lymphoma; or Nodular Lymphocyte Predominant HL.

In certain embodiments the disorder is an immunomodulatory condition. Non-limiting examples of immunomodulatory conditions include: arthritis, lupus, celiac disease, inflammatory bowel disease, ulcerative colitis, Sjogren's syndrome, polymyalgia rheumatia, multiple sclerosis, ankylosing spondylitis, type 1 diabetes, alopecia areata, vasculitis, and temporal arteritis.

In certain embodiments, the condition treated as described herein is a disorder related to abnormal cellular proliferation. Abnormal cellular proliferation, notably hyperproliferation, can occur as a result of a wide variety of factors, including genetic mutation, infection, exposure to toxins, autoimmune disorders, and benign or malignant tumor induction.

Abnormal proliferation of B-cells, T-cells, and/or NK cells can result in a wide range of diseases such as cancer, proliferative disorders and inflammatory/immune diseases. A host, for example a human, afflicted with any of these disorders can be treated with an effective amount of a compound as described herein to achieve a decrease in symptoms (palliative agent) or a decrease in the underlying disease (a disease modifying agent).

In certain embodiments, a compound or its corresponding pharmaceutically acceptable salt, isotopic derivative, or prodrug as described herein can be administered in an effective amount to treat a host, for example a human, with a specific B-cell lymphoma or proliferative disorder such as, but not limited to: multiple myeloma; Diffuse large B cell lymphoma; Follicular lymphoma; Mucosa-Associated Lymphatic Tissue lymphoma (MALT); Small cell lymphocytic lymphoma; diffuse poorly differentiated lymphocytic lymphoma; Mediastinal large B cell lymphoma; Nodal marginal zone B cell lymphoma (NMZL); Splenic marginal zone lymphoma (SMZL); Intravascular large B-cell lymphoma; Primary effusion lymphoma; or Lymphomatoid granulomatosis; B-cell prolymphocytic leukemia; Hairy cell leukemia; Splenic lymphoma/leukemia, unclassifiable; Splenic diffuse red pulp small B-cell lymphoma; Hairy cell leukemia-variant; Lymphoplasmacytic lymphoma; Heavy chain diseases, for example, Alpha heavy chain disease, Gamma heavy chain disease, Mu heavy chain disease; Plasma cell myeloma; Solitary plasmacytoma of bone; Extraosseous plasmacytoma; Primary cutaneous follicle center lymphoma; T cell/histiocyte rich large B-cell lymphoma; DLBCL associated with chronic inflammation; Epstein-Barr virus (EBV)+ DLBCL of the elderly; Primary mediastinal (thymic) large B-cell lymphoma; Primary cutaneous DLBCL, leg type; ALK+ large B-cell lymphoma; Plasmablastic lymphoma; Large B-cell lymphoma arising in HHV8-associated multicentric; Castleman disease; B-cell lymphoma, unclassifiable, with features intermediate between diffuse large B-cell lymphoma; or B-cell lymphoma, unclassifiable, with features intermediate between diffuse large B-cell lymphoma and classical Hodgkin lymphoma.

In certain embodiments, a compound or its corresponding pharmaceutically salt, isotopic derivative, or prodrug as described herein can be administered in an effective amount to treat a host, for example a human, with a T-cell or NK-cell lymphoma such as, but not limited to: anaplastic lymphoma kinase (ALK) positive, ALK negative anaplastic large cell lymphoma, or primary cutaneous anaplastic large cell lymphoma; angioimmunoblastic lymphoma; cutaneous T-cell lymphoma, for example mycosis fungoides, Sézary syndrome, primary cutaneous anaplastic large cell lymphoma, primary cutaneous CD30+ T-cell lymphoproliferative disorder; primary cutaneous aggressive epidermotropic CD8+ cytotoxic T-cell lymphoma; primary cutaneous gamma-delta T-cell lymphoma; primary cutaneous small/medium CD4+ T-cell lymphoma, and lymphomatoid papulosis; Adult T-cell Leukemia/Lymphoma (ATLL); Blastic NK-cell Lymphoma; Enteropathy-type T-cell lymphoma; Hematosplenic gamma-delta T-cell Lymphoma; Lymphoblastic Lymphoma; Nasal NK/T-cell Lymphomas; Treatment-related T-cell lymphomas; for example lymphomas that appear after solid organ or bone marrow transplantation; T-cell prolymphocytic leukemia; T-cell large granular lymphocytic leukemia; Chronic lymphoproliferative disorder of NK-cells; Aggressive NK cell leukemia; Systemic EBV+ T-cell lymphoproliferative disease of childhood (associated with chronic active EBV infection); Hydroa vacciniforme-like lymphoma; Adult T-cell leukemia/lymphoma; Enteropathy-associated T-cell lymphoma; Hepatosplenic T-cell lymphoma; or Subcutaneous panniculitis-like T-cell lymphoma.

In certain embodiments, a compound or its corresponding pharmaceutically acceptable salt, isotopic derivative, or prodrug as described herein can be administered to treat a host, for example a human, with leukemia. For example, the host may be suffering from an acute or chronic leukemia of a lymphocytic or myelogenous origin, such as, but not limited to: Acute lymphoblastic leukemia (ALL); Acute myelogenous leukemia (AML); Chronic lymphocytic leukemia (CLL); Chronic myelogenous leukemia (CML); juvenile myelomonocytic leukemia (JMML); hairy cell leukemia (HCL); acute promyelocytic leukemia (a subtype of AML); large granular lymphocytic leukemia; or Adult T-cell chronic leukemia. In certain embodiments, the patient suffers from an acute myelogenous leukemia, for example an undifferentiated AML (MO); myeloblastic leukemia (M1; with/without minimal cell maturation); myeloblastic leukemia (M2; with cell maturation); promyelocytic leukemia (M3 or M3 variant [M3V]); myelomonocytic leukemia (M4 or M4 variant with eosinophilia [M4E]); monocytic leukemia (M5); erythroleukemia (M6); or megakaryoblastic leukemia (M7).

There are a number of skin disorders associated with cellular hyperproliferation. Psoriasis, for example, is a benign disease of human skin generally characterized by plaques covered by thickened scales. The disease is caused by increased proliferation of epidermal cells of unknown cause. Chronic eczema is also associated with significant hyperproliferation of the epidermis. Other diseases caused by hyperproliferation of skin cells include atopic dermatitis, lichen planus, warts, pemphigus vulgaris, actinic keratosis, basal cell carcinoma and squamous cell carcinoma.

Other hyperproliferative cell disorders include blood vessel proliferation disorders, fibrotic disorders, autoimmune disorders, graft-versus-host rejection, tumors and cancers.

Blood vessel proliferative disorders include angiogenic and vasculogenic disorders. Proliferation of smooth muscle cells in the course of development of plaques in vascular tissue cause, for example, restenosis, retinopathies and atherosclerosis. Both cell migration and cell proliferation play a role in the formation of atherosclerotic lesions.

Fibrotic disorders are often due to the abnormal formation of an extracellular matrix. Examples of fibrotic disorders include hepatic cirrhosis and mesangial proliferative cell disorders. Hepatic cirrhosis is characterized by the increase in extracellular matrix constituents resulting in the formation of a hepatic scar. Hepatic cirrhosis can cause diseases such as cirrhosis of the liver. An increased extracellular matrix resulting in a hepatic scar can also be caused by viral infection such as hepatitis. Lipocytes appear to play a major role in hepatic cirrhosis.

Mesangial disorders are brought about by abnormal proliferation of mesangial cells. Mesangial hyperproliferative cell disorders include various human renal diseases, such as glomerulonephritis, diabetic nephropathy, malignant nephrosclerosis, thrombotic micro-angiopathy syndromes, transplant rejection, and glomerulopathies.

Another disease with a proliferative component is rheumatoid arthritis. Rheumatoid arthritis is generally considered an autoimmune disease that is thought to be associated with activity of autoreactive T cells, and to be caused by autoantibodies produced against collagen and IgE.

Other disorders that can include an abnormal cellular proliferative component include Bechet's syndrome, acute respiratory distress syndrome (ARDS), ischemic heart disease, post-dialysis syndrome, leukemia, acquired immune deficiency syndrome, vasculitis, lipid histiocytosis, septic shock and inflammation in general.

A compound or its pharmaceutically acceptable salt, isotopic analog, or prodrug as described herein can be administered in an effective amount to treat a host, for example a human, with a proliferative condition such as myeloproliferative disorder (MPD), polycythemia vera (PV), essential thrombocythemia (ET), myeloid metaplasia with myelofibrosis (MMM), chronic myelomonocytic leukemia (CMML), hypereosinophilic syndrome (HES), system mast cell disease (SMCD), and the like. In another embodiment, a compound provided herein is useful for the treatment of primary myelofibrosis, post-polycythemia vera myelofibrosis, post-essential thrombocythemia myelofibrosis, and secondary acute myelogenous leukemia.

In certain embodiments, a compound or its pharmaceutically acceptable salt, isotopic analog, or prodrug as described herein can be administered in an effective amount to treat a host, for example a human, with a myelodysplastic syndrome (MDS) such as, but not limited to: refractory cytopenia with unilineage dysplasia, refractory anemia with ring sideroblasts (RARS), refractory anemia with ring sideroblasts—thrombocytosis (RARS-t), refractory cytopenia with multilineage dysplasia (RCMD) including RCMD with multilineage dysplasia and ring sideroblasts (RCMD-RS), Refractory amenias with excess blasts I (RAEB-I) and II (RAEB-II), 5q-syndrome, refractory cytopenia of childhood, and the like.

The term “neoplasia” or “cancer” is used to refer to the pathological process that results in the formation and growth of a cancerous or malignant neoplasm, i.e., abnormal tissue that grows by cellular proliferation, often more rapidly than normal and continues to grow after the stimuli that initiated the new growth cease. Malignant neoplasms show partial or complete lack of structural organization and functional coordination with the normal tissue and most invade surrounding tissues, metastasize to several sites, and are likely to recur after attempted removal and to cause the death of the patient unless adequately treated. As used herein, the term neoplasia is used to describe all cancerous disease states and embraces or encompasses the pathological process associated with malignant hematogenous, ascitic and solid tumors. Exemplary cancers which may be treated by the present compounds either alone or in combination with at least one additional anti-cancer agent include squamous-cell carcinoma, basal cell carcinoma, adenocarcinoma, hepatocellular carcinomas, and renal cell carcinomas, cancer of the bladder, bowel, breast, cervix, colon, esophagus, head, kidney, liver, lung, neck, ovary, pancreas, prostate, and stomach; leukemias; benign and malignant lymphomas, particularly Burkitt's lymphoma and Non-Hodgkin's Lymphoma; benign and malignant melanomas; myeloproliferative diseases; sarcomas, including Ewing's sarcoma, hemangiosarcoma, Kaposi's sarcoma, liposarcoma, myosarcomas, peripheral neuroepithelioma, synovial sarcoma, gliomas, astrocytomas, oligodendrogliomas, ependymomas, gliobastomas, neuroblastomas, ganglioneuromas, gangliogliomas, medulloblastomas, pineal cell tumors, meningiomas, meningeal sarcomas, neurofibromas, and Schwannomas; bowel cancer, breast cancer, prostate cancer, cervical cancer, uterine cancer, lung cancer, ovarian cancer, testicular cancer, thyroid cancer, astrocytoma, esophageal cancer, pancreatic cancer, stomach cancer, liver cancer, colon cancer, melanoma; carcinosarcoma, Hodgkin's disease, Wilms' tumor and teratocarcinomas. Additional cancers which may be treated using compounds according to the present invention include, for example, T-lineage Acute lymphoblastic Leukemia (T-ALL), T-lineage lymphoblastic Lymphoma (T-LL), Peripheral T-cell lymphoma, Adult T-cell Leukemia, Pre-B ALL, Pre-B Lymphomas, Large B-cell Lymphoma, Burkitts Lymphoma, B-cell ALL, Philadelphia chromosome positive ALL and Philadelphia chromosome positive CML.

Additional cancers which may be treated using the disclosed compounds according to the present invention include, for example, acute granulocytic leukemia, acute lymphocytic leukemia (ALL), acute myelogenous leukemia (AML), adenocarcinoma, adenosarcoma, adrenal cancer, adrenocortical carcinoma, anal cancer, anaplastic astrocytoma, angiosarcoma, appendix cancer, astrocytoma, Basal cell carcinoma, B-Cell lymphoma, bile duct cancer, bladder cancer, bone cancer, bone marrow cancer, bowel cancer, brain cancer, brain stem glioma, breast cancer, triple (estrogen, progesterone and HER-2) negative breast cancer, double negative breast cancer (two of estrogen, progesterone and HER-2 are negative), single negative (one of estrogen, progesterone and HER-2 is negative), estrogen-receptor positive, HER2-negative breast cancer, estrogen receptor-negative breast cancer, estrogen receptor positive breast cancer, metastatic breast cancer, luminal A breast cancer, luminal B breast cancer, Her2-negative breast cancer, HER2-positive or negative breast cancer, progesterone receptor-negative breast cancer, progesterone receptor-positive breast cancer, recurrent breast cancer, carcinoid tumors, cervical cancer, cholangiocarcinoma, chondrosarcoma, chronic lymphocytic leukemia (CLL), chronic myelogenous leukemia (CML), colon cancer, colorectal cancer, craniopharyngioma, cutaneous lymphoma, cutaneous melanoma, diffuse astrocytoma, ductal carcinoma in situ (DCIS), endometrial cancer, ependymoma, epithelioid sarcoma, esophageal cancer, ewing sarcoma, extrahepatic bile duct cancer, eye cancer, fallopian tube cancer, fibrosarcoma, gallbladder cancer, gastric cancer, gastrointestinal cancer, gastrointestinal carcinoid cancer, gastrointestinal stromal tumors (GIST), germ cell tumor glioblastoma multiforme (GBM), glioma, hairy cell leukemia, head and neck cancer, hemangioendothelioma, Hodgkin lymphoma, hypopharyngeal cancer, infiltrating ductal carcinoma (IDC), infiltrating lobular carcinoma (ILC), inflammatory breast cancer (IBC), intestinal Cancer, intrahepatic bile duct cancer, invasive/infiltrating breast cancer, Islet cell cancer, jaw cancer, Kaposi sarcoma, kidney cancer, laryngeal cancer, leiomyosarcoma, leptomeningeal metastases, leukemia, lip cancer, liposarcoma, liver cancer, lobular carcinoma in situ, low-grade astrocytoma, lung cancer, lymph node cancer, lymphoma, male breast cancer, medullary carcinoma, medulloblastoma, melanoma, meningioma, Merkel cell carcinoma, mesenchymal chondrosarcoma, mesenchymous, mesothelioma metastatic breast cancer, metastatic melanoma metastatic squamous neck cancer, mixed gliomas, monodermal teratoma, mouth cancer mucinous carcinoma, mucosal melanoma, multiple myeloma, Mycosis Fungoides, myelodysplastic syndrome, nasal cavity cancer, nasopharyngeal cancer, neck cancer, neuroblastoma, neuroendocrine tumors (NETs), Non-Hodgkin's Lymphoma, non-small cell lung cancer (NSCLC), oat cell cancer, ocular cancer, ocular melanoma, oligodendroglioma, oral cancer, oral cavity cancer, oropharyngeal cancer, osteogenic sarcoma, osteosarcoma, ovarian cancer, ovarian epithelial cancer ovarian germ cell tumor, ovarian primary peritoneal carcinoma, ovarian sex cord stromal tumor, Paget's disease, pancreatic cancer, papillary carcinoma, paranasal sinus cancer, parathyroid cancer, pelvic cancer, penile cancer, peripheral nerve cancer, peritoneal cancer, pharyngeal cancer, pheochromocytoma, pilocytic astrocytoma, pineal region tumor, pineoblastoma, pituitary gland cancer, primary central nervous system (CNS) lymphoma, prostate cancer, rectal cancer, renal cell carcinoma, renal pelvis cancer, rhabdomyosarcoma, salivary gland cancer, soft tissue sarcoma, bone sarcoma, sarcoma, sinus cancer, skin cancer, small cell lung cancer (SCLC), small intestine cancer, spinal cancer, spinal column cancer, spinal cord cancer, squamous cell carcinoma, stomach cancer, synovial sarcoma, T-cell lymphoma, testicular cancer, throat cancer, thymoma/thymic carcinoma, thyroid cancer, tongue cancer, tonsil cancer, transitional cell cancer, tubal cancer, tubular carcinoma, undiagnosed cancer, ureteral cancer, urethral cancer, uterine adenocarcinoma, uterine cancer, uterine sarcoma, vaginal cancer, vulvar cancer, T-cell lineage acute lymphoblastic leukemia (T-ALL), T-cell lineage lymphoblastic lymphoma (T-LL), peripheral T-cell lymphoma, Adult T-cell leukemia, Pre-B ALL, Pre-B lymphomas, large B-cell lymphoma, Burkitts lymphoma, B-cell ALL, Philadelphia chromosome positive ALL, Philadelphia chromosome positive CML, juvenile myelomonocytic leukemia (JMML), acute promyelocytic leukemia (a subtype of AML), large granular lymphocytic leukemia, Adult T-cell chronic leukemia, diffuse large B cell lymphoma, follicular lymphoma; Mucosa-Associated Lymphatic Tissue lymphoma (MALT), small cell lymphocytic lymphoma, mediastinal large B cell lymphoma, nodal marginal zone B cell lymphoma (NMZL); splenic marginal zone lymphoma (SMZL); intravascular large B-cell lymphoma; primary effusion lymphoma; or lymphomatoid granulomatosis; B-cell prolymphocytic leukemia; splenic lymphoma/leukemia, unclassifiable, splenic diffuse red pulp small B-cell lymphoma; lymphoplasmacytic lymphoma; heavy chain diseases, for example, Alpha heavy chain disease, Gamma heavy chain disease, Mu heavy chain disease, plasma cell myeloma, solitary plasmacytoma of bone; extraosseous plasmacytoma; primary cutaneous follicle center lymphoma, T cell/histocyte rich large B-cell lymphoma, DLBCL associated with chronic inflammation; Epstein-Barr virus (EBV)+ DLBCL of the elderly; primary mediastinal (thymic) large B-cell lymphoma, primary cutaneous DLBCL, leg type, ALK+ large B-cell lymphoma, plasmablastic lymphoma; large B-cell lymphoma arising in HHV8-associated multicentric, Castleman disease; B-cell lymphoma, unclassifiable, with features intermediate between diffuse large B-cell lymphoma, or B-cell lymphoma, unclassifiable, with features intermediate between diffuse large B-cell lymphoma and classical Hodgkin lymphoma. In certain embodiments, the disorder is adenoid cystic carcinoma. In certain embodiments, the disorder is NUT midline carcinoma.

In another embodiment, a compound or its pharmaceutically acceptable salt, isotopic derivative or prodrug as described herein can be used in an effective amount to treat a host, for example a human, with an autoimmune disorder. Examples include, but are not limited to: Acute disseminated encephalomyelitis (ADEM); Addison's disease; Agammaglobulinemia; Alopecia areata; Amyotrophic lateral sclerosis (Also Lou Gehrig's disease; Motor Neuron Disease); Ankylosing Spondylitis; Antiphospholipid syndrome; Antisynthetase syndrome; Atopic allergy; Atopic dermatitis; Autoimmune aplastic anemia; Autoimmune arthritis; Autoimmune cardiomyopathy; Autoimmune enteropathy; Autoimmune granulocytopenia; Autoimmune hemolytic anemia; Autoimmune hepatitis; Autoimmune hypoparathyroidism; Autoimmune inner ear disease; Autoimmune lymphoproliferative syndrome; Autoimmune myocarditis; Autoimmune pancreatitis; Autoimmune peripheral neuropathy; Autoimmune ovarian failure; Autoimmune polyendocrine syndrome; Autoimmune progesterone dermatitis; Autoimmune thrombocytopenic purpura; Autoimmune thyroid disorders; Autoimmune urticarial; Autoimmune uveitis; Autoimmune vasculitis; Balo disease/Balo concentric sclerosis; Behçet's disease; Berger's disease; Bickerstaff s encephalitis; Blau syndrome; Bullous pemphigoid; Cancer; Castleman's disease; Celiac disease; Chagas disease; Chronic inflammatory demyelinating polyneuropathy; Chronic inflammatory demyelinating polyneuropathy; Chronic obstructive pulmonary disease; Chronic recurrent multifocal osteomyelitis; Churg-Strauss syndrome; Cicatricial pemphigoid; Cogan syndrome; Cold agglutinin disease; Complement component 2 deficiency; Contact dermatitis; Cranial arteritis; CREST syndrome; Crohn's disease; Cushing's Syndrome; Cutaneous leukocytoclastic angiitis; Dego's disease; Dercum's disease; Dermatitis herpetiformis; Dermatomyositis; Diabetes mellitus type 1; Diffuse cutaneous systemic sclerosis; Discoid lupus erythematosus; Dressler's syndrome; Drug-induced lupus; Eczema; Endometriosis; Enthesitis-related arthritis; Eosinophilic fasciitis; Eosinophilic gastroenteritis; Eosinophilic pneumonia; Epidermolysis bullosa acquisita; Erythema nodosum; Erythroblastosis fetalis; Essential mixed cryoglobulinemia; Evan's syndrome; Extrinsic and intrinsic reactive airways disease (asthma); Fibrodysplasia ossificans progressive; Fibrosing alveolitis (or Idiopathic pulmonary fibrosis); Gastritis; Gastrointestinal pemphigoid; Glomerulonephritis; Goodpasture's syndrome; Graves' disease; Guillain-Barre syndrome (GBS); Hashimoto's encephalopathy; Hashimoto's thyroiditis; Hemolytic anemia; Henoch-Schonlein purpura; Herpes gestationis (Gestational Pemphigoid); Hidradenitis suppurativa; Hughes-Stovin syndrome; Hypogammaglobulinemia; Idiopathic inflammatory demyelinating diseases; Idiopathic pulmonary fibrosis; Idiopathic thrombocytopenic purpura; IgA nephropathy; Immune glomerulonephritis; Immune nephritis; Immune pneumonitis; Inclusion body myositis; inflammatory bowel disease; Interstitial cystitis; Juvenile idiopathic arthritis aka Juvenile rheumatoid arthritis; Kawasaki's disease; Lambert-Eaton myasthenic syndrome; Leukocytoclastic vasculitis; Lichen planus; Lichen sclerosus; Linear IgA disease (LAD); Lupoid hepatitis aka Autoimmune hepatitis; Lupus erythematosus; Majeed syndrome; microscopic polyangiitis; Miller-Fisher syndrome; mixed connective tissue disease; Morphea; Mucha-Habermann disease aka Pityriasis lichenoides et varioliformis acuta; Multiple sclerosis; Myasthenia gravis; Myositis; Menibre's disease; Narcolepsy; Neuromyelitis optica (also Devic's disease); Neuromyotonia; Occular cicatricial pemphigoid; Opsoclonus myoclonus syndrome; Ord's thyroiditis; Palindromic rheumatism; PANDAS (pediatric autoimmune neuropsychiatric disorders associated with Streptococcus); Paraneoplastic cerebellar degeneration; Paroxysmal nocturnal hemoglobinuria (PNH); Parry Romberg syndrome; Pars planitis; Parsonage-Turner syndrome; Pemphigus vulgaris; Perivenous encephalomyelitis; Pernicious anaemia; POEMS syndrome; Polyarteritis nodosa; Polymyalgia rheumatic; Polymyositis; Primary biliary cirrhosis; Primary sclerosing cholangitis; Progressive inflammatory neuropathy; Psoriasis; Psoriatic arthritis; pure red cell aplasia; Pyoderma gangrenosum; Rasmussen's encephalitis; Raynaud phenomenon; Reiter's syndrome; relapsing polychondritis; restless leg syndrome; retroperitoneal fibrosis; rheumatic fever; rheumatoid arthritis; Sarcoidosis; Schizophrenia; Schmidt syndrome; Schnitzler syndrome; Scleritis; Scleroderma; Sclerosing cholangitis; serum sickness; Sjögren's syndrome; Spondyloarthropathy; Stiff person syndrome; Still's disease; Subacute bacterial endocarditis (SBE); Susac's syndrome; Sweet's syndrome; Sydenham chorea; sympathetic ophthalmia; systemic lupus erythematosus; Takayasu's arteritis; temporal arteritis (also known as “giant cell arteritis”); thrombocytopenia; Tolosa-Hunt syndrome; transverse myelitis; ulcerative colitis; undifferentiated connective tissue disease; undifferentiated spondyloarthropathy; urticarial vasculitis; vasculitis; vitiligo; viral diseases such as Epstein Barr Virus (EBV), Hepatitis B, Hepatitis C, HIV, HTLV 1, Varicella-Zoster Virus (VZV) and Human Papilloma Virus (HPV); or Wegener's granulomatosis. In some embodiments, the autoimmune disease is an allergic condition, including those from asthma, food allergies, atopic dermatitis, chronic pain, and rhinitis.

Cutaneous contact hypersensitivity and asthma are just two examples of immune responses that can be associated with significant morbidity. Others include atopic dermatitis, eczema, Sjogren's Syndrome, including keratoconjunctivitis sicca secondary to Sjogren's Syndrome, alopecia areata, allergic responses due to arthropod bite reactions, Crohn's disease, aphthous ulcer, iritis, conjunctivitis, keratoconjunctivitis, ulcerative colitis, cutaneous lupus erythematosus, scleroderma, vaginitis, proctitis, and drug eruptions. These conditions may result in any one or more of the following symptoms or signs: itching, swelling, redness, blisters, crusting, ulceration, pain, scaling, cracking, hair loss, scarring, or oozing of fluid involving the skin, eye, or mucosal membranes.

In atopic dermatitis, and eczema in general, immunologically mediated leukocyte infiltration (particularly infiltration of mononuclear cells, lymphocytes, neutrophils, and eosinophils) into the skin importantly contributes to the pathogenesis of these diseases. Chronic eczema also is associated with significant hyperproliferation of the epidermis. Immunologically mediated leukocyte infiltration also occurs at sites other than the skin, such as in the airways in asthma and in the tear producing gland of the eye in keratoconjunctivitis sicca.

A compound or its pharmaceutically acceptable salt, isotopic variant, or prodrug as described herein can be administered in an effective amount to treat a host, for example a human, with a skin disorder such as psoriasis (for example, psoriasis vulgaris), atopic dermatitis, skin rash, skin irritation, skin sensitization (e.g., contact dermatitis or allergic contact dermatitis). For example, certain substances including some pharmaceuticals when topically applied can cause skin sensitization. In some embodiments, the skin disorder is treated by topical administration of compounds known in the art in combination with the compounds disclosed herein. In one non-limiting embodiment the disorder of the present invention is selected from contact dermatitis, atopic dermatitis, eczematous dermatitis, psoriasis, Sjogren's Syndrome, including keratoconjunctivitis sicca secondary to Sjogren's Syndrome, alopecia areata, allergic responses due to arthropod bite reactions, Crohn's disease, aphthous ulcer, iritis, conjunctivitis, keratoconjunctivitis, ulcerative colitis, asthma, allergic asthma, cutaneous lupus erythematosus, scleroderma, vaginitis, proctitis, and drug eruptions.

Disease states of conditions which may be treated according to the present invention include, for example, asthma, autoimmune diseases such as multiple sclerosis, various cancers, ciliopathies, cleft palate, diabetes, heart disease, hypertension, inflammatory bowel disease, mental retardation, mood disorder, obesity, refractive error, infertility, Angelman syndrome, Canavan disease, Coeliac disease, Charcot-Marie-Tooth disease, Cystic fibrosis, Duchenne muscular dystrophy, Haemochromatosis, Hemophilia, Klinefelter's syndrome, Neurofibromatosis, Phenylketonuria, Polycystic kidney disease 1 (PKD1) or 2 (PKD2) Prader-Willi syndrome, Sickle-cell disease, Tay-Sachs disease, Turner syndrome.

Further disease states or conditions which may be treated according to the present invention include Alzheimer's disease, Amyotrophic lateral sclerosis (Lou Gehrig's disease), Anorexia nervosa, Anxiety disorder, Atherosclerosis, Attention deficit hyperactivity disorder, Autism, Bipolar disorder, Chronic fatigue syndrome, Chronic obstructive pulmonary disease, Crohn's disease, Coronary heart disease, Dementia, Depression, Diabetes mellitus type 1, Diabetes mellitus type 2, Epilepsy, Guillain-Barre syndrome, Irritable bowel syndrome, Lupus, Metabolic syndrome, Multiple sclerosis, Myocardial infarction, Obesity, Obsessive-compulsive disorder, Panic disorder, Parkinson's disease, Psoriasis, Rheumatoid arthritis, Sarcoidosis, Schizophrenia, Stroke, Thromboangiitis obliterans, Tourette syndrome, Vasculitis.

Still additional disease states or conditions which can be treated according to the present invention include aceruloplasminemia, Achondrogenesis type IL, achondroplasia, Acrocephaly, Gaucher disease type 2, acute intermittent porphyria, Canavan disease, Adenomatous Polyposis Coli, ALA dehydratase deficiency, adenylosuccinate lyase deficiency, Adrenogenital syndrome, Adrenoleukodystrophy, ALA-D porphyria, ALA dehydratase deficiency, Alkaptonuria, Alexander disease, Alkaptonuric ochronosis, alpha 1-antitrypsin deficiency, alpha-1 proteinase inhibitor, emphysema, amyotrophic lateral sclerosis Alstrom syndrome, Alexander disease, Amelogenesis imperfecta, ALA dehydratase deficiency, Anderson-Fabry disease, androgen insensitivity syndrome, Anemia Angiokeratoma Corporis Diffusum, Angiomatosis retinae (von Hippel-Lindau disease) Apert syndrome, Arachnodactyly (Marfan syndrome), Stickler syndrome, Arthrochalasis multiplex congenital (Ehlers-Danlos syndrome#arthrochalasia type) ataxia telangiectasia, Rett syndrome, primary pulmonary hypertension, Sandhoff disease, neurofibromatosis type IL, Beare-Stevenson cutis gyrata syndrome, Mediterranean fever, familial, Benjamin syndrome, beta-thalassemia, Bilateral Acoustic Neurofibromatosis (neurofibromatosis type II), factor V Leiden thrombophilia, Bloch-Sulzberger syndrome (incontinentia pigmenti), Bloom syndrome, X-linked sideroblastic anemia, Bonnevie-Ullrich syndrome (Turner syndrome), Bourneville disease (tuberous sclerosis), prion disease, Birt-Hogg-Dube syndrome, Brittle bone disease (osteogenesis imperfecta), Broad Thumb-Hallux syndrome (Rubinstein-Taybi syndrome), Bronze Diabetes/Bronzed Cirrhosis (hemochromatosis), Bulbospinal muscular atrophy (Kennedy's disease), Burger-Grutz syndrome (lipoprotein lipase deficiency), CGD Chronic granulomatous disorder, Campomelic dysplasia, biotinidase deficiency, Cardiomyopathy (Noonan syndrome), Cri du chat, CAVD (congenital absence of the vas deferens), Caylor cardiofacial syndrome (CBAVD), CEP (congenital erythropoietic porphyria), cystic fibrosis, congenital hypothyroidism, Chondrodystrophy syndrome (achondroplasia), otospondylomegaepiphyseal dysplasia, Lesch-Nyhan syndrome, galactosemia, Ehlers-Danlos syndrome, Thanatophoric dysplasia, Coffin-Lowry syndrome, Cockayne syndrome, (familial adenomatous polyposis), Congenital erythropoietic porphyria, Congenital heart disease, Methemoglobinemia/Congenital methaemoglobinaemia, achondroplasia, X-linked sideroblastic anemia, Connective tissue disease, Conotruncal anomaly face syndrome, Cooley's Anemia (beta-thalassemia), Copper storage disease (Wilson's disease), Copper transport disease (Menkes disease), hereditary coproporphyria, Cowden syndrome, Craniofacial dysarthrosis (Crouzon syndrome), Creutzfeldt-Jakob disease (prion disease), Cockayne syndrome, Cowden syndrome, Curschmann-Batten-Steinert syndrome (myotonic dystrophy), Beare-Stevenson cutis gyrata syndrome, primary hyperoxaluria, spondyloepimetaphyseal dysplasia (Strudwick type), muscular dystrophy, Duchenne and Becker types (DBMD), Usher syndrome, Degenerative nerve diseases including de Grouchy syndrome and Dejerine-Sottas syndrome, developmental disabilities, distal spinal muscular atrophy, type V, androgen insensitivity syndrome, Diffuse Globoid Body Sclerosis (Krabbe disease), Di George's syndrome, Dihydrotestosterone receptor deficiency, androgen insensitivity syndrome, Down syndrome, Dwarfism, erythropoietic protoporphyria Erythroid 5-aminolevulinate synthetase deficiency, Erythropoietic porphyria, erythropoietic protoporphyria, erythropoietic uroporphyria, Friedreich's ataxia-familial paroxysmal polyserositis, porphyria cutanea tarda, familial pressure sensitive neuropathy, primary pulmonary hypertension (PPH), Fibrocystic disease of the pancreas, fragile X syndrome, galactosemia, genetic brain disorders, Giant cell hepatitis (Neonatal hemochromatosis), Gronblad-Strandberg syndrome (pseudoxanthoma elasticum), Gunther disease (congenital erythropoietic porphyria), haemochromatosis, Hallgren syndrome, sickle cell anemia, hemophilia, hepatoerythropoietic porphyria (HEP), Hippel-Lindau disease (von Hippel-Lindau disease), Huntington's disease, Hutchinson-Gilford progeria syndrome (progeria), Hyperandrogenism, Hypochondroplasia, Hypochromic anemia, Immune system disorders, including X-linked severe combined immunodeficiency, Insley-Astley syndrome, Jackson-Weiss syndrome, Joubert syndrome, Lesch-Nyhan syndrome, Jackson-Weiss syndrome, Kidney diseases, including hyperoxaluria, Klinefelter's syndrome, Kniest dysplasia, Lacunar dementia, Langer-Saldino achondrogenesis, ataxia telangiectasia, Lynch syndrome, Lysyl-hydroxylase deficiency, Machado-Joseph disease, Metabolic disorders, including Kniest dysplasia, Marfan syndrome, Movement disorders, Mowat-Wilson syndrome, cystic fibrosis, Muenke syndrome, Multiple neurofibromatosis, Nance-Insley syndrome, Nance-Sweeney chondrodysplasia, Niemann-Pick disease, Noack syndrome (Pfeiffer syndrome), Osler-Weber-Rendu disease, Peutz-Jeghers syndrome, Polycystic kidney disease, polyostotic fibrous dysplasia (McCune-Albright syndrome), Peutz-Jeghers syndrome, Prader-Labhart-Willi syndrome, hemochromatosis, primary hyperuricemia syndrome (Lesch-Nyhan syndrome), primary pulmonary hypertension, primary senile degenerative dementia, prion disease, progeria (Hutchinson Gilford Progeria Syndrome), progressive chorea, chronic hereditary (Huntington) (Huntington's disease), progressive muscular atrophy, spinal muscular atrophy, propionic acidemia, protoporphyria, proximal myotonic dystrophy, pulmonary arterial hypertension, PXE (pseudoxanthoma elasticum), Rb (retinoblastoma), Recklinghausen disease (neurofibromatosis type I), Recurrent polyserositis, Retinal disorders, Retinoblastoma, Rett syndrome, RFALS type 3, Ricker syndrome, Riley-Day syndrome, Roussy-Levy syndrome, severe achondroplasia with developmental delay and acanthosis nigricans (SADDAN), Li-Fraumeni syndrome, sarcoma, breast, leukemia, and adrenal gland (SBLA) syndrome, sclerosis tuberose (tuberous sclerosis), SDAT, SED congenital (spondyloepiphyseal dysplasia congenita), SED Strudwick (spondyloepimetaphyseal dysplasia, Strudwick type), SEDc (spondyloepiphyseal dysplasia congenita) SEMD, Strudwick type (spondyloepimetaphyseal dysplasia, Strudwick type), Shprintzen syndrome, Skin pigmentation disorders, Smith-Lemli-Opitz syndrome, South-African genetic porphyria (variegate porphyria), infantile-onset ascending hereditary spastic paralysis, Speech and communication disorders, sphingolipidosis, Tay-Sachs disease, spinocerebellar ataxia, Stickler syndrome, stroke, androgen insensitivity syndrome, tetrahydrobiopterin deficiency, beta-thalassemia, Thyroid disease, Tomaculous neuropathy (hereditary neuropathy with liability to pressure palsies), Treacher Collins syndrome, Triplo X syndrome (triple X syndrome), Trisomy 21 (Down syndrome), Trisomy X, VHL syndrome (von Hippel-Lindau disease), Vision impairment and blindness (Alstrom syndrome), Vrolik disease, Waardenburg syndrome, Warburg Sjo Fledelius Syndrome, Wolf-Hirschhorn syndrome, Wolff Periodic disease, Weissenbacher-Zweymuller syndrome and Xeroderma pigmentosum, among others.

In certain embodiments, a treatment is provided for multiple myeloma comprising administering to a patient an effective amount of a compound described herein, or a pharmaceutically acceptable salt, isotopic analog, or prodrug thereof, optionally in a pharmaceutically acceptable carrier to form a composition. In another embodiment, a compound described herein, or a pharmaceutically acceptable salt, isotopic analog, or prodrug thereof, optionally in a pharmaceutically acceptable carrier to form a composition, for use in a treatment of multiple myeloma, wherein the treatment comprises administering the compound to a patient.

In certain embodiments, a treatment is provided for managing the progression of multiple myeloma comprising administering to a patient an effective amount of a compound described herein, or a pharmaceutically acceptable salt, isotopic analog, or prodrug thereof, optionally in a pharmaceutically acceptable carrier to form a composition. In another embodiment, a compound described herein, or a pharmaceutically acceptable salt, isotopic analog, or prodrug thereof, optionally in a pharmaceutically acceptable carrier to form a composition, for use in a treatment for managing the progression of multiple myeloma, wherein the treatment comprises administering the compound to a patient.

Treatments are also provided for patients who have been previously treated for multiple myeloma but are non-responsive to standard therapies in addition to those who have not been previously treated. Additional treatments are provided for patients who have undergone surgery in an attempt to treat multiple myeloma in addition to those who have not undergone surgery. Treatments are also provided for patients who have previously undergone transplant therapy in addition to those who have not.

In certain embodiments, the disorder treated by the present invention is a wild-type cancer, wherein the term “wild-type” refers to a cancer that has not developed resistance to a previously effective treatment (i.e. a relapsed cancer) and does not have any resistance imparting mutations (i.e. a refractory cancer). In certain embodiments, the disorder treated by the present invention is a relapsed cancer. In certain embodiments, the disorder treated by the present invention is a refractory cancer. In certain embodiments, the disorder treated by the present invention is a relapsed and refractory cancer.

In certain embodiments the cancer is not resectable. In other embodiments the cancer is resectable. When the cancer is resectable the method described herein can be conducted with or without a surgical operation to resect the cancer. In certain embodiments the cancer patient has a cancer that is histologically and/or cytologically-confirmed, for example histologically or cytologically-confirmed non-Hodgkin's lymphoma or multiple myeloma.

A compound described herein, for example, Compound 1 or a pharmaceutically acceptable salt thereof, can be administered in the treatment or management of multiple myeloma or Non-Hodgkin's Lymphoma that is relapsed, refractory, or resistant. In some embodiments, the disorder is primary, secondary, tertiary, quadruply or quintuply relapsed. In certain embodiments, the compounds described herein may be used to reduce, maintain, or eliminate minimal residual disease (MRD).

The types of multiple myeloma that may be treated with Compound 1 include, but are not limited to: monoclonal gammopathy of undetermined significance (MGUS); low risk, intermediate risk, or high risk multiple myeloma; newly diagnosed multiple myeloma, including low risk, intermediate risk, or high risk newly diagnosed multiple myeloma); transplant eligible and transplant ineligible multiple myeloma; smoldering (indolent) multiple myeloma (including low risk, intermediate risk, or high risk smoldering multiple myeloma); active multiple myeloma; solitary plasmocytoma; plasma cell leukemia; central nervous system multiple myeloma; light chain myeloma; non-secretory myeloma; Immunoglobulin D myeloma; and Immunoglobulin E myeloma. Additional types of multiple myeloma that may be treated with Compound 1 include Immunoglobulin A myeloma, Immunoglobulin M myeloma, and Immunoglobulin G myeloma,

In certain embodiments, a treatment is provided for managing multiple myeloma comprising administering to a patient an effective amount of a compound described herein, or a pharmaceutically acceptable salt, isotopic analog, or prodrug thereof, optionally in a pharmaceutically acceptable carrier to form a composition, as induction therapy.

In certain embodiments, a treatment is provided for treating or managing multiple myeloma comprising administering to a patient an effective amount of a compound described herein, or a pharmaceutically acceptable salt, isotopic analog, or prodrug thereof, optionally in a pharmaceutically acceptable carrier to form a composition, as consolidation therapy.

In certain embodiments, a method is provided for treating or managing multiple myeloma comprising administering to a patient an effective amount of a compound described herein, or a pharmaceutically acceptable salt, isotopic analog, or prodrug thereof, optionally in a pharmaceutically acceptable carrier to form a composition, as maintenance therapy.

In certain embodiments, the multiple myeloma is plasma cell leukemia.

In certain embodiments, the multiple myeloma is high risk multiple myeloma. In some embodiments, the high-risk multiple myeloma is relapsed or refractory. In certain embodiments, the high-risk multiple myeloma has relapsed within 12 months of the first treatment. In another embodiment, the high-risk multiple myeloma is characterized by genetic abnormalities, for example, one or more of del(17/17p) and t(14;16)(q32;q32). In some embodiments, the high risk multiple myeloma is relapsed or refractory to one, two or three previous treatments.

In some embodiments, the multiple myeloma is transplant eligible newly diagnosed multiple myeloma. In other embodiments, the multiple myeloma is transplant ineligible newly diagnosed multiple myeloma.

In some embodiments, the multiple myeloma shows early progression (for example less than 12 months) following initial treatment. In other embodiments, the multiple myeloma shows early progression (for example less than 12 months) following autologous stem cell transplant. In another embodiment, the multiple myeloma is refractory to lenalidomide. In another embodiment, the multiple myeloma is refractory to pomalidomide. In some such embodiments, the multiple myeloma is predicted to be refractory to pomalidomide (for example, by molecular characterization). In another embodiment, the multiple myeloma is relapsed or refractory to 3 or more treatments and was exposed to a proteasome inhibitor (for example, bortezomib, carfilzomib, ixazomib, oprozomib, or marizomib) and an immunomodulatory compound (for example thalidomide, lenalidomide, pomalidomide, iberdomide, or avadomide), or double refractory to a proteasome inhibitor and an immunomodulatory compound. In still other embodiments, the multiple myeloma is relapsed or refractory to 3 or more prior therapies, including for example, a CD38 monoclonal antibody (CD38 mAb, for example, daratumumab or isatuximab), a proteasome inhibitor (for example, bortezomib, carfilzomib, ixazomib, or marizomib), and an immunomodulatory compound (for example thalidomide, lenalidomide, pomalidomide, iberdomide, or avadomide) or double refractory to a proteasome inhibitor or immunomodulatory compound and a CD38 mAb. In still other embodiments, the multiple myeloma is triple refractory, for example, the multiple myeloma is refractory to a proteasome inhibitor (for example, bortezomib, carfilzomib, ixazomib, oprozomib or marizomib), an immunomodulatory compound (for example thalidomide, lenalidomide, pomalidomide, iberdomide, or avadomide), and one other active agent, as described herein.

In another aspect the cancer has a downregulated level of cereblon. In certain embodiments the cancer has a downregulated concentration of cereblon after prior treatment with lenalidomide. In certain embodiments the cancer has a downregulated concentration of cereblon after prior treatment with pomalidomide.

In another aspect the multiple myeloma has a downregulated level of cereblon. In certain embodiments the multiple myeloma has a downregulated concentration of cereblon after prior treatment with lenalidomide. In certain embodiments the multiple myeloma has a downregulated concentration of cereblon after prior treatment with pomalidomide.

In another aspect the non-Hodgkin Lymphoma has a downregulated level of cereblon. In certain embodiments the non-Hodgkin Lymphoma has a downregulated concentration of cereblon after prior treatment with lenalidomide. In certain embodiments the non-Hodgkin Lymphoma has a downregulated concentration of cereblon after prior treatment with pomalidomide.

In certain embodiments, a treatment is provided for managing relapsed or refractory multiple myeloma in patients with impaired renal function or a symptom thereof comprising administering to a patient an effective amount of a compound described herein, or a pharmaceutically acceptable salt, isotopic analog, or prodrug thereof, optionally in a pharmaceutically acceptable carrier to form a composition.

In another embodiment, a treatment is provided for managing relapsed or refractory multiple myeloma in frail patients comprising administering to a patient an effective amount of a compound described herein, or a pharmaceutically acceptable salt, isotopic analog, or prodrug thereof, optionally in a pharmaceutically acceptable carrier to form a composition, wherein the frail patient is characterized by ineligibility for induction therapy or intolerance to dexamethasone treatment. In other embodiments, the frail patient is elderly, for example, older than 65 years old.

In another embodiment, a treatment is provided for managing fourth line relapsed or refractory multiple myeloma comprising administering to a patient an effective amount of a compound described herein, or a pharmaceutically acceptable salt, isotopic analog, or prodrug thereof, optionally in a pharmaceutically acceptable carrier to form a composition.

In another embodiment, a treatment is provided for managing newly diagnosed, transplant-ineligible multiple myeloma comprising administering to a patient an effective amount of a compound described herein, or a pharmaceutically acceptable salt, isotopic analog, or prodrug thereof, optionally in a pharmaceutically acceptable carrier to form a composition.

In another embodiment, a treatment is provided for managing newly diagnosed, transplant-ineligible multiple myeloma comprising administering to a patient an effective amount of a compound described herein, or a pharmaceutically acceptable salt, isotopic analog, or prodrug thereof, optionally in a pharmaceutically acceptable carrier to form a composition, as maintenance therapy after another therapy or transplant.

In another embodiment, a treatment is provided for managing high risk multiple myeloma that is relapsed or refractory to one, two, or three previous treatments comprising administering to a patient an effective amount of a compound described herein, or a pharmaceutically acceptable salt, isotopic analog, or prodrug thereof, optionally in a pharmaceutically acceptable carrier to form a composition.

In certain embodiments, the disorder treated by the present invention is a relapsed and/or refractory Non-Hodgkin's Lymphoma. In certain embodiments, the disorder treated by the present invention is a relapsed Non-Hodgkin's Lymphoma. In certain embodiments, the disorder treated by the present invention is a refractory Non-Hodgkin's Lymphoma. In certain embodiments, the disorder treated by the present invention is a relapsed and refractory Non-Hodgkin's Lymphoma.

In certain embodiments, the disorder treated by the present invention is a relapsed and/or refractory multiple myeloma. In certain embodiments, the disorder treated by the present invention is a relapsed multiple myeloma. In certain embodiments, the disorder treated by the present invention is a refractory multiple myeloma. In certain embodiments, the disorder treated by the present invention is a relapsed and refractory multiple myeloma.

In certain embodiments, the disorder treated by the present invention is a relapsed and/or refractory non-Hodgkin's lymphoma. In certain embodiments, the disorder treated by the present invention is a relapsed non-Hodgkin's lymphoma. In certain embodiments, the disorder treated by the present invention is a refractory non-Hodgkin's lymphoma. In certain embodiments, the disorder treated by the present invention is a relapsed and refractory non-Hodgkin's lymphoma.

In certain embodiments, the disorder treated by the present invention is a relapsed and/or refractory peripheral T-cell lymphoma. In certain embodiments, the disorder treated by the present invention is a relapsed peripheral T-cell lymphoma. In certain embodiments, the disorder treated by the present invention is a refractory peripheral T-cell lymphoma. In certain embodiments, the disorder treated by the present invention is a relapsed and refractory peripheral T-cell lymphoma.

In certain embodiments, Compound 1 is administered for the treatment of PTCL-NOS (i.e. PTCL that is not otherwise specified). In other embodiments Compound 1 is administered for the treatment of PTCL with a specified subtype for example anaplastic large cell lymphoma (ALCL), angioimmunoblastic T-Cell lymphoma (AITL), enteropathy-type T-cell lymphoma, or extranodal natural killer (NK) cell/T-cell lymphoma.

In certain embodiments, the disorder treated by the present invention is a relapsed and/or refractory systemic anaplastic large cell lymphoma (ALK⁺). In certain embodiments, the disorder treated by the present invention is a relapsed systemic anaplastic large cell lymphoma (ALK⁺). In certain embodiments, the disorder treated by the present invention is a refractory systemic anaplastic large cell lymphoma (ALK⁺). In certain embodiments, the disorder treated by the present invention is a relapsed and refractory systemic anaplastic large cell lymphoma (ALK⁺).

In certain embodiments, the disorder treated by the present invention is a relapsed and/or refractory systemic anaplastic large cell lymphoma (ALK⁻). In certain embodiments, the disorder treated by the present invention is a relapsed systemic anaplastic large cell lymphoma (ALK⁻). In certain embodiments, the disorder treated by the present invention is a refractory systemic anaplastic large cell lymphoma (ALK⁻). In certain embodiments, the disorder treated by the present invention is a relapsed and refractory systemic anaplastic large cell lymphoma (ALK⁻).

In certain embodiments, the disorder treated by the present invention is a relapsed and/or refractory angioimmunoblastic T-cell lymphoma. In certain embodiments, the disorder treated by the present invention is a relapsed angioimmunoblastic T-cell lymphoma. In certain embodiments, the disorder treated by the present invention is a refractory angioimmunoblastic T-cell lymphoma. In certain embodiments, the disorder treated by the present invention is a relapsed and refractory angioimmunoblastic T-cell lymphoma.

In certain embodiments, the disorder treated by the present invention is a relapsed and/or refractory anaplastic large cell lymphoma. In certain embodiments, the disorder treated by the present invention is a relapsed anaplastic large cell lymphoma. In certain embodiments, the disorder treated by the present invention is a refractory anaplastic large cell lymphoma. In certain embodiments, the disorder treated by the present invention is a relapsed and refractory anaplastic large cell lymphoma.

In certain embodiments, the disorder treated by the present invention is a relapsed and/or refractory mantle cell lymphoma. In certain embodiments, the disorder treated by the present invention is a relapsed mantle cell lymphoma. In certain embodiments, the disorder treated by the present invention is a refractory mantle cell lymphoma. In certain embodiments, the disorder treated by the present invention is a relapsed and refractory mantle cell lymphoma.

In certain embodiments, the disorder treated by the present invention is a relapsed and/or refractory follicular lymphoma. In certain embodiments, the disorder treated by the present invention is a relapsed follicular lymphoma. In certain embodiments, the disorder treated by the present invention is a refractory follicular lymphoma. In certain embodiments, the disorder treated by the present invention is a relapsed and refractory follicular lymphoma.

In certain embodiments, the disorder treated by the present invention is a relapsed and/or refractory follicular T-cell lymphoma. In certain embodiments, the disorder treated by the present invention is a relapsed follicular T-cell lymphoma. In certain embodiments, the disorder treated by the present invention is a refractory follicular T-cell lymphoma. In certain embodiments, the disorder treated by the present invention is a relapsed and refractory follicular T-cell lymphoma.

In certain embodiments, the disorder treated by the present invention is a relapsed and/or refractory peripheral T-cell lymphoma. In certain embodiments, the disorder treated by the present invention is a relapsed peripheral T-cell lymphoma. In certain embodiments, the disorder treated by the present invention is a refractory peripheral T-cell lymphoma. In certain embodiments, the disorder treated by the present invention is a relapsed and refractory peripheral T-cell lymphoma.

In certain embodiments, the disorder treated by the present invention is a relapsed and/or refractory diffuse large B-cell lymphoma. In certain embodiments, the disorder treated by the present invention is a relapsed diffuse large B-cell lymphoma. In certain embodiments, the disorder treated by the present invention is a refractory diffuse large B-cell lymphoma. In certain embodiments, the disorder treated by the present invention is a relapsed and refractory diffuse large B-cell lymphoma.

In certain embodiments, Compound 1 is administered to a patient in need thereof in an effective amount to treat follicular T-cell lymphoma. In certain embodiments, Compound 1 is administered to a patient in need thereof in an effective amount to treat angioimmunoblastic T-cell lymphoma. In certain embodiments, Compound 1 is administered to a patient in need thereof in an effective amount to treat systemic anaplastic large cell lymphoma (ALK⁻). In certain embodiments, Compound 1 is administered to a patient in need thereof in an effective amount to treat systemic anaplastic large cell lymphoma (ALK⁺).

Additional examples of Non-Hodgkin's Lymphomas that can be treated with a compound described herein include Double hit lymphoma, triple hit lymphoma, extranodal marginal zone B-cell lymphoma of MALT, extranodal NK/T-cell lymphoma, and myeloid-lineage lymphoma.

Treatment of IKZF1/IKZF3 Mediated Cancers with Prominent Mutations

In certain embodiments, Compound 1 is administered in an effective amount to treat a cancer that is mediated by a protein with one or more mutations, for example a multiple myeloma mediated by a protein with one or more mutations.

In some embodiments, the compounds described herein may be administered in an effective amount in the treatment or management of multiple myeloma characterized by a genetic abnormality, for example but not limited to: Cyclin D translocations (for example, t(11;14)(q13;q32); t(6;14)(p21;32); t(12;14)(p13;q32); or t(6;20);); MMSET translocations (for example t(4;14)(p16;q32); MAF translocations (for example t(14;16)(q32;a32); t(20;22); t(16;22)(q11;q13); or t(14;20)(q32;q11); or other chromosome factor (for example deletion of 17p13 or chromosome 13; del(17/17p), nonhyperdiploid, and gain (1q)).

In certain embodiments, the multiple myeloma has a p53 mutation. In certain embodiments, the p53 mutation is a Q331 mutation. In certain embodiments, the p53 mutation is a R273H mutation. In certain embodiments, the p53 mutation is a K132 mutation. In certain embodiments, the p53 mutation is a K132N mutation. In certain embodiments, the p53 mutation is a R337 mutation. In certain embodiments, the p53 mutation is a R337L mutation. In certain embodiments, the p53 mutation is a W146 mutation. In certain embodiments, the p53 mutation is a S261 mutation. In certain embodiments, the p53 mutation is a S261T mutation. In certain embodiments, the p53 mutation is a E286 mutation. In certain embodiments, the p53 mutation is a E286K mutation. In certain embodiments, the p53 mutation is a R175 mutation. In certain embodiments, the p53 mutation is a R175H mutation. In certain embodiments, the p53 mutation is a E258 mutation. In certain embodiments, the p53 mutation is a E258K mutation. In certain embodiments, the p53 mutation is a A161 mutation. In certain embodiments, the p53 mutation is a A161T mutation.

In certain embodiments, the multiple myeloma has a homozygous deletion of p53. In certain embodiments, the multiple myeloma has a homozygous deletion of wild-type p53. In certain embodiments, the multiple myeloma has wild-type p53.

In certain embodiments, the multiple myeloma shows activation of one or more oncogenic drivers. In certain embodiments, the one or more oncogenic drivers are selected from the group consisting of C-MAF, MAFB, FGFR3, MMset, Cyclin D1, and Cyclin D. In certain embodiments, the multiple myeloma shows activation of C-MAF. In certain embodiments, the multiple myeloma shows activation of MAFB. In certain embodiments, the multiple myeloma shows activation of FGFR3 and MMset. In certain embodiments, the multiple myeloma shows activation of C-MAF, FGFR3, and MMset. In certain embodiments, the multiple myeloma shows activation of Cyclin D1. In certain embodiments, the multiple myeloma shows activation of MAFB and Cyclin D1. In certain embodiments, the multiple myeloma shows activation of Cyclin D.

In certain embodiments, the multiple myeloma has one or more chromosomal translocations. In certain embodiments, the chromosomal translocation is t(14;16). In certain embodiments, the chromosomal translocation is t(14;20). In certain embodiments, the chromosomal translocation is t(4; 14). In certain embodiments, the chromosomal translocations are t(4;14) and t(14;16). In certain embodiments, the chromosomal translocation is t(11;14). In certain embodiments, the chromosomal translocation is t(6;20). In certain embodiments, the chromosomal translocation is t(20;22). In certain embodiments, the chromosomal translocations are t(6;20) and t(20;22). In certain embodiments, the chromosomal translocation is t(16;22). In certain embodiments, the chromosomal translocations are t(14;16) and t(16;22). In certain embodiments, the chromosomal translocations are t(14;20) and t(11;14).

In certain embodiments, the multiple myeloma has a Q331 p53 mutation, activation of C-MAF, and a chromosomal translocation at t(14; 16). In certain embodiments, the multiple myeloma has homozygous deletion of p53, activation of C-MAF, and a chromosomal translocation at t(14; 16). In certain embodiments, the multiple myeloma has a K132N p53 mutation, activation of MAFB, and a chromosomal translocation at t(14;20). In certain embodiments, the multiple myeloma has wild type p53, activation of FGFR3 and MMset, and a chromosomal translocation at t(4; 14). In certain embodiments, the multiple myeloma has wild type p53, activation of C-MAF, and a chromosomal translocation at t(14;16). In certain embodiments, the multiple myeloma has homozygous deletion of p53, activation of FGFR3, MMset, and C-MAF, and chromosomal translocations at t(4;14) and t(14;16). In certain embodiments, the multiple myeloma has homozygous deletion of p53, activation of Cyclin D1, and a chromosomal translocation at t(11;14). In certain embodiments, the multiple myeloma has a R337L p53 mutation, activation of Cyclin D1, and a chromosomal translocation at t(11;14). In certain embodiments, the multiple myeloma has a W146 p53 mutation, activation of FGFR3 and MMset, and a chromosomal translocation at t(4; 14). In certain embodiments, the multiple myeloma has a S261T p53 mutation, activation of MAFB, and chromosomal translocations at t(6;20) and t(20;22). In certain embodiments, the multiple myeloma has a E286K p53 mutation, by activation of FGFR3 and MMset, and a chromosomal translocation at t(4; 14). In certain embodiments, the multiple myeloma has a R175H p53 mutation, activation of FGFR3 and MMset, and a chromosomal translocation at t(4; 14). In certain embodiments, the multiple myeloma has a E258K p53 mutation, activation of C-MAF, and chromosomal translocations at t(14;16) and t(16;22). In certain embodiments, the multiple myeloma has wild type p53, activation of MAFB and Cyclin D1, and chromosomal translocations at t(14;20) and t(11;14). In certain embodiments, the multiple myeloma has a A161T p53 mutation, activation of Cyclin D, and a chromosomal translocation at t(11;14).

### Combination Therapy

Compound 1 Form I or a pharmaceutical composition comprising or prepared from Compound 1 Form I, or Compound 1 as part of a new dosage regimen described herein can be administered in an effective amount alone or in combination to treat a host such as a human with a disorder as described herein. In certain embodiments, a compound described herein is administered with an additional bioactive agent.

The term “bioactive agent” is used to describe an agent, other than the compound according to the present invention, which can be administered in combination or alternation with a compound of the present invention to achieve a desired result of therapy. In certain embodiments, the morphic form or pharmaceutical composition of the present invention and the bioactive agent are administered in a manner that they are active in vivo during overlapping time periods, for example, have time-period overlapping Cmax, Tmax, AUC or another pharmacokinetic parameter. In another embodiment, the morphic form or pharmaceutical composition of the present invention and the bioactive agent are administered to a host in need thereof that do not have overlapping pharmacokinetic parameter, however, one has a therapeutic impact on the therapeutic efficacy of the other.

As used herein when a compound is administered in combination with another this combination can be as one dosage form or multiple dosage forms at the same time or different times. Additionally, the compounds administered in combination can be administered with different dosing schedules. For example, the combination of Compound 1 with dexamethasone may be accomplished with a treatment regimen where Compound 1 is administered once a day for 21 consecutive days in a 28-day treatment cycle and dexamethasone is administered once a week during the treatment cycle. Alternative dosing schedules include Compound 1 administration once a day for 14 consecutive days in a 28-day treatment cycle and dexamethasone is administered once a week during the treatment cycle or Compound 1 administration three times a week for two or three consecutive weeks in a 28-day treatment cycle and dexamethasone is administered once a week during the treatment cycle.

### Corticosteroids

In certain embodiments, the morphic form or pharmaceutical composition of the present invention is administered in combination with a corticosteroid. In certain embodiments, the corticosteroid is dexamethasone.

| Study | Dosage | Dose | | Route of |
|---|---|---|---|---|
| Drug(s) | Formulation | Level(s) | Frequency | Administration |
| Compound 1 | Capsule | Table A | QD 21/7 | Oral |
| | or Tablet | | or QD 14/14 | |
| ≤75 years old | Tablet | 40 mg | Once/week | Oral |
| Dexamethasone | | | (QW) on days | |
| | | | 1, 8, 15, 22 | |
| >75 years old | Tablet | 20 mg | Once/week | Oral |
| Dexamethasone | | | (QW) on days | |
| | | | 1, 8, 15, | |
| | | | and 22 | |

In certain aspects Compound 1 is dosed QD on a dosing 14/14 or 21/7 schedule for each 28-day cycle in combination with another bioactive agent, for example once a week dosing of dexamethasone. In certain embodiments, the dexamethasone dose for an adult ≤75 years old is 40 mg QW on days 1, 8, 15, and 22 of a 28-day cycle. In certain embodiments, the dexamethasone dose for an adult >75 years old is 20 mg QW on days 1, 8, 15, and 22 of a 28-day cycle. In certain aspects Compound 1 is dosed QD on a dosing 7/14 schedule for each 21-day cycle in combination with another bioactive agent, for example once a week dosing of dexamethasone. In certain embodiments, the dexamethasone dose for an adult ≤75 years old is 40 mg QW on days 1, 8, and 15 of a 21-day cycle. In certain embodiments, the dexamethasone dose for an adult >75 years old is 20 mg QW on days 1, 8, and 15 of a 21-day cycle.

In certain embodiments, a morphic form or pharmaceutical composition described herein is administered in combination with a corticosteroid. Non-limiting examples of corticosteroids include dexamethasone, prednisone, fludrocortisone, hydrocortisone, cortisone, betamethasone, methylprednisolone.

In certain embodiments, the corticosteroid is dexamethasone.

Additional non-limiting examples of corticosteroids include corticosterone, aldosterone, prednisolone, triamcinolone, budesonide, deflazacort, flugestone, fluorometholone, medrysone, prebediolone acetate, chloroprednisone, cloprednol, difluprednate, fluocinolone, fluperolone, fluperolone acetate, fluprednisolone, loteprednol, prednicarbate, tixocortol, alclometasone, alclometasone dipropionate, beclometasone, clobetasol, clobetasone, clocortolone, desoximetasone, diflorasone, diflorasone diacetate, difluocortolone, difluocortolone valerate, fluprednidene, fluprednidene acetate, fluticasone, fluticasone furoate, halometasone, meprednisone, mometasone, mometasone furoate, paramethasone, prednylidene, rimexolone, ulobetasol, amcinonide, ciclesonide, desonide, formocortal, fluclorolone, fluclorolone acetonide, fludroxycortide, flunisolide, fluocinolone, fluocinolone acetonide, fluocinonide, halcinonide, triamcinolone, triamcinolone acetonide, cortivazol, RU-28362, dexamethasone acefurate, dexamethasone acetate, dexamethasone cipecilate, dexamethasone diethylaminoacetate, dexamethasone dipropionate, dexamethasone isonicotinate, dexamethasone linoleate, dexamethasone metasulphobenzoate, dexamethasone palmitate, dexamethasone phosphate, dexamethasone pivalate, dexamethasone succinate, dexamethasone sulfate, dexamethasone tebutate, dexamethasone troxundate, and dexamethasone valerate.

### Kinase Inhibitors

In certain embodiments, the bioactive agent is a kinase inhibitor, for example a Bruton's tyrosine kinase (BTK) inhibitor. In certain embodiments, the kinase inhibitor is selected from a phosphoinositide 3-kinase (PI3K) inhibitor, a Bruton's tyrosine kinase (BTK) inhibitor, or a spleen tyrosine kinase (Syk) inhibitor, or a combination thereof.

In certain embodiments, the morphic form or pharmaceutical composition of the present invention is administered in combination with a BTK inhibitor. In certain embodiments, the BTK inhibitor is ibrutinib. In certain embodiments, the embodiments the BTK inhibitor is acalabrutinib. In certain embodiments, Compound 1 is administered in combination with a BTK inhibitor. In certain embodiments, BTK inhibitor is zanubrutinib. In certain embodiments, Compound 1 is administered in combination with ibrutinib. In certain embodiments, Compound 1 is administered in combination with zanubrutinib. In certain embodiments, Compound 1 is administered in combination with acalabrutinib.

In certain embodiments Compound 1 is administered to a patient with a cancer that has metastasized to the brain. In certain embodiments Compound 1 is administered in combination with ibrutinib to treat a cancer that has metastasized to the brain. In certain embodiments Compound 1 is administered in combination with rituximab to treat a cancer that has metastasized to the brain.

Examples of BTK inhibitors include ibrutinib (also known as PCI-32765)(Imbruvica™)(1-[(3R)-3-[4-amino-3-(4-phenoxy-phenyl)pyrazolo[3,4-d]pyrimidin-1-yl]piperidin-1-yl]prop-2-en-1-one), dianilinopyrimidine-based inhibitors such as AVL-101 and AVL-291/292 (N-(3-((5-fluoro-2-((4-(2-methoxyethoxy)phenyl)amino)pyrimidin-4-yl)amino)phenyl)acrylamide) (Avila Therapeutics) (see US Patent Publication No 2011/0117073, incorporated herein in its entirety), dasatinib ([N-(2-chloro-6-methylphenyl)-2-(6-(4-(2-hydroxyethyl)piperazin-1-yl)-2-methylpyrimidin-4-ylamino)thiazole-5-carboxamide]), LFM-A13 (alpha-cyano-beta-hydroxy-beta-methyl-N-(2,5-ibromophenyl) propenamide), GDC-0834 ([R-N-(3-(6-(4-(1,4-dimethyl-3-oxopiperazin-2-yl)phenylamino)-4-methyl-5-oxo-4,5-dihydropyrazin-2-yl)-2-methylphenyl)-4,5,6,7-tetrahydrobenzo[b]thiophene-2-carboxamide]), CGI-560 4-(tert-butyl)-N-(3-(8-(phenylamino)imidazo[1,2-a]pyrazin-6-yl)phenyl)benzamide, CGI-1746 (4-(tert-butyl)-N-(2-methyl-3-(4-methyl-6-((4-(morpholine-4-carbonyl)phenyl)amino)-5-oxo-4,5-dihydropyrazin-2-yl)phenyl)benzamide), CNX-774 (4-(4-((4-((3-acrylamidophenyl)amino)-5-fluoropyrimidin-2-yl)amino)phenoxy)-N-methylpicolinamide), CTA056 (7-benzyl-1-(3-(piperidin-1-yl)propyl)-2-(4-(pyridin-4-yl)phenyl)-1H-imidazo[4,5-g]quinoxalin-6(5H)-one), GDC-0834 ((R)-N-(3-(6-((4-(1,4-dimethyl-3-oxopiperazin-2-yl)phenyl)amino)-4-methyl-5-oxo-4,5-dihydropyrazin-2-yl)-2-methylphenyl)-4,5,6,7-tetrahydrobenzo[b]thiophene-2-carboxamide), GDC-0837 ((R)-N-(3-(6-((4-(1,4-dimethyl-3-oxopiperazin-2-yl)phenyl)amino)-4-methyl-5-oxo-4,5-dihydropyrazin-2-yl)-2-methylphenyl)-4,5,6,7-tetrahydrobenzo[b]thiophene-2-carboxamide), HM-71224, ACP-196, ONO-4059 (Ono Pharmaceuticals), PRT062607 (4-((3-(2H-1,2,3-triazol-2-yl)phenyl)amino)-2-(((1R,2S)-2-aminocyclohexyl)amino)pyrimidine-5-carboxamide hydrochloride), QL-47 (1-(1-acryloylindolin-6-yl)-9-(1-methyl-1H-pyrazol-4-yl)benzo[h][1,6]naphthyridin-2(1H)-one), and RN486 (6-cyclopropyl-8-fluoro-2-(2-hydroxymethyl-3-{1-methyl-5-[5-(4-methyl-piperazin-1-yl)-pyridin-2-ylamino]-6-oxo-1,6-dihydro-pyridin-3-yl}-phenyl)-2H-isoquinolin-1-one), and other molecules capable of inhibiting BTK activity, for example those BTK inhibitors disclosed in Akinleye et ah, Journal of Hematology & Oncology, 2013, 6:59, the entirety of which is incorporated herein by reference.

In certain embodiments, the BTK inhibitor is selected from acalabrutinib, spebrutinib, zanubrutinib, LOXO-305, evobrutinib, TG-1701, tolebrutinib, BIIB091, DZD-9008, HZ-A-018, orelabrutinib, AC0058TA, SN1011, rilzabrutinib, ARQ 531, DTRMWXHS-12, JNJ-64264681, branebrutinib, and fenebrutinib. In certain embodiments, Compound 1 is administered in combination with an BTK inhibitor selected from acalabrutinib, spebrutinib, zanubrutinib, LOXO-305, evobrutinib, TG-1701, tolebrutinib, BIIB091, DZD-9008, HZ-A-018, orelabrutinib, AC0058TA, SN1011, rilzabrutinib, ARQ 531, DTRMWXHS-12, JNJ-64264681, branebrutinib, and fenebrutinib.

Examples of PI3 kinase inhibitors include but are not limited to wortmannin, demethoxyviridin, perifosine, idelalisib, pictilisib, Palomid 529, ZSTK474, PWT33597, CUDC-907, and AEZS-136, duvelisib, GS-9820, BKM120, GDC-0032 (taselisib) (2-[4-[2-(2-Isopropyl-5-methyl-1,2,4-triazol-3-yl)-5,6-dihydroimidazo[1,2-d][1,4]benzoxazepin-9-yl]pyrazol-1-yl]-2-methylpropanamide), MLN-1117 ((2R)-1-Phenoxy-2-butanyl hydrogen (S)-methylphosphonate; or Methyl(oxo) {[(2R)-1-phenoxy-2-butanyl]oxy}phosphonium)), BYL-719 ((2S)-N1-[4-Methyl-5-[2-(2,2,2-trifluoro-1,1-dimethylethyl)-4-pyridinyl]-2-thiazolyl]-1,2-pyrrolidinedicarboxamide), GSK2126458 (2,4-Difluoro-N-{2-(methyloxy)-5-[4-(4-pyridazinyl)-6-quinolinyl]-3-pyridinyl}benzenesulfonamide) (omipalisib), TGX-221 ((±)-7-Methyl-2-(morpholin-4-yl)-9-(1-phenylaminoethyl)-pyrido[1,2-a]-pyrimidin-4-one), GSK2636771 (2-Methyl-1-(2-methyl-3-(trifluoromethyl)benzyl)-6-morpholino-1H-benzo[d]imidazole-4-carboxylic acid dihydrochloride), KIN-193 ((R)-2-((1-(7-methyl-2-morpholino-4-oxo-4H-pyrido[1,2-a]pyrimidin-9-yl)ethyl)amino)benzoic acid), TGR-1202/RP5264, GS-9820 ((S)-1-(4-((2-(2-aminopyrimidin-5-yl)-7-methyl-4-mohydroxypropan-1-one), GS-1101 (5-fluoro-3-phenyl-2-([S)]-1-[9H-purin-6-ylamino]-propyl)-3H-quinazolin-4-one), AMG-319, GSK-2269557, SAR245409 (N-(4-(N-(3-((3,5-dimethoxyphenyl)amino)quinoxalin-2-yl)sulfamoyl)phenyl)-3-methoxy-4 methylbenzamide), BAY80-6946 (2-amino-N-(7-methoxy-8-(3-morpholinopropoxy)-2,3-dihydroimidazo[1,2-c]quinaz), AS 252424 (5-[l-[5-(4-Fluoro-2-hydroxy-phenyl)-furan-2-yl]-meth-(Z)-ylidene]-thiazolidine-2,4-dione), CZ 24832 (5-(2-amino-8-fluoro-[1,2,4]triazolo[1,5-a]pyridin-6-yl)-N-tert-butylpyridine-3-sulfonamide), buparlisib (5-[2,6-Di(4-morpholinyl)-4-pyrimidinyl]-4-(trifluoromethyl)-2-pyridinamine), GDC-0941 (2-(1H-Indazol-4-yl)-6-[[4-(methylsulfonyl)-1-piperazinyl]methyl]-4-(4-morpholinyl)thieno[3,2-d]pyrimidine), GDC-0980 ((S)-1-(4-((2-(2-aminopyrimidin-5-yl)-7-methyl-4-morpholinothieno[3,2-d]pyrimidin-6 yl)methyl)piperazin-1-yl)-2-hydroxypropan-1-one (also known as RG7422)), SF1126 ((8S,14S,17S)-14-(carboxymethyl)-8-(3-guanidinopropyl)-17-(hydroxymethyl)-3,6,9,12,15-pentaoxo-1-(4-(4-oxo-8-phenyl-4H-chromen-2-yl)morpholino-4-ium)-2-oxa-7,10,13,16-tetraazaoctadecan-18-oate), PF-05212384 (N-[4-[[4-(Dimethylamino)-1-piperidinyl]carbonyl]phenyl]-N′-[4-(4,6-di-4-morpholinyl-1,3,5-triazin-2-yl)phenyl]urea) (gedatolisib), LY3023414, BEZ235 (2-Methyl-2-{4-[3-methyl-2-oxo-8-(quinolin-3-yl)-2,3-dihydro-1H-imidazo[4,5-c]quinolin-1-yl]phenyl}propanenitrile) (dactolisib), XL-765 (N-(3-(N-(3-(3,5-dimethoxyphenylamino)quinoxalin-2-yl)sulfamoyl)phenyl)-3-methoxy-4-methylbenzamide), and GSK1059615 (5-[[4-(4-Pyridinyl)-6-quinolinyl]methylene]-2,4-thiazolidenedione), PX886 ([(3aR,6E,9S,9aR,10R,11aS)-6-[[bis(prop-2-enyl)amino]methylidene]-5-hydroxy-9-(methoxymethyl)-9a,11a-dimethyl-1,4,7-trioxo-2,3,3a,9,10,11-hexahydroindeno[4,5h]isochromen-10-yl] acetate (also known as sonolisib)), LY294002, AZD8186, PF-4989216, pilaralisib, GNE-317, PI-3065, PI-103, NU7441 (KU-57788), HS 173, VS-5584 (SB2343), CZC24832, TG100-115, A66, YM201636, CAYl0505, PIK-75, PIK-93, AS-605240, BGT226 (NVP-BGT226), AZD6482, voxtalisib, alpelisib, IC-87114, TGI100713, CH5132799, PKI-402, copanlisib (BAY 80-6946), XL 147, PIK-90, PIK-293, PIK-294, 3-MA (3-methyladenine), AS-252424, AS-604850, apitolisib (GDC-0980; RG7422), and the structure described in WO2014/071109.

Syk inhibitors include, for example, cerdulatinib (4-(cyclopropylamino)-2-((4-(4-(ethylsulfonyl)piperazin-1-yl)phenyl)amino)pyrimidine-5-carboxamide), entospletinib (6-(1H-indazol-6-yl)-N-(4-morpholinophenyl)imidazo[1,2-a]pyrazin-8-amine), fostamatinib ([6-({5-Fluoro-2-[(3,4,5-trimethoxyphenyl)amino]-4-pyrimidinyl}amino)-2,2-dimethyl-3-oxo-2,3-dihydro-4H-pyrido[3,2-b][1,4]oxazin-4-yl]methyl dihydrogen phosphate), fostamatinib disodium salt (sodium (6-((5-fluoro-2-((3,4,5-trimethoxyphenyl)amino)pyrimidin-4-yl)amino)-2,2-dimethyl-3-oxo-2H-pyrido[3,2-b][1,4]oxazin-4(3H)-yl)methyl phosphate), BAY 61-3606 (2-(7-(3,4-Dimethoxyphenyl)-imidazo[1,2-c]pyrimidin-5-ylamino)-nicotinamide HCl), RO9021 (6-[(1R,2S)-2-Amino-cyclohexylamino]-4-(5,6-dimethyl-pyridin-2-ylamino)-pyridazine-3-carboxylic acid amide), imatinib (Gleevac; 4-[(4-methylpiperazin-1-yl)methyl]-N-(4-methyl-3-{[4-(pyridin-3-yl)pyrimidin-2-yl]amino}phenyl)benzamide), staurosporine, GSK143 (2-(((3R,4R)-3-aminotetrahydro-2H-pyran-4-yl)amino)-4-(p-tolylamino)pyrimidine-5-carboxamide), PP2 (1-(tert-butyl)-3-(4-chlorophenyl)-1H-pyrazolo[3,4-d]pyrimidin-4-amine), PRT-060318 (2-(((1R,2S)-2-aminocyclohexyl)amino)-4-(m-tolylamino)pyrimidine-5-carboxamide), PRT-062607 (4-((3-(2H-1,2,3-triazol-2-yl)phenyl)amino)-2-(((1R,2S)-2-aminocyclohexyl)amino)pyrimidine-5-carboxamide hydrochloride), R112 (3,3′-((5-fluoropyrimidine-2,4-diyl)bis(azanediyl))diphenol), R348 (3-Ethyl-4-methylpyridine), R406 (6-((5-fluoro-2-((3,4,5-trimethoxyphenyl)amino)pyrimidin-4-yl)amino)-2,2-dimethyl-2H-pyrido[3,2-b][1,4]oxazin-3(4H)-one), piceatannol (3-Hydroxyresveratol), YM193306 (see Singh et al. Discovery and Development of Spleen Tyrosine Kinase (SYK) Inhibitors, J. Med. Chem. 2012, 55, 3614-3643), 7-azaindole, piceatannol, ER-27319 (see Singh et al. Discovery and Development of Spleen Tyrosine Kinase (SYK) Inhibitors, J. Med. Chem. 2012, 55, 3614-3643 incorporated in its entirety herein), Compound D (see Singh et al. Discovery and Development of Spleen Tyrosine Kinase (SYK) Inhibitors, J. Med. Chem. 2012, 55, 3614-3643 incorporated in its entirety herein), PRT060318 (see Singh et al. Discovery and Development of Spleen Tyrosine Kinase (SYK) Inhibitors, J. Med. Chem. 2012, 55, 3614-3643 incorporated in its entirety herein), luteolin (see Singh et al. Discovery and Development of Spleen Tyrosine Kinase (SYK) Inhibitors, J. Med. Chem. 2012, 55, 3614-3643 incorporated in its entirety herein), apigenin (see Singh et al. Discovery and Development of Spleen Tyrosine Kinase (SYK) Inhibitors, J. Med. Chem. 2012, 55, 3614-3643 incorporated in its entirety herein), quercetin (see Singh et al. Discovery and Development of Spleen Tyrosine Kinase (SYK) Inhibitors, J. Med. Chem. 2012, 55, 3614-3643 incorporated in its entirety herein), fisetin (see Singh et al. Discovery and Development of Spleen Tyrosine Kinase (SYK) Inhibitors, J. Med. Chem. 2012, 55, 3614-3643 incorporated in its entirety herein), myricetin (see Singh et al. Discovery and Development of Spleen Tyrosine Kinase (SYK) Inhibitors, J. Med. Chem. 2012, 55, 3614-3643 incorporated in its entirety herein), morin (see Singh et al. Discovery and Development of Spleen Tyrosine Kinase (SYK) Inhibitors, J. Med. Chem. 2012, 55, 3614-3643 incorporated in its entirety herein).

### Proteasome Inhibitors

In certain embodiments, the morphic form or pharmaceutical composition of the present invention is administered in combination with a proteasome inhibitor. In certain embodiments, the proteasome inhibitor is bortezomib. In certain embodiments, the proteasome inhibitor is ixazomib. In certain embodiments, the proteasome inhibitor is carfilzomib. In certain embodiments, Compound 1 is administered in combination with a proteasome inhibitor. In certain embodiments, Compound 1 is administered in combination with bortezomib. In certain embodiments, Compound 1 is administered in combination with ixazomib. In certain embodiments, Compound 1 is administered in combination with carfilzomib. In certain embodiments, Compound 1 is administered in combination with carfilzomib and daratumumab.

Additional examples of proteasome inhibitors include ixazomib citrate, oprozomib, delanzomib, lactacystin, epoxomicin, MG132, MG-262, CEP-18770, NEOSH101, TQB3602, and KZR-616. In certain embodiments, Compound 1 is administered in combination with a proteasome inhibitor selected from ixazomib citrate, oprozomib, delanzomib, lactacystin, epoxomicin, MG132, MG-262, CEP-18770, NEOSH101, TQB3602, VLX1570, and KZR-616.

### HDAC Inhibitors

In certain embodiments, the morphic form or pharmaceutical composition of the present invention is administered in combination with an HDAC inhibitor. In certain embodiments, the HDAC inhibitor is vorinostat. In certain embodiments, the HDAC inhibitor is romidepsin. In certain embodiments, the HDAC inhibitor is panobinostat. In certain embodiments, the HDAC inhibitor is belinostat. In certain embodiments, Compound 1 is administered in combination with an HDAC inhibitor. In certain embodiments, Compound 1 is administered in combination with vorinostat. In certain embodiments, Compound 1 is administered in combination with romidepsin. In certain embodiments, Compound 1 is administered in combination with panobinostat. In certain embodiments, Compound 1 is administered in combination with belinostat.

In certain embodiments, the HDAC inhibitor is selected from trapoxin B, sodium phenylbutyrate, tacedinaline, mocetinostat, BRD73954, BG45, domatinostat, cay10603, HPOB, TMP269, nexturastat A, santacruzamate A, splitomicin, LMK-235, sodium butyrate, pivaloyloxymethyl butyrate, pyroxamide, abexinostat, resminostat, givinostat, quisinostat, psammaplin A, KD5170, 1-Alaninechiamydocin, depudecin, and CUDC-101. In certain embodiments, Compound 1 is administered in combination with an HDAC inhibitor selected from trapoxin B, sodium phenylbutyrate, tacedinaline, mocetinostat, BRD73954, BG45, domatinostat, cay10603, HPOB, TMP269, nexturastat A, santacruzamate A, splitomicin, LMK-235, sodium butyrate, pivaloyloxymethyl butyrate, pyroxamide, abexinostat, resminostat, givinostat, quisinostat, psammaplin A, KD5170, 1-Alaninechlamydocin, depudecin, and CUDC-101.

### Immunomodulatory Imide Drums

In certain embodiments, the morphic form or pharmaceutical composition of the present invention is administered in combination with an immunomodulatory imide drug (ID), also referred to as IMiD®. In certain embodiments, the IID is thalidomide. In certain embodiments, the IID is lenalidomide. In certain embodiments, the IID is pomalidomide. In certain embodiments, Compound 1 is administered in combination with thalidomide. In certain embodiments, Compound 1 is administered in combination with lenalidomide. In certain embodiments, Compound 1 is administered in combination with pomalidomide.

In certain embodiments, the IID is CC-90009. In certain embodiments, the IID is CC-99282. In certain embodiments, the IID is mezigdomide, also known as CC-92480. In certain embodiments, Compound 1 is administered in combination with CC-90009. In certain embodiments, Compound 1 is administered in combination with CC-99282. In certain embodiments, Compound 1 is administered in combination with CC-92480. In certain embodiments, Compound 1 is administered in combination with iberdomide, also known as CC-220.

In certain embodiments, the IID is selected from CC-90009, CC-99282, mezigdomide (CC-92480), and iberdomide (CC-220).

### Antibodies

In certain embodiments, the morphic form or pharmaceutical composition of the present invention is administered in combination with an antibody targeting CD20, CD30, or CD38. In certain embodiments, the targeted antibody is rituximab. In certain embodiments, the targeted antibody is daratumumab. In certain embodiments, the targeted antibody is elotuzumab. In certain embodiments, the targeted antibody is isatuximab. In certain embodiments, Compound 1 is administered in combination with an antibody targeting CD20, CD30, or CD38. In certain embodiments, Compound 1 is administered in combination with rituximab. In certain embodiments, Compound 1 is administered in combination with raratumumab. In certain embodiments, Compound 1 is administered in combination with elotuzumab. In certain embodiments, Compound 1 is administered in combination with isatuximab.

In certain embodiments, the morphic form or pharmaceutical composition of the present invention is administered in combination with an antibody-drug conjugate. In certain embodiments, the antibody-drug conjugate is brentuximab vedotin. In certain embodiments, the antibody-drug conjugate is ibritumomab tiuxetan. In certain embodiments, the antibody-drug conjugate is mogamulizumab. In certain embodiments, the antibody-drug conjugate is obinutuzumab. In certain embodiments, the antibody-drug conjugate is polatuzumab vedotin. In certain embodiments, the antibody-drug conjugate is belantamab mafodotin (GSK2857916). In certain embodiments, the antibody-drug conjugate is MEDI2228. In certain embodiments, the antibody-drug conjugate is CC-99712. In certain embodiments, Compound 1 is administered in combination with an antibody-drug conjugate. In certain embodiments, Compound 1 is administered in combination with brentuximab vedotin. In certain embodiments, Compound 1 is administered in combination with ibritumomab tiuxetan. In certain embodiments, Compound 1 is administered in combination with mogamulizumab. In certain embodiments, Compound 1 is administered in combination with obinutuzumab. In certain embodiments, Compound 1 is administered in combination with polatuzumab vedotin. In certain embodiments, Compound 1 is administered in combination with belantamab mafodotin (GSK2857916). In certain embodiments, Compound 1 is administered in combination with MEDI2228. In certain embodiments, Compound 1 is administered in combination with CC-99712. In certain embodiments, Compound 1 is administered in combination with tafasitamab.

In certain embodiments, the morphic form or pharmaceutical composition of the present invention is administered in combination with a bispecific antibody. In certain embodiments, the bispecific antibody is PF-06863135. In certain embodiments, the bispecific antibody is TNB-383B. In certain embodiments, the bispecific antibody is REGN5458. In certain embodiments, the bispecific antibody is JNJ-64007957. In certain embodiments, Compound 1 is administered in combination with a bispecific antibody. In certain embodiments, Compound 1 is administered in combination with PF-06863135. In certain embodiments, Compound 1 is administered in combination with TNB-383B. In certain embodiments, Compound 1 is administered in combination with REGN5458. In certain embodiments, Compound 1 is administered in combination with JNJ-64007957.

In certain embodiments, the bispecific antibody is selected from PF-06863135, TNB-383B, REGN5458, and JNJ-64007957.

In certain embodiments, the morphic form or pharmaceutical composition of the present invention is administered in combination with a naked monoclonal antibody (mAb). In certain embodiments, the naked mAb is SEA-BCMA. In certain embodiments, Compound 1 is administered in combination with a naked mAb. In certain embodiments, Compound 1 is administered in combination with SEA-BCMA.

Additional non-limiting examples of CD38 antibodies include felzartamab, GBR 1342, TAK-573, CID-103, OKT10, STI-6129, SGX301, and mezagitamab. In certain embodiments, Compound 1 is administered in combination with a CD38 antibody selected from felzartamab, GBR 1342, TAK-573, CID-103, OKT10, STI-6129, SGX301, TAK-079, and mezagitamab.

### CAR T-Cell Therapy

Chimeric antigen receptor T-cells (CAR-Ts) are T lymphocytes that have been genetically engineered to express synthetic CAR molecules on their surface. CAR molecules endow T lymphocytes with the proficiency to recognize cell surface target antigens of interest and mediate exclusive cytotoxicity against cells expressing these antigens (S. Kozani et al. “Nanobody-based CAR-T cells for cancer immunotherapy”. Biomarker Research (2022) 10:24.) CAR targets include B-cell maturation antigen (BCMA), SLAMF7 (also known as CS1), CD19, CD38, CD138, and GPRC5D. CD38 is highly expressed on MM cells. BCMA is mainly expressed on plasma cells and plasmacytoid dendritic cells, but not other normal cells and considered to be a target for CAR-T cells in the treatment of r/r MM. (M. Kleber et al., “BCMA in Multiple Myeloma—A Promising Key to Therapy”. J. Clin. Med. 2021, 10, 4088; Nobari et al. “B-cell maturation antigen targeting strategies in multiple myeloma treatment, advantages and disadvantages” J. Transl. Med. (2022) 20:82.). Recently, bispecific BCMA-CD38 CAR-T cells containing a BCMA targeting domain and CD38 targeting domain have been reported. (H. Mei et al. “A bispecific CAR-T cell therapy targeting BCMA and CD38 in relapsed or refractory multiple myeloma”. J. Hematol. Oncol. (2021) 14:161; Y. Tang et al. “High efficacy and safety of CD38 and BCMA bispecific CAR-T in relapsed or refractory multiple myeloma”. J. Exp. Clin. Cancer Res. (2022) 41:2.). BCMA/CS1 bispecific CAR-T cells have also been shown to be effective in targeting BCMA- or CS1-expressing MM cells. (Zah, E., et al. “Systematically optimized BCMA/CS1 bispecific CAR-T cells robustly control heterogeneous multiple myeloma”. Nat. Commun. (2020), 11, 2283; V. Golubovskaya et al. “Novel CS1 CAR-T Cells and Bispecific CS1-BCMA CAR-T Cells Effectively Target Multiple Myeloma”. Biomedicines. 2021 October; 9(10): 1422.) Bispecific BCMA-CD19 and BCMA-GPRC5D CAR-Ts have also been described recently. (G. Roex et al., “Two for one: targeting BCMA and CD19 in B-cell malignancies with off-the-shelf dual-CAR NK-92 cells”. J. Transl. Med. (2022) 20:124; C. F. de Larrea et al., “Defining an Optimal Dual-Targeted CAR T-cell Therapy Approach Simultaneously Targeting BCMA and GPRC5D to Prevent BCMA Escape-Driven Relapse in Multiple Myeloma”. Blood Cancer Discov. 2020 September; 1(2):146-154.)

In certain embodiments, the morphic form or pharmaceutical composition of the present invention is administered in combination with a CAR T-cell therapy. In certain embodiments, the CAR T-cell therapy is Axicabtagene ciloleucel. In certain embodiments, the CAR T-cell therapy is ALLO-715. In certain embodiments, the CAR T-cell therapy is bb21217. In certain embodiments, the CAR T-cell therapy is BCMA CAR-T. In certain embodiments, the CAR T-cell therapy is CD138 CAR-T. In certain embodiments, the CAR T-cell therapy is CD19 CAR-T. In certain embodiments, the CAR T-cell therapy is Tisagenlecleucel. In certain embodiments, the CAR T-cell therapy is idecabtagene vicleucel (ide-cel; bb2121; ABECMA® (Celgene)). In certain embodiments, the CAR T-cell therapy is LCAR-B38M (JNJ-4528; JNJ-68284528). In certain embodiments, the CAR T-cell therapy is P-BCMA-101. In certain embodiments, the CAR T-cell therapy is PBCAR269A. In certain embodiments, the CAR T-cell therapy is bb21217. In certain embodiments, the CAR T-cell therapy is JCARK125 (orva-cel; orvacabtagene autoleucel). In certain embodiments, the CAR T-cell therapy is ALLO-715. In certain embodiments, the CAR T-cell therapy is Descartes-08. In certain embodiments, the CAR T-cell therapy is FCARH143. In certain embodiments, the CAR T-cell therapy is CT053.

In certain embodiments, Compound 1 is administered in combination with Axicabtagene ciloleucel. In certain embodiments, Compound 1 is administered in combination with Tisagenlecleucel. In certain embodiments, Compound 1 is administered in combination with Idecabtagene vicleucel (ide-cel; bb2121). In certain embodiments, Compound 1 is administered in combination with LCAR-B38M (JNJ-4528; JNJ-68284528). In certain embodiments, Compound 1 is administered in combination with P-BCMA-101. In certain embodiments, Compound 1 is administered in combination with PBCAR269A. In certain embodiments, Compound 1 is administered in combination with bb21217. In certain embodiments, Compound 1 is administered in combination with JCARK125 (orva-cel; orvacabtagene autoleucel). In certain embodiments, Compound 1 is administered in combination with ALLO-715. In certain embodiments, Compound 1 is administered in combination with Descartes-08. In certain embodiments, Compound 1 is administered in combination with FCARH143. In certain embodiments, Compound 1 is administered in combination with CT053.

In certain embodiments, the CAR T-cell therapy is selected from Axicabtagene ciloleucel, Tisagenlecleucel, Idecabtagene vicleucel (ide-cel; bb2121), LCAR-B38M (JNJ-4528; JNJ-68284528), and P-BCMA-101. In certain embodiments, the CAR T-cell therapy is selected from PBCAR269A, bb21217, JCARK125 (orva-cel; orvacabtagene autoleucel), ALLO-715, Descartes-08, FCARH143, and CT053.

In certain embodiments the CAR T-cell Therapy is selected from ALLO-715, bb21217, BCMA CAR-T, CD138 CAR-T, CD19 CAR-T, ciltacabtagene autoleucel, CS1 (SLAMF7) CAR-T, CT053, Descartes-11, idecabtagene vicleucel, NKG2D CAR-T, orvacabtagene autoleucel, P-BCMA-101, and UCARTCS1.

In certain embodiments, the CAR T-cell Therapy is idecabtagene vicleucel, for example ABECMA®.

In certain embodiments, the CAR T-cell Therapy is lisocabtagene maraleucel, for example BREYANZI®.

In certain embodiments, Compound 1 is administered in in combination with lisocabtagene maraleucel, for example BREYANZI®.

In certain embodiments, the CAR T-cell Therapy is ciltacabtagene autolecuel, for example CARVYKTI™.

In certain embodiments, the CAR T-cell Therapy is tisagenelecleucel, for example KYMRIAH™.

In certain embodiments, the CAR T-cell Therapy is brexucabtagene autoleucel, for example TECARTUS™.

In certain embodiments, Compound 1 is administered in combination with brexucabtagene autoleucel, for example TECARTUS™.

In certain embodiments, the CAR T-cell Therapy is axicabtagene ciloleucel, for example YESCARTA™.

In certain embodiments, the CAR T-cell therapy is a bispecific CAR T-cell therapy. In certain embodiments, the bispecific CAR T-cell therapy is a BCMA-CD38 CAR T-cell therapy. In certain embodiments, the bispecific CAR T-cell therapy is a BCMA-CS1 CAR T-cell therapy. In certain embodiments, the bispecific CAR T-cell therapy is a BCMA-CD19 CAR T-cell therapy. In certain embodiments, the bispecific CAR T-cell therapy is a BCMA-GPRC5D CAR T-cell therapy. In certain embodiments, the bispecific CAR T-cell therapy is a C19-C20 CAR T-cell therapy. In certain embodiments, the bispecific CAR T-cell therapy is a CD138-CD38 CAR T-cell therapy. In certain embodiments, the bispecific CAR T-cell therapy is a CD19-CD138 CAR T-cell therapy.

In certain embodiments, Compound 1 is administered in combination with bispecific CAR T-cell therapy. In certain embodiments, Compound 1 is administered in combination with a BCMA-CD38 CAR T-cell therapy. In certain embodiments, Compound 1 is administered in combination with a BCMA-CS1 CAR T-cell therapy. In certain embodiments, Compound 1 is administered in combination with a BCMA-CD19 CAR T-cell therapy. In certain embodiments, Compound 1 is administered in combination with a BCMA-GPRC5D CAR T-cell therapy. In certain embodiments, Compound 1 is administered in combination with a C19-C20 CAR T-cell therapy. In certain embodiments, Compound 1 is administered in combination with a CD138-CD38 CAR T-cell therapy. In certain embodiments, Compound 1 is administered in combination with a CD19-CD138 CAR T-cell therapy.

### Cellular Therapy

In certain embodiments, the morphic form or pharmaceutical composition of the present invention is administered in combination with a cellular therapy. In certain embodiments Compound 1 is used in combination with a cellular therapy.

Non-limiting examples of cellular therapy include allo-HSCT, allo-NKT, auto-HSCT, and auto-NKT.

### T-Cell Engagers

In certain embodiments, the morphic form or pharmaceutical composition of the present invention is administered in combination with a bi-specific T-cell engagers (BiTEs). In certain embodiments, the BiTE is blinatumomab. In certain embodiments, the BiTE is CC-93268. In certain embodiments, the BiTE is AMG 420. In certain embodiments, the BiTE is AMG 701. In certain embodiments, Compound 1 is administered in combination with a bi-specific T-cell engagers (BiTEs). In certain embodiments, Compound 1 is administered in combination with blinatumomab. In certain embodiments, Compound 1 is administered in combination with AMG 420. In certain embodiments, Compound 1 is administered in combination with CC-93269. In certain embodiments, Compound 1 is administered in combination with AMG 701.

In certain embodiments, the BiTE is selected from blinatumomab, AMG 420, CC-93269, and AMG 4701. In certain embodiments the BiTE is BCMA-CD38.

In certain embodiments Compound 1 is used in combination with a bispecific antibody selected from AMG 420, AMG 701, BFCR4350A, blinatumomab, CC-93269, elranatamab, EM801, REGN5458, talquetamab, teclistamab, and TNB-383B.

### Immune Modulators Checkpoint Inhibitors

In certain embodiments, the morphic form or pharmaceutical composition of the present invention is administered in combination with a checkpoint inhibitor. In certain embodiments, the morphic form or pharmaceutical composition of the present invention is administered in combination with a PD-1 checkpoint inhibitor. In certain embodiments, the morphic form or pharmaceutical composition of the present invention is administered in combination with a PD-L1 checkpoint inhibitor. In certain embodiments, the morphic form or pharmaceutical composition of the present invention is administered in combination with an IFNAR checkpoint inhibitor. In certain embodiments, the checkpoint inhibitor is nivolumab. In certain embodiments, the checkpoint inhibitor is pembrolizumab. In certain embodiments, the checkpoint inhibitor is interferon alfa-2b. In certain embodiments, Compound 1 is administered in combination with a checkpoint inhibitor. In certain embodiments, Compound 1 is administered in combination with a PD-1 checkpoint inhibitor. In certain embodiments, Compound 1 is administered in combination with a PD-L1 checkpoint inhibitor. In certain embodiments, Compound 1 is administered in combination with an IFNAR checkpoint inhibitor. In certain embodiments, Compound 1 is administered in combination with nivolumab. In certain embodiments, Compound 1 is administered in combination with pembrolizumab. In certain embodiments, Compound 1 is administered in combination with interferon alfa-2b.

PD-1 inhibitors that blocks the interaction of PD-1 and PD-L1 by binding to the PD-1 receptor, and in turn inhibit immune suppression include, for example, nivolumab (OPDIVO® (Bristol-Myers Squibb)), pembrolizumab (KEYTRUDA® (Merck & Co)), pidilizumab, AMP-224 (AstraZeneca and MedImmune), PF-06801591 (Pfizer), MEDI0680 (AstraZeneca), PDR001 (Novartis), REGN2810 (Regeneron), SHR-12-1 (Jiangsu Hengrui Medicine Company and Incyte Corporation), TSR-042 (Tesaro Inc.), and the PD-L1/VISTA inhibitor CA-170 (Curis Inc.). PD-L1 inhibitors that block the interaction of PD-1 and PD-L1 by binding to the PD-L1 receptor, and in turn inhibits immune suppression, include for example, atezolizumab (TECENTRIQ® (Genentech)), durvalumab (AstraZeneca and MedImmune), KN035 (Alphamab Oncology), and BMS-936559 (Bristol-Myers Squibb). CTLA-4 checkpoint inhibitors that bind to CTLA-4 and inhibits immune suppression include, but are not limited to, ipilimumab, tremelimumab (AstraZeneca and MedImmune), AGEN1884 and AGEN2041 (Agenus). LAG-3 checkpoint inhibitors, include, but are not limited to, BMS-986016 (Bristol-Myers Squibb), GSK2831781 (GlaxoSmithKline), IMP321 (Prima BioMed), LAG525 (Novartis), and the dual PD-1 and LAG-3 inhibitor MGD013 (MacroGenics). An example of a TIM-3 inhibitor is TSR-022 (Tesaro).

In certain embodiments, the checkpoint inhibitor is selected from nivolumab/OPDIVO®; pembrolizumab/KEYTRUDA®; and pidilizumab/CT-011, MPDL3280A/RG7446; MEDI4736; MSB0010718C; BMS 936559, a PDL2/lg fusion protein such as AMP 224 or an inhibitor of B7-H3 (e.g., MGA271), B7-H4, BTLA, HVEM, TIM3, GAL9, LAG 3, VISTA, KIR, 2B4, CD160, CGEN-15049, CHK 1, CHK2, A2aR, B-7 family ligands, or a combination thereof.

In certain embodiments, the PD-1 inhibitor is BGB-A317. In certain embodiments, the PD-L1 inhibitor is MED14736. In certain embodiments, the PD-L2 inhibitor is rHIgM12B7A.

In certain embodiments, the checkpoint inhibitor is a B7 inhibitor, for example a B7-H3 inhibitor or a B7-H4 inhibitor. In certain embodiments, the B7-H3 inhibitor is MGA271.

In certain embodiments, the checkpoint inhibitor is an OX40 agonist. In certain embodiments, the checkpoint inhibitor is an anti-OX40 antibody, for example anti-OX-40 or MEDI6469.

In certain embodiments, the checkpoint inhibitor is a GITR agonist. In certain embodiments, the GITR agonist is an anti-GITR antibody, for example TRX518.

In certain embodiments, the checkpoint inhibitor is a CD137 agonist. In certain embodiments, the CD137 agonist is an anti-CD137 antibody, for example PF-05082566.

In certain embodiments, the checkpoint inhibitor is a CD40 agonist. In certain embodiments, the CD40 agonist is an anti-CD40 antibody, for example CF-870,893.

In certain embodiments, the checkpoint inhibitor is an IDO inhibitor, for example INCB24360 or indoximod.

In certain embodiments the checkpoint inhibitor is selected from atezolizumab, avelumab, durvalumab, nivolumab, and pembrolizumab.

### Additional Bioactive Agents

In another embodiment, an active compound described herein can be administered in an effective amount for the treatment of abnormal tissue of the male reproductive system such as prostate or testicular cancer, in combination or alternation with an effective amount of an androgen (such as testosterone) inhibitor including but not limited to a selective androgen receptor modulator, a selective androgen receptor degrader, a complete androgen receptor degrader, or another form of partial or complete androgen antagonist. In certain embodiments, the prostate or testicular cancer is androgen-resistant. Non-limiting examples of anti-androgen compounds are provided in WO 2011/156518 and U.S. Pat. Nos. 8,455,534 and 8,299,112. Additional non-limiting examples of anti-androgen compounds include: enzalutamide, apalutamide, cyproterone acetate, chlormadinone acetate, spironolactone, canrenone, drospirenone, ketoconazole, topilutamide, abiraterone acetate, and cimetidine.

In certain embodiments, the bioactive agent is an ALK inhibitor. Examples of ALK inhibitors include but are not limited to crizotinib, alectinib, ceritinib, TAE684 (NVP-TAE684), GSK1838705A, AZD3463, ASP3026, PF-06463922, entrectinib (RXDX-101), and AP26113.

In certain embodiments, the bioactive agent is an EGFR inhibitor. Examples of EGFR inhibitors include erlotinib (TARCEVA® (Genentech)), gefitinib (IRESSA® AstraZeneca)), afatinib (GILOTRIF® (Boehringer Ingelheim)), rociletinib (CO-1686), osimertinib (TAGRISSO® (AstraZeneca)), olmutinib (OLITA™ (Hanmi Pharm Co.)), naquotinib (ASP8273), nazartinib (EGF816), PF-06747775 (Pfizer), icotinib (BPI-2009), neratinib (HKI-272; PB272); avitinib (AC0010), EAI045, tarloxotinib (TH-4000; PR-610), PF-06459988 (Pfizer), tesevatinib (XL647; EXEL-7647; KD-019), transtinib, WZ-3146, WZ8040, CNX-2006, and dacomitinib (PF-00299804; Pfizer).

In certain embodiments, the bioactive agent is an HER-2 inhibitor. Examples of HER-2 inhibitors include trastuzumab, lapatinib, ado-trastuzumab emtansine, and pertuzumab.

In certain embodiments, the bioactive agent is a CD20 inhibitor. Examples of CD20 inhibitors include obinutuzumab, rituximab, fatumumab, ibritumomab, tositumomab, and ocrelizumab.

In certain embodiments, the bioactive agent is a JAK3 inhibitor. Examples of JAK3 inhibitors include tasocitinib.

In certain embodiments the bioactive agent is a JAK inhibitor, for example ruxolitinib.

In certain embodiments, the bioactive agent is a BCL-2 inhibitor. Examples of BCL-2 inhibitors include venetoclax, ABT-199 (4-[4-[[2-(4-Chlorophenyl)-4,4-dimethylcyclohex-1-en-1-yl]methyl]piperazin-1-yl]-N-[[3-nitro-4-[[(tetrahydro-2H-pyran-4-yl)methyl]amino]phenyl]sulfonyl]-2-[(1H-pyrrolo[2,3-b]pyridin-5-yl)oxy]benzamide), ABT-737 (4-[4-[[2-(4-chlorophenyl)phenyl]methyl]piperazin-1-yl]-N-[4-[[(2R)-4-(dimethylamino)-1-phenylsulfanylbutan-2-yl]amino]-3-nitrophenyl]sulfonylbenzamide) (navitoclax), ABT-263 ((R)-4-(4-((4′-chloro-4,4-dimethyl-3,4,5,6-tetrahydro-[1,1′-biphenyl]-2-yl)methyl)piperazin-1-yl)-N-((4-((4-morpholino-1-(phenylthio)butan-2-yl)amino)-3((trifluoromethyl)sulfonyl)phenyl)sulfonyl)benzamide), GX15-070 (obatoclax mesylate, (2Z)-2-[(5Z)-5-[(3,5-dimethyl-1H-pyrrol-2-yl)methylidene]-4-methoxypyrrol-2-ylidene]indole; methanesulfonic acid))), 2-methoxy-antimycin A3, YC137 (4-(4,9-dioxo-4,9-dihydronaphtho[2,3-d]thiazol-2-ylamino)-phenyl ester), pogosin, ethyl 2-amino-6-bromo-4-(1-cyano-2-ethoxy-2-oxoethyl)-4H-chromene-3-carboxylate, Nilotinib-d3, TW-37 (N-[4-[[2-(1,1-Dimethylethyl)phenyl]sulfonyl]phenyl]-2,3,4-trihydroxy-5-[[2-(1-methylethyl)phenyl]methyl]benzamide), Apogossypolone (ApoG2), HA14-1, AT101, sabutoclax, gambogic acid, or G3139 (Oblimersen).

In certain embodiments the bioactive agent is venetoclax.

In certain embodiments, the bioactive agent is a MEK inhibitor. MEK inhibitors are well known, and include, for example, trametinib/GSK1120212 (N-(3-{3-cyclopropyl-5-[(2-fluoro-4-iodophenyl)amino]-6,8-dimethyl-2,4,7-trioxo-3,4,6,7-tetrahydropyrido[4,3-d]pyrimidin-1(2H-yl}phenyl)acetamide), selumetinib (6-(4-bromo-2-chloroanilino)-7-fluoro-N-(2-hydroxyethoxy)-3-methylbenzimidazole-5-carboxamide), pimasertib/AS703026/MSC 1935369 ((S)-N-(2,3-dihydroxypropyl)-3-((2-fluoro-4-iodophenyl)amino)isonicotinamide), XL-518/GDC-0973 (1-({3,4-difluoro-2-[(2-fluoro-4-iodophenyl)amino]phenyl}carbonyl)-3-[(2S)-piperidin-2-yl]-azetidin-3-ol), refametinib/BAY869766/RDEA119 (N-(3,4-difluoro-2-(2-fluoro-4-iodophenylamino)-6-methoxyphenyl)-1-(2,3-dihydroxypropyl)cyclopropane-1-sulfonamide), PD-0325901 (N-[(2R)-2,3-dihydroxypropoxy]-3,4-difluoro-2-[(2-fluoro-4-iodophenyl)amino]-benzamide), TAK733 ((R)-3-(2,3-dihydroxypropyl)-6-fluoro-5-(2-fluoro-4-iodophenylamino)-8-methylpyrido[2,3-d]pyrimidine-4,7(3H,8H)-dione), MEK162/ARRY438162 (5-[(4-bromo-2-fluorophenyl)amino]-4-fluoro-N-(2-hydroxyethoxy)-1-methyl-1H-benzimidazole-6-carboxamide), R05126766 (3-[[3-fluoro-2-(methylsulfamoylamino)-4-pyridyl]methyl]-4-methyl-7-pyrimidin-2-yloxychromen-2-one), WX-554, R04987655/CH4987655 (3,4-difluoro-2-((2-fluoro-4-iodophenyl)amino)-N-(2-hydroxyethoxy)-5-((3-oxo-1,2-oxazinan-2yl)methyl)benzamide), or AZD8330 (2-((2-fluoro-4-iodophenyl)amino)-N-(2-hydroxyethoxy)-1,5-dimethyl-6-oxo-1,6-dihydropyridine-3-carboxamide), U0126-EtOH, PD184352 (CI-1040), GDC-0623, BI-847325, cobimetinib, PD98059, BIX 02189, BIX 02188, binimetinib, SL-327, TAK-733, PD318088.

In certain embodiments, the bioactive agent is a Raf inhibitor. Raf inhibitors are known and include, for example, vemurafinib (N-[3-[[5-(4-chlorophenyl)-1H-pyrrolo[2,3-b]pyridin-3-yl]carbonyl]-2,4-difluorophenyl]-1-propanesulfonamide), sorafenib tosylate (4-[4-[[4-chloro-3-(trifluoromethyl)phenyl]carbamoylamino]phenoxy]-N-methylpyridine-2-carboxamide;4-methylbenzenesulfonate), AZ628 (3-(2-cyanopropan-2-yl)-N-(4-methyl-3-(3-methyl-4-oxo-3,4-dihydroquinazolin-6-ylamino)phenyl)benzamide), NVP-BHG712 (4-methyl-3-(1-methyl-6-(pyridin-3-yl)-1H-pyrazolo[3,4-d]pyrimidin-4-ylamino)-N-(3-(trifluoromethyl)phenyl)-benzamide), RAF-265 (1-methyl-5-[2-[5-(trifluoromethyl)-1H-imidazol-2-yl]pyridin-4-yl]oxy-N-[4-(trifluoromethyl)phenyl]benzimidazol-2-amine), 2-bromoaldisine (2-bromo-6,7-dihydro-1H,5H-pyrrolo[2,3-c]azepine-4,8-dione), Raf Kinase Inhibitor IV (2-chloro-5-(2-phenyl-5-(pyridin-4-yl)-1H-imidazol-4-yl)phenol), sorafenib N-oxide (4-[4-[[[[4-chloro-3-(trifluoromethyl)phenyl]amino]carbonyl]amino]phenoxy]-N-methyl-2-pyridinecarboxamide 1-oxide), PLX-4720, dabrafenib (GSK2118436), GDC-0879, RAF265, AZ 628, SB590885, ZM336372, GW5074, TAK-632, CEP-32496, LY3009120, and GX818 (encorafenib).

In certain embodiments, the bioactive agent is an AKT inhibitor, including but not limited to, MK-2206, GSK690693, perifosine, (KRX-0401), GDC-0068, triciribine, AZD5363, honokiol, PF-04691502, and miltefosine, a FLT-3 inhibitor, including but not limited to, P406, dovitinib, quizartinib (AC220), amuvatinib (MP-470), tandutinib (MLN518), ENMD-2076, and KW-2449, or a combination thereof.

In certain embodiments, the bioactive agent is an mTOR inhibitor. Examples of mTOR inhibitors include but are not limited to rapamycin and its analogs, everolimus (AFINITOR (Novartis)), temsirolimus, ridaforolimus, sirolimus, and deforolimus.

In certain embodiments, the bioactive agent is a RAS inhibitor. Examples of RAS inhibitors include but are not limited to Reolysin and siG12D LODER.

In certain embodiments, the bioactive agent is a HSP inhibitor. HSP inhibitors include but are not limited to Geldanamycin or 17-N-Allylamino-17-demethoxygeldanamycin (17AAG), and Radicicol.

In certain embodiments the bioactive agent is a biphosphonate. Examples of biphosphonates include but are not limited to clodronate, pamidronate, and zoledronic acid.

Additional bioactive compounds include, for example, everolimus, trabectedin, abraxane, TLK 286, AV-299, DN-101, pazopanib, GSK690693, RTA 744, ON 0910.Na, AZD 6244 (ARRY-142886), AMN-107, TKI-258, GSK461364, AZD 1152, enzastaurin, vandetanib, ARQ-197, MK-0457, MLN8054, PHA-739358, R-763, AT-9263, a FLT-3 inhibitor, a VEGFR inhibitor, an aurora kinase inhibitor, a PIK-1 modulator, an HDAC inhbitor, a c-MET inhibitor, a PARP inhibitor, a Cdk inhibitor, an IGFR-TK inhibitor, an anti-HGF antibody, a focal adhesion kinase inhibitor, a Map kinase (mek) inhibitor, a VEGF trap antibody, pemetrexed, panitumumab, amrubicin, oregovomab, Lep-etu, nolatrexed, azd2171, batabulin, ofatumumab, zanolimumab, edotecarin, tetrandrine, rubitecan, tesmilifene, oblimersen, ticilimumab, ipilimumab, gossypol, Bio 111, 131-I-TM-601, ALT-110, BIO 140, CC 8490, cilengitide, gimatecan, IL13-PE38QQR, INO 1001, IPdR₁ KRX-0402, lucanthone, LY317615, neuradiab, vitespan, Rta 744, Sdx 102, talampanel, atrasentan, Xr 311, romidepsin, ADS-100380, sunitinib, 5-fluorouracil, vorinostat, etoposide, gemcitabine, doxorubicin, liposomal doxorubicin, 5′-deoxy-5-fluorouridine, vincristine, temozolomide, ZK-304709, seliciclib; PD0325901, AZD-6244, capecitabine, L-Glutamic acid, N-[4-[2-(2-amino-4,7-dihydro-4-oxo-1H-pyrrolo[2,3-d]pyrimidin-5-yl)ethyl]benzoyl]-, disodium salt, heptahydrate, camptothecin, PEG-labeled irinotecan, tamoxifen, toremifene citrate, anastrazole, exemestane, letrozole, DES (diethylstilbestrol), estradiol, estrogen, conjugated estrogen, bevacizumab, IMC-1C11, CHIR-258); 3-[5-(methylsulfonylpiperadinemethyl)-indolyl-quinolone, vatalanib, AG-013736, AVE-0005, goserelin acetate, leuprolide acetate, triptorelin pamoate, medroxyprogesterone acetate, hydroxyprogesterone caproate, megestrol acetate, raloxifene, bicalutamide, flutamide, nilutamide, megestrol acetate, CP-724714; TAK-165, HKI-272, erlotinib, lapatanib, canertinib, ABX-EGF antibody, erbitux, EKB-569, PKI-166, GW-572016, Ionafarnib, BMS-214662, tipifarnib; amifostine, NVP-LAQ824, suberoyl analide hydroxamic acid, valproic acid, trichostatin A, FK-228, SUl1248, sorafenib, KRN951, aminoglutethimide, arnsacrine, anagrelide, L-asparaginase, Bacillus Calmette-Guerin (BCG) vaccine, adriamycin, bleomycin, buserelin, busulfan, carboplatin, carmustine, chlorambucil, cisplatin, cladribine, clodronate, cyproterone, cytarabine, dacarbazine, dactinomycin, daunorubicin, diethylstilbestrol, epirubicin, fludarabine, fludrocortisone, fluoxymesterone, flutamide, gleevec, gemcitabine, hydroxyurea, idarubicin, ifosfamide, imatinib, leuprolide, levamisole, lomustine, mechlorethamine, melphalan, 6-mercaptopurine, mesna, methotrexate, mitomycin, mitotane, mitoxantrone, nilutamide, octreotide, oxaliplatin, pamidronate, pentostatin, plicamycin, porfimer, procarbazine, raltitrexed, rituximab, streptozocin, teniposide, testosterone, thalidomide, thioguanine, thiotepa, tretinoin, vindesine, 13-cis-retinoic acid, phenylalanine mustard, uracil mustard, estramustine, altretamine, floxuridine, 5-deooxyuridine, cytosine arabinoside, 6-mecaptopurine, deoxycoformycin, calcitriol, valrubicin, mithramycin, vinblastine, vinorelbine, topotecan, razoxin, marimastat, COL-3, neovastat, BMS-275291, squalamine, endostatin, SU5416, SU6668, EMD121974, interleukin-12, IM862, angiostatin, vitaxin, droloxifene, idoxyfene, spironolactone, finasteride, cimitidine, trastuzumab, denileukin diftitox, gefitinib, bortezomib, paclitaxel, cremophor-free paclitaxel, docetaxel, epithilone B, BMS-247550, BMS-310705, droloxifene, 4-hydroxytamoxifen, pipendoxifene, ERA-923, arzoxifene, fulvestrant, acolbifene, lasofoxifene, idoxifene, TSE-424, HMR-3339, ZK186619, topotecan, PTK787/ZK 222584, VX-745, PD 184352, rapamycin, 40-O-(2-hydroxyethyl)-rapamycin, temsirolimus, AP-23573, RAD001, ABT-578, BC-210, LY294002, LY292223, LY292696, LY293684, LY293646, wortmannin, ZM336372, L-779,450, PEG-filgrastim, darbepoetin, erythropoietin, granulocyte colony-stimulating factor, zolendronate, prednisone, cetuximab, granulocyte macrophage colony-stimulating factor, histrelin, pegylated interferon alfa-2a, interferon alfa-2a, pegylated interferon alfa-2b, interferon alfa-2b, azacitidine, PEG-L-asparaginase, lenalidomide, gemtuzumab, hydrocortisone, interleukin-11, dexrazoxane, alemtuzumab, all-transretinoic acid, ketoconazole, interleukin-2, megestrol, immune globulin, nitrogen mustard, methylprednisolone, ibritgumomab tiuxetan, androgens, decitabine, hexamethylmelamine, bexarotene, tositumomab, arsenic trioxide, cortisone, editronate, mitotane, cyclosporine, liposomal daunorubicin, Edwina-asparaginase, strontium 89, casopitant, netupitant, an NK-1 receptor antagonist, palonosetron, aprepitant, diphenhydramine, hydroxyzine, metoclopramide, lorazepam, alprazolam, haloperidol, droperidol, dronabinol, dexamethasone, methylprednisolone, prochlorperazine, granisetron, ondansetron, dolasetron, tropisetron, pegfilgrastim, erythropoietin, epoetin alfa, darbepoetin alfa and mixtures thereof.

In certain embodiments, the bioactive agent is selected from, but are not limited to, imatinib mesylate (GLEEVAC®), dasatinib (SPRYCEL®), nilotinib (TASIGNA®), bosutinib (BOSULIF®), trastuzumab (HERCEPTIN®), trastuzumab-DM1, pertuzumab (PERJETA®), lapatinib (TYKERB®), gefitinib (IRESSA®), erlotinib (TARCEVA®), cetuximab (ERBITUX®), panitumumab (VECTIBIX®), vandetanib (CAPRELSA®), vemurafenib (ZELBORAF®), vorinostat (ZOLINZA®), romidepsin (ISTODAX®), bexarotene (TAGRETIN®), alitretinoin (PANRETIN®), tretinoin (VESANOID®), carfilizomib (KYPROLIS®), pralatrexate (FOLOTYN®), bevacizumab (AVASTIN®), ziv-aflibercept (ZALTRAP®), sorafenib (NEXAVAR®), sunitinib (SUTENT®), pazopanib (VOTRIENT®), regorafenib (STIVARGA®), and cabozantinib (COMETRIQ®).

In certain aspects, the bioactive agent is an anti-inflammatory agent, a chemotherapeutic agent, a radiotherapeutic, an additional therapeutic agent, or an immunosuppressive agent.

Suitable chemotherapeutic bioactive agents include, but are not limited to, a radioactive molecule, a toxin, also referred to as cytotoxin or cytotoxic agent, which includes any agent that is detrimental to the viability of cells, and liposomes or other vesicles containing chemotherapeutic compounds. General anti-cancer pharmaceutical agents include: vincristine (ONCOVIN®) or liposomal vincristine (MARQIBO®), daunorubicin (daunomycin or CERUBIDINE®) or doxorubicin (ADRIAMYCIN®), cytarabine (cytosine arabinoside, ara-C, or CYTOSAR®), L-asparaginase (ELSPAR®) or PEG-L-asparaginase (pegaspargase or ONCASPAR®), etoposide (VP-16), teniposide (VUMON®), 6-mercaptopurine (6-MP or PURINETHOL®), methotrexate, cyclophosphamide (CYTOXAN®), prednisone, dexamethasone (Decadron), imatinib (GLEEVEC®), dasatinib (SPRYCEL®), nilotinib (TASIGNA®), bosutinib (BOSULIF®), and ponatinib (ICLUSIG®). Examples of additional suitable chemotherapeutic agents include but are not limited to 1-dehydrotestosterone, 5-fluorouracil decarbazine, 6-mercaptopurine, 6-thioguanine, actinomycin D, adriamycin, aldesleukin, an alkylating agent, allopurinol sodium, altretamine, amifostine, anastrozole, anthramycin (AMC)), an anti-mitotic agent, cis-dichlorodiamine platinum (II) (DDP) cisplatin), diamino dichloro platinum, anthracycline, an antibiotic, an antimetabolite, asparaginase, BCG live (intravesical), betamethasone sodium phosphate and betamethasone acetate, bicalutamide, bleomycin sulfate, busulfan, calcium leucouorin, calicheamicin, capecitabine, carboplatin, lomustine (CCNU), carmustine (BSNU), chlorambucil, cisplatin, cladribine, colchicin, conjugated estrogens, cyclophosphamide, cyclothosphamide, cytarabine, cytarabine, cytochalasin B, cytoxan, dacarbazine, dactinomycin (formerly actinomycin), daunirubicin HCL, daunorucbicin citrate, denileukin diftitox, dexrazoxane, dibromomannitol, dihydroxy anthracin dione, docetaxel, dolasetron mesylate, doxorubicin HCL, dronabinol, E. coli L-asparaginase, emetine, epoetin-α, Erwinia L-asparaginase, esterified estrogens, estradiol, estramustine phosphate sodium, ethidium bromide, ethinyl estradiol, etidronate, etoposide citrororum factor, etoposide phosphate, filgrastim, floxuridine, fluconazole, fludarabine phosphate, fluorouracil, flutamide, folinic acid, gemcitabine HCL, glucocorticoids, goserelin acetate, gramicidin D, granisetron HCL, hydroxyurea, idarubicin HCL, ifosfamide, interferon α-2b, irinotecan HCL, letrozole, leucovorin calcium, leuprolide acetate, levamisole HCL, lidocaine, lomustine, maytansinoid, mechlorethamine HCL, medroxyprogesterone acetate, megestrol acetate, melphalan HCL, mercaptipurine, mesna, methotrexate, methyltestosterone, mithramycin, mitomycin C, mitotane, mitoxantrone, nilutamide, octreotide acetate, ondansetron HCL, paclitaxel, pamidronate disodium, pentostatin, pilocarpine HCL, plimycin, polifeprosan 20 with carmustine implant, porfimer sodium, procaine, procarbazine HCL, propranolol, rituximab, sargramostim, streptozotocin, tamoxifen, taxol, teniposide, tenoposide, testolactone, tetracaine, thioepa chlorambucil, thioguanine, thiotepa, topotecan HCL, toremifene citrate, trastuzumab, tretinoin, valrubicin, vinblastine sulfate, vincristine sulfate, and vinorelbine tartrate.

In some embodiments, the morphic form or pharmaceutical composition of the present invention is administered in combination with a chemotherapeutic agent (e.g., a cytotoxic agent or another chemical compound useful in the treatment of cancer). Examples of chemotherapeutic agents include alkylating agents, antimetabolites, folic acid analogs, pyrimidine analogs, purine analogs and related inhibitors, vinca alkaloids, epipodopyyllotoxins, antibiotics, L-Asparaginase, topoisomerase inhibitors, interferons, platinum coordination complexes, anthracenedione substituted urea, methyl hydrazine derivatives, adrenocortical suppressant, adrenocorticosteroides, progestins, estrogens, antiestrogen, androgens, antiandrogen, and gonadotropin-releasing hormone analog. Also included is 5-fluorouracil (5-FU), leucovorin (LV), irenotecan, oxaliplatin, capecitabine, paclitaxel, and doxetaxel. Non-limiting examples of chemotherapeutic agents include alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gammall and calicheamicin omegall (see, e.g., Agnew, Chem. Inti. Ed Engl. 33:183-186 (1994)); dynemicin, including dynemicin A; bisphosphonates, such as clodronate; an esperamicin; as well as neocarzinostatin chromophore and related chromoprotein enediyne antiobiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN® (doxorubicin, including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfomithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK® polysaccharide complex (JHS Natural Products, Eugene, OR); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL® (paclitaxel; Bristol-Myers Squibb Oncology, Princeton, NJ), ABRAXANE®, cremophor-free, albumin-engineered nanoparticle formulation of paclitaxel (American Pharmaceutical Partners, Schaumberg, IL), and TAXOTERE® doxetaxel (Rhone-Poulenc Rorer, Antony, France); chloranbucil; GEMZAR® gemcitabine; 6-thioguanine; mercaptopurine; methotrexate; platinum coordination complexes such as cisplatin, oxaliplatin and carboplatin; vinblastine; platinum; etoposide (VP-16); ifosfamide; mitoxantrone; vincristine; NAVELBINE® vinorelbine; novantrone; teniposide; edatrexate; daunomycin; aminopterin; xeloda; ibandronate; irinotecan (e.g., CPT-1 1); topoisomerase inhibitor RFS 2000; difluoromethylornithine (DMFO); retinoids such as retinoic acid; capecitabine; and pharmaceutically acceptable salts, acids or derivatives of any of the above. Two or more chemotherapeutic agents can be used in a cocktail to be administered in combination with the morphic form or pharmaceutical composition of the present invention. Suitable dosing regimens of combination chemotherapies are known in the ar. For example, combination dosing regimens are described in Saltz et al., Proc. Am. Soc. Clin. Oncol. 18:233a (1999) and Douillard et al., Lancet 355(9209): 1041-1047 (2000).

Additional therapeutic agents that can be administered in combination with a compound disclosed herein can include bevacizumab, sutinib, sorafenib, 2-methoxyestradiol or 2ME2, finasunate, vatalanib, vandetanib, aflibercept, volociximab, etaracizumab (MEDI-522), cilengitide, erlotinib, cetuximab, panitumumab, gefitinib, trastuzumab, dovitinib, figitumumab, atacicept, rituximab, alemtuzumab, aldesleukine, atlizumab, tocilizumab, temsirolimus, everolimus, lucatumumab, dacetuzumab, HLL1, huN901-DM1, atiprimod, natalizumab, bortezomib, carfilzomib, marizomib, tanespimycin, saquinavir mesylate, ritonavir, nelfinavir mesylate, indinavir sulfate, belinostat, panobinostat, mapatumumab, lexatumumab, dulanermin, ABT-737, oblimersen, plitidepsin, talmapimod, P276-00, enzastaurin, tipifarnib, perifosine, imatinib, dasatinib, lenalidomide, thalidomide, simvastatin, celecoxib, bazedoxifene, AZD4547, rilotumumab, oxaliplatin (Eloxatin), PD0332991, ribociclib (LEE011), amebaciclib (LY2835219), HDM201, fulvestrant (Faslodex), exemestane (Aromasin), PIM447, ruxolitinib (INC424), BGJ398, necitumumab, pemetrexed (Alimta), and ramucirumab (IMC-1121B).

In certain embodiments, the additional therapy is a monoclonal antibody (MAb). Some MAbs stimulate an immune response that destroys cancer cells. Similar to the antibodies produced naturally by B cells, these MAbs may “coat” the cancer cell surface, triggering its destruction by the immune system. For example, bevacizumab targets vascular endothelial growth factor (VEGF), a protein secreted by tumor cells and other cells in the tumor's microenvironment that promotes the development of tumor blood vessels. When bound to bevacizumab, VEGF cannot interact with its cellular receptor, preventing the signaling that leads to the growth of new blood vessels. Similarly, cetuximab and panitumumab target the epidermal growth factor receptor (EGFR), and trastuzumab targets the human epidermal growth factor receptor 2 (HER-2). MAbs that bind to cell surface growth factor receptors prevent the targeted receptors from sending their normal growth-promoting signals. They may also trigger apoptosis and activate the immune system to destroy tumor cells.

In one aspect of the present invention, the bioactive agent is an immunosuppressive agent. The immunosuppressive agent can be a calcineurin inhibitor, e.g. a cyclosporin or an ascomycin, e.g. Cyclosporin A (NEORAL®), FK506 (tacrolimus), pimecrolimus, a mTOR inhibitor, e.g. rapamycin or a derivative thereof, e.g. Sirolimus (RAPAMUNE®), Everolimus (CERTICAN®), temsirolimus, zotarolimus, biolimus-7, biolimus-9, a rapalog, e.g.ridaforolimus, azathioprine, campath 1H, a SiP receptor modulator, e.g. fingolimod or an analogue thereof, an anti IL-8 antibody, mycophenolic acid or a salt thereof, e.g. sodium salt, or a prodrug thereof, e.g. Mycophenolate Mofetil (CELLCEPT®), OKT3 (ORTHOCLONE OKT3®), Prednisone, ATGAM®, THYMOGLOBULN4®, Brequinar Sodium, OKT4, T10B9.A-3A, 33B3.1, 15-deoxyspergualin, tresperimus, Leflunomide ARAVA®, CTLAI-Ig, anti-CD25, anti-IL2R, basiliximab (SIMULECT®), daclizumab (ZENAPAX®), mizorbine, methotrexate, dexamethasone, ISAtx-247, SDZ ASM 981 (pimecrolimus, ELIDEL®), CTLA4lg (Abatacept), belatacept, LFA3lg, etanercept (sold as ENBREL by Immunex), adalimumab (HUMIRA), infliximab (REMICADE®), an anti-LFA-1 antibody, natalizumab (ANTEGREN®), enlimomab, gavilimomab, antithymocyte immunoglobulin, siplizumab, alefacept efalizumab, pentasa, mesalazine, asacol, codeine phosphate, benorylate, fenbufen, naprosyn, diclofenac, etodolac and indomethacin, aspirin and ibuprofen.

In some embodiments, the bioactive agent is a therapeutic agent which is a biologic such a cytokine (e.g., interferon or an interleukin (e.g., IL-2)) used in cancer treatment. In some embodiments the biologic is an anti-angiogenic agent, such as an anti-VEGF agent, e.g., bevacizumab (AVASTIN®). In some embodiments the biologic is an immunoglobulin-based biologic, e.g., a monoclonal antibody (e.g., a humanized antibody, a fully human antibody, an Fc fusion protein or a functional fragment thereof) that agonizes a target to stimulate an anti-cancer response, or antagonizes an antigen important for cancer. Such agents include RITUXAN® (rituximab); ZENAPAX® (daclizumab); SIMULECT® (basiliximab); SYNAGIS® (palivizumab); REMICADE® (infliximab); HERCEPTIN® (trastuzumab); MYLOTARG® (gemtuzumab ozogamicin); CAMPATH® (alemtuzumab); ZEVALN® (ibritumomab tiuxetan); HUMIRA® (adalimumab); XOLAIR (omalizumab); BEXXAR® (tositumomab-I-131); RAPTIVA® (efalizumab); ERBITUX® (cetuximab); AVASTIN® (bevacizumab); TYSABRI® (natalizumab); ACTEMRA® (tocilizumab); VECTIBIX® (panitumumab); LUCENTIS® (ranibizumab); SOURIS® (eculizumab); CIMZIA® (certolizumab pegol); SIMPONI® (golimumab); ILARIS® (canakinumab); STELARA® (ustekinumab); ARZERRA® (ofatumumab); PROLIA® (denosumab); NUMAX® (motavizumab); ABTHRAX® (raxibacumab); BENLYSTA® (belimumab); YERVOY® (ipilimumab); ADCETRIS® (brentuximab vedotin); PERJETA® (pertuzumab); KADCYLA (ado-trastuzumab emtansine); and GAZYVA (obinutuzumab). Also included are antibody-drug conjugates.

In certain embodiments, the additional therapy is bendamustine. In certain embodiments, the additional therapy is obinutuzmab. In certain embodiments, the additional therapy is a proteasome inhibitor, for example ixazomib or oprozomib. In certain embodiments, the additional therapy is a histone deacetylase inhibitor, for example ACY241. In certain embodiments, the additional therapy is a BET inhibitor, for example GSK525762A, OTX015, BMS-986158, TEN-010, CPI-0610, INCB54329, BAY1238097, FT-1101, ABBV-075, BI 894999, GS-5829, GSK1210151A (I-BET-151), CPI-203, RVX-208, XD46, MS436, PFI-1, RVX2135, ZEN3365, XD14, ARV-771, MZ-1, PLX5117, 4-[2-(cyclopropylmethoxy)-5-(methanesulfonyl)phenyl]-2-methylisoquinolin-1(2H)-one, EP11313 and EP11336. In certain embodiments, the additional therapy is an MCL-1 inhibitor, for example AZD5991, AMG176, MIK665, S64315, or S63845. In certain embodiments, the additional therapy is an LSD-1 inhibitor, for example ORY-1001, ORY-2001, INCB-59872, IMG-7289, TAK-418, GSK-2879552, 4-[2-(4-amino-piperidin-1-yl)-5-(3-fluoro-4-methoxy-phenyl)-1-methyl-6-oxo-1,6-dihydropyrimidin-4-yl]-2-fluoro-benzonitrile or a salt thereof. In certain embodiments, the additional therapy is a CS1 antibody, for example elotuzumab. In certain embodiments, the additional therapy is a CD38 antibody, for example daratumumab or isatuximab. In certain embodiments, the additional therapy is a BCMA antibody or antibody-conjugate, for example GSK2857916 or BI 836909.

In certain embodiments, the bioactive agent is selinexor. In certain embodiments, Compound 1 is administered in combination with selinexor. In certain embodiments, Compound 1 is administered in combination with aspirin.

In other embodiments a compound described herein is administered in combination with a drug selected from selinexor, oxaphenamide, belantamab mafodotin, denosumab, zoledronic acid, plerixafor, eltrombopag, ipilumumab, palbociclib, ricolinostat, afuresertib, dinaciclib, filanesib, indatuximab ravtansine, masitinib, sonidegib, sotatercept, ulocuplumab, and urelumab.

In certain embodiments Compound 1 is used in combination with a chemokine receptor antagonist, for example plerixafor.

In certain embodiments Compound 1 is used in combination with a vaccine, for example PVX-410.

In certain embodiments Compound 1 is used in combination with a chemotherapeutic agent, for example bendamustine, busulfan, carmustine, cyclophosphamide, doxorubicin, etoposide, fludarabine, melphalan, or vincristine.

In certain embodiments Compound 1 is used in combination with a conjugated antibody, for example belantamab mafodotin, CC-99712, HDP-101, or MEDI2228.

In certain embodiments Compound 1 is used in combination with a phospholipid-drug conjugate, for example CLR 131.

In certain embodiments a compound described herein is used in combination with a chemokine receptor antagonist, for example plerixafor.

In certain embodiments a compound described herein is used in combination with a vaccine, for example PVX-410.

In certain embodiments a compound described herein is used in combination with a chemotherapeutic agent, for example bendamustine, busulfan, carmustine, cyclophosphamide, doxorubicin, etoposide, fludarabine, melphalan, or vincristine.

In certain embodiments a compound described herein is used in combination with a conjugated antibody, for example belantamab mafodotin, CC-99712, HDP-101, or MEDI2228.

In certain embodiments a compound described herein is used in combination with a phospholipid-drug conjugate, for example CLR 131.

In certain embodiments a compound described herein is used in combination with a CHOP regimen (cyclophosphamide, vincristine, prednisone, and daunorubicin). In certain embodiments Compound 1 is used in combination with a CHOP regimen (cyclophosphamide, vincristine, prednisone, and daunorubicin). In certain embodiments Compound 1, rituximab, and CHOP are administered in combination.

In certain embodiments a compound described herein is used in combination with a CVP regimen (cyclophosphamide, vincristine, and prednisone). In certain embodiments Compound 1 is used in combination with a CVP regimen (cyclophosphamide, vincristine, and prednisone). In certain embodiments Compound 1, rituximab, and CVP are administered in combination.

In certain embodiments a compound described herein is used in combination with a romidepsin, belinostat, or brentuximab to treat cancer, for example T-NHL. In certain embodiments Compound 1 is used in combination with a romidepsin, belinostat, or brentuximab to treat cancer, for example T-NHL.

In certain embodiments a compound described herein is used in combination with a polatuzumab, tafastamab, CAR-T, a BTK inhibitor, or a PI3Kinase inhibitor to treat cancer, for example B-NHL. In certain embodiments Compound 1 is used in combination with a polatuzumab, tafastamab, CAR-T, a BTK inhibitor, or a PI3Kinase inhibitor to treat cancer, for example B-NHL In certain embodiments Compound 1 is used in the treatment of relapsed and/or refractory mantle cell leukemia. In certain embodiments Compound 1 and rituximab are used in the treatment of relapsed and/or refractory mantle cell leukemia. In certain embodiments Compound 1, rituximab, and bendamustine are used in the treatment of relapsed and/or refractory mantle cell leukemia. In certain embodiments Compound 1, rituximab, and ibrutinib are used in the treatment of relapsed and/or refractory mantle cell leukemia.

In certain embodiments the cancer is a primary central nervous system lymphoma (PCNSL).

In certain embodiments Compound 1 is used in the treatment of relapsed and/or refractory marginal zone leukemia. In certain embodiments Compound 1 and rituximab are used in the treatment of relapsed and/or refractory marginal zone leukemia. In certain embodiments Compound 1, rituximab, and dexamethasone are used in the treatment of relapsed and/or refractory marginal zone leukemia.

In certain embodiments Compound 1 is used in combination with rituximab and DHAP (dexamethasone, cytarabine, and cisplatin). In certain embodiments Compound 1 is used in combination with DHAP (dexamethasone, cytarabine, and cisplatin). In certain embodiments Compound 1 is used in combination with rituximab and ICE (ifosfamide, carboplatin, and etoposide). In certain embodiments Compound 1 is used in combination with ICE (ifosfamide, carboplatin, and etoposide). In certain embodiments Compound 1 is used in combination with rituximab and GemOx (gemcitabine and oxaliplatin). In certain embodiments Compound 1 is used in combination with GemOx (gemcitabine and oxaliplatin). In certain embodiments Compound 1 is used in combination with polatuzumab and brentuximab. In certain embodiments Compound 1 is used in combination with polatuzumab. In certain embodiments Compound 1 is used in combination with tafasitimab. In certain embodiments Compound 1 is used in combination with tafasitimab and lenolidomide. In certain embodiments Compound 1 is used in combination with rituximab and lenolidomide. In certain embodiments Compound 1 is used in combination with anti CD19 CART (for example axicabtagene, lisocabtagene, tisafenlucleucel, loncastuximab, or tesirine). In certain embodiments one of the above combinations is used in the treatment of DLBCL or B-NHL.

In certain embodiments Compound 1 is used in combination with praltrexate. In certain embodiments Compound 1 is used in combination with bendamustine. In certain embodiments Compound 1 is used in combination with brexucabtagene. In certain embodiments Compound 1 is used in combination with bortezomib and rituximab. In certain embodiments Compound 1 is used in combination with bendamustine and rituximab. In certain embodiments Compound 1 is used in combination with VR-CAP (rituximab, cyclophospharmide, doxorubicin, prednisone, and bortzomib).

In certain embodiments Compound 1 is used in combination with carfilzomib and daratumumab.

In certain embodiments Compound 1 is used in combination with abecma. In certain embodiments Compound 1 is used in combination with melflufen. In certain embodiments Compound 1 is used in combination with ciltacabtagene autoleucel.

Non-limiting examples of combination therapies or mono therapies that include Compound 1 Form I, a pharmaceutically acceptable composition prepared from Compound 1 Form I, or anew dosage regimen of Compound 1 for specific cancers are described below.

| Disease | Characterization | Agent(s) |
|---|---|---|
| MCL | Relapsed and/or | Compound 1 |
| | refractory | |
| MCL | Relapsed and/or | Compound 1 and |
| | refractory | rituximab |
| MCL | Relapsed and/or | Compound 1, rituximab, |
| | refractory | and bendamustine |
| MCL | Relapsed and/or | Compound 1, rituximab, |
| | refractory | and ibrutinib |
| Follicular | Relapsed and/or | Compound 1 |
| or marginal | refractory | |
| zone lymphoma | | |
| Follicular, marginal | Relapsed and/or | Compound 1 and |
| zone, mucosa-associated | refractory | rituximab |
| lymphoid tissue, or | | |
| small lymphocytic, | | |
| lymphoma | | |
| Mantle cell | Relapsed and/or | Compound 1 and |
| lymphoma | refractory | rituximab |
| Follicular, marginal | Relapsed and/or | Compound 1, rituximab, |
| zone, mucosa-associated | refractory | and dexamethasone |
| lymphoid tissue, or | | |
| small lymphocytic, | | |
| lymphoma | | |
| Mantle cell lymphoma | Relapsed and/or | Compound 1, rituximab, |
| | refractory | and dexamethasone |
| Diffuse large b-cell, | Relapsed and/or | Compound 1 |
| mantle cell, or | refractory | |
| follicular lymphoma | | |
| Transformed: diffuse | Relapsed and/or | Compound 1 |
| large b-cell, mantle | refractory | |
| cell, or follicular | | |
| lymphoma | | |
| Diffuse large b-cell, | Relapsed and/or | Compound 1 and rituximab |
| mantle cell, or | refractory | |
| follicular lymphoma | | |
| Transformed: diffuse | Relapsed and/or | Compound 1, rituximab, |
| large b-cell, mantle | refractory | and benadmustine |
| cell, or follicular | | |
| lymphoma | | |
| MCL | Frontline | Compound 1 |
| MCL | Frontline | Compound 1 and rituximab |
| MCL | Frontline | Compound 1, rituximab, |
| | | and bendamustine |
| Follicular, marginal | Frontline | Compound 1 and |
| zone, or small | | rituximab |
| lymphocytic lymphoma | | |
| Follicular, marginal | Frontline | Compound 1, rituximab, |
| zone, or small | | and CHOP |
| lymphocytic lymphoma | | |
| Diffuse large b-cell | Frontline | Compound 1, rituximab, |
| lymphoma | | and CHOP |

Additional, non-limiting examples of combination therapies or mono therapies that include Compound 1 Form L a pharmaceutically acceptable composition prepared from Compound 1 Form I, or a new dosage regimen of Compound 1 for specific cancers are described below.

| Disease | Combination or Therapy |
|---|---|
| MM | Compound 1, a CD38 antibody, and dexamethasone |
| MM | Compound 1 |
| MM | Compound 1 and dexamethasone |
| MM | Compound 1 and a monoclonal antibody |
| MM | Compound 1 and a B-cell maturation antigen (BCMA) |
| MM | Compound 1 and daratumamab |
| MM | Compound 1 and CAR-T |
| MM | Compound 1 and a bispecific CAR-T |
| MM | Compound 1 and a BCMA CD38 CAR-T |
| MM | Compound 1 and a bispecific T cell engager |
| MM | Compound 1, dexamethasone, and carfilizomib |
| PTCL | Compound 1 and CHOP |
| PTCL | Compound 1 and romidepsin |
| PTCL | Compound 1 |
| MCL | Compound 1 and rituximab |
| MCL | Compound 1 |
| DLBCL | Compound 1 and CHOP |
| DLBCL | Compound 1 |
| NHL | Compound 1 and polatuzumab |
| NHL | Compound 1 and CD20 bispecific antibody |
| NHL | Compound 1 and rituximab |
| NHL | Compound 1 and BTK inhibitor |
| NHL | Compound 1 and tafasitimab |
| NHL | Compound 1 and BTK inhibitor |
| NHL | Compound 1 and an antibody, cyclophosphamide, |
| | predinisone, and/or doxorubicin |
| NHL | Compound 1, bendamustine, and rituximab |
| NHL | Compound 1, monoclonal antibody, bispecific |
| | T cell engager, and/or CHP |

In certain embodiments a combination described above is used to treat a different cancer. In certain embodiments the cancer is doubly refractory, triply refractory, quad refractory, or penta refractory, for example penta refractory multiple myeloma.

In certain embodiments a monotherapy or combination described herein additionally comprises administering one or more additional therapeutic agents to decrease side effects of the therapy. For example, in certain embodiments Compound 1 or a combination comprising Compound 1 is administered concurrently, before, or after administration of an antineutropenia medication, antinausea medication, an antihistamine, and/or an antipain medication. Non-limiting examples of antineutropenia medications include growth factors for example a granulocyce colony stimulating factor (G-CSF). In certain embodiments a therapy in a table above is administered in combination with a G-CSF. G-CSF (or another active agent) can be given before, with after, or on different days than Compound 1.

Non-limiting examples of granulocyte colony stimulating factors include filgrastim (in the form of neupogen, zarxio, nivestym, or another form), CG-10639, and PEGF.

In certain embodiments the granulocyte colony stimulating factor is pegfilgrastim. In certain embodiments the granulocyte colony stimulating factor is Neulasta. In certain embodiments the granulocyte colony stimulating factor is selected from Ristempa, Tezmota, Fulphila, Pelgraz, Udenyca, Udenyca, Pelmeg, Ziextenzo, Grasustek, Ziextenzo, Lapelga, Neutropeg, Cegfila, Nyvepria, and Stimufend.

In certain embodiments the therapy described herein further comprises an antinausea medication. Non-limiting examples of antinauasea medications include aprepitant, dolasetron, granisetron, ondansetron, palonosetron, proclorperazine, promethazine, netupitant-palonosetron, rolapitant, lorazepam, metoclopramide, famotidine, dexamethasone, and ranitidine.

In certain embodiments the therapy described herein further comprises an antihistamine medication. Non-limiting examples of antihistamine medications include benadryl, cetirizine, loratadine, and fexofenadine.

In certain embodiments the therapy described herein further comprises an antipain medication. Non-limiting examples of antipain medications include tramadol, hydromorphone, methadone, morphine, oxycodone, hydrocodone, oxymorphone, fentanyl, and tapentadol.

### Pharmaceutical Compositions

In certain embodiments a pharmaceutical composition comprising an effective amount of Compound 1 Form I and one or more pharmaceutically acceptable excipients is provided. In other embodiments a pharmaceutical composition is prepared from Compound 1 Form I. For example Compound 1 Form I can be dissolved or suspended in an excipient and then the mixture can be used to fill gel caps or used as a liquid dosage form, for example a pharmaceutical composition for intravenous administration. In other embodiments Compound 1 Form I is dissolved in a solvent, optionally mixed with one or more excipients, and then dried or spray dried to form a solid. In other embodiments Compound 1 is used in a pharmaceutical composition for use in a new dosage regimen described herein. The pharmaceutical composition may contain a compound or salt as the only active agent, or, in an alternative embodiment, the compound and at least one additional active agent.

In certain embodiments, the pharmaceutical composition is in a dosage form that contains from about 1 μg to about 2000 μg, from about 10 μg to about 1000 μg, from about 50 μg to about 600 μg, or from about 100 μg to about 400 μg of the active compound. In another embodiment the pharmaceutical composition is in a dosage form that contains from about 1 μg to about 400 μg, from about 5 μg to about 400 μg, from about 10 mg to about 250 μg, or from about 25 μg to about 250 μg of the active compound. Examples are dosage forms with at least, or in some embodiments, not more than, 0.1, 1, 5, 10, 12.5, 25, 37.5, 50, 62.5, 75, 100, 200, 250, 300, 400, 500, 600, 700, or 750 μg of active compound, or its salt.

The pharmaceutical composition may also include a molar ratio of the active compound and an additional bioactive agent. For example, the pharmaceutical composition may contain a molar ratio of about 0.0005:1, about 0.001:1, about 0.01:1, about 0.05:1, or about 0.1:1 of active compound to an anti-inflammatory or immunosuppressing agent. For example, the pharmaceutical composition may contain a molar ratio of about 0.5:1, about 1:1, about 2:1, about 3:1 or from about 1.5:1 to about 4:1 of active compound to an anti-inflammatory or immunosuppressing agent. Compounds disclosed herein may be administered orally, topically, parenterally, by inhalation or spray, sublingually, via implant, including ocular implant, transdermally, via buccal administration, rectally, as an ophthalmic solution, injection, including ocular injection, intravenous, intra-aortal, intracranial, subdermal, intraperitioneal, subcutaneous, transnasal, sublingual, or rectal or by other means, in dosage unit formulations containing conventional pharmaceutically acceptable carriers. For ocular delivery, the compound can be administered, as desired, for example, via intravitreal, intrastromal, intracameral, sub-tenon, sub-retinal, retrobulbar, peribulbar, suprachorodial, conjunctival, subconjunctival, episcleral, periocular, transscleral, retrobulbar, posterior juxtascleral, circumcorneal, or tear duct injections, or through a mucus, mucin, or a mucosal barrier, in an immediate or controlled release fashion or via an ocular device.

The pharmaceutical composition may be formulated as any pharmaceutically useful form, e.g., as an aerosol, a cream, a gel, a pill, an injection or infusion solution, a capsule, a tablet, a syrup, a transdermal patch, a subcutaneous patch, a dry powder, an inhalation formulation, in a medical device, suppository, buccal, or sublingual formulation, parenteral formulation, or an ophthalmic solution. Some dosage forms, such as tablets and capsules, are subdivided into suitably sized unit doses containing appropriate quantities of the active components, e.g., an effective amount to achieve the desired purpose.

In certain embodiments the pharmaceutical composition is a tablet that is appropriate for oral administration.

Carriers include excipients and diluents and must be of sufficiently high purity and sufficiently low toxicity to render them suitable for administration to the patient being treated. The carrier can be inert or it can possess pharmaceutical benefits of its own. The amount of carrier employed in conjunction with the compound is sufficient to provide a practical quantity of material for administration per unit dose of the compound.

Classes of carriers include, but are not limited to binders, buffering agents, coloring agents, diluents, disintegrants, emulsifiers, flavorants, glidents, lubricants, preservatives, stabilizers, surfactants, tableting agents, and wetting agents. Some carriers may be listed in more than one class, for example vegetable oil may be used as a lubricant in some formulations and a diluent in others. Pharmaceutically acceptable carriers are carriers that do not cause any severe adverse reactions in the human body when dosed in the amount that would be used in the corresponding pharmaceutical composition. Exemplary pharmaceutically acceptable carriers include sugars, starches, celluloses, powdered tragacanth, malt, gelatin; talc, and vegetable oils. Optional active agents may be included in a pharmaceutical composition, which do not substantially interfere with the activity of the morphic form or pharmaceutical composition of the present invention.

Formulations suitable for rectal administration are typically presented as unit dose suppositories. These may be prepared by admixing the active compound with one or more conventional solid carriers, for example, cocoa butter, and then shaping the resulting mixture.

Formulations suitable for topical application to the skin preferably take the form of an ointment, cream, lotion, paste, gel, spray, aerosol, or oil. Carriers which may be used include petroleum jelly, lanoline, polyethylene glycols, alcohols, transdermal enhancers, and combinations of two or more thereof.

Formulations suitable for transdermal administration may be presented as discrete patches adapted to remain in intimate contact with the epidermis of the recipient for a prolonged period of time. Formulations suitable for transdermal administration may also be delivered by iontophoresis (see, for example, Pharmaceutical Research 3 (6):318 (1986)) and typically take the form of an optionally buffered aqueous solution of the active compound. In certain embodiments, microneedle patches or devices are provided for delivery of drugs across or into biological tissue, particularly the skin. The microneedle patches or devices permit drug delivery at clinically relevant rates across or into skin or other tissue barriers, with minimal or no damage, pain, or irritation to the tissue.

Formulations suitable for administration to the lungs can be delivered by a wide range of passive breath driven and active power driven single/multiple dose dry powder inhalers (DPI). The devices most commonly used for respiratory delivery include nebulizers, metered-dose inhalers, and dry powder inhalers. Several types of nebulizers are available, including jet nebulizers, ultrasonic nebulizers, and vibrating mesh nebulizers. Selection of a suitable lung delivery device depends on parameters, such as nature of the drug and its formulation, the site of action, and pathophysiology of the lung.

In certain embodiments, a compound described herein, for example Compound 1, is formulated as described in Table A.

The pharmaceutical compositions/combinations can be formulated for oral administration. These compositions can contain any amount of active compound that achieves the desired result, for example between 0.1 and 99 weight % (wt. %) of the compound and usually at least about 5 wt. % of the compound. Some embodiments contain from about 25 wt. % to about 50 wt. % or from about 5 wt. % to about 75 wt. % of the compound.

In certain embodiments the pharmaceutical composition is a tablet comprising 12.5 μg of Compound 1 or a pharmaceutically acceptable salt thereof.

In certain embodiments the pharmaceutical composition is a tablet comprising 25 μg of Compound 1 or a pharmaceutically acceptable salt thereof.

In certain embodiments the pharmaceutical composition is a tablet comprising 37.5 μg of Compound 1 or a pharmaceutically acceptable salt thereof.

In certain embodiments the pharmaceutical composition is a tablet comprising 50 μg of Compound 1 or a pharmaceutically acceptable salt thereof.

In certain embodiments the pharmaceutical composition is a tablet comprising 62.5 μg of Compound 1 or a pharmaceutically acceptable salt thereof.

In certain embodiments the pharmaceutical composition is a tablet comprising 75 μg of Compound 1 or a pharmaceutically acceptable salt thereof.

In certain embodiments the pharmaceutical composition is a tablet comprising 87.5 μg of Compound 1 or a pharmaceutically acceptable salt thereof.

In certain embodiments the pharmaceutical composition is a tablet comprising 100 μg of Compound 1 or a pharmaceutically acceptable salt thereof.

### Liquid Filled and Gel Capsules

In certain embodiments a liquid filled or gel filled capsule (as used herein gel capsule refers to either a liquid filled or gel filled capsule) is provided comprising an effective amount of Compound 1 Form I as a suspension and one or more pharmaceutically acceptable excipients. In other embodiments a gel capsule is prepared from Compound 1 Form I. In certain embodiments the gel capsule comprises Compound 1 Form I as a suspension or Compound 1 as a solution and a liquid excipient. Non-limiting examples of liquid excipients include polyethylene glycol, water surfactants such as polysorbates, alcohols such as ethanol, and glycerin.

In certain embodiments the gel capsule is a hydrogel capsule. Hydrogels provide a unique morphology including swelling and elasticity. The rate at which the hydrogel swells can be modulated to change the release rate of the drug and the elasticity of the system can be modulated to change stability and delay release. Hydrogel capsules can provide both spatial and temporal control over the release of Compound 1 Form I or Compound 1. In other embodiments Compound 1 Form I or Compound 1 is administered as hydrogel outside of a capsule. For example, as a liquid solution or suspension that forms a hydrogel upon administration to the body. Non-limiting examples of hydrogels include various macroscopic hydrogels such as in situ-gelling hydrogels (e.g., a liquid solution that forms a gel after injection into the body), shear-thinning hydrogels (e.g., a hydrogel that can be injected into the body via shear stress force), microporous hydrogels (hydrogels with large, interconnected pores that can be collapsed and then recovered upon mechanical force). Alternatively, a non-macroscopic hydrogel can be used for example a microgels or nanogels.

In certain embodiments Compound 1 Form I is a suspended solid in the hydrogel.

TABLE A
Example Drug Product Composition

| | | A | B | C |
|---|---|---|---|---|
| Item # | Ingredients | mg/ca | mg/ca | mg/caps |
| 1 | Compound 1 | 0.40 | 0.10 | 0.025 |
| 2 | PEG 400 | 1187.4 | 296.87 | 249.21 |
| | TOTAL | 1200 | 300 | 250 |

In certain embodiments the concentration of Compound 1 in Table A is instead about 0.20, 0.15, 0.10, 0.05, or 0.01 mg/capsule and the concentration of PEG 400 is adjusted to maintain a total volume of 1200, 300, or 250 mg/capsule.

In certain embodiments, the present invention provides a pharmaceutical composition comprising Compound 1, a nonaqueous solvent, a preservative, and/or a pH control agent.

In certain embodiments, a compound described herein, for example Compound 1, is formulated as described herein for example as described in Table B or Table C.

Non-limiting examples of preservatives include alcohols (for example, ethanol, benzyl alcohol), aluminum acetate, benzalkonium chloride, benzethonium chloride, benzoic acid and salts thereof (for example, potassium benzoate, sodium benzoate), boric acid and salts thereof (for example, sodium borate), bronopol, butylene glycol, butylated hydroxyanisole, calcium acetate, calcium chloride, cetrimide, chlorhexidine, chlorobutanol, chlorocresol, chloroxylenol, citric acid monohydrate, cresol, dimethyl ether, edetic acid and salts thereof, gelatin, glycerin, hexetidine, imidurea, lactic acid and salts thereof (for example, calcium lactate, sodium lactate), monothioglycerol, parabens (for example, butylparaben, ethylparaben methylparaben, propylparaben, propylparaben sodium), pentetic acid and salts thereof, phenol, phenoxyethanol, phenylethyl alcohol, phenylmercuric acetate, phenylmercuric borate, phenylmercuric nitrate, propyl gallate, potassium metabisulfite, sorbic acid and salts thereof (for example, potassium sorbate), propionic acid and salts thereof (for example, sodium propionate), propylene glycol, sodium acetate, sodium sulfite, sulfur dioxide, thimerosal, and xylitol.

The term pH control agent can be used interchangeably with pH adjustment agent, pH regulator, and/or buffering agent. Non-limiting examples of pH control agents include acetic acid, glacial acetic acid, adipic acid, strong ammonia solution and salts thereof (for example, ammonium carbonate, ammonium chloride, ammonium phosphate), arginine, boric acid and salts thereof (for example, sodium borate), calcium carbonate, calcium hydroxide, calcium lactate, calcium phosphate, tribasic, citric acid (anhydrous or monohydrate) and salts thereof (for example, potassium citrate, sodium citrate), diethanolamine, fumaric acid and salts thereof, glycine, gluconic acid, hydrochloric acid, diluted hydrochloric acid, alpha-lactalbumin, lactic acid and salts thereof (for example, sodium lactate solution), lysine hydrochloride, maleic acid, malic acid, methionine, monoethanolamine, monosodium glutamate, meglumine, nitric acid, phosphoric acid and salts thereof (for example, dibasic sodium phosphate, monobasic sodium phosphate, potassium phosphate, potassium hydrogen phosphate, dibasic, potassium phosphate), diluted phosphoric acid, potassium bicarbonate, potassium hydroxide, potassium metaphosphate, monobasic, propionic acid, racemethionine, sodium acetate, sodium bicarbonate, sodium carbonate, sodium hydroxide, succinic acid, sulfuric acid, tartaric acid, trolamine, a hydroxide, an amine, and salts thereof.

Non-limiting examples of buffering agents include, but are not limited to, adipic acid, ammonia solution, boric acid, calcium carbonate, calcium hydroxide, calcium lactate, calcium phosphate, citric acid, sodium phosphate, diethanolamine, maleic acid, malic acid, methionine, monoethanolamine, sodium glutamate, phosphoric acid, potassium citrate, sodium acetate, sodium bicarbonate, sodium borate, sodium carbonate, sodium citrate, sodium hydroxide, sodium lactate and triethanolamine.

In certain embodiments, the buffering agent is selected from carbonates, citrates, gluconates, lactates, phosphates, tartrates, potassium metaphosphate, potassium phosphate, monobasic, sodium acetate, sodium citrate, anhydrous and dihydrate.

In certain embodiments, the pharmaceutical composition includes water.

In certain embodiments, the pharmaceutical composition includes a non-aqueous or anhydrous solvent.

Nonlimiting examples of non-aqueous solvents include alcohols (for example, ethanol), PEG 300, PEG 400, propylene glycol, cremophor, caplex 355, Capryol™ 90, Lauroglycol™ 90, TranscutolHP, butylated hydroxytoluene, benzyl alcohol, citric acid, triacetin, propylene glycol, fats and oils.

TABLE B
25 μg strength formulation

| | mg/capsule | Percent | |
|---|---|---|---|
| Ingredient | (25 μg) | w/w | Function |
| Compound 1 | 0.0250 | 0.010 | Drug Substance |
| PEG 400 | 247.4125 | 98.965 | Nonaqueous solvent |
| Butylated Hydroxytoluene | 0.0625 | 0.025 | Preservative |
| Citric Acid, Anhydrous | 2.500 | 1.000 | pH control |
| Total | 250.0000 | 100.000 | |

TABLE C
100 μg strength formulation

| | mg/capsule | Percent | |
|---|---|---|---|
| Ingredient | (100 μg) | w/w | Function |
| Compound 1 | 0.100 | 0.033 | Drug Substance |
| PEG 400 | 296.825 | 98.942 | Nonaqueous solvent |
| Butylated | 0.075 | 0.025 | Preservative |
| Hydroxytoluene, | | | |
| Citric Acid, Anhydrous | 3.000 | 1.000 | pH control |
| Total | 300.0000 | 100.000 | |

In certain embodiments the concentration of Compound 1 in Table A is instead about 0.20, 0.15, 0.10, 0.05, or 0.01 mg/capsule and the concentration of the other excipients is adjusted to maintain or achieve a total volume of 1200, 300, or 250 mg/capsule.
1. In certain embodiments, Compound 1 is provided in a liquid filled capsule comprising a solvent, preservative, and pH control agent, wherein Compound 1 was Compound 1 Form I as described herein before being dissolved.
2. The pharmaceutical composition of embodiment 1, wherein the capsule is filled with a solution comprising about 0.001, 0.005, 0.01, 0.015, 0.02, 0.025, 0.03, 0.033, 0.035, 0.04, 0.045, 0.05, 0.06, 0.07, 0.08, 0.09, 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, 1, 1.5, 2, 2.5, 3, 3.5, 4, 4.5, or 5% Compound 1 by weight.
3. The pharmaceutical composition of embodiment 1, wherein the capsule is filled with a solution comprising at least about 0.001, 0.005, 0.01, 0.015, 0.02, 0.025, 0.03, 0.033, 0.035, 0.04, 0.045, 0.05, 0.06, 0.07, 0.08, 0.09, 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, 1, 1.5, 2, 2.5, 3, 3.5, 4, 4.5, or 5% Compound 1 by weight.
4. The pharmaceutical composition of any one of embodiments 1-3, wherein the solvent is not aqueous.
5. The pharmaceutical composition of embodiment 4, wherein the solvent is PEG.
6. The pharmaceutical composition of embodiment 5, wherein the solvent is PEG 400.
7. The pharmaceutical composition of any one of embodiments 1-6, wherein the capsule is filled with a solution comprising about 95, 95.5, 96, 96.5, 97, 97.5, 98, 98.1, 98.2, 98.3, 98.4, 98.5, 98.6, 98.65, 98.7, 98.75, 98.8, 98.85, 98.9, 99, 99.1, 99.2, 99.3, 99.4, or 99.5% solvent by weight.
8. The pharmaceutical composition of any one of embodiments 1-6, wherein the capsule is filled with a solution comprising at least about 85, 90, 95, 95.5, 96, 96.5, 97, 97.5, 98, 98.1, 98.2, 98.3, 98.4, 98.5, 98.6, 98.65, 98.7, 98.75, 98.8, 98.85, 98.9, 99, 99.1, 99.2, 99.3, 99.4, or 99.5% solvent by weight.
9. The pharmaceutical composition of any one of embodiments 1-8, wherein the preservative is butylated hydroxytoluene.
10. The pharmaceutical composition of any one of embodiments 1-9, wherein the capsule is filled with a solution comprising at least about 0.01, 0.05, 0.1, 0.15, 0.2, 0.25, 0.3, 0.35, 0.4, 0.45, 0.5, 0.55, 0.6, 0.65, 0.8, 0.85, 0.9, 0.95, or 1% preservative by weight.
11. The pharmaceutical composition of any one of embodiments 1-9, wherein the capsule is filled with a solution comprising about 0.01, 0.05, 0.1, 0.15, 0.2, 0.25, 0.3, 0.35, 0.4, 0.45, 0.5, 0.55, 0.6, 0.65, 0.8, 0.85, 0.9, 0.95, or 1% preservative by weight.
12. The pharmaceutical composition of any one of embodiments 1-11, wherein the pH control agent is citric acid.
13. The pharmaceutical composition of any one of embodiments 1-11, wherein the pH control agent is anhydrous citric acid.
14. The pharmaceutical composition of any one of embodiments 1-13, wherein the capsule is filled with a solution comprising at least about 0.01, 0.05, 0.1, 0.15, 0.2, 0.25, 0.3, 0.35, 0.4, 0.45, 0.5, 0.55, 0.6, 0.65, 0.8, 0.85, 0.9, 0.95, 1, 1.1, 1.2, 1.3, 1.4, 1.5, 1.6, 1.7, 1.8, 1.9, 2, 2.25, 2.5, 2.75, or 3% pH control agent by weight.
15. The pharmaceutical composition of any one of embodiments 1-13, wherein the capsule is filled with a solution comprising about 0.01, 0.05, 0.1, 0.15, 0.2, 0.25, 0.3, 0.35, 0.4, 0.45, 0.5, 0.55, 0.6, 0.65, 0.8, 0.85, 0.9, 0.95, 1, 1.1, 1.2, 1.3, 1.4, 1.5, 1.6, 1.7, 1.8, 1.9, 2, 2.25, 2.5, 2.75, or 3% pH control agent by weight.
16. The pharmaceutical composition of any one of embodiments 1-15, wherein the pharmaceutical composition comprises about 12.5, about 25, about 37.5, about 50, about 62.5, or about 75 μg of Compound 1.
17. The pharmaceutical composition of any one of embodiments 1-15, wherein the pharmaceutical composition comprises about 12.5 μg of Compound 1.
18. The pharmaceutical composition of any one of embodiments 1-15, wherein the pharmaceutical composition comprises about 25 μg of Compound 1.
19. The pharmaceutical composition of any one of embodiments 1-15, wherein the pharmaceutical composition comprises about 37.5 μg of Compound 1.
20. The pharmaceutical composition of any one of embodiments 1-15, wherein the pharmaceutical composition comprises about 50 μg of Compound 1.
21. The pharmaceutical composition of any one of embodiments 1-15, wherein the pharmaceutical composition comprises about 62.5 μg of Compound 1.
22. The pharmaceutical composition of any one of embodiments 1-15, wherein the pharmaceutical composition comprises about 75 μg of Compound 1.

In other embodiments, Compound 1 is provided tablet with one or more pharmaceutically acceptable excipients. In certain embodiments Compound 1. In certain embodiments the tablet comprises about 12.5, about 25, about 37.5, about 50, about 62.5, or about 75 μg of Compound 1. In certain embodiments the tablet comprises about 12.5 μg of Compound 1. In certain embodiments the tablet comprises about 25 μg of Compound 1. In certain embodiments the tablet comprises about 37.5 μg of Compound 1. In certain embodiments the tablet comprises about 50 μg of Compound 1. In certain embodiments the tablet comprises about 62.5 μg of Compound 1. In certain embodiments the tablet comprises about 75 μg of Compound 1. In certain embodiments the tablet comprises about 87.5 μg of Compound 1. In certain embodiments the tablet comprises about 100 μg of Compound 1.

### Additional Excipients

In certain embodiments, a compound described herein is administered as a pharmaceutical composition comprising one or more excipients from the Handbook of Pharmaceutical Excipients 9^{th} Edition (or earlier).

In certain embodiments, a compound described herein is dissolved or dispersed in oil or fat components. Upon oral administration, the oil or fat component may form a micro-emulsion in the digestive system so that the compound is more quickly absorbed in the body.

In certain embodiments, the oil is an edible oil, a medicament oil, a pharmaceutically acceptable fat and oil, or a food-acceptable fats and oil. For example, the pharmaceutically acceptable fat and oil, or a food-acceptable fats and oil includes, but is not limited, to a vegetable oil, an animal oil, a fish oil or a mineral oil.

In certain embodiments, the edible oil is selected from the group consisting of medium chain fatty acid triglyceride, amaranth oil, apricot oil, apple oil, argan oil, artichokes oil, avocado oil, almond oil, acai berry extract, arachis oil, buffalo pumpkin oil, borage seed oil, borage oil, babassu oil, coconut oil, corn oil, cottonseed oil (cotton seed oil), cashew oil, carob oil, Coriander oil, camellia oil (Camellia oil), Cauliflower oil, cape chestnut oil, cassis oil, deer oil, evening primrose oil, grape syrup Oila oil (hibiscus oil), grape seed oil, gourd oil, hazelnut oil, hemp oil, kapok oil, krill oil, linseed oil, macadamia nut oil, Mongolia oil, moringa oil, malula oil, meadowfoam oil, mustard oil, niger seed oil, olive oil, okrao oil Hibiscus oil), palm oil, palm kernel oil, peanut oil, pecan oil, pine oil, pistachio oil, pumpkin oil, papaya oil, perilla oil (perilla oil), poppy seed oil, prune oil, saw palm oil, quinoa oil, rapeseed oil, rice germ oil, rice bran oil, rice oil, rarelman cheer oil, Safflower oil (safflower oil), soybean oil, sesame oil, sunflower oil, thistle oil, tomato oil, wheat germ oil, walnut oil, watermelon oil, docosahexaenoic acid (DHA), eicosapentaenoic acid (EPA), and any combination thereof.

In certain embodiments, the fat-soluble oil is a vitamin including, but is not limited, to vitamin A oil, vitamin D oil, vitamin E oil, vitamin K oil, and derivatives thereof; and glycerophospholipids such as lecithin, and any combination thereof; can also be used as fats and oils in the present invention.

In certain embodiments, the oil in the present invention may be a liquid (such as a fat oil) or a solid (a fat or the like) at room temperature. In certain embodiments, the oil in the present invention is a liquid at the temperature in the living body (in particular, the temperature of the stomach, around 37° C.). In certain embodiments, the fat includes many saturated fatty acids (for example, palmitic acid, stearic acid), and/or fatty oils contain many unsaturated fatty acids (for example, oleic acid, linoleic acid, linolenic acid); and wherein these fatty acids and oils may be esterified.

In certain embodiments, the fatty acids having 2 to 4 carbon atoms are called short chain fatty acids (lower fatty acids), 5 to 12 fatty acids as medium chain fatty acids, and fatty acids with 12 or more carbon atoms as long chain fatty acids (higher fatty acids). Since fatty acids generally have a high hydrophilicity when the number of carbon atoms is small, oils of the present invention include medium chain fatty acids (for example, medium chain fatty acid triglycerides such as tri (caprylic/capric acid) glycerol and glyceryl tricaprylate) and long Chain fatty acid.

In certain embodiments, the fatty acid is used in combination with a saturated polyglycolated glyceride obtained by polyglycolysis of hydrogenated vegetable oil with polyethylene glycol, a mono-, di- or triglyceride, and a mono- or di-fatty acid ester of polyethylene glycol.

In certain embodiments, the pharmaceutical composition includes a diluent. Nonlimiting examples of the diluent used in capsules include, but are not limited to, calcium carbonate, calcium phosphate di or tri basic, kaolin, lactose, lactitol, mannitol, microcrystalline cellulose, powdered cellulose, cellulose acetate, sorbitol, starch, calcium sulfate, dextrates, dextrin, dextrose, maltodextrin, erythritol, glyceryl palmitostearate, isomalt, magnesium carbonate, magnesium oxide, mannitol, sodium chloride, sucrose, sulfobutylether b-cyclodextrin, talc, and xylitol.

### Dosage Form Packs

In certain embodiments the pharmaceutical composition described herein is administered as a part of a dosage form pack or kit, for example as part of a blister pack. For example, a blister pack with the daily dosage of the therapy in individual tablets or pills and optionally non-therapy tablets or pills for days on drug holiday. In certain embodiments the therapy tablets or pills are a different color, shape, or size than the non-therapy tablets or pills.

In certain embodiments other therapeutic agents are included in the blister pack as either separate tablets, pills, or capsules or as components of the same tablet, pill, or capsule as Compound 1. For example, a blister pack can be provided with a weekly dose of dexamethasone as part of the same or as part of a different tablet, pill, or capsule as Compound 1. In other embodiments multiple blister packs with different pharmaceutical compositions are used. For example a blister pack with tablets of Compound 1 and a separate blister pack with tablets or capsules of one or more additional therapeutic agents.

In certain embodiments the pharmaceutical composition described herein is administered as minitablets, for example a tablet with a diameter or less than about 9, 8, 7, 6, 5, 4, 3, 2, or 1 millimeter. In certain embodiments the minitablet is formulated for delayed release, immediate release, or intermittent release.

### Additional Embodiments of the Present Invention

1. In certain embodiments a crystalline compound of structure:

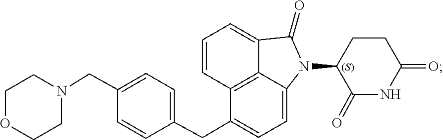

which is characterized by an X-ray powder diffraction (XRPD) pattern comprising at least three 2theta values selected from 4.8, 13.5, 16.0, 16.4, 17.8, 17.9, 19.2, 19.6, 20.9, 21.5, 21.8, 22.9, 23.2, 24.8, 26.5, and 31.3+/−0.4° 2theta is provided.

2. The crystalline compound of embodiment 1, which is characterized by an X-ray powder diffraction (XRPD) pattern comprising at least four 2theta values selected from 4.8, 13.5, 16.0, 16.4, 17.8, 17.9, 19.2, 19.6, 20.9, 21.5, 21.8, 22.9, 23.2, 24.8, 26.5, and 31.3+/−0.4° 2theta.

3. The crystalline compound of embodiment 1, which is characterized by an X-ray powder diffraction (XRPD) pattern comprising at least five 2theta values selected from 4.8, 13.5, 16.0, 16.4, 17.8, 17.9, 19.2, 19.6, 20.9, 21.5, 21.8, 22.9, 23.2, 24.8, 26.5, and 31.3+/−0.4° 2theta.

4. The crystalline compound of embodiment 1, which is characterized by an X-ray powder diffraction (XRPD) pattern comprising at least six 2theta values selected from 4.8, 13.5, 16.0, 16.4, 17.8, 17.9, 19.2, 19.6, 20.9, 21.5, 21.8, 22.9, 23.2, 24.8, 26.5, and 31.3+/−0.4° 2theta.

5. The crystalline compound of embodiment 1, which is characterized by an X-ray powder diffraction (XRPD) pattern comprising at least seven 2theta values selected from 4.8, 13.5, 16.0, 16.4, 17.8, 17.9, 19.2, 19.6, 20.9, 21.5, 21.8, 22.9, 23.2, 24.8, 26.5, and 31.3+/−0.4° 2theta.

6. The crystalline compound of embodiment 1, which is characterized by an X-ray powder diffraction (XRPD) pattern comprising at least eight 2theta values selected from 4.8, 13.5, 16.0, 16.4, 17.8, 17.9, 19.2, 19.6, 20.9, 21.5, 21.8, 22.9, 23.2, 24.8, 26.5, and 31.3+/−0.4° 2theta.

7. The crystalline compound of any one of embodiments 1-6, which is characterized by an X-ray powder diffraction (XRPD) pattern comprising at least three 2theta values selected from 4.8, 13.5, 16.0, 16.4, 17.8, 17.9, 19.2, 19.6, 20.9, 21.5, 21.8, 22.9, 23.2, 24.8, 26.5, and 31.3+/−0.3° 2theta.

8. The crystalline compound of any one of embodiments 1-6, which is characterized by an X-ray powder diffraction (XRPD) pattern comprising at least three 2theta values selected from 4.8, 13.5, 16.0, 16.4, 17.8, 17.9, 19.2, 19.6, 20.9, 21.5, 21.8, 22.9, 23.2, 24.8, 26.5, and 31.3+/−0.2° 2theta.

9. The crystalline compound of any one of embodiments 1-8, wherein the XRPD includes a peak at 4.8+/−0.4° 2theta.

10. The crystalline compound of any one of embodiments 1-9, wherein the XRPD includes a peak at 16.4+/−0.2° 2theta.

11. The crystalline compound of any one of embodiments 1-10, wherein the XRPD includes a peak at 19.2+/−0.2° 2theta.

12. The crystalline compound of any one of embodiments 1-11, wherein the XRPD includes a peak at 22.9+/−0.2° 2theta.

13. The crystalline compound of any one of embodiments 1-12, wherein the XRPD includes a peak at 13.5+/−0.2° 2theta.

14. The crystalline compound of any one of embodiments 1-13, wherein the XRPD includes a peak at 19.6+/−0.2° 2theta.

15. The crystalline compound of any one of embodiments 1-14, wherein the XRPD includes a peak at 23.2+/−0.2° 2theta.

16. The crystalline compound of any one of embodiments 1-15, wherein the XRPD includes a peak at 17.8+/−0.2° 2theta.

17. The crystalline compound of any one of embodiments 1-16, wherein the XRPD includes a peak at 26.5+/−0.2° 2theta.

18. The crystalline compound of any one of embodiments 1-17, wherein the XRPD includes a peak at 21.5+/−0.2° 2theta.

19. In certain embodiments a pharmaceutical composition comprising a crystalline compound of any one of embodiments 1-18 and one or more pharmaceutically acceptable excipients is provided.

20. In certain embodiments a pharmaceutical composition prepared from a crystalline compound of any one of embodiments 1-18 and one or more pharmaceutically acceptable excipients is provided.

21. The pharmaceutical composition of embodiment 19 or 20, wherein the composition is a solid dosage form.

22. The pharmaceutical composition of embodiment 21, wherein the composition is a capsule or a tablet.

23. The pharmaceutical composition of embodiment 19 or 20, wherein the composition is a liquid dosage form.

24. The pharmaceutical composition of embodiment 23, wherein the composition is a gel or liquid filled capsule.

25. The pharmaceutical composition of embodiment 20, wherein the crystalline compound is dissolved in polyethylene glycol.

26. The pharmaceutical composition of embodiment 25, wherein the crystalline compound is dissolved in PEG-400.

27. The pharmaceutical composition of any one of embodiments 19-26, wherein the composition comprises about 1 microgram of Compound 1.

28. The pharmaceutical composition of any one of embodiments 19-26, wherein the composition comprises about 5 microgram of Compound 1.

29. The pharmaceutical composition of any one of embodiments 19-26, wherein the composition comprises about 10 microgram of Compound 1.

30. The pharmaceutical composition of any one of embodiments 19-26, wherein the composition comprises about 15 microgram of Compound 1.

31. The pharmaceutical composition of any one of embodiments 19-26, wherein the composition comprises about 20 microgram of Compound 1.

32. The pharmaceutical composition of any one of embodiments 19-26, wherein the composition comprises about 25 microgram of Compound 1.

33. The pharmaceutical composition of any one of embodiments 19-26, wherein the composition comprises about 30 microgram of Compound 1.

34. The pharmaceutical composition of any one of embodiments 19-26, wherein the composition comprises about 35 microgram of Compound 1.

35. The pharmaceutical composition of any one of embodiments 19-26, wherein the composition comprises about 40 microgram of Compound 1.

36. The pharmaceutical composition of any one of embodiments 19-26, wherein the composition comprises about 45 microgram of Compound 1.

37. The pharmaceutical composition of any one of embodiments 19-26, wherein the composition comprises about 50 microgram of Compound 1.

38. In certain embodiments a pharmaceutical composition is provided, wherein the pharmaceutical composition is a liquid filled capsule comprising a solvent, preservative, and pH control agent, wherein Compound 1 was Compound 1 Form I as described herein before being dissolved.

39. The pharmaceutical composition of embodiment 38, wherein the capsule is filled with a solution comprising about 0.001, 0.005, 0.01, 0.015, 0.02, 0.025, 0.03, 0.033, 0.035, 0.04, 0.045, 0.05, 0.06, 0.07, 0.08, 0.09, 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, 1, 1.5, 2, 2.5, 3, 3.5, 4, 4.5, or 5% Compound 1 by weight.

40. The pharmaceutical composition of any one of embodiments 38-39, wherein the solvent is not aqueous.

41. The pharmaceutical composition of embodiment 40, wherein the solvent is PEG.

42. The pharmaceutical composition of embodiment 41, wherein the solvent is PEG 400.

43. The pharmaceutical composition of any one of embodiments 38-42, wherein the capsule is filled with a solution comprising about 95, 95.5, 96, 96.5, 97, 97.5, 98, 98.1, 98.2, 98.3, 98.4, 98.5, 98.6, 98.65, 98.7, 98.75, 98.8, 98.85, 98.9, 99, 99.1, 99.2, 99.3, 99.4, or 99.5% solvent by weight.

44. The pharmaceutical composition of any one of embodiments 38-43, wherein the preservative is butylated hydroxytoluene.

45. The pharmaceutical composition of any one of embodiments 38-44, wherein the capsule is filled with a solution comprising at least about 0.01, 0.05, 0.1, 0.15, 0.2, 0.25, 0.3, 0.35, 0.4, 0.45, 0.5, 0.55, 0.6, 0.65, 0.8, 0.85, 0.9, 0.95, or 1% preservative by weight.

46. The pharmaceutical composition of any one of embodiments 38-45, wherein the pH control agent is citric acid.

47. The pharmaceutical composition of any one of embodiments 38-45, wherein the pH control agent is anhydrous citric acid.

48. The pharmaceutical composition of any one of embodiments 38-47, wherein the capsule is filled with a solution comprising at least about 0.01, 0.05, 0.1, 0.15, 0.2, 0.25, 0.3, 0.35, 0.4, 0.45, 0.5, 0.55, 0.6, 0.65, 0.8, 0.85, 0.9, 0.95, 1, 1.1, 1.2, 1.3, 1.4, 1.5, 1.6, 1.7, 1.8, 1.9, 2, 2.25, 2.5, 2.75, or 3% pH control agent by weight.

49. In certain embodiments a method of treating a cancer is provided, comprising administering an effective amount of a crystalline compound of pharmaceutical composition of any one of embodiments 1-48 to a patient in need thereof.

50. In certain embodiments a method of treating a cancer is provided, comprising administering an effective amount of Compound 1 or a pharmaceutically acceptable salt or pharmaceutical composition thereof, or a crystalline compound or pharmaceutical composition of any one of embodiments 1-48 to a patient in need thereof (a) once a day for three selected days per week for fourteen days followed by a 14 day dose holiday in a 28 day treatment cycle or (b) once a day for 7 consecutive days followed by a 14 day dose holiday in a 21 day treatment cycle.

51. The method of embodiment 50, wherein the compound is administered once a day on Monday, Wednesday, and Friday for fourteen days followed by a 14 day dose holiday in a 28 day treatment cycle.

52. The method of embodiment 49, wherein the compound is administered for multiple days with a drug holiday in between subsequent treatment cycles.

53. The method of embodiment 52, wherein the compound is administered once or twice a day for at least 13, 14 15, 16, 17, 18, 19, 20, 21, 22, 23, 24, 25, 26 or 27 continuous days, and then the drug holiday is taken until the next 28-day cycle.

54. The method of embodiment 52, wherein the compound is administered once or twice a day for 21 days followed by a 7-day holiday.

55. The method of any one of embodiments 49-54, wherein the dose is less than or equal to about 400 μg.

56. The method of any one of embodiments 49-54, wherein the dose is less than or equal to about 300 μg.

57. The method of any one of embodiments 49-54, wherein the dose is less than or equal to about 200 μg.

58. The method of any one of embodiments 49-54, wherein the dose is less than or equal to about 100 μg.

59. The method of any one of embodiments 49-54, wherein the dose is less than or equal to about 50 μg.

60. The method of any one of embodiments 49-54, wherein the dose is less than or equal to about 25 μg.

61. The method of any one of embodiments 49-54, wherein the dose is less than or equal to about 10 μg.

62. The method of any one of embodiments 49-54, wherein the dose is less than or equal to about 5 μg.

63. The method of any one of embodiments 49-54, wherein the dose is less than or equal to about 1 μg.

64. The method of any one of embodiments 49-54, wherein the dose is about 50 μg.

65. The method of any one of embodiments 49-54, wherein the dose is about 25 μg.

66. The method of any one of embodiments 49-54, wherein the dose is about 10 μg.

67. The method of any one of embodiments 49-54, wherein the dose is about 5 μg.

68. The method of any one of embodiments 49-54, wherein the dose is about 1 μg.

69. The method of any one of embodiments 49-68, wherein the disorder is a diffuse large B-cell lymphoma.

70. The method of embodiment 69, wherein the diffuse large B-cell lymphoma is an activated B-cell lymphoma.

71. The method of embodiment 69, wherein the diffuse large B-cell lymphoma is a germinal center B-cell lymphoma.

72. The method of any one of embodiments 49-68, wherein the disorder is an anaplastic large cell lymphoma.

73. The method of any one of embodiments 49-68, wherein the disorder is a cutaneous T-cell lymphoma.

74. The method of any one of embodiments 49-68, wherein the disorder is mantle cell lymphoma.

75. The method of any one of embodiments 49-68, wherein the disorder is multiple myeloma.

76. The method of any one of embodiments 49-75, wherein the disorder is resistant to treatment with first generation immunomodulatory imide drugs.

77. The method of embodiment 76, wherein the disorder is resistant to treatment with thalidomide.

78. The method of embodiment 76, wherein the disorder is resistant to treatment with pomalidomide.

79. The method of embodiment 76, wherein the disorder is resistant to treatment with lenalidomide.

80. The method of embodiment 76, wherein the disorder is resistant to treatment with iberdomide.

81. The method of any one of embodiments 49-80, wherein a Bruton tyrosine kinase inhibitor is also administered to the patient.

82. The method of embodiment 81, wherein the Bruton tyrosine kinase inhibitor is ibrutinib.

83. The method of any one of embodiments 49-81, wherein a corticosteroid is also administered to the patient.

84. The method of embodiment 83, wherein the corticosteroid is dexamethasone.

85. The method of any one of embodiments 49-84, wherein CAR T-cell therapy is also administered to the patient.

86. The method of embodiment 85, wherein the CAR T-cell therapy is idecabtagene vicleucel.

87. The method of embodiment 85, wherein the CAR T-cell therapy is lisocabtagene maraleucel.

88. The method of embodiment 85, wherein the CAR T-cell therapy is ciltacabtagene autolecuel.

89. The method of embodiment 85, wherein the CAR T-cell therapy is tisagenelecleucel.

90. The method of embodiment 85, wherein the CAR T-cell therapy is brexucabtagene autoleucel.

91. The method of embodiment 85, wherein the CAR T-cell therapy is axicabtagene ciloleucel.

92. The method of embodiment 85, wherein the CAR T-cell therapy is a bispecific CAR T-cell therapy.

93. The method of embodiment 92, wherein the CAR T-cell therapy is BCMA-CD38 CAR T-cell therapy.

94. The method of any one of embodiments 49-93, wherein an antibody-drug conjugate is also administered to the patient.

95. The method of any one of embodiments 49-94, wherein BiTE therapy is also administered to the patient.

96. The method of any one of embodiments 49-95, wherein a bispecific antibody is also administered to the patient.

97. The method of any one of embodiments 49-96, wherein a monoclonal antibody is also administered to the patient.

98. The method of any one of embodiments 49-97, wherein a BTK inhibitor selected from acalabrutinib, spebrutinib, zanubrutinib, LOXO-305, evobrutinib, TG-1701, tolebrutinib, BII1B091, DZD-9008, HZ-A-018, orelabrutinib, AC0058TA, SN1011, rilzabrutinib, ARQ 531, DTRMWXHS-12, JNJ-64264681, branebrutinib, ibrutinib, and fenebrutinib is also administered to the patient.

99. The method of any one of embodiments 49-98, wherein a CD38 antibody selected from felzartamab, daratumumab, GBR 1342, TAK-573, CID-103, OKT10, STI-6129, SGX301, TAK-079, and mezagitamab is also administered to the patient.

100. The method of any one of embodiments 49-99, wherein a proteasome inhibitor selected from ixazomib citrate, oprozomib, delanzomib, lactacystin, bortezomib, carfilzomib, VLX1570, epoxomicin, MG132, MG-262, CEP-18770, NEOSH101, TQB3602, and KZR-616 is also administered to the patient.

101. The method of any one of embodiments 49-100, wherein an immunomodulatory imide drug selected from pomalidomide, lenalidomide, thalidomide, iberdomide CC-92480, CC-90009, and CC-99282 is also administered to the patient.

102. The method of any one of embodiments 49-101, wherein an HDAC inhibitor selected from trapoxin B, sodium phenylbutyrate, tacedinaline, mocetinostat, BRD73954, BG45, domatinostat, cay10603, HPOB, TMP269, nexturastat A, Santacruzamate A, splitomicin, LMK-235, sodium butyrate, pivaloyloxymethyl butyrate, pyroxamide, abexinostat, resminostat, givinostat, quisinostat, Psammaplin A, KD5170, 1-Alaninechlamydocin, depudecin, and CUDC-101 is also administered to the patient.

103. The method of any one of embodiments 49-102, wherein a compound selected from selinexor, oxaphenamide, belantamab mafodotin, denosumab, zoledronic acid, plerixafor, eltrombopag, ipilumumab, palbociclib, ricolinostat, afuresertib, dinaciclib, filanesib, indatuximab ravtansine, masitinib, sonidegib, sotatercept, ulocuplumab, and urelumab is also administered to the patient.

104. The method of any one of embodiments 49-103, wherein the disorder is Non-Hodgkin's Lymphoma.

105. The method of any one of embodiments 49-103, wherein the disorder is Multiple Myeloma.

106. The method of any one of embodiments 49-105, wherein the disorder is relapsed.

107. The method of any one of embodiments 49-106, wherein the disorder is refractory.

108. The method of any one of embodiments 49-107, wherein the cancer is mediated by Ikaros and/or Aiolos.

109. The method of any one of embodiments 49-108, comprising administering the compound once a day on Monday, Wednesday, and Friday for fourteen days followed by a 14-day dose holiday in a 28 day treatment cycle.

110. The method of any one of embodiments 49-109, comprising administering the compound once a day for 7 consecutive days followed by a 14-day dose holiday in a 21 day treatment cycle.

111. In certain embodiments a use of a compound or pharmaceutical composition of any one of embodiments 1-48 in the manufacture of a medicament to treat a cancer in a patient in need thereof is provided.

112. In certain embodiments a compound or pharmaceutical composition of any one of embodiments 1-48 for use in the treatment of a cancer in a patient in need thereof is provided.

113. In certain embodiments a process to prepare a compound of formula:

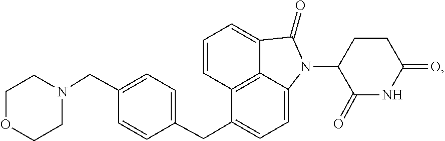 [SMILES: O=C1CCC(N2C(=O)c3cccc4c(Cc5ccc(CN6CCOCC6)cc5)ccc2c34)C(=O)N1]

is provided, wherein the process comprises the steps of:
(i) reacting an intermediate 1 of formula:

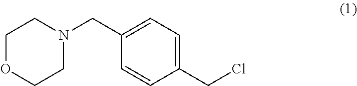 [SMILES: ClCc1ccc(CN2CCOCC2)cc1]

or a salt thereof
with an intermediate 2 of formula:

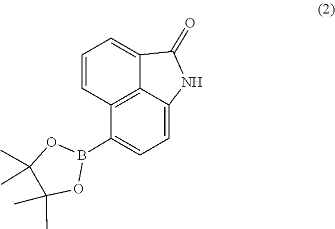 [SMILES: CC1(C)OB(c2ccc3c4c(cccc24)C(=O)N3)OC1(C)C]

in the presence of a base and a palladium catalyst in a solvent to afford an intermediate 3 of formula:

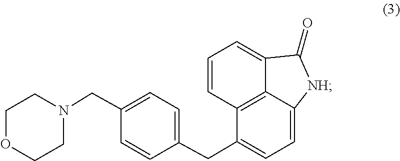 [SMILES: O=C1Nc2ccc(Cc3ccc(CN4CCOCC4)cc3)c3cccc1c23]

and
(ii) reacting the intermediate 3 of the step (i) with an intermediate 4 of formula:

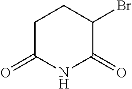

in the presence of a base in a solvent to afford the compound.

114. The process of embodiment 113, wherein the process additionally includes the step (iii) of a chiral separation by an isocratic chiral preparative high-pressure liquid chromatography to afford the compound as an S-enantiomer.

115. The process of embodiment 113 or 114, wherein in step (i):
the base is cesium carbonate;
the palladium catalyst is [1,1′-bis(diphenylphosphino)-ferrocene]dichloropalladium(II) dichloromethane complex or tris(dibenzylideneacetone)-dipalladium(0); and
the solvent is 1,4-dioxane, water, ethanol, toluene, benzene, isopropyl alcohol, N,N-dimethylacetamide, dichloromethane, tetrahydrofuran, N,N-dimethylformamide, acetonitrile, 4-dimethylaminopyridine, acetic acid, acetone, benzene, 1-butanol, 2-butanol, tert-butyl alcohol, carbon tetrachloride, chloroform, cyclohexane, hexane, diethyl ether, diglyme, dimethoxyethane, dimethylsulfoxide, ethyl acetate, ethylene glycol, glycerin, heptane, methanol, methyl tertiary butyl ether, n-methyl-2-pyrrolidone, pentane, pyridine, toluene, triethyl amine, or a mixture thereof.

116. The process of any one of embodiments 113-115, wherein in step (i):
the base is cesium carbonate;
the palladium catalyst is [1,1′-bis(diphenylphosphino)-ferrocene]dichloropalladium(II) dichloromethane complex; and
the solvent is a mixture of 1,4-dioxane and water.

117. The process of any one of embodiments 113-116, wherein in step (ii):
the base is lithium tert-butoxide; and
the solvent is tetrahydrofuran, 1,4-dioxane, water, ethanol, toluene, benzene, isopropyl alcohol, N,N-dimethylacetamide, dichloromethane, N,N-dimethylformamide, acetonitrile, 4-dimethylaminopyridine, acetic acid, acetone, benzene, 1-butanol, 2-butanol, tert-butyl alcohol, carbon tetrachloride, chloroform, cyclohexane, hexane, diethyl ether, diglyme, dimethoxyethane, dimethylsulfoxide, ethyl acetate, ethylene glycol, glycerin, heptane, methanol, methyl tertiary butyl ether, n-methyl-2-pyrrolidone, pentane, pyridine, toluene, or a mixture thereof.

118. The process according to any one of embodiments 113-117, wherein in step (ii):
the base is lithium tert-butoxide; and
the solvent is tetrahydrofuran.

119. The process according to any one of embodiments 113-118, wherein in step (iii):
the isocratic chiral preparative high-pressure liquid chromatography is performed using an eluent selected from a mixture of methanol and dichloromethane; ethanol and dichloromethane; and isopropanol and dichloromethane.

120. The process according to any one of embodiments 113-119, wherein in step (iii):
the eluent is about 20% ethanol and about 80% dichloromethane or about 20% methanol and about 80% dichloromethane.

### EXAMPLES

### Example 1: Manufacturing Process

### Step 1:

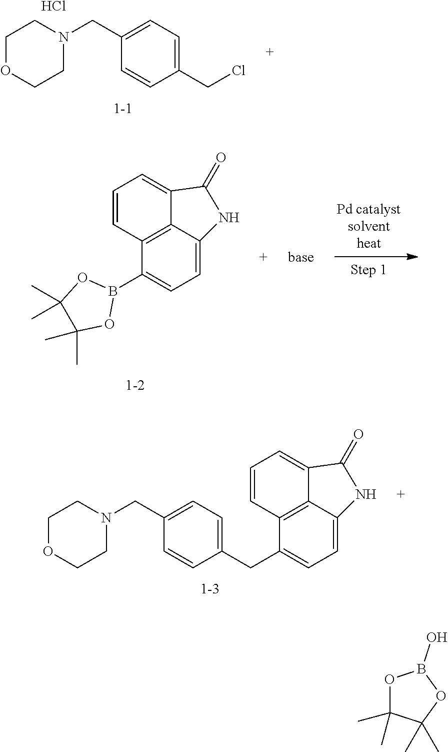

Starting materials 1-1 and 1-2 were coupled with a palladium catalyst (for example Pd(dppf)Cl₂·CH₂Cl₂) and abase (for example cesium carbonate) in a solvent or mixture of solvents (for example 1,4-dioxane and water) at an elevated temperature (for example 90-100° C.) to generate intermediate 1-3. After the reaction was complete, a palladium scavenger (for example trithiocyanuric acid (TMT)) was used to remove residual palladium. The crude intermediate 1-3 was then purified via salt formation with an acid (for example 1N HCl) followed by slurrying with EtOH. After filtration a subsequent salt break can be conducted with a base (for example aqueous Na₂CO₃) in an extraction solvent or mixture of solvents (for example THF/EtOAc).

In certain embodiments the palladium catalyst is [1,1′-bis(diphenylphosphino)ferrocene]dichloropalladium(II), complex with dichloromethane (Pd(dppf)Cl₂·CH₂Cl₂). In other embodiments the palladium catalyst is tris(dibenzylideneacetone)dipalladium(0).

In certain embodiments at least about 0.01, 0.02, 0.03, 0.04, 0.05, 0.06, 0.07, 08, 0.09, or 0.1 equivalents of palladium catalyst are used in Step 1.

In certain embodiments about 0.06 equivalents of catalyst are used in Step 1.

In certain embodiments the palladium scavenger is TMT. In other embodiments the palladium scavenger is X SiliaMets Thiol, active carbon, Si-TMT, Na-TMT, or Silica gel. In certain embodiments at least about 0.3, 0.4, 0.5, 0.6, 0.7, or 0.8 equivalents of Pd scavenger are used. In certain embodiments about 0.6 equivalents of Pd scavenger is used.

In certain embodiments the reaction solvent is selected from or a mixture of solvents selected from 1,4-dioxane, water, ethanol, toluene, benzene, isopropyl alcohol, N,N-dimethylacetamide, dichloromethane, tetrahydrofuran, N,N-dimethylformamide, acetonitrile, 4-dimethylaminopyridine, acetic acid, acetone, benzene, 1-butanol, 2-butanol, tert-butyl alcohol, carbon tetrachloride, chloroform, cyclohexane, hexane, diethyl ether, diglyme, dimethoxyethane, dimethylsulfoxide, ethyl acetate, ethylene glycol, glycerin, heptane, methanol, methyl tertiary butyl ether, n-methyl-2-pyrrolidone, pentane, pyridine, toluene, and triethyl amine.

In certain embodiments the extraction solvent is selected from or a mixture of solvents selected from tetrahydrofuran, ethyl acetate, 1,4-dioxane, water, ethanol, toluene, benzene, isopropyl alcohol, N,N-dimethylacetamide, dichloromethane, N,N-dimethylformamide, acetonitrile, 4-dimethylaminopyridine, acetic acid, acetone, benzene, 1-butanol, 2-butanol, tert-butyl alcohol, carbon tetrachloride, chloroform, cyclohexane, hexane, diethyl ether, diglyme, dimethoxyethane, dimethylsulfoxide, ethylene glycol, glycerin, heptane, methanol, methyl tertiary butyl ether, n-methyl-2-pyrrolidone, pentane, pyridine, toluene, and triethyl amine.

Step 1 can be conducted on manufacturing scale to prepare large quantities of 1-3 for use in the preparation of Compound 1. A non-limiting example of this scaled up reaction is provided below where this reaction sequence was conducted on 100 gram scale.

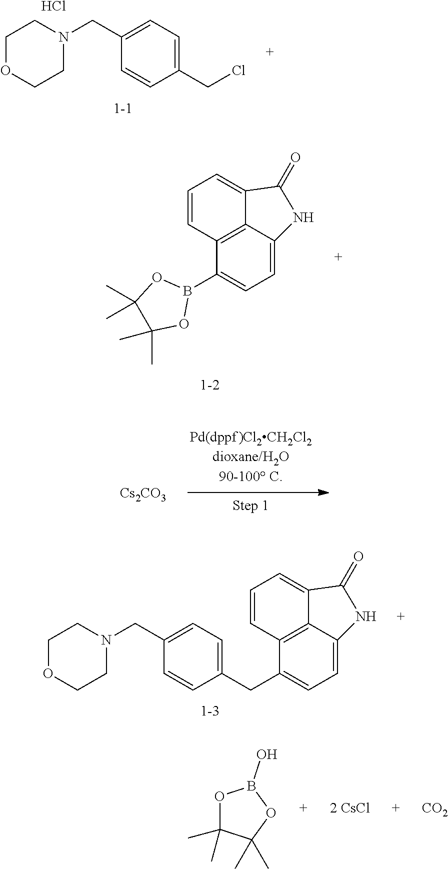

A non-limiting example of this scaled up reaction and the raw material inputs is provided below.

Raw Material List

| | | | | | Ratio |
|---|---|---|---|---|---|
| Material | MW | Weight (g) | Moles | eq. | (w/w) |
| 1-2 | 295.14 | 100.0 | 0.34 | 1.00 | 1.00 |
| 1-1 | 262.18 | 115.5 | 0.44 | 1.30 | 1.15 |
| Cs₂CO₃ | 325.82 | 364.3 | 1.12 | 3.30 | 3.64 |
| Pd(dppf)Cl₂•CH₂Cl₂ | 816.64 | 16.7 | 0.02 | 0.06 | 0.17 |
| 1,4-dioxane | / | 1100 | / | / | 11 |
| TMT | / | 50 | / | / | 0.5 |
| Celite | / | 150 | / | / | 1.5 |
| MTBE | / | 370 | / | / | 3.7 |
| EtOH | / | 340 | / | / | 3.4 |
| 1N HCl | / | 500 | / | / | 5.0 |
| Na₂CO₃ | / | 100 | / | / | 1.0 |
| 10% aq. Na₂SO₄ | / | 300 | / | / | 3.0 |
| EtOAc | / | 1440 | / | / | 14.4 |
| Purified water | / | 210 | | / | 2.1 |
| Process water | / | 2700 | / | / | 27.0 |
| THF | / | 712 | / | / | 7.1 |
| 1-3 | 358.43 | 42.5 | 0.12 | 0.35 | 0.43 |

Process Description
Operation Steps

| | |
|---|---|
| 1. | Charge 878.0 g 1,4-dioxane into R1. |
| 2. | Charge 100.0 g 1-2 into R1. |
| 3. | Charge 115.5 g 1-1 into R1. |
| 4. | Charge 210.0 g purified water into R1 |
| 5. | Add 364.3 g Cs₂CO₃ in several portions into R1 |
| 6. | Purge R1 with inert gas |
| 7. | Charge 16.7 g Pd(dppf)Cl₂•CH₂Cl₂ into R1 |
| 8. | Purge R1 with inert gas |
| 9. | Heat R1 |
| 10. | Stir R1 for 4-24 h |
| 11. | Cool R1 |
| 12. | Charge 800 g process water into R1 |
| 13. | Charge 720 g EtOAc into R1 |
| 14. | Stir R1 for 0.5-1 h |
| 15. | Filter R1 through 150 g celite and wash wet cake with |
| | 200 g EtOAc/1,4-dioxane (v/v) |
| 16. | Charge the filtrate into R1 |
| 17. | Stand R1 for 1-2 h |
| 18. | Separate the bottom aqueous layer to T1 |
| 19. | Charge 300 g 10% aq. Na₂SO₄ into R1 |
| 20. | Stir R1 for 0.5-1 h |
| 21. | Stand R1 for 0.5-2 h |
| 22. | Separate the bottom aqueous layer to T1 |
| 23. | Charge 50 g TMT into R1 |
| 24. | Stir R1 for 5-10 h |
| 25. | Filter R1 and wash wet cake with 150 g EtOAc/1,4-dioxane (v/v) |
| 26. | Charge filtrate into R1 |
| 27. | Charge 500 g 1N HCl into R1 dropwise |
| 28. | Stir R1 for 0.5-4 h |
| 29. | Filter and wash cake with filtrate |
| 30. | Charge filtrate into R1 |
| 31. | Stand R1 for 0.5-2 h |
| 32. | Separate the bottom aqueous layer to T2 |
| 33. | Separate the upper organic layer to T3 |
| 34. | Charge the aqueous layer from T2 into R1 |
| 35. | Adjust R1 to 20-30° C. |
| 36. | Charge 370 g (3.5-3.9X) MTBE into R1 dropwise |
| 37. | Stir R1 for 3-12 h |
| 38. | Filter and wash cake with filtrate |
| 39. | Charge 240 g EtOH |
| 40. | Charge the combined crude wet HCl salt into R1 |
| 41. | Stir R1 for 2-6 h |
| 42. | Filter |
| 43. | Rinse wet cake with 100 g EtOH |
| 44. | Charge wet cake to R1 |
| 45. | Charge 712 g THF into R1 |
| 46. | Charge 720 g EtOAc into R1 |
| 47. | Stir R1 for 15-30 min |
| 48. | Charge 500 g 10% aq. Na₂CO₃ into R1 |
| 49. | Stir R1 for 2-4 h |
| 50. | Stand R1 for 0.5-4 h |
| 51. | Separate the bottom aqueous layer to T4 |
| 52. | Charge 500 g 5% aq. Na₂CO₃ into R1 |
| 53. | Stir R1 for 1-4 h |
| 54. | Stand R1 for 0.5-4 h |
| 55. | Separate the bottom aqueous layer to T4 |
| 56. | Charge 500 g 5% aq. Na₂CO₃ into R1 |
| 57. | Stir R1 for 1-4 h |
| 58. | Stand R1 for 0.5-4 h |
| 59. | Separate the bottom aqueous layer to T4 |
| 60. | Charge 500 g process water into R1 |
| 61. | Stir R1 for 1-4 h |
| 62. | Stand R1 for 0.5-4 h |
| 63. | Separate the bottom aqueous layer to T4 |
| 64. | Concentrate the organic layer to dryness under vacuum |
| 65. | Dry the wet cake under vacuum with heat for 12-48 hr |

### Step 2:

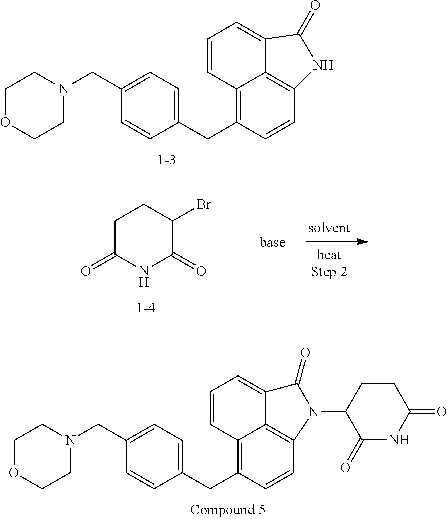 [SMILES: O=C1Nc2ccc(Cc3ccc(CN4CCOCC4)cc3)c3cccc1c23 ; O=C1CCC(Br)C(=O)N1 ; O=C1CCC(N2C(=O)c3cccc4c(Cc5ccc(CN6CCOCC6)cc5)ccc2c34)C(=O)N1]

Intermediate 1-3 was treated with a base (for example lithium tert-butoxide (t-BuOLi)), followed by bromoglutarimide (1-4) in a solvent or mixture of solvents (for example THF) with heat (for example 55-65° C.) to generate the racemate Compound 5. After the reaction was complete, the reaction was quenched with an acid (for example 1N aqueous solution of hydrochloric acid), neutralized with aqueous solution of sodium bicarbonate, and filtered to give Compound 5.

In certain embodiments Intermediate 1-3 is stirred with lithium tert-butoxide for deprotonation at an elevated temperature before addition of Intermediate 1-4. In certain embodiments the temperature for deprotonation is between about 30° C. and about 70° C. In certain embodiments the temperature for deprotonation is about 55-65° C. In certain embodiments the temperature for deprotonation is about 25-35° C. In certain embodiments the temperature for deprotonation is about 60-70° C. In certain embodiments the temperature for feeding 1-4 is 55-65° C. or 40-45° C. In preferred embodiments the temperature for deprotonation and feeding 1-4 is about 55-65° C.

In certain embodiments at least about 4.5, 5.5, 7 or 10 equivalents of t-BuOLi are used. In certain embodiments at least about 4, 4.5 or 5 equivalents of 1-4 are used. In preferred embodiments about 5 equivalents or 1-4 and about 10 equivalents of t-BuOLi are used.

In certain embodiments the reaction solvent is selected from or a mixture of solvents selected from tetrahydrofuran, 1,4-dioxane, water, ethanol, toluene, benzene, isopropyl alcohol, N,N-dimethylacetamide, dichloromethane, N,N-dimethylformamide, acetonitrile, 4-dimethylaminopyridine, acetic acid, acetone, benzene, 1-butanol, 2-butanol, tert-butyl alcohol, carbon tetrachloride, chloroform, cyclohexane, hexane, diethyl ether, diglyme, dimethoxyethane, dimethylsulfoxide, ethyl acetate, ethylene glycol, glycerin, heptane, methanol, methyl tertiary butyl ether, n-methyl-2-pyrrolidone, pentane, pyridine, and toluene.

Step 2 can be conducted on manufacturing scale to prepare large quantities of Compound 5 for use in the preparation of Compound 1. A non-limiting example of this scaled up reaction at 100 gram scale is given below.

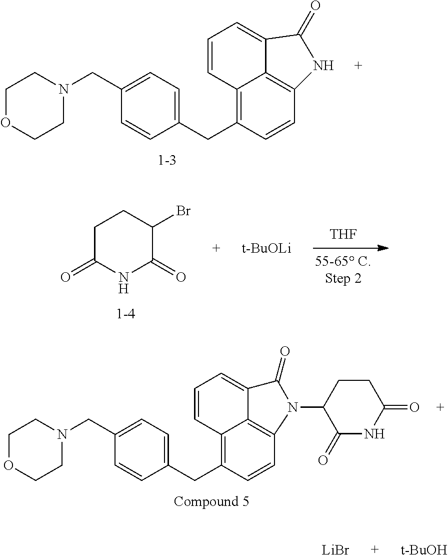

A non-limiting example of this scaled up reaction and the raw material inputs is provided below.

Raw Material List

| | | Weight | | | Ratio |
|---|---|---|---|---|---|
| Material | MW. | (g) | Moles | eq. | (w/w) |
| 1-3 | 358.43 | 100 | 0.279 | 1.0 | 1.00 |
| 1-4 | 192.01 | 267.9 | 1.396 | 5.0 | 2.68 |
| 1-4 | 192.01 | 10.7 | 0.056 | 0.2 | 0.11 |
| t-BuOLi | 80.06 | 223.4 | 2.790 | 10.0 | 2.23 |
| THF | 72.11 | 890 | / | / | 8.90 |
| THF | 72.11 | 890 | / | / | 8.90 |
| THF | 72.11 | 35.6 | / | / | 0.36 |
| Process water | 18.02 | 1450 | / | / | 14.5 |
| 35% HCl | 36.46 | 156.3 | 1.500 | 5.4 | 1.60 |
| Process water | 18.02 | 791 | / | / | 7.9 |
| NaHCO₃ | 84.01 | 59.5 | 0.708 | 2.5 | 0.60 |
| Process water | 18.02 | 3400 | / | / | 34.0 |
| NaHCO₃ | 84.01 | 75 | / | / | 0.75 |
| DMSO | 78.13 | 5500 | / | / | 55.0 |
| Purified water | 18.02 | 2500 | / | / | 25.0 |
| DMSO | 78.13 | 5500 | / | / | 55.0 |
| Purified water | 18.02 | 2500 | / | / | 25.0 |
| Compound 5 | 469.53 | 85.2 | 0.181 | 0.65 | 0.85 |

Process Description
Operation Steps

| | |
|---|---|
| 1. | Charge 100 g 1-3 into R1. |
| 2. | Charge 890 g THF into R1. |
| 3. | Stir R1 for 30-60 min. |
| 4. | Charge 223.4 g t-BuOLi into R1. |
| 5. | Stir R1 with heat for 2-6 h. |
| 6. | Charge 267.9 g 1-4 to R2. |
| 7. | Charge 890 g THF into R2. |
| 8. | Stir R2 for 30-60 min. |
| 9. | Transfer the mixture in R2 to R1 at elevated temperature. |
| 10. | Stir R1 at elevated temperature for 2-8 h. |
| 11. | Charge 10.71 g 1-4 to R2. |
| 12. | Charge 35.6 g THF into R2. |
| 13. | Stir R2 for 30-60 min. |
| 14. | Transfer the mixture in R2 to R1. |
| 15. | Stir R1 at elevated temperature for 2-6 h. |
| 16. | Charge 1300 g 1N HCl (11-15X) to R3. |
| 17. | Chill R3. |
| 18. | Transfer reaction mixture in R1 to R3 dropwise slowly. |
| 19. | Charge 650 g 7% NaHCO₃ to R3 to adjust pH to 7-8. |
| 20. | Warm R3. |
| 21. | Charge 3400 g process water into R3 slowly. |
| 22. | Stir R3 for 2-6 h. |
| 23. | Filter the slurry. |
| 24. | Wash wet cake with 500 g process water. |
| 25. | Charge the wet cake into R4. |
| 26. | Charge 5500 g DMSO into R4. |
| 27. | Stir R4 for 1-6 h. |
| 28. | Charge 2500 g purified water into R5. |
| 29. | Charge 75 g NaHCO₃ into R5. |
| 30. | Stir R5 1-3 h. |
| 31. | Drop the solution from R4 into R5 slowly. |
| 32. | Stir R5 for 1-3 h. |
| 33. | Filter R5. |
| 34. | Wash cake with 500 g purified water. |
| 35. | Charge wet cake into R5. |
| 36. | Charge 5500 g DMSO into R5. |
| 37. | Stir R5 for 1-3 h. |
| 38. | Transfer the solution from R5 to T2. |
| 39. | Charge 2500 g purified water into R5. |
| 40. | Drop the solution from T2 into R5 slowly. |
| 41. | Stir R5 for 1-3 h. |
| 42. | Filter R5. |
| 43. | Wash wet cake with 500 g purified water. |
| 44. | Dry the wet cake for 12-48 h in vacuum with heat. |

### Step 3:

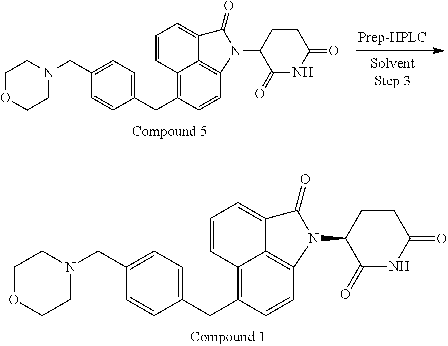 [SMILES: O=C1CCC(N2C(=O)c3cccc4c(Cc5ccc(CN6CCOCC6)cc5)ccc2c34)C(=O)N1 ; O=C1CC[C@H](N2C(=O)c3cccc4c(Cc5ccc(CN6CCOCC6)cc5)ccc2c34)C(=O)N1]

An isocratic, chiral, preparative high-pressure liquid chromatography (HPLC) separation using a chiral column (for example CHIRALPAK® IC stationary phase) and a solvent (for example methanol/dichloromethane or ethanol/dichloromethane mobile phase) was performed to separate Compound 1 (S enantiomer) from the R enantiomer. After concentration of solvents, Compound 1 was collected by filtration and dried.

In certain embodiments the eluent for preparative HPLC is IPA/DCM at a ratio of about 1/1. In certain embodiments the eluent for preparative HPLC is EtOH/DCM at a ratio of about 1/4. EtOH/DCM at a ratio of 1/4 avoids high column pressure, and the separation is completed within 30 min/injection compared to about 60 min/injection for IPA/DCM at a ratio of 1/1. In certain embodiments the eluent is MeOH/DCM at a ratio of about 1/4.

Step 3 can be conducted on manufacturing scale to prepare large quantities of Compound 1 for use in the preparation of Compound 1 drug substance (CFT7455). A non-limiting example of this scaled up reaction and the raw material inputs is provided below.

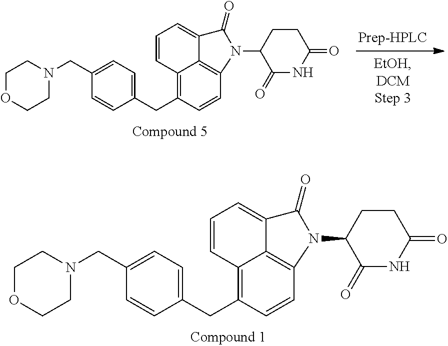 [SMILES: O=C1CCC(N2C(=O)c3cccc4c(Cc5ccc(CN6CCOCC6)cc5)ccc2c34)C(=O)N1 ; O=C1CC[C@H](N2C(=O)c3cccc4c(Cc5ccc(CN6CCOCC6)cc5)ccc2c34)C(=O)N1]

A non-limiting example of this scaled up reaction and the raw material inputs is provided below.

Raw Material List

| | | Weight | | | | Ratio |
|---|---|---|---|---|---|---|
| Material | MW. | (g) | | Moles | eq | (w/w) |
| Compound 5 | 469.53 | 100 | g | 0.213 | 1.0 | 1.0 |
| DCM | / | 5.3 | kg | / | / | 53 |
| EtOH | / | 790 | g | / | / | 7.9 |
| DCM | / | 106 | kg | / | / | 1060 |
| EtOH | / | 15.8 | kg | / | / | 158 |
| Compound 1 | 469.53 | 40 | g | 0.085 | 0.40 | 0.40 |

Process Description
Operation Steps

| | |
|---|---|
| 1. | Charge 790 g EtOH into R1. |
| 2. | Charge 5300 g DCM into R1. |
| 3. | Stir R1. |
| 4. | Transfer solution in R1 to T1. |
| 5. | Charge 100 g crude Compound 5 into R1. |
| 6. | Charge EtOH/DCM = 2/8 (v/v) solution to R1. |
| 7. | Stir R1 for 30-120 min. |
| 8. | Filter R1 via 0.22 μm micro filter to T2. |
| 9. | Package column: CHIRALPAK ® IC 10 μm, 25 × 10 cm. |
| 10. | Balance with EtOH/DCM = 2/8 (v/v). |
| 11. | Inject Compound 5 solution. |
| 12. | Eluent: EtOH/DCM = 2/8 (v/v) for NLT 30 min with 350 mL/min flow rate. |
| 13. | Concentrate combined fractions. |
| 14. | Dry wet cake in vacuum. |

### Step 4:

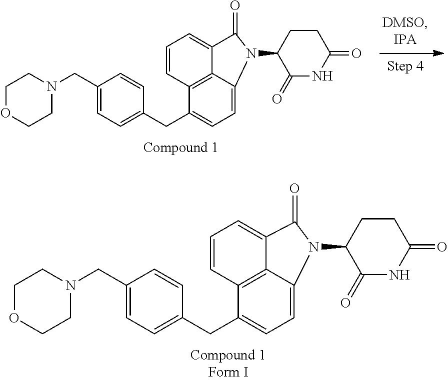 [SMILES: O=C1CC[C@H](N2C(=O)c3cccc4c(Cc5ccc(CN6CCOCC6)cc5)ccc2c34)C(=O)N1 ; O=C1CC[C@H](N2C(=O)c3cccc4c(Cc5ccc(CN6CCOCC6)cc5)ccc2c34)C(=O)N1]

Compound 1 was purified by crystallization as described herein (for example from dimethylsulfoxide (DMSO) and 2-propanol (IPA) at about 25° C.) to give Compound 1 Form I.

In certain embodiments Compound 1 is dissolved in DMSO and the anti-solvent used is water, 2-propanol, or ethanol.

In certain embodiments the ratio of DMSO used to dissolve Compound 1 to the anti-solvent used is about 6 vol./12 vol., about 3.5 vol./14 vol. or about 6.5 vol./26 vol.

Step 4 can be conducted on manufacturing scale to prepare large quantities Compound 1 Form I (for example in 10 gram batches or larger).

A non-limiting example of this scaled up reaction and the raw material inputs is provided below.

Raw Material List

| | | Weight | Moles | | Ratio |
|---|---|---|---|---|---|
| Material | MW. | (g) | (mol) | eq. | (w/w) |
| Compound 1 | 469.53 | 10 | 0.0213 | 1.0 | 1.0 X |
| Compound 1 | 469.53 | 0.1 | 0.00021 | 0.01 | 0.01 X |
| (seed) | | | | | |
| DMSO | 78.13 | 66.0 | / | / | 6.6 X |
| IPA | 60 | 268.6 | / | / | 26.9 X |

| | |
|---|---|
| 1. | Mix Compound 1 (10 g) into R1. |
| 2. | Charge 66.0 g DMSO into R1. |
| 3. | Stir R1 for 1-3 h. |
| 4. | Do polish filtration and transfer the solution into R2. |
| 5. | Charge 5.5 g DMSO into R1. |
| 6. | Charge 11.4 g IPA into R2. |
| 7. | Charge 0.1 g Compound 1 seed into R2. |
| 8. | Stir R2 for 2-4 h. |
| 9. | Charge 194 g IPA into R2. |
| 10. | Stir R2 for 6-30 h. |
| 11. | Filter the suspension in R2 |
| 12. | Wash the cake with IPA |
| 13. | Dry the cake |

### Morphic Form Evaluation of Compound 1

The following techniques were used to characterize Compound 1.

### Example 2: Approximate Solubility at 25° C. and 50° C.

About 5 mg of Compound 1 Form I (free form) was weighed to a 2 mL glass vial and aliquots of 20 μL of each solvent were added to determine solubility at 25° C.

About 10 mg of Compound 1 Form I (free form) was weighed to a 2 mL glass vial and aliquots of 20 μL of each solvent were added to determine solubility at 50° C. Max. volume of each solvent added was 1 mL. Approximate solubility was determined by visual observation.

The results are summarized in Table 1.

TABLE 1
Approximate solubility at 25° C. and 50° C.

Solubility (mg/mL)

| Solvent | 25° C. | 50° C. |
|---|---|---|
| Methanol | <5 | <10 |
| Ethanol | <5 | <10 |
| Acetone | <5 | <10 |
| Acetonitrile | <5 | <10 |
| THF | 8-13 | 16-25 |
| Ethyl acetate | <5 | <10 |
| MTBE | <5 | <10 |
| Heptane | <5 | <10 |
| DCM | 8-13 | 16-25 |
| Water | <5 | <10 |
| DMSO | 83-125 | 167-250 |
| DCM:MeOH (v:v = 1:1) | 17-20 | 29-33 |
| THF:Water (v:v = 95:5) | 17-20 | 40-50 |
| Ethanol:Water (8:2, v:v) | <5 | <10 |

### Example 3: Equilibration with Solvents at 25° C. for 2 Weeks

About 30 mg of Compound 1 Form I (free form) was equilibrated in 1 mL of solvent at 25° C. for 2 weeks with a stirring plate. The obtained suspension was filtered. The solid part (wet cake) was investigated b XRPD. The results are summarized in Table 2.

TABLE 2
Equilibration with solvents at 25° C. for 2 weeks

| Solvent | XRPD | Notes |
|---|---|---|
| THF | Form I | Solids |
| DCM | Form I | Solids |
| DCM:THF(v:v = 8:2) | Form I | Solids |
| DCM:MeOH (v:v = 1:1) | Form I | Solids |
| Acetone | Form I | Solids |
| EtOH | Form I | Solids |
| | | Melting onset: 239.0° C., |
| | | enthalpy: 107 J/g |
| | | 0.4% residual DCM |
| EA | Form I | Solids |
| ACN | Form I | Solids |
| THF:Water (v:v = 95:5) | Form I | Solids |
| MeOH | Form I | Solids |

### Example 4: Equilibration with Solvents at 50° C. for 1 Week

About 50 mg of Compound 1 Form I (free form) was equilibrated in 1 mL of solvent at 50° C. for 1 week with a stirring plate. The obtained suspension was filtered and the solid part (wet cake) was investigated by XRPD. The results are summarized in Table 3.

TABLE 3
Equilibration with solvents at 50° C. for 1 week

| Solvent | XRPD | Notes |
|---|---|---|
| THF | Form I | Solids |
| DCM | Form I | Solids |
| DCM:THF(v:v = 8:2) | Form I | Solids |
| DCM:MeOH (v:v = 1:1) | Form I | Solids |
| Acetone | Form I | Solids |
| EtOH | Form I | Solids |
| | | Melting onset: 239.5° C., |
| | | enthalpy: 109 J/g |
| | | 0.4% residual DCM |
| EA | Form I | Solids |
| ACN | Form I | Solids |
| THF:Water (v:v = 95:5) | Form I | Solids |
| MeOH | Form I | Solids |

### Example 5: Precipitation by Addition of Anti-Solvent

About 20 mg of Compound 1 Form I (free form) was dissolved in DMSO and anti-solvent was added into the obtained solution slowly. Precipitates were collected by filtration. The solid part (wet cake) was investigated by XRPD. Residual solvent of these solids was also analyzed by ¹H-NMR after vacuum dried at 80° C. for 2 hours. The results are summarized in Table 4.

TABLE 4
Precipitation by addition of anti-solvent

| Solvents | Anti-solvent | XRPD | Notes |
|---|---|---|---|
| DMSO | MeOH | − | Suspension |
| | | | 0.14 equiv. residual |
| | | | DMSO |
| DMSO | Acetone | // | Clear solution^{a} |
| DMSO | Water | − | Suspension |
| | | | 0.90 equiv. residual |
| | | | DMSO |
| DMSO | EtOH | − | Suspension |
| | | | 0.05 equiv. residual |
| | | | DMSO |
| DMSO | EtOAc | // | Clear solution |
| DMSO | Water | − | Suspension^{b} |

Explanation
“−”: No form change.
“//”: Not detected.
^{a}The clear solution was hold at 5° C. for 2 days, and no solid was obtained.
^{b}About 2 mg of seeds were added.

### Example 6: Crystallization at Room Temperature by Slow Evaporation

About 20 mg of Compound 1 Form I (free form) was dissolved and filtered by 0.45 μm nylon filter. Obtained solutions were evaporated slowly at ambient condition. Solid residues were examined for their polymorphic form. The results are summarized in Table 5.

TABLE 5
Crystallization at room temperature by slow evaporation

| Solvents | XRPD | Notes |
|---|---|---|
| THF | + | Form III |
| DCM | − | Form I |
| MeOH/DCM(v:v = 1:1) | // | // |
| MeOH/DCM(v:v = 1:1) | + | Form II |
| | | Form I was obtained after heating to |
| | | 150° C. |
| | | No form change at 25° C./92% RH |
| | | for 2 days |
| THF:Water (v:v = 95:5) | + | Form III |

Explanation
“−”: No form change.
“+”: Form changed detected.
“//”: Not detected.

### Example 7: Behavior Under Heating and Cooling

Polymorphic behavior of Compound 1 Form I (free form) was investigated by two different heating-cooling cycles of DSC. The results were summarized in Table 6.
HCH1: 30° C. to 300° C. at 10° C./min; 300° C. to −20° C. at 20° C./min; reheat to 300° C. at 10° C./min.
HCH2: 30° C. to 300° C. at 10° C./min; 300° C. to −20° C. at 2° C./min; reheat to 300° C. at 10° C./min.

TABLE 6
Behavior under heating and cooling

| Exp. ID | Heating rate | Thermal events |
|---|---|---|
| HCH1 | Cycle 1: 30° C. to 300° C. at 10° | Melting onset 231.4° |
| | C./min; 300° C. to −20° C. at 20° | C.; enthalpy 115 J/g |
| | C./min; reheat to 300° C. at 10° C./min. | |
| HCH2 | Cycle 2: 30° C. to 300° C. at 10° | Melting onset 231.3° |
| | C./min; 300° C. to −20° C. at 2° | C.; enthalpy 108 J/g |
| | C./min; reheat to 300° C. at 10° C./min. | |

### Example 8: Scale-Up Experiment (200 mg)

Scale-up 1: About 200 mg of Compound 1 Form I was dissolved in 10 mL THF/Water (95:5, v:v) at 40° C. The solution was filtered to obtain clear solution, then evaporated at 25° C. Compound 1 Form IV was obtained. It was subjected to characterization.

Scale-up 2: About 200 mg of Compound 1 Form I was dissolved in 10 mL THF/Water (95:5, v:v) at 40° C. The solution was filtered to obtain clear solution, about 1 mg of seeds were added, then evaporated at 25° C. It was subjected to characterization.

The instrumental methods are included in Example 23 and the results are summarized in Table 7.

TABLE 7
Characterization and study of new polymorphs

| Crystalline form | | Form I | Form II | Form III | Form IV |
|---|---|---|---|---|---|
| Parameters | Method | // | MeOH/DCM | THF/Water | THF/Water |
| | | | (v:v = 1:1) | (v:v = 95:5) | (v:v = 95:5) |
| Yield | // | // | // | // | 85.9% |
| Purity | HPLC | 99.0% | // | // | 96.2% |
| X-ray | 3-40° | Highly | Crystalline | Crystalline | Crystalline |
| diffraction | (2 | crystalline | (XRPD method | (XRPD method | (XRPD method |
| | theta) | (XRPD method | 1) | 1) | 1) |
| | | 1) | | | |
| DSC | DSC, | Melting onset | Methanol | Dehydration | Dehydration |
| melting | 10° C./ | 233.9° C.; | desolvation | before melting | before melting |
| onset | min | enthalpy 114 J/g | at 103.1° C. | | |
| Thermogravimetry | TGA, | About 2.0% | About 7.1% | About 5.8% | About 1.3% |
| | 10° C./ | weight loss at | weight loss | weight loss at | weight loss at |
| | min | 225° C. | at 125° C. | 120° C. (1.5 | 105° C. (0.4 |
| | | | Form I was | equiv. water) | equiv. water) |
| | | | obtained after | Heating to | Heating to |
| | | | heating to | 150° C., no | 150° C., no |
| | | | 150° C. | form change | form change |
| | | | No form change | | Form I was |
| | | | at 25° C./92% | | obtained after |
| | | | RH for 2 days | | heating by cycle |
| | | | | | DSC |
| | | | | | 30° C. to 120° C. |
| | | | | | at 10° C./min; |
| | | | | | 120° C. to 0° C. at |
| | | | | | 10° C./min; |
| | | | | | reheat to 232° C. |
| | | | | | at 10° C./min. |
| Stoichiometry | ¹H- | 0.6% DCM | 0.3% DCM and | No residual | No residual |
| | NMR | | 1.0 equiv. | solvent | solvent |
| | | | MeOH | | |

### Example 9: Water Activity Study at 25° C.

Water activity experiments were conducted at 25° C. in 7 different water activities solvent mixtures to determine critical water activity of Compound 1 Form I. About 20 mg of Compound 1 Form I was added to 1.0 mL of acetone/water or DMSO/water mixtures and stirred at 400 rpm and 25° C. for 1 week. The results are summarized in Table 8.

TABLE 8
Water activity study at 25° C. for 1 week

| | | |
|---|---|---|
| Solvents | a.w. | XRPD |

Form I

| | | |
|---|---|---|
| Water | 1 | Form I |
| Acetone/Water | 1 | Form I |
| (91:9, v:v) | | |
| Acetone/Water | 0.5 | Form I |
| (94:6, v:v) | | |
| Acetone/Water | 0.3 | Form I |
| (97:3, v:v) | | |
| Acetone/Water | 0.1 | Form I |
| (99:1, v:v) | | |
| Acetone | 0 | Form I |
| DMSO/Water | 0.5 | Form I |
| (64:36, v:v) | | |

### Example 10: Competitive Equilibration Experiment

About 10 mg of Compound 1 Form I and 10 mg Compound 1 Form IV were added to 0.8 mL saturated solution in selected solvent. Obtained suspensions were stirred at 400 rpm and 25° C. for 1 week. Solids obtained were separated by filtration and characterized by XRPD. The results are summarized in Table 9.

TABLE 9
Competitive equilibration experiment for 1 week

| | | XRPD |
|---|---|---|
| Solvents | a.w. | Form I + Form IV |
| THF/Water | 0.1 | Form I |
| (99.1:0.9, v:v) | | |
| THF/Water | 0.2 | Form I |
| (98.1:1.9, v:v) | | |
| THF/Water | 0.3 | Form I |
| (97.0:3.0, v:v) | | |
| THF/Water | 0.6 | Form IV |
| (92.3:7.7, v:v) | | |
| THF/Water | 0.8 | Form IV |
| (86.9:13.1, v:v) | | |
| THF/Water | 1.0 | Form IV |
| (63.9:36.1, v:v) | | |
| DMSO/Water | 0.5 | Physical mixture of Form |
| (64.2:35.8, v:v) | | I and Form IV, mainly |
| | | Form I |
| DMSO/Water | 0.6 | Physical mixture of Form |
| (57.0:43.0, v:v) | | I and Form IV, mainly |
| | | Form I |

### Example 11: Thermal Behavior Study

Compound 1 Form I samples after equilibration were characterized by DSC to see whether DCM if present could be removed after equilibration.

Compound 1 Form IV samples were characterized by DSC with a 2° C./min heating rate to see whether it could convert to Compound 1 Form I completely before melting. It was found that no new Form was observed upon heating, and Form I was obtained when heating beyond the first melting peak at about 230° C., although crystallinity was low. There is no evidence that the additional peak is due to new polymorphic form. It may be related to the decomposed product as there was weight loss due to decomposition starting from around 220° C.

### Example 12: Preparation of Compound 1 Form I

Compound 1 Form I was prepared by anti-solvent crystallization of Compound 1 in DMSO, when MeOH, water, ethanol, or 2-propanol (IPA) was used as anti-solvent.

A non-limiting example of this crystallization method is provided below.
1. Charge Compound 1 (1×, based on assay) to R1
2. Charge DMSO (6V) to R1
3. Stir R1 at 25° C. for 1-3 h
4. Polish filter the solution and transfer to R2
5. Rinse R1 with DMSO (0.5V) and transfer to R2
6. Charge IPA (1.5V) to R2 1.15
7. Charge seed (1%, Compound 1 Form I) into R2
8. Age slurry for 2 h while stirring at 25° C.
9. Charge IPA (24.5V) into R2 over 12 h at 25° C.
10. Stir R2 at 25° C. for 6-12 h
11. Sample from mother liquor for assay, sample from solid for XRPD and purity
12. Filter at 25° C. and rinse with IPA (2V) four times
13. Dry the cake under vacuum at 50° C. for 16-24 hours to obtain yellow solid.

### Morphic Form Evaluation of Compound 5

The following techniques were used to characterize Compound 5.

### Example 13: Approximate Solubility at 25° C.

About 5 mg of Compound 5 Form V was weighed to an 8 mL glass vial. 100 μL aliquots of each solvent were added to dissolve the drug substance at 25° C. Vortex and sonication were applied to assist dissolution. The maximum volume of each solvent added is 1 mL. Approximate solubility was determined by visual observation.

For low solubility samples: Aliquots of selected solvent were added to 5 mg of Compound 5 create a suspension. The suspensions were kept at 25° C. for 24 hours. The suspension was taken out and centrifuged. To eliminate solvent effect, 50 μL or 100 μL of supernatant was added into a 2 mL glass vial and evaporated at ambient condition (22-25° C., 30-60% RH). The obtained solids were dissolved by ACN:DMSO (v:v=3:2) and used for solubility test by HPLC.

The results are summarized in Table 10.

TABLE 10
Approximate solubility at 25° C.

| | Solubility | |
|---|---|---|
| Solvents | (mg/mL) at 25° C. | Comments |
| MeOH/DCM (v:v = 1:1) | ~5 | Tested by observation |
| DMSO | >50 | Tested by observation |
| 1,4-Dioxane | 2.3 | Tested by HPLC |
| Pyridine | 25-50 | Tested by observation |
| Propylene glycol methyl | 2.3 | Tested by HPLC |
| ether | | |
| Chloroform | 5-10 | Tested by observation |
| Methoxy- | 0 | Tested by HPLC |
| nonafluorobutane | | |
| 1-Methyl-2-pyrrolidinone | >50 | Tested by observation |
| Toluene | 0.2 | Tested by HPLC |
| Trifluoroethanol | 25-50 | Tested by observation |
| Anisole | 1.1 | Tested by HPLC |
| DMF | 25-50 | Tested by observation |
| Propylene glycol | 3.4 | Tested by HPLC |
| Acetic acid | 5.5 | Tested by HPLC |
| Benzyl alcohol | 12-25 | Tested by observation |
| Methanol | 0.2 | Tested by HPLC |
| Ethanol | 0.6 | Tested by HPLC |
| Acetone | 3.3 | Tested by HPLC |
| Acetonitrile | 2.2 | Tested by HPLC |
| Ethyl acetate | 0.1 | Tested by HPLC |
| THF | 3.8 | Tested by HPLC |
| 2-Me THF | 0.2 | Tested by HPLC |
| MTBE | 0 | Tested by HPLC |
| Heptane | 0 | Tested by HPLC |

### Example 14: Equilibration with Solvents at 25° C. for 1 Week

Based on approximate solubility results, about 50 mg of Compound 5 Form V was equilibrated in 0.2-0.5 mL of solvents at 25° C. for 1 week with a stirring bar on a magnetic stirring plate at a rate of 400 rpm. The obtained suspensions were centrifuged at 14,000 rpm. The solid parts (wet cakes) were investigated by XRPD. The results are summarized in Table 11.

TABLE 11
Equilibration with solvents at 25° C. for 1 week

| Solvents | XRPD |
|---|---|
| Pyridine | Form V |
| Trifluoroethanol | Form V |
| DMF | Form V |
| Benzyl alcohol | Form V |
| Acetone | Form V |
| THF | Form V |
| MeOH/DCM (v:v = 1:1) | Form V |
| Chloroform | Form V |

### Example 15: Crystallization at Room Temperature by Fast Evaporation

Based on approximate solubility results, about 20 mg of Compound 5 Form V was dissolved in 1-6 mL of solvents. Solutions were centrifuged and obtained clear solutions were fast evaporated at room temperature (about 20-25° C.) under a dry nitrogen flow. The solid residue was investigated by XRPD. For samples with different XRPD patterns, ¹H-NMR was performed. The results are summarized in Table 12.

TABLE 12
Crystallization at room temperature by fast evaporation

| Solvents | XRPD | Additional analysis |
|---|---|---|
| DMSO | Form VI | ¹H-NMR: 0.4 equiv. DMSO |
| Pyridine | Physical mixture of | // |
| | Form A and Form B | |
| Chloroform | Amorphous form | // |
| 1-Methyl-2- | Form VII | ¹H-NMR: 1.6 equiv. NMP; |
| pyrrolidinone | | 2 unknown peaks |
| Trifluoroethanol | // | Oil |
| DMF | Physical mixture of | // |
| | Form V and Form VI | |
| Benzyl alcohol | Form V | // |
| THF | Form V | // |

Explanation “//”: Not conducted.

### Example 16: Crystallization from Hot Saturated Solutions by Fast Cooling

Based on approximate solubility results, about 30 mg of Compound 5 Form V was dissolved in a minimal amount of selected solvent at 50° C. The solutions were centrifuged and the obtained clear solutions were put at the crystal screening platform at about 0° C. No precipitate came out by fast cooling. The results are summarized in Table 13.

TABLE 13
Crystallization from hot saturated solutions by fast cooling

| Solvents | Comments |
|---|---|
| Acetone | Clear solution |
| Pyridine | Clear solution |
| Chloroform | Clear solution |
| 1-Methyl-2-pyrrolidinone | Clear solution |
| Trifluoroethanol | Clear solution |
| DMF | Clear solution |
| Benzyl alcohol | Clear solution |
| THF | Clear solution |

### Example 17: Crystallization by Addition of Anti-Solvent

Based on approximate solubility results, about 50 mg of Compound 5 Form V was dissolved in the minimal amount of selected solvents at ambient temperature (about 20-25° C.). The obtained solutions were filtered through a 0.45 μm syringe membrane filter. 1-4 folds of anti-solvent were added into the clear solutions slowly until a large amount of solid precipitated out. Precipitates were investigated by XRPD. For samples with different XRPD patterns, ¹H-NMR was performed. The results are summarized in Table 14.

TABLE 14
Crystallization by addition of anti-solvent

| Solvents | Anti-solvent | | Additional | |
|---|---|---|---|---|
| (mL) | (mL) | XRPD | analysis | Comments |
| DMSO | H₂O (0.5mL) | Almost | // | — |
| (0.5 mL) | | amorphous form | | |
| Pyridine | Ethyl acetate | // | // | Hazy |
| (1.0 mL) | (4.0 mL) | | | suspension |
| Chloroform | MTBE (20 mL) | // | // | Hazy |
| (5 mL) | | | | suspension |
| 1-Methyl-2- | Ethyl acetate | Physical | // | — |
| pyrrolidinone | (2.0 mL) | mixture of Form | | |
| (0.5 mL) | | V and Form VI | | |
| Trifluoroethanol | Methoxy- | Almost | // | — |
| (1.5 mL) | nonafluorobutane | amorphous form | | |
| | (6.0 mL) | | | |
| DMF(1.0 mL) | H₂O (2.0 mL) | Form VIII | ¹H-NMR: | — |
| | | | 2 equiv. DMF | |
| Benzyl alcohol | Heptane (1.0 mL) | // | // | Hazy |
| (1.0 mL) | | | | suspension |
| THF (6.0 mL) | Heptane (12.0 mL) | // | // | Hazy |
| | | | | suspension |

Explanation
“//”: Not carried out.
“—”: No comments.

### Example 18: Approximate Solubility of Polymorphs in EtOH:DCM (v:v=1:4)

Four polymorphs (Form V, Form VI, Form VII and Form VIII) were weighed respectively and a suitable amount of solvent mixture EtOH:DCM (v:v=1:4) was added. The obtained samples were stirred at a rate of 400 rpm at 25° C. The observations are summarized and shown in Table 15. Form VI and Form VIII showed higher solubility than Form V and Form VII based on visual appearance.

TABLE 15
Approximate solubility of polymorphs in EtOH:DCM (v:v = 1:4)

| Concentration | | Form V | Form VI | Form VII | Form VIII |
|---|---|---|---|---|---|
| 20 | mg/mL | Suspension | Suspension | Suspension | Few solids |
| 10 | mg/mL | Suspension | Suspension | Suspension | Few solids |
| 5 | mg/mL | Suspension | Suspension | Suspension | // |

Explanation “//”: Not carried out.

### Example 19: Bulk Stability

Compound 5 Form VI and Form VIII were placed at 25° C./60% RH in an open container over 1 week. Samples after the stress were characterized by XRPD and HPLC and inspected for color change. The results are summarized in Table 16.

TABLE 16
Bulk stability

| Polymorph | Form VI | Form VIII |
|---|---|---|
| Initial purity | 93.1% | 95.6% |

| | | | |
|---|---|---|---|
| Purity | Color | Purity | Color |

Solid state, 25° C./60% RH, open container, 1 week

| | | | | |
|---|---|---|---|---|
| Bulk (HPLC) | 93.1% | No change of | 95.6% | No change of |
| | | color | | color |

| | | |
|---|---|---|
| Bulk (XRPD) | Form VI | Form V |

### Example 20: Solubility

11 mg (for 20 mg/mL target concentration) or 22 mg (for 50-60 mg/mL target concentration) of Compound 5 Form VI and Form VIII (equivalent to 10 mg or 20 mg of anhydrate free base) was weighed into a 4 mL glass vial, respectively. Up to 0.5 mL of potential eluent medium was added. The obtained suspensions/solutions were stirred at 25° C. at 400 rpm for 24 hours and then centrifuged at 25° C. at 14,000 rpm for 5 minutes. The residual solids were characterized by XRPD to determine the physical form. The supernatants (0.1 mL) were evaporated at about 22-25° C./30-60% RH. The residual solids were dissolved by ACN:DMSO (v:v=3:2) and analyzed by HPLC for solubility. The results are summarized in Table 17.

TABLE 17
Solubility
Method
Solubility at 25° C., equilibration for 24 hours, LOQ: 0.8 μg/mL;
Target conc.: 50-60 mg/mL
Polymorph

| | Form VI | | Form VIII | |
|---|---|---|---|---|
| Solubility | Solubility | XRPD of | Solubility | XRPD of |
| media | (mg/mL) | residual solids | (mg/mL) | residual solids |
| MeOH/DCM | 30 | Almost of | 30 | Form V |
| (v:v = 1:2) | | amorphous form | | |
| EtOH/DCM | 27 | Almost | 39 | Form V and |
| (v:v = 1:4) | | amorphous form | | Form VI |

Method
Solubility at 25° C., equilibration for 24 hours; Target conc.: 20 mg/mL

| | | | | |
|---|---|---|---|---|
| THF/IPA | 0.9 | Form VI | 1 | Form VI |
| (v:v = 1:2) | | | | |
| THF/Heptane | 0.1 | Form VI | 0.1 | Form VI |
| (v:v = 1:2) | | | | |
| THF/water | 4 | Form VI | 0.5 | Form V |
| (v:v = 1:2.5) | | | | |

### Example 21: Characterization of Polymorphs and Pseudo-Polymorphs

The instrumental methods are included in Example 23 and the results are summarized in Table 18.

TABLE 18
Characterization of polymorphs and pseudo-polymorphs

| | Form V | Form VI | Form VII | Form VIII |
|---|---|---|---|---|
| | Potential | Potential | Potential NMP | Potential |
| | hydrate | hydrate | solvate | hydrate |
| Crystallinity | Crystalline | Crystalline | Crystalline | Crystalline |
| (by XRPD) | (XRPD method 2) | (XRPD method 2) | (XRPD method 2) | (XRPD method 2) |
| Melting onset | Dehydration at | Dehydration at | // | Dehydration and |
| and Enthalpy | Tₒₙₛₑₜ 38.5° C.; | about 6° C.; | | evaporation of |
| (by DSC, ° C. or | Endothermic | Evaporation of | | solvent from |
| J/g) | onset at 220.0° C. | solvent from | | about 8° C.; |
| | | about 154° C.; | | Tₒₙₛₑₜ 231.4° C., |
| | | Tₒₙₛₑₜ 226.4° C., | | 105 J/g |
| | | 35 J/g | | |
| Weight loss | 5.1% at 140° C. | 9.7% at 215° C. | // | 4.7% at 200° C. |
| (by TGA) | | | | |
| Residual | 0.4% residual of | 0.6 equiv. (9.0% | 1.6 equiv. NMP; | 0.1 equiv. (1.6% |
| solvent | THF | by weight) | 2 unknown peaks | by weight) DMF |
| (by ¹H-NMR) | | DMSO | | |
| Residual ion | // | Li⁺: 0.17 equiv.; | // | Li⁺: 0.04 equiv.; |
| (by IC) | | Na₊: 0.02 equiv. | | Na⁺: 0.01 equiv. |
| | | Cl⁻: 0.08 equiv.; | | Cl⁻: 0.005 equiv.; |
| | | Br⁻: 0.22 equiv. | | Br⁻: 0.01 equiv. |

Explanation “//”: Not conducted.

### Example 22

### Preparation of Compound 5 Form VI

About 300 mg of Compound 5 Form V was dissolved by 7 mL of DMSO. (Almost clear solution) The obtained solution was filtered through a 0.45 μm syringe membrane filter. (Clear solution) The clear solution was fast evaporated at about 20-25° C. under a dry nitrogen flow for 5 days. Solids were dried under vacuum at 50° C. for 4 h. Compound 5 Form VI (290 mg) was obtained as an off-gray solid in 88% yield.

### Preparation of Compound 5 Form VIII

About 300 mg of Compound 5 Form V was dissolved by 8 mL of DMF. (Almost clear solution) The obtained solution was filtered through a 0.45 μm syringe membrane filter. (Clear solution) Water (16 mL) was added into the clear solution slowly. (Clear solution to suspension) The suspension was kept stirring at 25° C. for about 24 hours (suspension). Solids were collected by suction filtration and then dried at ambient condition (20-25° C., 30-60% RH) for 2 days. Compound 5 Form VIII (210 mg) was obtained as an off-gray solid in 70% yield.

### Example 23

X-ray Powder Diffractometer (XRPD)

| | |
|---|---|
| Instrument | Bruker D8 Advance |

XRPD method 1

| | |
|---|---|
| Detector | LYNXEYE_XE_T(1D mode) |
| Open angle | 2.94° |
| Scan mode | Continuous PSD fast |
| Radiation | Cu/K-Alpha1 (λ = 1.5418 Å) |
| X-ray generator power | 40 kV, 40 mA |
| Step size | 0.02° |
| Time per step | 0.3 second per step |
| Scan range | 2° to 40° |
| Primary beam path slits | Twin_Primary motorized slit 10.0 mm by sample length; |
| | SollerMount axial soller 2.5° |
| Secondary beam path slits | Detector OpticsMount soller slit 2.5°; Twin_Secondary motorized |
| | slit 5.2 mm |
| Sample rotation speed | 15 rpm |

XRPD method 2

| | |
|---|---|
| Detector | LYNXEYE_XE_T(1D mode) |
| Open angle | 2.94° |
| Scan mode | Continuous PSD fast |
| Radiation | Cu/K-Alpha1 (λ = 1.5418 Å) |
| X-ray generator power | 40 kV, 40 mA |
| Step size | 0.02° |
| Time per step | 0.12 second per step |
| Scan range | 3° to 40° |
| Primary beam path slits | Twin_Primary motorized slit 10.0 mm by sample length; |
| | SollerMount axial soller 2.5° |
| Secondary beam path slits | Detector OpticsMount soller slit 2.5°; Twin_Secondary motorized |
| | slit 5.2 mm |
| Sample rotation speed | 15 rpm |

Differential Scanning Calorimetric (DSC)

| | |
|---|---|
| Instrument | TA Discovery 2500 or Q2000 |
| Sample pan | Tzero pan and Tzero hermetic lid with a pin hole |
| Temperature range | 30 to 250° C. |
| Heating rate | 10° C./min |
| Nitrogen flow | 50 mL/min |
| Sample mass | About 1-2 mg |

Thermal Gravimetric Analysis (TGA)

| | |
|---|---|
| Instrument | Discovery 5500 |
| Sample pan | Aluminum, open |
| Nitrogen flow | Balance 10 mL/min; sample 25 mL/min |
| Start temperature | Ambient condition (below 35° C.) |
| Final temperature | 300° C. |
| Heating rate | 10° C./min |
| Sample mass | About 2-10 mg |

Nuclear Magnetic Resonance (NMR)

| | |
|---|---|
| Instrument | Bruker Avance-AV 400M |
| Frequency | 400 MHz |
| Probe | 5 mm PABBO BB-1H/D |
| Number of scan | 8 |
| Temperature | 297.6 K |
| Relaxation delay | 1 second |

Ion Chromatography (IC)

| | |
|---|---|
| Instrument | Metrohm 940 professional IC |
| Sample center | 889 IC |
| Detector | Conductivity detector |
| Eluent (anion) | 3.2 mmol/L Na₂CO₃ + 1.0 mmol/L NaHCO₃ |
| Eluent (cation) | 2.5 mmol/L MSA |
| Suppressor solutions | 0.5% H₂SO₄ |
| Column: | Anion A SUPP 5-150 or Cation Column C4-150 |
| Column temperature: | 30° C. |
| Flow rate: | 0.7 mL/min (anion) or 0.9 mL/min (cation) |
| Diluent: | ACN |
| Injection volume: | 20 μL |

High Performance Liquid Chromatograph (HPLC)

| Instrument | LC-20ADXR Shimadzu | | |
|---|---|---|---|
| HPLC method | Wavelength: 220 nm | | |
| | Column: Zorbax SB-C18, 4.6 mm*150 mm 3.5 μm-Micron | | |
| | Detector: SPD-M20A 230V Shimadzu | | |
| | Flow rate: 1.0 mL/min | | |
| | Mobile phase A: 0.05% TFA in water | | |
| | Mobile phase B: 0.05% TFA in ACN | | |
| | Diluent: MeOH | | |
| | Injection volume: 5 uL | | |
| | Gradient: | | |
| | Time (min) | Mobile Phase A (%) | Mobile Phase B (%) |
| | 0.01 | 90 | 10 |
| | 6.00 | 80 | 20 |
| | 20.00 | 65 | 35 |
| | 26.00 | 5 | 95 |
| | 28.00 | 5 | 95 |
| | 28.10 | 90 | 10 |
| | 33.00 | stop | |
| Instrument | Agilent 1260 Infinity II Binary Pump | | |
| HPLC method | Wave length: 220 | | |
| | Column: Waters Xbridge C18, 4.6 × 150 mm, 3.5 μm, PN: | | |
| | 186003034 | | |
| | Detector: DAD | | |
| | Column temperature: 20° C. | | |
| | Flow rate: 1.0 mL/min | | |
| | Mobile phase A: 0.05% TFA in water | | |
| | Mobile phase B: 0.05% TFA in ACN | | |
| | Injection volume: 10 μL | | |
| | Gradient: | | |
| | Time (min) | Mobile Phase A (%) | Mobile Phase B (%) |
| | 0.01 | 90 | 10 |
| | 6.00 | 80 | 20 |
| | 20.00 | 65 | 35 |
| | 25.00 | 5 | 95 |
| | 28.00 | 5 | 95 |
| | 28.01 | 90 | 10 |
| | 33.00 | Stop | |

### Example 24: Compound 1 Anti-Cancer Effects in Human Patients

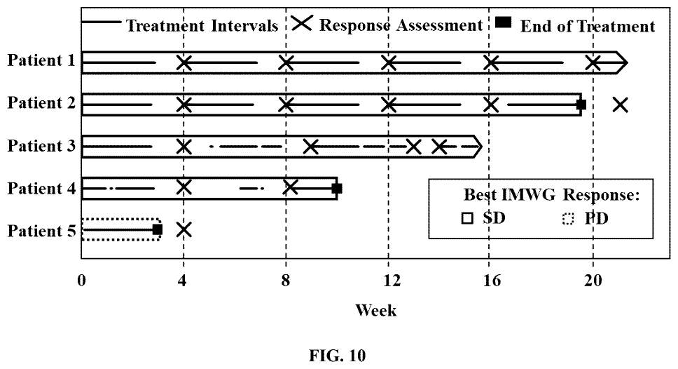

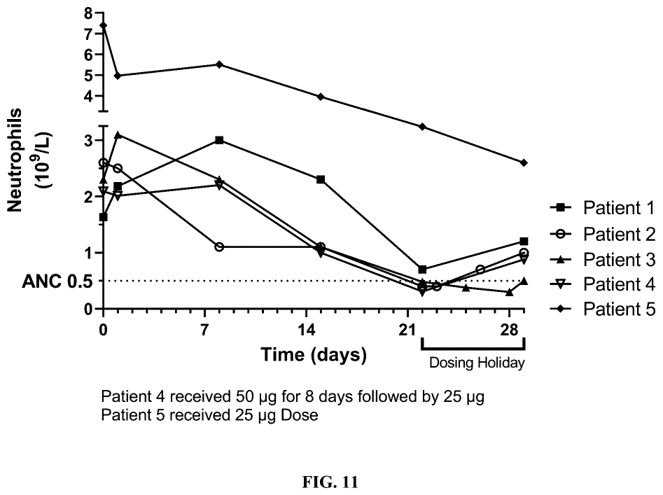

Five patients were orally administered either 50 μg or 25 μg of Compound 1 for the treatment intervals identified in FIG. 10. The first dose and patient's extramedullary disease state if present is provided in Table 19 below:

TABLE 19

| | First Dose | Extramedullary | % Change at |
|---|---|---|---|
| Patient | (μg) | Disease | Nadir in dFLC* |
| Patient 1 | 50 | No | ∇ 48.2 |
| Patient 2 | 50 | Multiple Plasmacytomas | ∇ 78.1 |
| Patient 3 | 50 | Lytic Bone Lesions | ∇ 41.0 |
| Patient 4 | 50 | No | Δ 56.3 |
| Patient 5 | 25 | Plasmacytomas and | N/A |
| | | Bone Lesions | |

*Patients 1-4 had unmeasurable M-protein (myeloma protein), resulting in % change in dFLC as the driver of response criteria based on IMWG for Multiple Myeloma (MM) response criteria

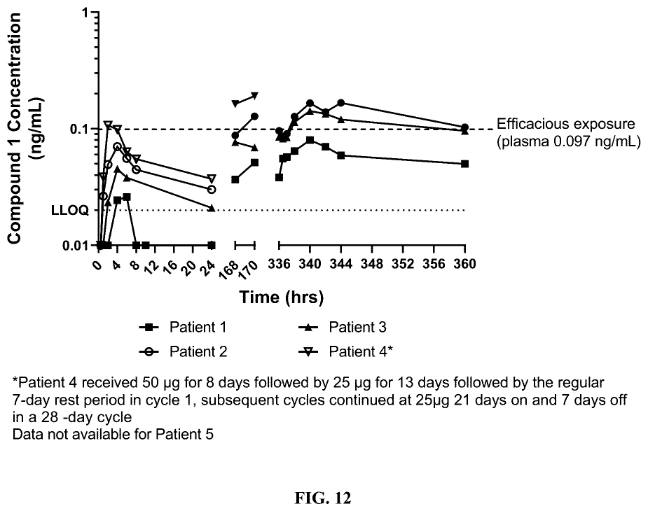

Periodic blood samples and response measurements were taken using standard clinical practice. The results of these measurements are summarized in FIG. 10 and FIG. 11. FIG. 12 shows concentration of Compound 1 established using this dosing regimen in Patient 1, Patient 2, Patient 3, and Patient 4.

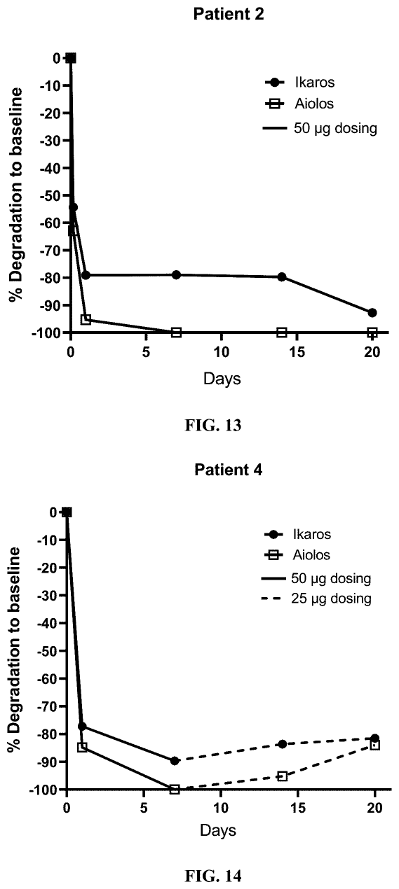

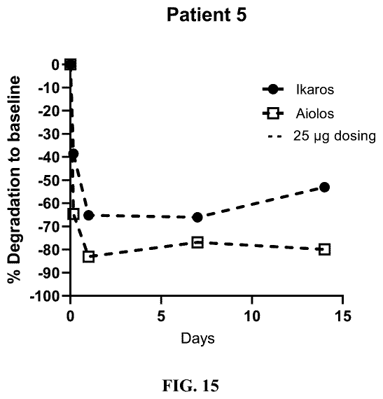

Ikaros (also known as IKZF1) and Aiolos (also known as IKZF3) degradation was evaluated using mass spectrometry analysis of total peripheral blood mononuclear cell (PBMC) population extracted from whole blood samples of Patient 2, Patient 4, and Patient 5 taken on the first day of the first cycle (cycle 1, day 1 or C1D1) prior to treatment, 4 hours post Compound 1 treatment, and on day 7, day 14, and day 21 of the treatment cycle. As shown in FIGS. 13-15, deep degradation was observed in these patients treated with 50 μg/day or 25 μg/day.

### Example 25 Reduction in dFLC after Single Agent Exposure to Compound 1

Using samples collecting in Example 24, the difference between involved free light chains and uninvolved free light changes, or dFLC, for Patients 1-3 were calculated using the following formula:

[Abnormal light chain_{baseline}−normal light chain_{baseline}]−[Abnormal light chain_{nadir}−normal light chain_{nadir}]/[Abnormal light chain_{baseline}−normal light chain_{baseline}]*100 dFLC equation:

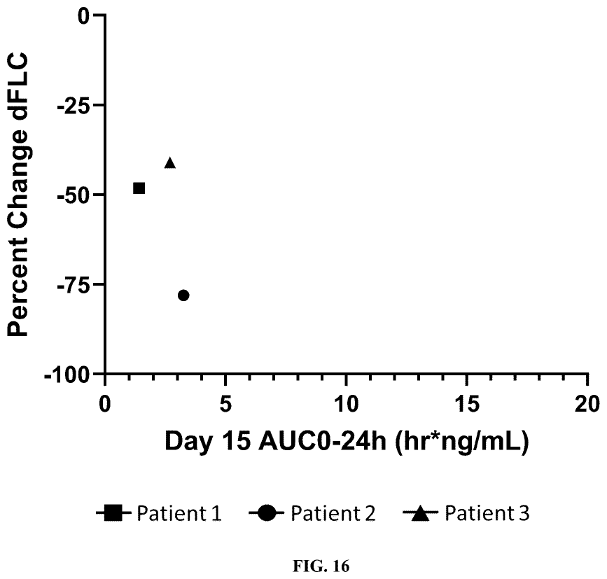

The resulting data is shown in FIG. 16 and Table 19. Decreased dFLC levels in treated versus untreated patients indicates that the cellular proliferation of multiple myeloma cells has been decreased.

### Example 26 NCI-H929 Xenograft Study

A xenograft study was conducted in female NOD SCID mice bearing NCI-H929 MM tumors. Female SCID mice were inoculated subcutaneously in the right flank with 10×10⁶ tumor cells in 0.2 mL of PBS supplemented with matrigel (PBS:matrigel=1:1). Tumor volume was measured twice weekly in two dimensions using calipers, and volume was calculated using the formula: (w2×1)/2=mm³, assuming 1 mg is equivalent to 1 mm³ of tumor volume. For the efficacy portion of this study, once the tumors reached an average volume of 419 mm³ mice randomized into 4 groups with 4 mice each. For the pharmacodynamic and pharmacokinetic portion of the study, once tumors reached an average tumor volume of 620 mm³ mice were randomized into groups of 4 with 12 mice per group.

For the efficacy portion of the study Compound 1 (100 μg/kg), CC-92480 (1000 μg/kg), pomalidomide (3000 μg/kg) and the vehicle control were administered to mice bearing NCI-H929 tumors on day 0 and dosed PO daily for 18 days. Compound 1, CC-92480, and pomalidomide, were formulated in PEG400 (30% v/v)+70% v/v HPMC (1% w/v) in citrate buffer (pH 5), which was also used as the vehicle control. Body weight and MTV was measured on a 2× weekly schedule. The study end point for the vehicle control group was day 18 when the average tumor volume of the group exceeded 2000 mm³. Mice in the CC-92480 group were removed from the study on Day 21. Mice in the Compound 1 group were monitored for tumor regrowth after the dosing period of 18 days. The study endpoint for this group was Day 63. Data are expressed as MTV±SEM.

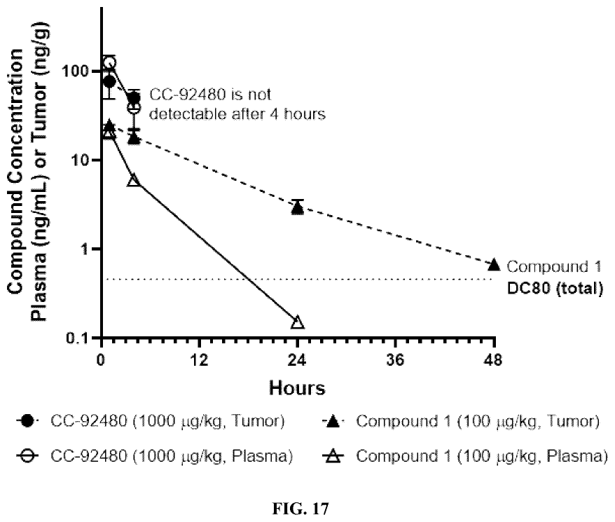

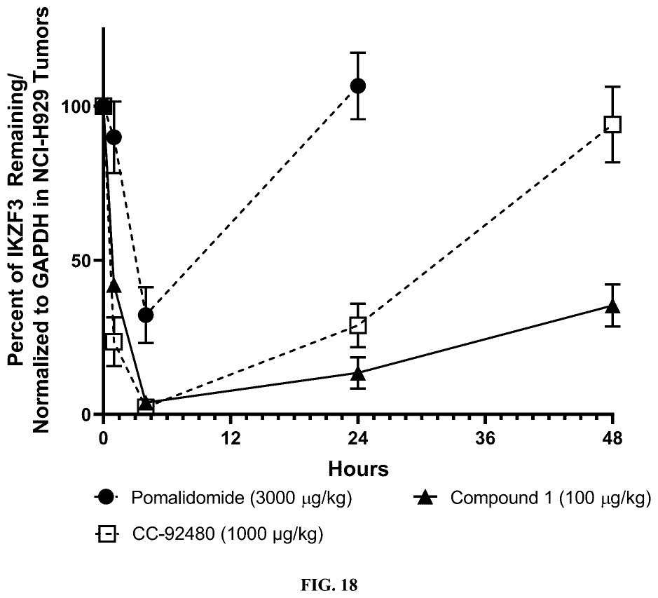

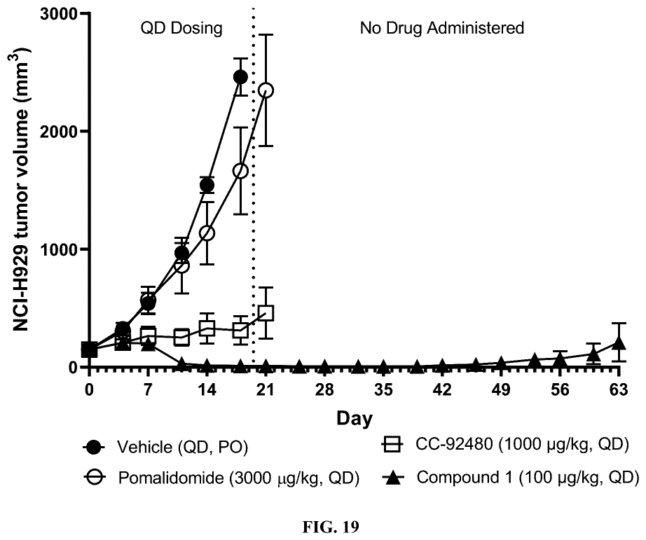

For the pharmacodynamic and pharmacokinetic portion of this study 3 mice were sacrificed per time point with tumors and plasma harvested, with mice in the vehicle control group being sacrificed 24 hours post single dose. Mice in the study Compound 1 (100 μg/kg), CC-92480 (1000 μg/kg), pomalidomide (3000 μg/kg) were sampled at 1, 4, 24, and 48 hours post a single dose with 3 mice sampled per time point. Tumors were then mechanically homogenized, and protein extracted using RIPA buffer (Sigma Aldrich). Protein concentration was quantified using a Pierce™ BCA Protein Assay Kit, samples were reduced, and equal protein amounts were then loaded onto a western blot gel for analysis. Tumors were analyzed for IKZF3 (CST, 15103) expression. The intensity of individual bands was measured for data analysis using Image Studio NIR software. Protein expression was quantitated in relation to the reference protein, GAPDH, to control for total protein concentration. The data was then normalized to the amount of target in the Compound 1 treated samples in comparison to the vehicle control samples. Data is represented as percent of target present in the vehicle control and normalized for total protein. For pharmacokinetic analysis of plasma and tumor samples, tumor samples were mechanically homogenized using homogenizing solution (MeOH/15 mM PBS (1:2, v:v)) and for plasma protein precipitation performed. Samples were quenched and compared to a standard curve by LC-MS/MS analysis. Error bars represent ±SEM values. The resulting data is shown in FIG. 17, FIG. 18, and FIG. 19.

### Example 27 OCI-LY10 Human DLBCL Intracranial Xenograft Study

A xenograft study was conducted to evaluate the in vivo efficacy of Compound 1, ibrutinib, and rituximab in female CB17 SCID mice bearing OCI-LY10 human DLBCL intracranial xenografts. Agents were tested in the OCI-LY10 human DLBCL intracranial model. Compound 1 and ibrutinib were administered to CB17 SCID mice bearing OCI-LY10 intracranial xenografts from day 1 via p.o. once per day. Compound 1 was dosed at 0.01 mg/kg as single agent or combination with ibrutinib or rituximab. Ibrutinib was dosed at 12.5 or 25 mg/kg. Rituximab was administered from day 1 via i.v. once per week. Rituximab was dosed at 10 and 30 mg/kg. Individual mouse was euthanized once its body weight loss was over 20%.

### 1. Experimental Design

### Groups and Treatments for the Study

TABLE 20
Study Design for the Study

| | | | Dose | Dosing | |
|---|---|---|---|---|---|
| Group | N^{a} | Treatment^{b} | (mg/kg) | Route | Schedule^{c} |
| 1 | 8 | Vehicle 1 + | — | p.o. + i.v. | QD + QW |
| | | Vehicle 2 | | | |
| 2 | 8 | Compound 1 | 0.01 | p.o. | QD |
| 3 | 8 | ibrutinib | 12.5 | p.o. | QD |
| 4 | 8 | ibrutinib | 25 | p.o. | QD |
| 5 | 8 | rituximab | 10 | p.o. | QW |
| 6 | 8 | rituximab | 30 | p.o. | QW |
| 7 | 8 | Compound 1 + | 0.01 + 12.5 | p.o. + p.o. | QD + QD |
| | | ibrutinib | | | |
| 8 | 8 | Compound 1 + | 0.01 + 25 | p.o. + p.o. | QD + QD |
| | | ibrutinib | | | |
| 9 | 8 | Compound 1 + | 0.01 + 10 | p.o. + i.v. | QD + QW |
| | | rituximab | | | |
| 10 | 8 | Compound 1 + | 0.01 + 30 | p.o. + i.v. | QD + QW |
| | | rituximab | | | |

Note:
^{a}N: animal number; Dosing volume: adjust dosing volume based on body weight 5 μL/g for Compound 1 and ibrutinib, 10 μL/g for rituximab.
^{b}The formulation for Vehicle and Compound 1 was 30% PEG400 + 70% (1% HPMC in 100 mM citrate buffer (pH = 5.0)). The formulation for ibrutinib was 0.5% MC + 0.4% EL + 0.1% SDS in ddH₂O. The formulation for rituximab was saline.
^{c}Individual mice were euthanized once its body weight loss was over 20%.

### 2. Materials

### Animals and Housing Condition:

### Animals:

Species: Mus Musculus
Strain: CB17 SCID
Age: 6-8 weeks
Sex: Female
Body weight: 18-22 g
Number of animals: 80 mice plus spare
Animal supplier: Beijing Vital River Laboratory Animal Technology Co., Ltd.

### Housing Condition:

The mice were kept in individual ventilation cages at constant temperature and humidity with 4 animals in each cage.
Temperature: 20˜26° C.
Humidity 40-70%.
Cages: Made of polycarbonate. The size is 375 mm×215 mm×180 mm. The bedding material was corn cob, which was changed twice per week.
Diet: Animals had free access to irradiation sterilized dry granule food during the entire study period.
Water: Animals had free access to sterile drinking water.
Cage identification: The identification labels for each cage contained the following information: number of animals, sex, strain, date received, treatment, study number, group number and the starting date of the treatment.
Animal identification: Animals were marked by ear coding.

### 3. Experimental Methods and Procedures

### Cell Culture

The OCI-LY10 DLBCL cells were maintained in vitro in IMDM medium supplemented with 20% fetal bovine serum, 1% penicillin streptomycin and 0.05 mM 2-mercaptoethanol at 37° C. in an atmosphere of 5% CO₂ in air. The tumor cells were routinely subcultured twice weekly. The cells growing in an exponential growth phase were harvested and counted for tumor inoculation.

The details of reagents are shown as Table 21.

TABLE 21
Vendor and Cat No. of Cell Line and Reagents

| Cell Line/Reagent | Vendor |
|---|---|
| OCI-LY10 | National Cancer Institute |
| IMDM | Gibco |
| Penicillin Streptomycin | Gibco |
| 2-mercaptoethanol | Invitrogen |
| FBS | Corning |
| DPBS | Corning |
| Matrigel Matrix | Corning |

### Tumor Inoculation

99 CB17 SCID mice were inoculated by intracranial injection with OCI-LY10 cells (1×10⁶) for tumor development.

Tumor inoculation: Intraperitoneally injected avertin at dosage of 240 mg/kg to anaesthetize the CB17 SCID mice. Fixed the head of the anaesthetized mice to the brain solid positioner, sterilized the head skin with 70% alcohol. About 1 cm sagittal cut was made above the parietal and occipital bone with sterilized scalpel, the skull was revealed and cleaned with cotton bud, in this case the anterior fontanel could be available. Punched a hole about 2 mm right of the anterior fontanel and 1 mm ahead the coronal on the skull with a 25 G needle. Cell suspensions were mixed and certain amount was sucked into the 5 μL injector slowly to avoid bubble formation. The cells were swapped around the needle with alcohol to avoid extracranial tumor genesis. The needle was stuck vertically into the formed hold and 5 μL of cell suspension containing 1×10⁶ OCI-LY10 cells was slowly injected for over 1 minute. The injector was then held for another 1 minute and then pulled back to decrease the drainage of the cells suspension. The skin was closed with tweezers and then sewed. Meloxicam was then injected 30 min after the surgery at dosage of 5 mg/kg to relieve the pain. The animals were kept under monitoring until they were awake.

Animals were grouped for treatment on day 7 after tumor inoculation. The testing articles were administrated to the mice according to the regimen as shown in the experimental design of Table 20.

### Reagents Used in the Study

TABLE 22
Vendor and Cat No. of Reagents Used for Formulation Preparation

| Reagent | Vendor |
|---|---|
| PEG400 | Sigma |
| MC | Aladdin |
| Tween-80 | Sigma |
| Citric acid | Sigma |
| Sodium citrate | Sigma |
| tribasic dihydrate | |
| HPMC | Sigma |
| DMSO | Sigma |
| PBS | CORNING |
| Kolliphor EL | Sigma |
| SDS | Sinopharm Chemical Reagent Co. |
| | LTD |
| Saline | Zhejiang Dubang Pharmaceutical |
| | Co. LTD |

### Vehicle Preparation

Preparation of 1% HPMC in 100 mM Citrate Buffer (pH=5.0)

1) Solution 1: 7.37 g of sodium citrate tribasic dihydrate was dissolved into 250 mL of ddH₂O, stirred to obtain sodium citrate solution at 100 mM.

2) Solution 2: 3.85 g of citric acid (anhydrous) was dissolved into 200 mL of ddH₂O, stirred to obtain citric acid solution at 100 mM.

3) Citrate buffer: Slowly added Solution 1 to Solution 2 until the pH of the buffer reached 5.0.

4) Added 1 g of HPMC to 100 mL of 100 mM citrate buffer (pH 5.0), stirred to mix well.

### Preparation of 0.5% MC+0.4% Kolliphor EL+0.1% SDS Solution

1) Accurately weighed 1 g of MC into 250 mL volumetric flask. Added 180 mL of ddH₂O, stirred overnight to mix well.

2) Added 0.8 mL Kolliphor EL to the solution, stirred for a while to mix well.

3) Accurately weighed 0.2 g SDS into the solution, stirred to mix well.

4) Added ddH₂O to 200 mL, then stirred to mix well.

### Testing Article Formulation Preparation

TABLE 23
Preparation of Compound 1 High Concentration Formulation

| | | | | | 1% HPMC in | |
|---|---|---|---|---|---|---|
| | | | | PEG400 | 100 mM citrate | |
| | | | | to be | buffer(pH = 5.0) | |
| | | Conc. of | Compound | added | to be added | Dosing |
| | Dose | solution | amount | (mL) | (mL) | solution |
| Compound^{a} | (mg/kg) | (mg/mL) | (mg) | (30%) | (70%) | observations |
| Compound 1 | 1 | 0.2 | 1.02 | 1.470 | 3.431 | Solution |

Note:
^{a}Solution was prepared once every 3 or 4 days.

TABLE 24
Preparation of Compound 1 Low Concentration Formulation

| | | | | Volume of high | | |
|---|---|---|---|---|---|---|
| | | | Conc. of high | concentration | Vehicleb | |
| | | Conc. of | concentration | formulation | to be | Dosing |
| | Dose | solution | formulation | to be added | added | solution |
| Compound^{a} | (mg/kg) | (mg/mL) | (mg/mL) | (mL) | (mL) | observations |
| Compound 1 | 0.01 | 0.002 | 0.2 | 0.250 | 24.750 | Solution |

Note:
^{a}Dosing solutions were prepared once every 3 or 4 days.
bVehicle was 30% PEG400 + 70% (1% HPMC in 100 mM citrate (pH = 5.0)).

TABLE 25
Preparation of Rituximab Dosing Solution

| | | | Conc. of | Volume of stock | | |
|---|---|---|---|---|---|---|
| | | Conc. of | stock | solution | Saline to | Dosing |
| | Dose | solution | solution | to be added | be added | solution |
| Compound^{a} | (mg/kg) | (mg/mL) | (mg/mL) | (mL) | (mL) | observations |
| Rituximab | 10 | 1 | 10 | 0.600 | 5.400 | Solution |
| Rituximab | 30 | 3 | 10 | 1.800 | 4.200 | Solution |

Note:
^{a}Dosing solutions were prepared once every week.

TABLE 26
Preparation of Ibrutinib Dosing Solution

| | | | Com- | Vehicleb | |
|---|---|---|---|---|---|
| | | Conc. of | pound | to be | Dosing |
| Com- | Dose | solution | amount | added | solution |
| pound^{a} | (mg/kg) | (mg/mL) | (mg) | (mL) | observations |
| Ibrutinib | 12.5 | 2.5 | 23.62 | 9.397 | Suspension |
| Ibrutinib | 25 | 5 | 45.34 | 9.019 | Suspension |

Note:
^{a}Dosing solutions were prepared once every 3 or 4 days.
bVehicle was 0.5% MC + 0.4% EL + 0.1% SDS in ddH₂O.

### Observations

At the time of routine monitoring, the animals were daily checked for any effects of tumor growth and treatments on normal behavior such as mobility, food and water consumption (by looking only), body weight gain/loss (body weights were measured twice weekly), eye/hair matting and any other abnormal effect as stated in the protocol. Death and observed clinical signs were recorded on the basis of the numbers of animals within each subset.

### Results

### Body Weight Change

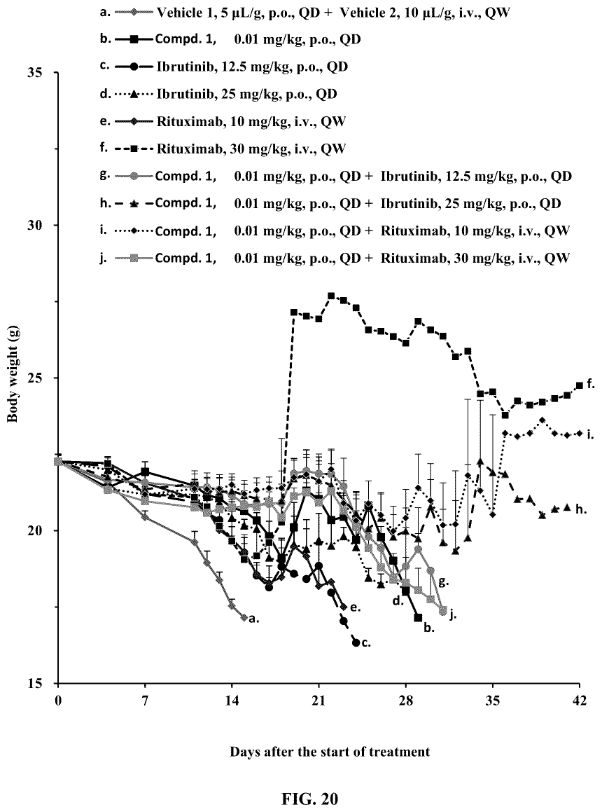

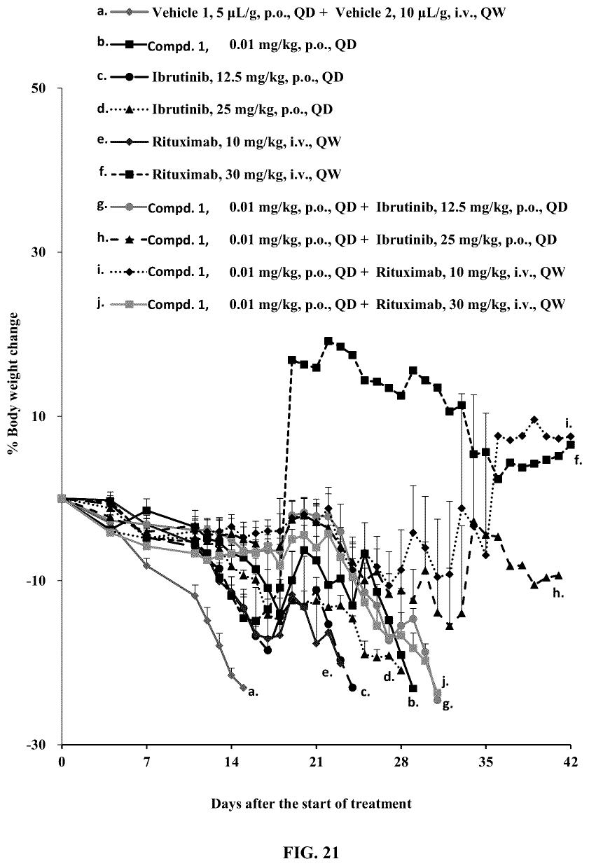

Animal body weight was monitored regularly as an indirect measurement of toxicity. Body weight and body weight changes after administration of the test articles are shown in FIG. 20 and FIG. 21.

### Survival Days and Survival Curve

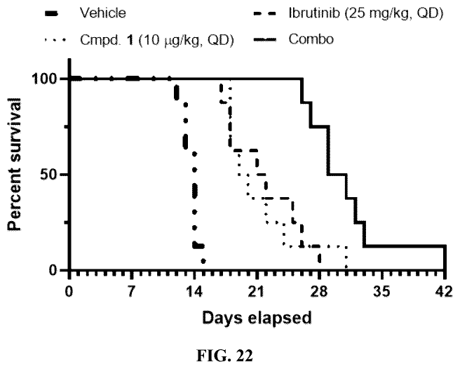

The survival days of the mice on the day of euthanasia or death are shown in Table 27. The Kaplan-Meier survival curves of animals in each groups are shown in FIG. 22.

TABLE 27
The Survival Days at the End Point

| | Animal | Euthanized/ | Animal | Euthanized/ |
|---|---|---|---|---|
| Treatment | ID | Died date | ID | Died date |
| Vehicle 1, p.o., QD + | 1-1 | PG-D12 | 2-1 | PG-D13 |
| Vehicle 2, i.v., QW | 1-2 | PG-D14 | 2-2 | PG-D13 |
| | 1-3 | PG-D14 | 2-3 | PG-D14 |
| | 1-4 | PG-D15 | 2-4 | PG-D14 |
| Compound 1, | 3-1 | PG-D18 | 4-1 | PG-D17 |
| 0.01 mg/kg, p.o., QD | 3-2 | PG-D20 | 4-2 | PG-D18 |
| | 3-3 | PG-D19 | 4-3 | PG-D22 |
| | 3-4 | PG-D29 | 4-4 | PG-D24 |
| Ibrutinib, | 5-1 | PG-D24 | 6-1 | PG-D17 |
| 12.5 mg/kg, p.o., QD | 5-2 | PG-D17 | 6-2 | PG-D17 |
| | 5-3 | PG-D19 | 6-3 | PG-D18 |
| | 5-4 | PG-D16 | 6-4 | PG-D17 |
| Ibrutinib, | 7-1 | PG-D22 | 8-1 | PG-D26 |
| 12.5 mg/kg, p.o., QD | 7-2 | PG-D21 | 8-2 | PG-D18 |
| | 7-3 | PG-D18 | 8-3 | PG-D28 |
| | 7-4 | PG-D17 | 8-4 | PG-D25 |
| Rituximab, | 9-1 | PG-D17 | 10-1 | PG-D18 |
| 10 mg/kg, i.v., QW | 9-2 | PG-D17 | 10-2 | PG-D16 |
| | 9-3 | PG-D23 | 10-3 | PG-D17 |
| | 9-4 | PG-D21 | 10-4 | PG-D16 |
| Rituximab, | 11-1 | PG-D15 | 12-1 | PG-D17 |
| 30 mg/kg, i.v., QW | 11-2 | PG-D16 | 12-2 | PG-D18 |
| | 11-3 | PG-D15 | 12-3 | PG-D16 |
| | 11-4 | PG-D18 | 12-4 | PG-D43 |
| Compound 1, 0.01 | 13-1 | PG-D28 | 14-1 | PG-D30 |
| mg/kg, p.o., QD + | 13-2 | PG-D27 | 14-2 | PG-D27 |
| Ibrutinib, 12.5 mg/kg, | 13-3 | PG-D18 | 14-3 | PG-D31 |
| p.o., QD | 13-4 | PG-D28 | 14-4 | PG-D27 |
| Compound 1, 0.01 | 15-1 | PG-D26 | 16-1 | PG-D31 |
| mg/kg, p.o., QD + | 15-2 | PG-D29 | 16-2 | PG-D32 |
| Ibrutinib, 25 mg/kg, | 15-3 | PG-D27 | 16-3 | PG-D42 |
| p.o., QD | 15-4 | PG-D29 | 16-4 | PG-D33 |
| Compound 1, 0.01 | 17-1 | PG-D24 | 18-1 | PG-D28 |
| mg/kg, p.o., QD + | 17-2 | PG-D43 | 18-2 | PG-D31 |
| Rituximab, 10 mg/kg, | 17-3 | PG-D32 | 18-3 | PG-D35 |
| i.v., QW | 17-4 | PG-D27 | 18-4 | PG-D32 |
| Compound 1, 0.01 | 19-1 | PG-D30 | 20-1 | PG-D18 |
| mg/kg, p.o., QD + | 19-2 | PG-D28 | 20-2 | PG-D26 |
| Rituximab, 30 mg/kg, | 19-3 | PG-D29 | 20-3 | PG-D31 |
| i.v., QW | 19-4 | PG-D27 | 20-4 | PG-D27 |

Note:
a. Mice #12-4 and #17-2 were euthanized on PG-D43.

### Survival Statistical Analysis

Median survival and statistical results are shown in Table 28.

TABLE 28
Median Survival and Statistical Results

| | | 95% Confidence | | Survival | Log |
|---|---|---|---|---|---|
| | Median | Interval | | time | Rank |
| | Survival | Lower | Upper | extension | p^{b} |
| Treatment | (Days)^{a} | Bound | Bound | (%) | Value |
| Vehicle 1, p.o., QD + | 14.00 ± 0.23 | 13.542 | 14.458 | — | — |
| Vehicle 2, i.v., QW | | | | | |
| Compound 1, 0.01 mg/kg, | 19.00 ± 1.41 | 16.228 | 21.772 | 35.7 | 0.001 |
| p.o., QD | | | | | |
| Ibrutinib, 12.5 mg/kg, p.o., QD | 17.00 ± 0.34 | 16.329 | 17.671 | 21.4 | <0.001 |
| Ibrutinib, 25 mg/kg, p.o., QD | 21.00 ± 2.83 | 15.456 | 26.544 | 50.0 | 0.001 |
| Rituximab, 10 mg/kg, i.v., QW | 17.00 ± 0.46 | 16.105 | 17.895 | 21.4 | 0.001 |
| Rituximab, 30 mg/kg, i.v., QW | 16.00 ± 0.94 | 14.152 | 17.848 | 14.3 | 0.085 |
| Compound 1, 0.01 mg/kg, p.o., | 27.00 ± 2.83 | 21.456 | 32.544 | 92.9 | <0.001 |
| QD + ibrutinib, 12.5 mg/kg, | | | | | |
| p.o., QD | | | | | |
| Compound 1, 0.01 mg/kg, p.o., | 31.00 ± 1.89 | 25.304 | 32.696 | 121.4 | <0.001 |
| QD + ibrutinib, 25 mg/kg, | | | | | |
| p.o., QD | | | | | |
| Compound 1, 0.01 mg/kg, p.o., | 27.00 ± 0.94 | 25.152 | 28.848 | 92.9 | <0.001 |
| QD + rituximab, 10 mg/kg, | | | | | |
| i.v., QW | | | | | |
| Compound 1, 0.01 mg/kg, p.o., | 21.00 ± 2.44 | 16.219 | 25.781 | 50.0 | <0.001 |
| QD + rituximab, 30 mg/kg, | | | | | |
| i.v., QW | | | | | |

Note:
^{a}Medium Survival Time ± SEM.
^{b}p value was calculated based on different groups of survival time using vehicle group as the control.

### CONCLUSION

In the study, the therapeutic efficacy of test compounds in the treatment of female CB17 SCID mice bearing OCI-LY10 intracranial xenografts was evaluated.

Animal body weight was monitored regularly as an indirect measurement of toxicity. Body weight changes after administration of compounds are shown in FIG. 20 and FIG. 21. All mice showed slight body weight loss at the first week. The mice were added extra nutrient when their body weight loss was over 10%, and the mice were euthanized once their body weight loss was over 20%.

The survival days of the mice are shown in Table 27. The Kaplan-Meier survival curves of animals in each groups are shown in FIG. 22. Median survival and statistical results are shown in Table 28. The extension of survival time were 35.7% (p=0.001) for Compound 1 0.01 mg/kg, 21.4% (p<0.001) for ibrutinib 12.5 mg/kg, 50% (p=0.001) for ibrutinib 25 mg/kg, 21.4% (p=0.001) for rituximab 10 mg/kg, 14.3% (p=0.085) for rituximab 30 mg/kg, 92.9% (p<0.001) for Compound 10.01 mg/kg+ibrutinib 12.5 mg/kg, 121.4% (p<0.001) for Compound 10.01 mg/kg+ibrutinib 25 mg/kg, 92.9% (p<0.001) for Compound 1 0.01 mg/kg+rituximab 10 mg/kg and 50% (p<0.001) for Compound 1 0.01 mg/kg+rituximab 30 mg/kg, respectively.

All publications and patent applications cited in this specification are herein incorporated by reference as if each individual publication or patent application were specifically and individually indicated to be incorporated by reference.

Although the foregoing invention has been described in some detail by way of illustration and example for the purposes of clarity of understanding, it will be readily apparent to one of ordinary skill in the art in light of the teaching of this invention that certain changes and modification may be made thereto without departing from the spirit or scope of the invention as defined in the claims.

We claim:

1. A crystalline compound of structure: 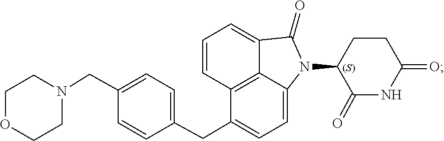
which is characterized by an X-ray powder diffraction (XRPD) pattern comprising at least three 2theta values selected from the group consisting of 4.8, 13.5, 16.0, 16.4, 17.8, 17.9, 19.2, 19.6, 20.9, 21.5, 21.8, 22.9, 23.2, 24.8, 26.5, and 31.3+/−0.4° 2theta.

2. The crystalline compound of claim 1, which is characterized by an X-ray powder diffraction (XRPD) pattern comprising at least four 2theta values selected from the group consisting of 4.8, 13.5, 16.0, 16.4, 17.8, 17.9, 19.2, 19.6, 20.9, 21.5, 21.8, 22.9, 23.2, 24.8, 26.5, and 31.3+/−0.4° 2theta.

3. The crystalline compound of claim 1, which is characterized by an X-ray powder diffraction (XRPD) pattern comprising at least five 2theta values selected from the group consisting of 4.8, 13.5, 16.0, 16.4, 17.8, 17.9, 19.2, 19.6, 20.9, 21.5, 21.8, 22.9, 23.2, 24.8, 26.5, and 31.3+/−0.4° 2theta.

4. The crystalline compound of claim 1, which is characterized by an X-ray powder diffraction (XRPD) pattern comprising at least six 2theta values selected from the group consisting of 4.8, 13.5, 16.0, 16.4, 17.8, 17.9, 19.2, 19.6, 20.9, 21.5, 21.8, 22.9, 23.2, 24.8, 26.5, and 31.3+/−0.4° 2theta.

5. The crystalline compound of claim 1, which is characterized by an X-ray powder diffraction (XRPD) pattern comprising at least seven 2theta values selected from the group consisting of 4.8, 13.5, 16.0, 16.4, 17.8, 17.9, 19.2, 19.6, 20.9, 21.5, 21.8, 22.9, 23.2, 24.8, 26.5, and 31.3+/−0.4° 2theta.

6. The crystalline compound of claim 1, which is characterized by an X-ray powder diffraction (XRPD) pattern comprising at least eight 2theta values selected from the group consisting of 4.8, 13.5, 16.0, 16.4, 17.8, 17.9, 19.2, 19.6, 20.9, 21.5, 21.8, 22.9, 23.2, 24.8, 26.5, and 31.3+/−0.4° 2theta.

7. The crystalline compound of claim 1, which is characterized by an X-ray powder diffraction (XRPD) pattern comprising at least three 2theta values selected from the group consisting of 4.8, 13.5, 16.0, 16.4, 17.8, 17.9, 19.2, 19.6, 20.9, 21.5, 21.8, 22.9, 23.2, 24.8, 26.5, and 31.3+/−0.3° 2theta.

8. The crystalline compound of claim 1, which is characterized by an X-ray powder diffraction (XRPD) pattern comprising at least three 2theta values selected from the group consisting of 4.8, 13.5, 16.0, 16.4, 17.8, 17.9, 19.2, 19.6, 20.9, 21.5, 21.8, 22.9, 23.2, 24.8, 26.5, and 31.3+/−0.2° 2theta.

9. The crystalline compound of claim 1, wherein the XRPD includes a peak at 4.8+/−0.4° 2theta.

10. The crystalline compound of claim 1, wherein the XRPD includes a peak at 16.4+/−0.2° 2theta.

11. The crystalline compound of claim 1, wherein the XRPD includes a peak at 19.2+/−0.2° 2theta.

12. The crystalline compound of claim 1, wherein the XRPD includes a peak at 22.9+/−0.2° 2theta.

13. The crystalline compound of claim 1, wherein the XRPD includes a peak at 13.5+/−0.2° 2theta.

14. The crystalline compound of claim 1, wherein the XRPD includes a peak at 19.6+/−0.2° 2theta.

15. A pharmaceutical composition comprising a crystalline compound of claim 1 and one or more pharmaceutically acceptable excipients.

16. A pharmaceutical composition prepared from a crystalline compound of claim 1 and one or more pharmaceutically acceptable excipients.

17. A method of treating a cancer that is mediated by IKZF1 and/or IKZF3 comprising administering an effective amount of a crystalline compound of claim 1 to a human in need thereof.

18. The method of claim 17, wherein the cancer is multiple myeloma.

19. The method of claim 17, wherein the cancer is non-Hodgkin's lymphoma.

20. The method of claim 17, wherein the cancer is a B-cell lymphoma.
